(19) United States
(12) Patent Application Publication
(10) Pub. No.: US 2025/0250345 A1
(43) Pub. Date: Aug. 7, 2025

(54) SIL-6R AND CTGF BINDING PROTEINS AND METHODS OF USE THEREOF

(71) Applicant: EBBIL, LTD., Surrey (GB)

(72) Inventors: Mohammed AL-MAADHEED, Doha (QA); Vidya MOHAMED-ALI, London (GB)

(73) Assignee: EBBIL, LTD., Surrey (GB)

(21) Appl. No.: 18/856,161

(22) PCT Filed: Oct. 13, 2023
(86) PCT No.: PCT/EP2023/078467

§ 371 Date: Oct. 11, 2024

Related U.S. Application Data
(60) Provisional application No. 63/416,135, filed on Oct. 14, 2022.

(51) Int. Cl. C07K 16/28; A61K 9/00; A61K 39/00; A61P 9/10; A61P 29/00; C07K 16/22

(52) U.S. Cl. CPC C07K 16/2866; A61K 9/0019; A61P 9/10; A61P 29/00; C07K 16/22; C07K 16/2881; A61K 2039/505; A61K 2039/54; C07K 2317/31; C07K 2317/565; C07K 2317/569; C07K 2317/76; C07K 2317/77; C07K 2317/92

(57) ABSTRACT

Provided herein are soluble interleukin-6 receptor (sIL-6R) and CTGF binding proteins, multispecific binding proteins thereof, conjugates thereof, pharmaceutical compositions thereof, and methods of use thereof. The specific binding proteins can be single heavy chain antibodies (VHH), and the multispecific binding protein can include two variable heavy chain (VHH) immunoglobulin domains targeting sIL-6R and CTGF. The conjugates can include a binding protein or multispecific binding protein, conjugated with an anti-transferrin receptor antibody. The pharmaceutical compositions can include one or more binding proteins and/or multispecific binding proteins. The methods of use include the administration of binding proteins and/or multispecific binding protein for the treatment of neurological diseases.

### PRIORITY

This application claims the benefit of U.S. provisional application Ser. No. 63/416,135, filed on Oct. 14, 2022, which is incorporated herein by reference in its entirety.

### FIELD OF THE DISCLOSURE

The present disclosure relates generally to binding proteins, and more specifically to Soluble interleurkin-6 receptor (sIL-6R) and connective tissue growth factor (CTGF) binding proteins, multispecific binding proteins thereof, conjugates thereof, and to methods of use thereof.

### BACKGROUND INFORMATION

Interleukin-6 (IL-6) is a key cytokine involved in neurogenesis, influencing both neurons and glial cells, and in the response of mature neurons and glial cells in normal conditions and following a wide range of injury models. IL-6 behaves in a neurotrophin-like fashion and belongs to the cytokine family known as neuropoietins.

CTGF is involved in the repair processes of the central nervous system. The expression of CTGF protein in both sporadic and familial amyotrophic lateral sclerosis (ALS) patients is increased, and especially in reactive astrocytes. Increased expression is also observed in the cytoplasm of motor neurons of ALS patients with long duration of the disease. A role for CTGF in the complex reactive process that is associated with the progression of ALS spinal cord damage is indicated. Altered CTGF expression in neurons might represent an additional mechanism involved in motor neuron dysfunction and changes in glial-neuronal communication in the course of the neurodegenerative process.

Provided herein are dromedary camel VHH fragment antibodies targeting pro-inflammatory/pro-fibrotic cytokines or their signaling, such as sIL-6R and CTGF

### SUMMARY OF THE DISCLOSURE

Provided herein are sIL-6R and CTGF binding proteins, multispecific binding proteins thereof, conjugates thereof, pharmaceutical compositions thereof, and methods of use thereof.

An aspect provides multispecific binding protein comprising a) a first binding protein that specifically binds to human soluble interleurkin-6 receptor (sIL-6R); and b) a second binding protein that is operatively linked to the first binding protein and that specifically binds to human connective tissue growth factor (CTGF). The first binding protein and the second binding protein can comprise a VHH, humanized VHH, VH, single domain antibody, or variable new antigen receptor (VNAR). The multispecific binding protein can comprise an antibody or an antigen-binding fragment thereof, a chimeric antibody, a humanized antibody, or a camelid antibody. The multispecific binding protein can further comprise an anti-transferrin receptor binding protein. The multispecific binding protein can be mutated by polycationic resurfacing.

An embodiment provides a binding protein that selectively and/or specifically binds to human sIL-6R.

The binding protein can comprise complementarity-determining regions (CDRs) as set forth in SEQ ID NOs: 2-4, SEQ ID NOS: 6-8, SEQ ID NOs: 10-12, SEQ ID NOS: 14-16, SEQ ID NOs: 18-20, SEQ ID NOs: 22-24, SEQ ID NOs: 26-28, SEQ ID NOs: 30-32, SEQ ID NOs: 34-36, SEQ ID NOs: 38-40, SEQ ID NOs: 42-44, SEQ ID NOs: 46-48, SEQ ID NOs: 50-52, SEQ ID NOs: 54-56, SEQ ID NOs: 58-60, SEQ ID NOs: 62-64, SEQ ID NOs: 66-68, or SEQ ID NO: 138-194 or a binding protein having CDRs with 80% or greater identity to the CDRs of the binding protein comprising SEQ ID NOs: 2-4, SEQ ID NOs: 6-8, SEQ ID NOs: 10-12, SEQ ID NOs: 14-16, SEQ ID NOs: 18-20, SEQ ID NOs: 22-24, SEQ ID NOs: 26-28, SEQ ID NOs: 30-32, SEQ ID NOs: 34-36, SEQ ID NOs: 38-40, SEQ ID NOs: 42-44, SEQ ID NOs: 46-48, SEQ ID NOs: 50-52, SEQ ID NOs: 54-56, SEQ ID NOs: 58-60, SEQ ID NOs: 62-64, SEQ ID NOs: 66-68, or SEQ ID NO: 138-194 and the antigen binding specificity thereof. The antibody can have a better binding affinity to soluble IL-6R as compared to a binding affinity to membrane bound IL-6R.

An embodiment provides a binding protein that selectively and/or specifically binds to human CTGF.

The binding protein can comprise CDRs as set forth in SEQ ID NOs: 70-72, SEQ ID NOs: 74-76, SEQ ID NOs: 78-80, SEQ ID NOS: 82-84, SEQ ID NOs: 86-88, SEQ ID NOs: 90-92, SEQ ID NOs: 233-286, or SEQ ID NOs: 291-293 or a binding protein having CDRs with 80% or greater identity to the CDRs of the binding protein comprising SEQ ID NOs: 70-72, SEQ ID NOs: 74-76, SEQ ID NOs: 78-80, SEQ ID NOs: 82-84, SEQ ID NOs: 86-88, SEQ ID NOs: 90-92, SEQ ID NOs: 233-286, or SEQ ID NOs: 291-293 and the antigen binding specificity thereof.

Any of the binding proteins can comprise a VHH, humanized VHH, VH, single domain antibody, or variable new antigen receptor (VNAR). Any of the binding proteins can comprise an antibody or an antigen-binding fragment thereof, a chimeric antibody, a humanized antibody, or a camelid antibody.

The binding protein can comprise the amino acid sequence of SEQ ID NO:1, SEQ ID NO: 5, SEQ ID NO:9, SEQ ID NO: 13, SEQ ID NO: 17, SEQ ID NO:21, SEQ ID NO:25, SEQ ID NO: 29, SEQ ID NO: 33, SEQ ID NO:37, SEQ ID NO:41, SEQ ID NO:45, SEQ ID NO:49, SEQ ID NO: 53, SEQ ID NO: 57, SEQ ID NO:61, SEQ ID NO: 65, SEQ ID NO:69, SEQ ID NO:73, SEQ ID NO: 77, SEQ ID NO:81, SEQ ID NO:85, SEQ ID NO:89, SEQ ID NOS: 119-137 or SEQ ID NOs: 214-232. The binding protein can be conjugated to an anti-transferrin receptor antibody. The antibody can be mutated by polycationic resurfacing.

An embodiment provides a multispecific binding protein that selectively and/or specifically binds to human sIL-6R and to human CTGF.

The multispecific binding protein can comprise CDRs as set forth in: (i) SEQ ID NOs: 2-4 and SEQ ID NOs: 70-72, SEQ ID NOs: 6-8 and SEQ ID NOs: 70-72, SEQ ID NOs: 10-12 and SEQ ID NOs: 70-72, SEQ ID NOs: 14-16 and SEQ ID NOs: 70-72, SEQ ID NOs: 18-20 and SEQ ID NOs: 70-72, SEQ ID NOs: 22-24 and SEQ ID NOs: 70-72, SEQ ID NOs: 26-28 and SEQ ID NOs: 70-72, SEQ ID NOs: 30-32 and SEQ ID NOs: 70-72, SEQ ID NOs: 34-36 and SEQ ID NOs: 70-72, SEQ ID NOs: 38-40 and SEQ ID NOs: 70-72, SEQ ID NOs: 42-44 and SEQ ID NOs: 70-72, SEQ ID NOs: 46-48 and SEQ ID NOs: 70-72, SEQ ID NOs: 50-52 and SEQ ID NOs: 70-72, SEQ ID NOs: 54-56 and SEQ ID NOs: 70-72, SEQ ID NOs: 58-60 and SEQ ID NOs: 70-72, SEQ ID NOs: 62-64 and SEQ ID NOs: 70-72 or SEQ ID NOs: 66-68 and SEQ ID NOs: 70-72 (furthermore any of the sIL-6R CDR sets shown in SEQ ID NOs: 138-194 can similarly be combined with SEQ ID NO:70-72 and any of the CTGF CDR sets as set forth in SEQ ID NOs: 233-286 can be used in place of SEQ ID NOs: 70-72);
(ii) SEQ ID NOs: 2-4 and SEQ ID NOs: 74-76, SEQ ID NOs: 6-8 and SEQ ID NOs: 74-76, SEQ ID NOs: 10-12 and SEQ ID NOs: 74-76, SEQ ID NOs: 14-16 and SEQ ID NOs: 74-76, SEQ ID NOs: 18-20 and SEQ ID NOs: 74-76, SEQ ID NOs: 22-24 and SEQ ID NOs: 74-76, SEQ ID NOs: 26-28 and SEQ ID NOs: 74-76, SEQ ID NOs: 30-32 and SEQ ID NOs: 74-76, SEQ ID NOs: 34-36 and SEQ ID NOs: 74-76, SEQ ID NOs: 38-40 and SEQ ID NOs: 74-76, SEQ ID NOs: 42-44 and SEQ ID NOs: 74-76, SEQ ID NOs: 46-48 and SEQ ID NOs: 74-76, SEQ ID NOs: 50-52 and SEQ ID NOs: 74-76, SEQ ID NOs: 54-56 and SEQ ID NOs: 74-76, SEQ ID NOs: 58-60 and SEQ ID NOs: 74-76, SEQ ID NOs: 62-64 and SEQ ID NOs: 74-76, or SEQ ID NOs: 66-68 and SEQ ID NOs: 74-76 (furthermore any of the sIL-6R CDR sets shown in SEQ ID NOs: 138-194 can similarly be combined with SEQ ID NO: 74-76 and any of the CTGF CDR sets as set forth in SEQ ID NOs: 233-286 can be used in place of SEQ ID NOs: 74-76);
(iii) SEQ ID NOs: 2-4 and SEQ ID NOs: 78-80, SEQ ID NOs: 6-8 and SEQ ID NOs: 78-80, SEQ ID NOs: 10-12 and SEQ ID NOs: 78-80, SEQ ID NOs: 14-16 and SEQ ID NOs: 78-80, SEQ ID NOs: 18-20 and SEQ ID NOs: 78-80, SEQ ID NOs: 22-24 and SEQ ID NOs: 78-80, SEQ ID NOs: 26-28 and SEQ ID NOs: 78-80, SEQ ID NOs: 30-32 and SEQ ID NOs: 78-80, SEQ ID NOs: 34-36 and SEQ ID NOs: 78-80, SEQ ID NOs: 38-40 and SEQ ID NOs: 78-80, SEQ ID NOs: 42-44 and SEQ ID NOs: 78-80, SEQ ID NOs: 46-48 and SEQ ID NOs: 78-80, SEQ ID NOs: 50-52 and SEQ ID NOs: 78-80, SEQ ID NOs: 54-56 and SEQ ID NOs: 78-80, SEQ ID NOs: 58-60 and SEQ ID NOs: 78-80, SEQ ID NOs: 62-64 and SEQ ID NOs: 78-80, or SEQ ID NOs: 66-68 and SEQ ID NOs: 78-80 (furthermore any of the sIL-6R CDR sets shown in SEQ ID NOs: 138-194 can similarly be combined with SEQ ID NO: 78-80 and any of the CTGF CDR sets as set forth in SEQ ID NOs: 233-286 can be used in place of SEQ ID NOs: 78-80);
(iv) SEQ ID NOs: 2-4 and SEQ ID NOs: 82-84, SEQ ID NOs: 6-8 and SEQ ID NOs: 82-84, SEQ ID NOs: 10-12 and SEQ ID NOs: 82-84, SEQ ID NOs: 14-16 and SEQ ID NOs: 82-84, SEQ ID NOs: 18-20 and SEQ ID NOs: 82-84, SEQ ID NOs: 22-24 and SEQ ID NOs: 82-84, SEQ ID NOs: 26-28 and SEQ ID NOs: 82-84, SEQ ID NOs: 30-32 and SEQ ID NOs: 82-84, SEQ ID NOs: 34-36 and SEQ ID NOs: 82-84, SEQ ID NOs: 38-40 and SEQ ID NOs: 82-84, SEQ ID NOs: 42-44 and SEQ ID NOs: 82-84, SEQ ID NOs: 46-48 and SEQ ID NOs: 82-84, SEQ ID NOs: 50-52 and SEQ ID NOs: 82-84, SEQ ID NOs: 54-56 and SEQ ID NOs: 82-84, SEQ ID NOs: 58-60 and SEQ ID NOs: 82-84, SEQ ID NOs: 62-64 and SEQ ID NOs: 82-84 or SEQ ID NOs: 66-68 and SEQ ID NOs: 82-84 (furthermore any of the sIL-6R CDR sets shown in SEQ ID NOs: 138-194 can similarly be combined with SEQ ID NO: 82-84 and any of the CTGF CDR sets as set forth in SEQ ID NOs: 233-286 can be used in place of SEQ ID NOs: 82-84);
(v) SEQ ID NOs: 2-4 and SEQ ID NOs: 86-88, SEQ ID NOs: 6-8 and SEQ ID NOs: 86-88, SEQ ID NOs: 10-12 and SEQ ID NOs: 86-88, SEQ ID NOs: 14-16 and SEQ ID NOs: 86-88, SEQ ID NOs: 18-20 and SEQ ID NOs: 86-88, SEQ ID NOs: 22-24 and SEQ ID NOs: 86-88, SEQ ID NOs: 26-28 and SEQ ID NOs: 86-88, SEQ ID NOs: 30-32 and SEQ ID NOs: 86-88, SEQ ID NOs: 34-36 and SEQ ID NOs: 86-88, SEQ ID NOs: 38-40 and SEQ ID NOs: 86-88, SEQ ID NOs: 42-44 and SEQ ID NOs: 86-88, SEQ ID NOs: 46-48 and SEQ ID NOs: 86-88, SEQ ID NOs: 50-52 and SEQ ID NOs: 86-88, SEQ ID NOs: 54-56 and SEQ ID NOs: 86-88, SEQ ID NOs: 58-60 and SEQ ID NOs: 86-88, SEQ ID NOs: 62-64 and SEQ ID NOs: 86-88 or SEQ ID NOs: 66-68 and SEQ ID NOs: 86-88 (furthermore any of the sIL-6R CDR sets shown in SEQ ID NOs: 138-194 can similarly be combined with SEQ ID NO: 86-88 and any of the CTGF CDR sets as set forth in SEQ ID NOs: 233-286 can be used in place of SEQ ID NOs: 86-88); or
(vi) SEQ ID NOs: 2-4 and SEQ ID NOs: 90-92, SEQ ID NOs: 6-8 and SEQ ID NOs: 90-92, SEQ ID NOs: 10-12 and SEQ ID NOs: 90-92, SEQ ID NOs: 14-16 and SEQ ID NOs: 90-92, SEQ ID NOs: 18-20 and SEQ ID NOs: 90-92, SEQ ID NOs: 22-24 and SEQ ID NOs: 90-92, SEQ ID NOs: 26-28 and SEQ ID NOs: 90-92, SEQ ID NOs: 30-32 and SEQ ID NOs: 90-92, SEQ ID NOs: 34-36 and SEQ ID NOs: 90-92, SEQ ID NOs: 38-40 and SEQ ID NOs: 90-92, SEQ ID NOs: 42-44 and SEQ ID NOs: 90-92, SEQ ID NOs: 46-48 and SEQ ID NOs: 90-92, SEQ ID NOs: 50-52 and SEQ ID NOs: 90-92, SEQ ID NOs: 54-56 and SEQ ID NOs: 90-92, SEQ ID NOs: 58-60 and SEQ ID NOs: 90-92, SEQ ID NOs: 62-64 and SEQ ID NOs: 90-92 or SEQ ID NOs: 66-68 and SEQ ID NOs: 90-92 (furthermore any of the sIL-6R CDR sets shown in SEQ ID NOs: 138-194 can similarly be combined with SEQ ID NO: 90-92 and any of the CTGF CDR sets as set forth in SEQ ID NOs: 233-286 can be used in place of SEQ ID NOs: 90-92).

The multispecific binding protein can comprise the amino acid sequence of: (i) SEQ ID NO: 1 and SEQ ID NO: 69, SEQ ID NO:5 and SEQ ID NO:69, SEQ ID NO:9 and SEQ ID NO:69, SEQ ID NO: 13 and SEQ ID NO:69, SEQ ID NO: 17 and SEQ ID NO:69, SEQ ID NO:21 and SEQ ID NO: 69, SEQ ID NO:25 and SEQ ID NO:69, SEQ ID NO:29 and SEQ ID NO:69, SEQ ID NO:33 and SEQ ID NO: 69, SEQ ID NO:37 and SEQ ID NO:69, SEQ ID NO:41 and SEQ ID NO:69, SEQ ID NO: 45 and SEQ ID NO:69, SEQ ID NO:49 and SEQ ID NO:69, SEQ ID NO:53 and SEQ ID NO: 69, SEQ ID NO:57 and SEQ ID NO:69, SEQ ID NO:61 and SEQ ID NO:69, or SEQ ID NO:65 and SEQ ID NO:69 (furthermore any of the sIL-6R VHHs shown in SEQ ID NOs: 119-137 can similarly be combined with SEQ ID NO:69 and any of SEQ ID NO:214-232 can be used in place of SEQ ID NO:69);
(ii) SEQ ID NO: 1 and SEQ ID NO:73, SEQ ID NO:5 and SEQ ID NO:73, SEQ ID NO:9 and SEQ ID NO: 73, SEQ ID NO: 13 and SEQ ID NO:73, SEQ ID NO: 17 and SEQ ID NO:73, SEQ ID NO:21 and SEQ ID NO:73, SEQ ID NO:25 and SEQ ID NO:73, SEQ ID NO:29 and SEQ ID NO:73, SEQ ID NO: 33 and SEQ ID NO:73, SEQ ID NO:37 and SEQ ID NO:73, SEQ ID NO:41 and SEQ ID NO: 73, SEQ ID NO:45 and SEQ ID NO:73, SEQ ID NO:49 and SEQ ID NO:73, SEQ ID NO:53 and SEQ ID NO:73, SEQ ID NO:57 and SEQ ID NO:73, SEQ ID NO:61 and SEQ ID NO:73, or SEQ ID NO:65 and SEQ ID NO:73 (furthermore any of the sIL-6R VHHs shown in SEQ ID NOs: 119-137 can similarly be combined with SEQ ID NO:73 and any of SEQ ID NO:214-232 can be used in place of SEQ ID NO:73);
(iii) SEQ ID NO:1 and SEQ ID NO:77, SEQ ID NO:5 and SEQ ID NO:77, SEQ ID NO:9 and SEQ ID NO: 77, SEQ ID NO: 13 and SEQ ID NO:77, SEQ ID NO: 17 and SEQ ID NO:77, SEQ ID NO:21 and SEQ ID NO:77, SEQ ID NO:25 and SEQ ID NO:77, SEQ ID NO:29 and SEQ ID NO:77, SEQ ID NO: 33 and SEQ ID NO:77, SEQ ID NO:37 and SEQ ID NO:77, SEQ ID NO:41 and SEQ ID NO: 77, SEQ ID NO:45 and SEQ ID NO:77, SEQ ID NO:49 and SEQ ID NO:77, SEQ ID NO:53 and SEQ ID NO:77, SEQ ID NO:57 and SEQ ID NO:77, SEQ ID NO:61 and SEQ ID NO:77, or SEQ ID NO:65 and SEQ ID NO:77 (furthermore any of the sIL-6R VHHs shown in SEQ ID NOs: 119-137 can similarly be combined with SEQ ID NO:77 and any of SEQ ID NO:214-232 can be used in place of SEQ ID NO:77);
(iv) SEQ ID NO:1 and SEQ ID NO:81, SEQ ID NO:5 and SEQ ID NO:81, SEQ ID NO:9 and SEQ ID NO: 81, SEQ ID NO: 13 and SEQ ID NO:81, SEQ ID NO: 17 and SEQ ID NO:81, SEQ ID NO:21 and SEQ ID NO:81, SEQ ID NO:25 and SEQ ID NO:81, SEQ ID NO:29 and SEQ ID NO:81, SEQ ID NO: 33 and SEQ ID NO:81, SEQ ID NO:37 and SEQ ID NO:81, SEQ ID NO:41 and SEQ ID NO: 81, SEQ ID NO:45 and SEQ ID NO:81, SEQ ID NO:49 and SEQ ID NO:81, SEQ ID NO:53 and SEQ ID NO:81, SEQ ID NO:57 and SEQ ID NO:81, SEQ ID NO:61 and SEQ ID NO:81, or SEQ ID NO:65 and SEQ ID NO:81 (furthermore any of the sIL-6R VHHs shown in SEQ ID NOs: 119-137 can similarly be combined with SEQ ID NO:81 and any of SEQ ID NO:214-232 can be used in place of SEQ ID NO:81);
(v) SEQ ID NO: 1 and SEQ ID NO:85, SEQ ID NO:5 and SEQ ID NO:85, SEQ ID NO:9 and SEQ ID NO: 85, SEQ ID NO: 13 and SEQ ID NO:85, SEQ ID NO: 17 and SEQ ID NO:85, SEQ ID NO:21 and SEQ ID NO: 85, SEQ ID NO:25 and SEQ ID NO:85, SEQ ID NO:29 and SEQ ID NO:85, SEQ ID NO: 33 and SEQ ID NO:85, SEQ ID NO:37 and SEQ ID NO:85, SEQ ID NO:41 and SEQ ID NO: 85, SEQ ID NO:45 and SEQ ID NO:85, SEQ ID NO:49 and SEQ ID NO:85, SEQ ID NO:53 and SEQ ID NO:85, SEQ ID NO:57 and SEQ ID NO:85, SEQ ID NO:61 and SEQ ID NO:85, or SEQ ID NO:65 and SEQ ID NO:85 (furthermore any of the sIL-6R VHHs shown in SEQ ID NOs: 119-137 can similarly be combined with SEQ ID NO:85 and any of SEQ ID NO:214-232 can be used in place of SEQ ID NO:85);
(vi) SEQ ID NO: 1 and SEQ ID NO: 89, SEQ ID NO:5 and SEQ ID NO:89, SEQ ID NO:9 and SEQ ID NO: 89, SEQ ID NO: 13 and SEQ ID NO:89, SEQ ID NO: 17 and SEQ ID NO:89, SEQ ID NO:21 and SEQ ID NO: 89, SEQ ID NO:25 and SEQ ID NO:89, SEQ ID NO:29 and SEQ ID NO:89, SEQ ID NO: 33 and SEQ ID NO:89, SEQ ID NO:37 and SEQ ID NO:89, SEQ ID NO:41 and SEQ ID NO: 89, SEQ ID NO:45 and SEQ ID NO:89, SEQ ID NO:49 and SEQ ID NO:89, SEQ ID NO:53 and SEQ ID NO:89, SEQ ID NO:57 and SEQ ID NO:89, SEQ ID NO:61 and SEQ ID NO:89, or SEQ ID NO:65 and SEQ ID NO:89 (furthermore any of the sIL-6R VHHs shown in SEQ ID NOs: 119-137 can similarly be combined with SEQ ID NO: 89 and any of SEQ ID NO:214-232 can be used in place of SEQ ID NO:89).

The multispecific binding protein can have a better binding affinity to soluble IL-6R as compared to a binding affinity to membrane bound IL-6R. The multispecific binding protein can be conjugated to an anti-transferrin receptor antibody. The multispecific binding protein can be mutated by polycationic resurfacing.

An embodiment provides a fusion protein comprising a first binding protein that selectively and/or specifically binds to human sIL-6R linked to a second binding protein that selectively and/or specifically binds to human CTGF.

The first binding protein and the second binding protein can be VHH antibodies. The fusion protein can further comprise an anti-transferrin receptor binding protein. The VHH antibodies can comprise CDRs as set forth in:
(i) SEQ ID NOs: 2-4 and SEQ ID NOs: 70-72, SEQ ID NOs: 6-8 and SEQ ID NOs: 70-72, SEQ ID NOs: 10-12 and SEQ ID NOs: 70-72, SEQ ID NOs: 14-16 and SEQ ID NOs: 70-72, SEQ ID NOs: 18-20 and SEQ ID NOs: 70-72, SEQ ID NOs: 22-24 and SEQ ID NOs: 70-72, SEQ ID NOs: 26-28 and SEQ ID NOs: 70-72, SEQ ID NOs: 30-32 and SEQ ID NOs: 70-72, SEQ ID NOs: 34-36 and SEQ ID NOs: 70-72, SEQ ID NOs: 38-40 and SEQ ID NOs: 70-72, SEQ ID NOs: 42-44 and SEQ ID NOs: 70-72, SEQ ID NOs: 46-48 and SEQ ID NOs: 70-72, SEQ ID NOs: 50-52 and SEQ ID NOs: 70-72, SEQ ID NOs: 54-56 and SEQ ID NOs: 70-72, SEQ ID NOs: 58-60 and SEQ ID NOs: 70-72, SEQ ID NOs: 62-64 and SEQ ID NOs: 70-72 or SEQ ID NOs: 66-68 and SEQ ID NOs: 70-72 (furthermore any of the sIL-6R CDR sets shown in SEQ ID NOs: 138-194 can similarly be combined with SEQ ID NO: 70-72 and any of the CTGF CDR sets as set forth in SEQ ID NOs: 233-286 can be used in place of SEQ ID NOs: 70-72);
(ii) SEQ ID NOs: 2-4 and SEQ ID NOs: 74-76, SEQ ID NOs: 6-8 and SEQ ID NOs: 74-76, SEQ ID NOs: 10-12 and SEQ ID NOs: 74-76, SEQ ID NOs: 14-16 and SEQ ID NOs: 74-76, SEQ ID NOs: 18-20 and SEQ ID NOs: 74-76, SEQ ID NOs: 22-24 and SEQ ID NOs: 74-76, SEQ ID NOs: 26-28 and SEQ ID NOs: 74-76, SEQ ID NOs: 30-32 and SEQ ID NOs: 74-76, SEQ ID NOs: 34-36 and SEQ ID NOs: 74-76, SEQ ID NOs: 38-40 and SEQ ID NOs: 74-76, SEQ ID NOs: 42-44 and SEQ ID NOs: 74-76, SEQ ID NOs: 46-48 and SEQ ID NOs: 74-76, SEQ ID NOs: 50-52 and SEQ ID NOs: 74-76, SEQ ID NOs: 54-56 and SEQ ID NOs: 74-76, SEQ ID NOs: 58-60 and SEQ ID NOs: 74-76, SEQ ID NOs: 62-64 and SEQ ID NOs: 74-76 or SEQ ID NOs: 66-68 and SEQ ID NOs: 74-76 (furthermore any of the sIL-6R CDR sets shown in SEQ ID NOs: 138-194 can similarly be combined with SEQ ID NO: 70-72 and any of the CTGF CDR sets as set forth in SEQ ID NOs: 233-286 can be used in place of SEQ ID NOs: 74-76);
(iii) SEQ ID NOs: 2-4 and SEQ ID NOs: 78-80, SEQ ID NOs: 6-8 and SEQ ID NOs: 78-80, SEQ ID NOs: 10-12 and SEQ ID NOs: 78-80, SEQ ID NOs: 14-16 and SEQ ID NOs: 78-80, SEQ ID NOs: 18-20 and SEQ ID NOs: 78-80, SEQ ID NOs: 22-24 and SEQ ID NOs: 78-80, SEQ ID NOs: 26-28 and SEQ ID NOs: 78-80, SEQ ID NOs: 30-32 and SEQ ID NOs: 78-80, SEQ ID NOs: 34-36 and SEQ ID NOs: 78-80, SEQ ID NOs: 38-40 and SEQ ID NOs: 78-80, SEQ ID NOs: 42-44 and SEQ ID NOs: 78-80, SEQ ID NOs: 46-48 and SEQ ID NOs: 78-80, SEQ ID NOs: 50-52 and SEQ ID NOs: 78-80, SEQ ID NOs: 54-56 and SEQ ID NOs: 78-80, SEQ ID NOs: 58-60 and SEQ ID NOs: 78-80, SEQ ID NOs: 62-64 and SEQ ID NOs: 78-80 or SEQ ID NOs: 66-68 and SEQ ID NOs: 78-80 (furthermore any of the sIL-6R CDR sets shown in SEQ ID NOs: 138-194 can similarly be combined with SEQ ID NO: 70-72 and any of the CTGF CDR sets as set forth in SEQ ID NOs: 233-286 can be used in place of SEQ ID NOs: 78-80);
(iv) SEQ ID NOs: 2-4 and SEQ ID NOs: 82-84, SEQ ID NOs: 6-8 and SEQ ID NOs: 82-84, SEQ ID NOs: 10-12 and SEQ ID NOs: 82-84, SEQ ID NOs: 14-16 and SEQ ID NOs: 82-84, SEQ ID NOs: 18-20 and SEQ ID NOs: 82-84, SEQ ID NOs: 22-24 and SEQ ID NOs: 82-84, SEQ ID NOs: 26-28 and SEQ ID NOs: 82-84, SEQ ID NOs: 30-32 and SEQ ID NOs: 82-84, SEQ ID NOs: 34-36 and SEQ ID NOs: 82-84, SEQ ID NOs: 38-40 and SEQ ID NOs: 82-84, SEQ ID NOs: 42-44 and SEQ ID NOs: 82-84, SEQ ID NOs: 46-48 and SEQ ID NOs: 82-84, SEQ ID NOs: 50-52 and SEQ ID NOs: 82-84, SEQ ID NOs: 54-56 and SEQ ID NOs: 82-84, SEQ ID NOs: 58-60 and SEQ ID NOs: 82-84, SEQ ID NOs: 62-64 and SEQ ID NOs: 82-84 or SEQ ID NOs: 66-68 and SEQ ID NOs: 82-84 (furthermore any of the sIL-6R CDR sets shown in SEQ ID NOs: 138-194 can similarly be combined with SEQ ID NO: 70-72 and any of the CTGF CDR sets as set forth in SEQ ID NOs: 233-286 can be used in place of SEQ ID NOs: 82-84);
(v) SEQ ID NOs: 2-4 and SEQ ID NOs: 86-88, SEQ ID NOs: 6-8 and SEQ ID NOs: 86-88, SEQ ID NOs: 10-12 and SEQ ID NOs: 86-88, SEQ ID NOs: 14-16 and SEQ ID NOs: 86-88, SEQ ID NOs: 18-20 and SEQ ID NOs: 86-88, SEQ ID NOs: 22-24 and SEQ ID NOs: 86-88, SEQ ID NOs: 26-28 and SEQ ID NOs: 86-88, SEQ ID NOs: 30-32 and SEQ ID NOs: 86-88, SEQ ID NOs: 34-36 and SEQ ID NOs: 86-88, SEQ ID NOs: 38-40 and SEQ ID NOs: 86-88, SEQ ID NOs: 42-44 and SEQ ID NOs: 86-88, SEQ ID NOs: 46-48 and SEQ ID NOs: 86-88, SEQ ID NOs: 50-52 and SEQ ID NOs: 86-88, SEQ ID NOs: 54-56 and SEQ ID NOs: 86-88, SEQ ID NOs: 58-60 and SEQ ID NOs: 86-88, SEQ ID NOs: 62-64 and SEQ ID NOs: 86-88 or SEQ ID NOs: 66-68 and SEQ ID NOs: 86-88 (furthermore any of the sIL-6R CDR sets shown in SEQ ID NOs: 138-194 can similarly be combined with SEQ ID NO: 70-72 and any of the CTGF CDR sets as set forth in SEQ ID NOs: 233-286 can be used in place of SEQ ID NOs: 86-88); or
(vi) SEQ ID NOs: 2-4 and SEQ ID NOs: 90-92, SEQ ID NOs: 6-8 and SEQ ID NOs: 90-92, SEQ ID NOs: 10-12 and SEQ ID NOs: 90-92, SEQ ID NOs: 14-16 and SEQ ID NOs: 90-92, SEQ ID NOs: 18-20 and SEQ ID NOs: 90-92, SEQ ID NOs: 22-24 and SEQ ID NOs: 90-92, SEQ ID NOs: 26-28 and SEQ ID NOs: 90-92, SEQ ID NOs: 30-32 and SEQ ID NOs: 90-92, SEQ ID NOs: 34-36 and SEQ ID NOs: 90-92, SEQ ID NOs: 38-40 and SEQ ID NOs: 90-92, SEQ ID NOs: 42-44 and SEQ ID NOs: 90-92, SEQ ID NOs: 46-48 and SEQ ID NOs: 90-92, SEQ ID NOs: 50-52 and SEQ ID NOs: 90-92, SEQ ID NOs: 54-56 and SEQ ID NOs: 90-92, SEQ ID NOs: 58-60 and SEQ ID NOs: 90-92, SEQ ID NOs: 62-64 and SEQ ID NOs: 90-92 or SEQ ID NOs: 66-68 and SEQ ID NOs: 90-92 (furthermore any of the sIL-6R CDR sets shown in SEQ ID NOs: 138-194 can similarly be combined with SEQ ID NO: 70-72 and any of the CTGF CDR sets as set forth in SEQ ID NOs: 233-286 can be used in place of SEQ ID NOs: 90-92);
The fusion protein can comprise the amino acid sequence of:
(i) SEQ ID NO: 1 and SEQ ID NO:69, SEQ ID NO:5 and SEQ ID NO:69, SEQ ID NO:9 and SEQ ID NO:69, SEQ ID NO: 13 and SEQ ID NO:69, SEQ ID NO: 17 and SEQ ID NO:69, SEQ ID NO: 21 and SEQ ID NO: 69, SEQ ID NO:25 and SEQ ID NO:69, SEQ ID NO:29 and SEQ ID NO:69, SEQ ID NO:33 and SEQ ID NO:69, SEQ ID NO:37 and SEQ ID NO:69, SEQ ID NO:41 and SEQ ID NO: 69, SEQ ID NO:45 and SEQ ID NO:69, SEQ ID NO:49 and SEQ ID NO:69, SEQ ID NO:53 and SEQ ID NO:69, SEQ ID NO:57 and SEQ ID NO:69, SEQ ID NO:61 and SEQ ID NO:69, or SEQ ID NO:65 and SEQ ID NO:69 (furthermore any of the sIL-6R VHHs shown in SEQ ID NOs: 119-137 can similarly be combined with SEQ ID NO:69 and any of SEQ ID NO:214-232 can be used in place of SEQ ID NO:69);
(ii) SEQ ID NO:1 and SEQ ID NO:73, SEQ ID NO:5 and SEQ ID NO:73, SEQ ID NO:9 and SEQ ID NO:73, SEQ ID NO: 13 and SEQ ID NO:73, SEQ ID NO: 17 and SEQ ID NO:73, SEQ ID NO: 21 and SEQ ID NO:73, SEQ ID NO:25 and SEQ ID NO:73, SEQ ID NO:29 and SEQ ID NO: 73, SEQ ID NO:33 and SEQ ID NO:73, SEQ ID NO:37 and SEQ ID NO:73, SEQ ID NO:41 and SEQ ID NO: 73, SEQ ID NO: 45 and SEQ ID NO:73, SEQ ID NO:49 and SEQ ID NO:73, SEQ ID NO: 53 and SEQ ID NO:73, SEQ ID NO:57 and SEQ ID NO:73, SEQ ID NO:61 and SEQ ID NO: 73, or SEQ ID NO:65 and SEQ ID NO: 73 (furthermore any of the sIL-6R VHHs shown in SEQ ID NOs: 119-137 can similarly be combined with SEQ ID NO:73 and any of SEQ ID NO:214-232 can be used in place of SEQ ID NO:73);
(iii) SEQ ID NO:1 and SEQ ID NO:77, SEQ ID NO:5 and SEQ ID NO:77, SEQ ID NO:9 and SEQ ID NO:77, SEQ ID NO: 13 and SEQ ID NO:77, SEQ ID NO: 17 and SEQ ID NO:77, SEQ ID NO: 21 and SEQ ID NO:77, SEQ ID NO:25 and SEQ ID NO:77, SEQ ID NO:29 and SEQ ID NO: 77, SEQ ID NO:33 and SEQ ID NO:77, SEQ ID NO:37 and SEQ ID NO:77, SEQ ID NO:41 and SEQ ID NO:77, SEQ ID NO: 45 and SEQ ID NO:77, SEQ ID NO:49 and SEQ ID NO:77, SEQ ID NO: 53 and SEQ ID NO:77, SEQ ID NO:57 and SEQ ID NO:77, SEQ ID NO:61 and SEQ ID NO: 77, or SEQ ID NO: 65 and SEQ ID NO: 77 (furthermore any of the sIL-6R VHHs shown in SEQ ID NOs: 119-137 can similarly be combined with SEQ ID NO:77 and any of SEQ ID NO:214-232 can be used in place of SEQ ID NO:77);
(iv) SEQ ID NO: 1 and SEQ ID NO:81, SEQ ID NO:5 and SEQ ID NO:81, SEQ ID NO:9 and SEQ ID NO:81, SEQ ID NO: 13 and SEQ ID NO:81, SEQ ID NO: 17 and SEQ ID NO:81, SEQ ID NO: 21 and SEQ ID NO:81, SEQ ID NO:25 and SEQ ID NO:81, SEQ ID NO:29 and SEQ ID NO: 81, SEQ ID NO:33 and SEQ ID NO:81, SEQ ID NO:37 and SEQ ID NO:81, SEQ ID NO:41 and SEQ ID NO:81, SEQ ID NO:45 and SEQ ID NO:81, SEQ ID NO:49 and SEQ ID NO:81, SEQ ID NO: 53 and SEQ ID NO:81, SEQ ID NO:57 and SEQ ID NO:81, SEQ ID NO:61 and SEQ ID NO: 81, or SEQ ID NO: 65 and SEQ ID NO: 81 (furthermore any of the sIL-6R VHHs shown in SEQ ID NOs: 119-137 can similarly be combined with SEQ ID NO:81 and any of SEQ ID NO:214-232 can be used in place of SEQ ID NO:81);
(v) SEQ ID NO:1 and SEQ ID NO:85, SEQ ID NO:5 and SEQ ID NO:85, SEQ ID NO:9 and SEQ ID NO:85, SEQ ID NO: 13 and SEQ ID NO:85, SEQ ID NO: 17 and SEQ ID NO:85, SEQ ID NO: 21 and SEQ ID NO:85, SEQ ID NO:25 and SEQ ID NO:85, SEQ ID NO:29 and SEQ ID NO: 85, SEQ ID NO:33 and SEQ ID NO:85, SEQ ID NO:37 and SEQ ID NO:85, SEQ ID NO:41 and SEQ ID NO: 85, SEQ ID NO:45 and SEQ ID NO:85, SEQ ID NO:49 and SEQ ID NO:85, SEQ ID NO: 53 and SEQ ID NO:85, SEQ ID NO:57 and SEQ ID NO:85, SEQ ID NO:61 and SEQ ID NO: 85, or SEQ ID NO: 65 and SEQ ID NO: 85 (furthermore any of the sIL-6R VHHs shown in SEQ ID NOs: 119-137 can similarly be combined with SEQ ID NO:85 and any of SEQ ID NO:214-232 can be used in place of SEQ ID NO:85); or
(vi) SEQ ID NO:1 and SEQ ID NO:89, SEQ ID NO:5 and SEQ ID NO:89, SEQ ID NO:9 and SEQ ID NO: 89, SEQ ID NO: 13 and SEQ ID NO:89, SEQ ID NO: 17 and SEQ ID NO: 89, SEQ ID NO: 21 and SEQ ID NO:89, SEQ ID NO:25 and SEQ ID NO:89, SEQ ID NO:29 and SEQ ID NO: 89, SEQ ID NO: 33 and SEQ ID NO: 89, SEQ ID NO:37 and SEQ ID NO:89, SEQ ID NO:41 and SEQ ID NO:89, SEQ ID NO:45 and SEQ ID NO: 89, SEQ ID NO:49 and SEQ ID NO:89, SEQ ID NO: 53 and SEQ ID NO:89, SEQ ID NO:57 and SEQ ID NO:89, SEQ ID NO:61 and SEQ ID NO: 89, or SEQ ID NO: 65 and SEQ ID NO: 89 (furthermore any of the sIL-6R VHHs shown in SEQ ID NOs: 119-137 can similarly be combined with SEQ ID NO:89 and any of SEQ ID NO:214-232 can be used in place of SEQ ID NO:89).

An embodiment provides an isolated nucleic acid molecule encoding any one of the binding proteins, the multispecific binding proteins, or the fusion proteins described herein.

An embodiment provides an expression vector comprising any one of the isolated nucleic acid molecules described herein.

An embodiment provides an isolated host cell comprising any one of the expression vectors described herein.

An embodiment provides an isolated amino acid sequence as set forth in any one of SEQ ID NOs: 1-92 or SEQ ID NO: 100-286.

An embodiment provides a pharmaceutical composition comprising one or more binding proteins, one or more multispecific binding proteins, and/or one or more of any one of the fusion proteins described herein, and a pharmaceutically acceptable carrier.

An embodiment provides a method of treating a neurological disease or condition in a subject comprising administering to the subject a therapeutically effective amount of any one of the binding peptides, any one of the multispecific binding protein, any one of the fusion proteins described herein, or any one of the pharmaceutical compositions described herein, thereby treating the neurological disease or condition in the subject.

The neurological disease or condition can be an inflammatory neurological disease or condition. The neurological disease or condition can be ALS, Parkinson disease, multiple sclerosis, Alzheimer's disease or Huntington's disease. Administering can comprise intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal, intrasternal, oral, sublingual buccal or nasal administrations, infusion, inhalation, or nebulization. Administering can comprise crossing blood brain barrier and delivering to intracellular space enter the brain hippocampal region. Treating the neurological disease can comprise improving glial-neuronal communication and retarding neurodegenerative process. Treating the neurological disease can comprise reducing cerebral inflammation, inhibiting can cerebral fibrosis and/or improving cerebral vascular function. Reducing inflammation can comprise inhibiting reactive cells production of inflammatory cytokines and/or free radicals. Inflammatory cytokines can comprise IL-6 and monocyte chemoattractant protein-1 (MCP-1). Reducing inflammation can comprise reducing IL-6 levels. Reducing IL-6 levels can comprise suppressing pSTAT3 expression in healthy astroglial cells. Inhibiting cerebral fibrosis can comprise inhibiting reactive cells production of serum amyloid. Reactive cells can comprise non neuronal cells selected from the group consisting of astrocytes, microglia, macrophages and T lymphocytes. Improving cerebral vascular function can comprise inhibiting cytokine-induced vasoconstriction, inducing vasodilation and/or increasing cerebral blood flow. Inhibiting cytokine-induced vasoconstriction can comprise inhibiting IL-6/AT1R mediated vasoconstriction of cerebral micro vessels.

An embodiment provides a method of treating a neurological disease or condition in a subject comprising administering to the subject a therapeutically effective amount of a fusion protein comprising a first binding protein that selectively and/or specifically binds to human sIL-6R linked to a second binding protein that selectively and/or specifically binds to human CTGF, thereby treating the neurological disease.

The first binding protein and the second binding protein can be VHH antibodies or nanobodies. The fusion protein can further comprise an anti-transferrin receptor binding protein.

An embodiment provides an isolated variable domain of a single heavy chain antibody (VHH) that selectively and/or specifically binds to human sIL-6R.

The VHH can comprise CDRs as set forth in SEQ ID NOs: 2-4, SEQ ID NOS: 6-8, SEQ ID NOs: 10-12, SEQ ID NOs: 14-16, SEQ ID NOs: 18-20, SEQ ID NOS: 22-24, SEQ ID NOS: 26-28, SEQ ID NOs: 30-32, SEQ ID NOs: 34-36, SEQ ID NOs: 38-40, SEQ ID NOs: 42-44, SEQ ID NOs: 46-48, SEQ ID NOs: 50-52, SEQ ID NOs: 54-56, SEQ ID NOs: 58-60, SEQ ID NOs: 62-64, SEQ ID NOs: 66-68, or SEQ ID NO: 138-194 or a binding protein having CDRs with 80% or greater identity to the CDRs of the binding protein comprising SEQ ID NOs: 2-4, SEQ ID NOs: 6-8, SEQ ID NOs: 10-12, SEQ ID NOs: 14-16, SEQ ID NOs: 18-20, SEQ ID NOs: 22-24, SEQ ID NOs: 26-28, SEQ ID NOs: 30-32, SEQ ID NOs: 34-36, SEQ ID NOs: 38-40, SEQ ID NOS: 42-44, SEQ ID NOs: 46-48, SEQ ID NOs: 50-52, SEQ ID NOs: 54-56, SEQ ID NOs: 58-60, SEQ ID NOs: 62-64, SEQ ID NOs: 66-68, or SEQ ID NO: 138-194 and the antigen binding specificity thereof.

An embodiment provides an isolated variable domain of a single heavy chain antibody (VHH) that selectively and/or specifically binds to human CTGF. The VHH can comprise CDRs as set forth in SEQ ID NOs: 70-72, SEQ ID NOs: 74-76, SEQ ID NOs: 78-80, SEQ ID NOs: 82-84, SEQ ID NOs: 86-88, SEQ ID NOs: 90-92, SEQ ID NOs: 233-286, or SEQ ID NOs: 291-293 or a binding protein having CDRs with 80% or greater identity to the CDRs of the binding protein comprising SEQ ID NOs: 70-72, SEQ ID NOs: 74-76, SEQ ID NOs: 78-80, SEQ ID NOs: 82-84, SEQ ID NOs: 86-88, SEQ ID NOs: 90-92, SEQ ID NOs: 233-286, or SEQ ID NOs: 291-293 and the antigen binding specificity thereof.

### BRIEF DESCRIPTION OF THE FIGURES

FIG. 1 is a schematic representation of the role of sIL-6R and CTGF in astroglial health.

FIGS. 2A-2B provide the sequences of the immunogens used. FIG. 2A shows the amino acid sequence of CTGF (SEQ ID NO:93) and the amino acid sequence of the immunogen of CTGF used (SEQ ID NO:94). FIG. 2B shows the amino acid sequence of the immunogen of sIL-6R used (SEQ ID NO:95).

FIG. 3 is a table illustrating the schedule of immunization of camel with CTGF and sIL-6R.

FIGS. 4A-4B show antibody responses in camel as measured by ELISA. FIG. 4A is a graph illustrating anti-CTGF sera before and after immunization. FIG. 4B is a graph illustrating anti-sIL-6R sera before and after immunization (with post-immune sera showing greater absorbance).

FIGS. 5A-5B illustrate the amplification of VHH for phage display library construction. FIG. 5A illustrates the experimental design. FIG. 5B is a photograph of the electrophoresis gels showing the PCR products.

FIGS. 6A-6C illustrate the cloning strategy of VHH fragment antibody genes in phage display vector. FIG. 6A is a schematic representation of the cloning strategy. FIG. 6B is a photograph of an electrophoresis gel showing the PCR products. FIG. 6C is a photograph showing the bacteria colony formation after transformation with the empty vector (left) or with the vector comprising the insert (right).

FIGS. 7A-7F show CTGF immune library colony screening for the presence of VHH fragment antibody gene. FIG. 7A is a photograph of the electrophoresis gel showing the PCR products for colonies 1-12. FIG. 7B is a photograph of the electrophoresis gel showing the PCR products for colonies 13-25. FIG. 7C is a photograph of the electrophoresis gel showing the PCR products for colonies 26-38. FIG. 7D is a photograph of the electrophoresis gel showing the PCR products for colonies 39-51. FIG. 7E is a photograph of the electrophoresis gel showing the PCR products for colonies 52-63. FIG. 7F is a photograph of the electrophoresis gel showing the PCR products for colonies 64-76.

FIGS. 8A-8B illustrate the screening for the VHH fragment antibody expression in whole cell lysate/periplasmic extract. FIG. 8A is a photograph of an immunoblot for clones 1-3. FIG. 8B is a photograph of an immunoblot for clones 4-7.

FIG. 9 is a photograph of an immunoblot illustrating the specific recognition of CTGF by VHH fragment antibody.

FIG. 10 is a graph illustrating the effect of anti-sIL-6R fragment on IL-6 stimulated expression of pSTAT3 (a cellular inflammatory marker).

FIGS. 11A-11B illustrate the internalization of anti-sIL-6R fragment in HepG2 cells. FIG. 11A is a photograph showing the intracellular detection of the anti-sIL-6R fragment in cells treated with the VHH fragment antibody, as detected by immunofluorescence. FIG. 11B is a photograph showing a control experiment, in the absence of the anti-sIL-6R fragment.

FIG. 12 illustrates a dot blot analysis of cerebrospinal fluid, showing that anti-sIL-6R fragment crosses the blood-brain barrier (BBB) and is detected in the hippocampus, using VHH-anti-TfR conjugate.

FIGS. 13A-13E illustrate that the anti-sIL-6R fragment crosses the blood-brain barrier and accumulated in the cerebrospinal fluid, by showing the detection of the VHH fragment and of the anti-transferrin receptor antibody in brain section, as detected by immunofluorescence. FIG. 13A is a photograph showing the immunofluorescence detected in brain section incubated with VHH fragment alone. FIG. 13B is a photograph showing the immunofluorescence detected in brain section incubated with VHH fragment conjugated with anti-TfR. FIG. 13C is a photograph showing the immunofluorescence detected in brain section incubated with anti-TfR alone. FIG. 13D is a photograph showing the immunofluorescence detected in brain section incubated with conjugation buffer alone. FIG. 13E is a photograph showing the immunofluorescence detected in brain section incubated with fragment buffer alone.

FIGS. 14A-14B illustrate the role of IL-6/IL-6R in cerebral micro-vessels. FIG. 14A is a photograph illustrating that the anti-sIL-6R fragment binds to cerebral blood vessels. FIG. 14B is a schematic representation of the pathway target for anti sIL-6R fragment to reverse the vascular effect of IL-6.

### DETAILED DESCRIPTION

The present disclosure provides sIL-6R and CTGF binding proteins, multispecific binding proteins thereof, conjugates thereof, pharmaceutical compositions thereof, and methods of use thereof. The binding proteins can be VHH antibodies. The methods of use include methods of treating neurological diseases and disorders such as inflammatory neurological diseases and disorders.

### Overview

Early in the neurodegenerative process changes in innate immunity can lead to increased production and release of significant levels of cytokines. IL-6 and CTGF are elevated in ALS and mediate the activation of astrocytes and microglia, as well as increase production of serum amyloid and free radicals. CTGF is also found in the cytoplasm of neurons. There is still an unmet need for therapies directly targeting these properties.

Both IL-6 and CTGF are elevated in neurological diseases such as ALS and mediate the activation of astrocytes and microglia, as well as increase production of serum amyloid and free radicals. Therefore, therapies directly targeting these properties can be of therapeutic importance.

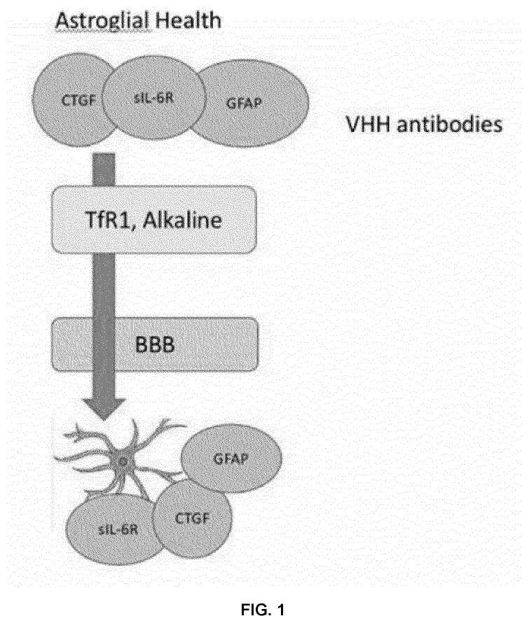

In order to limit the inflammatory process which underlies these neurological disorders camel VHH fragment antibodies were developed (FIG. 1). The strategy includes the generation of dromedary camel VHH fragment antibodies to pro-inflammatory/pro-fibrotic cytokines or their signaling, specifically sIL-6R, CTGF, which marks hippocampal astroglial cells and transferrin receptors (TfR) to facilitate antibodies crossing the blood brain barrier (BBB).

The present disclosure provides two groups of VHH fragments that can be used to treat neurological diseases. One group targets sIL-6R while the second group targets CTGF. As discussed below, these fragments have been tested both in vitro and in vivo, they can be delivered alone or in conjugation with an anti-transferrin receptor antibody, they can cross the blood brain barrier, enter cells and possess anti-inflammatory properties. They can be delivered through cation resurfacing, as fusion proteins and by molecular conjugation.

The sIL-6R antibodies are anti-inflammatory, can be delivered into the intercellular space and cross the BBB, even without the assistance of being chaperoned by anti-TfR antibodies. Furthermore, the anti-sIL-6R VHH fragments generated show greater selectivity to the soluble receptor compared to the membrane bound receptor. The basis for this selective binding is being investigated but is thought to be vested in the ‘convex binding’ of the VHH fragment antibodies to their target, as opposed to the ‘concave binding’ of the conventional monoclonal antibodies.

Since the anti sIL-6R and anti CTGF fragments bind different cellular targets, there is no competition for binding or steric hindrance, when administered together.

sIL-6R and CTGF binding proteins

Provided herein are sIL-6R and CTGF binding proteins, multispecific binding proteins thereof, protein conjugates thereof, pharmaceutical compositions thereof, and methods of use thereof.

Interleukin 6 (IL-6) is an interleukin that acts as both a pro-inflammatory cytokine and an anti-inflammatory myokine. In humans, it is encoded by the IL6 gene. IL-6 signals through Interleukin 6 receptor (IL6R), a cell-surface type I cytokine receptor complex consisting of the ligand-binding IL-6Ra chain, also known as cluster of differentiation 126 (CD126), and the signal-transducing component gp130 (also called CD130). CD130 is the common signal transducer for several cytokines including leukemia inhibitory factor (LIF), ciliary neurotropic factor, oncostatin M, IL-11 and cardiotrophin-1, and is almost ubiquitously expressed in most tissues. In contrast, the expression of CD126 is restricted to certain tissues. As IL-6 interacts with its receptor, it triggers the gp130 and IL-6R proteins to form a complex, thus activating the receptor. These complexes bring together the intracellular regions of gp130 to initiate a signal transduction cascade through certain transcription factors, janus kinases (JAKs) and signal transducers and activators of transcription (STATs).

IL-6 is probably the best-studied of the cytokines that use gp130, also known as IL-6 signal transducer (IL6ST), in their signaling complexes. Other cytokines that signal through receptors containing gp130 are Interleukin 11 (IL-11), Interleukin 27 (IL-27), ciliary neurotrophic factor (CNTF), cardiotrophin-1 (CT-1), cardiotrophin-like cytokine (CLC), leukemia inhibitory factor (LIF), oncostatin M (OSM), Kaposi's sarcoma-associated herpesvirus interleukin 6-like protein (KSHV-IL6). These cytokines are commonly referred to as the IL-6 like or gp130 utilizing cytokines.

In addition to the membrane-bound receptor, a soluble form of IL-6R (sIL-6R) has been purified from human serum and urine. Many neuronal cells are unresponsive to stimulation by IL-6 alone, but differentiation and survival of neuronal cells can be mediated through the action of SIL-6R. The sIL-6R/IL-6 complex can stimulate neurites outgrowth and promote survival of neurons and, hence, may be important in nerve regeneration through remyelination.

IL-6R is expressed in a cell- and tissue-specific manner and only found on hepatocytes and some leukocytes like neutrophils and T cells. Signaling via the membrane-bound IL-6R is mostly regenerative and anti-inflammatory.

IL-6R is a typical type-I transmembrane protein that is made up of an immunoglobulin (lg)—like domain (“D1”), a cytokine-binding module (CBM) residing in two fibronectin-type-Ill domains (“D2” and “D3”), and a 55 amino-acid residue-long flexible stalk region that is followed by a transmembrane and an intracellular region. A minimal length of 22 amino acids of the stalk region is required for efficient IL-6 classic signaling, which corresponds to a stalk length of approximately 83 Å.

Soluble forms of the IL-6R (sIL-6R) are found in human serum at concentrations of about 30-70 ng/ml. IL-6 binds to membrane-bound and sIL-6R with similar affinity and signaling of IL-6/sIL-6R has been termed IL-6 trans-signaling and is causative for the proinflammatory properties of IL-6. Specific inhibition of IL-6 trans-signaling holds the promise to be as effective as total IL-6 blockade, but with reduced side effects like enhanced susceptibility to bacterial infections. One mechanism that contributes to sIL-6R generation is alternative splicing of the IL6R mRNA, which results in a unique C-terminus of ten amino-acid residues, and only this form of the sIL-6R has been detected in human serum. However, only 1%-20% of the total sIL-6R is generated by alternative splicing, and the generation mechanism of the majority remains unknown. In vitro, sIL-6R can be efficiently generated by limited proteolysis, predominantly by the metalloproteases ADAM 10 and ADAM17.

CTGF, also known as CCN2, is a 38 kDa, cysteine-rich (22 cysteines in the N-terminal and 16 cysteines in the C-terminal region), extracellular matrix protein that belongs to the CCN family of proteins. The term ‘connective tissue growth factor’ was introduced to describe a polypeptide growth factor that stimulates DNA synthesis and chemotaxis in fibroblasts. There are five other CCN gene family genes: CCN1 (Cyr61), CCN3 (NOV), CCN4 (WISP1), CCN5 (WISP2), and CCN6 (WISP3). The CCN acronym was introduced from the names of the first three members of the family to be discovered: Cyr61 (cysteine-rich protein 61), CTGF, and NOV (nephroblastoma overexpressed gene). Expression of CTGF is crucial to embryonic development in childhood, for example, mice with CTGF knockout have multiple skeletal dysmorphisms and perinatal lethality.

Abnormal expression of CTGF can be detected in several adulthood diseases including fibrosis and malignancy in major organs and tissues.

CTGF expression was initially discovered in endothelial cells and fibroblasts associated with connective tissue regeneration and wound healing, and then was detected in many tissues. CTGF expression is higher in blood vessels and lungs compared to other organs or tissues, which emphasize the role of CTGF in the development of blood vessels and lungs. Low expression of CTGF mRNA can be observed in brain tissues, however, the adult cerebral cortex strongly expresses CTGF mRNA.

The proper expression of CTGF is essential for the physiological process of multiple organs such as bone, brain, heart, and lung. High expression of CTGF negatively regulates myelination during development, which can be implicated in a range of neurodevelopmental disorders. CTGF mRNA is highly expressed in developing blood vessels and large blood vessels of the adult heart, suggesting that it may be involved in the maintenance of blood vessel integrity during adulthood. The absence of CTGF and/or its protein product, CTGF, may induce pulmonary hypoplasia by disrupting basic lung developmental processes.

CTGF (6q23.2) is a relatively short gene and consists of 5 exons that code for a 349-amino acid protein, the first exon codes for a signal peptide (for secretion) and exons 2-5 code for each of the four different domains. The four functional domains are insulin-like growth factor binding protein (IGFBP), von Willebrand factor type C repeat (VWC), thrombospondin type-1 repeat (TSP1 or TSR), and cysteine knot-containing domain (CT). IGFBP and VWC domains constitute the N-terminal half of CTGF which is separated from the C-terminal half that contains TSP1 and CT domains by a ‘hinge’ region. The functions of CTGF domains are different because of their distinct bindings with specific proteins in various signaling pathways. Since these binding proteins participate in a number of physiological processes, CTGF has been shown to regulate a wide range of important functional pathways, including adhesion, mitogenesis, and chemotaxis, cell survival, differentiation, angiogenesis, chondrogenesis, tumorigenesis, and wound healing. Although some biological functions are directly related to an individual functional domain, many functions are demonstrated to be the consequence of domains acting in concert. For example, truncated CTGF domains (N-terminal fragment and C-terminal fragment), can function independently to stimulate differentiation or proliferation of fibroblast and to increase collagen synthesis.

An embodiment provides a binding protein that selectively and/or specifically binds to human sIL-6R. Another embodiment provides a binding protein that selectively and/or specifically binds to human CTGF.

The terms “peptide”, “polypeptide” and “protein” are used interchangeably herein and refer to any chain of at least two amino acids, linked by a covalent chemical bound. As used herein polypeptide can refer to the complete amino acid sequence coding for an entire protein or to a portion thereof. A “protein coding sequence” or a sequence that “encodes” a particular polypeptide or peptide, is a nucleic acid molecule that is transcribed (in the case of DNA) and is translated (in the case of mRNA) into a polypeptide in vitro or in vivo when placed under the control of appropriate regulatory sequences. The boundaries of the coding sequence can be determined by a start codon at the 5′ (amino) terminus and a translation stop codon at the 3′ (carboxyl) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

The term “binding peptide”, “binding protein”, “multispecific binding protein” and the like, as used herein are meant to refer to any peptide or polypeptide (including proteins and fusion proteins) that can bind to a protein of interest. A binding protein can comprise, for example, an scFv, a Fab, a Fab′, a Fv, a F (ab′)₂), a minibody, a diabody, a triabody, a tetrabody, a tandem di-scFv, a tandem tri-scFv, an immunoglobulin single variable domain (ISV) (e.g., a VHH (including humanized VHH), a camelized VH, a single domain antibody, a domain antibody, a Variable New Antigen Receptor (VNAR), or a dAb), a VH, a Fab, a Fab′, a F(ab′)2, a variable fragment (Fv), a disulfide-stabilized Fv (dsFv), a single-chain Fv (scFv), a nanobody, a diabody, a triabody, or an AFFIBODY®. A multispecific binding protein can comprise two or more binding proteins.

Binding selectivity or selective binding can be defined with respect to the binding of a binding protein or antibody to a substrate (e.g., a protein like sIL-6R, IL-6R, or CTGF) forming a complex. Binding selectivity describes how a binding protein can bind more preferentially to one protein (e.g. sIL-6R) than another protein (e.g., membrane bound IL-6R). A selectivity coefficient is the equilibrium constant for the reaction of displacement by one binding protein of another binding protein in a complex with the protein. The concept of selectivity is used to quantify the extent to which one chemical substance, A (e.g., a binding protein such as those described herein), binds each of other proteins, B and C. The interactions can be characterized by equilibrium constants KAB and Kac. This selectivity coefficient being in fact the equilibrium constant for the displacement reaction. The greater the selectivity coefficient, the more the protein C (e.g., SIL-6R) will displace the protein B (e.g., IL-6R) from the complex formed with the binding protein A (e.g. one of the binding proteins or antibodies described herein that selectively bind sIL-6R over IL-6R). An alternative interpretation is that the greater the selectivity coefficient, the lower the concentration of C that is needed to displace B from AB.

With selective binding a binding protein can bind to its target antigen (e.g. sIL-6R) with a higher affinity than to any cross-reactive antigen (e.g., membrane bound IL-6) as determined using experimental techniques, such as radioimmunoassays (RIA) and enzyme linked immunosorbent assays (ELISAs).

Selective binding of a binding protein to a target protein (e.g. sIL-6R) can be indicated by a measured fluorescent binding intensity (MFI) value, as assessed by cell flow cytometry or other suitable method, of at least 2-fold, at least 3-fold, at least 4-fold, at least 5-fold, at least 6-fold, at least 7-fold, at least 8-fold, at least 10-fold, at least 15-fold, at least 20-fold or greater, as compared with binding of the antibody to a cross-reactive antigen (e.g., membrane bound IL-6R). In an embodiment, selectively binding proteins exhibit an MFI value of from 2.0-fold to 25-fold, or from 2-fold to 20-fold, or from 3-fold to 15-fold, or from 4-fold to 8-fold, or from 2-fold to 10-fold, or from 2-fold to 5-fold or more greater than the MFI value of the same antibody for binding a cross-reactive antigen.

The term “specific binding” or “antigen binding specificity” refers to binding proteins (e.g., anti-sIL-6R and anti-CTGF antibodies) binding to a predetermined antigen (i.e., sIL-6R or CTGF). Typically, the binding protein binds with an affinity corresponding to a K_{D} of about 10⁻⁸ M or less and binds to the predetermined antigen with an affinity (as expressed by K_{D}) that is at least 10-fold less, and in some aspects at least 100-fold less (e.g., at least 10-, 20-, 30-, 40-, 50-, 60-, 70-, 80-, 90- or 100-fold less) than its affinity for binding to a non-specific antigen (e.g., BSA, casein) other than the predetermined antigen or a closely related antigen. Alternatively, the binding protein can bind with an affinity corresponding to a KA of about 10⁶ M⁻¹, or about 10⁷ M⁻¹, or about 10⁸ M-1, or 10⁹ M-1 or higher, and binds to the predetermined antigen with an affinity (as expressed by KA) that is at least 10-fold higher, and in some aspects at least 100-fold higher (e.g., at least 10-, 20-, 30-, 40-, 50-, 60-, 70-, 80-, 90- or 100-fold higher) than its affinity for binding to a non-specific antigen (e.g., BSA, casein) other than the predetermined antigen or a closely-related antigen. K_{D} and KA can be determined using experimental techniques, such as radioimmunoassays (RIA) and enzyme linked immunosorbent assays (ELISAs).

The binding proteins described herein can include at least one CDR region. By “CDR region” it is meant that the binding protein include one or more of the three segments called CDRs or hypervariable regions and a more highly conserved portions of the variable domains of an antibody that specifically recognize the protein of interest. The binding proteins can be an antibody, an antibody fragment, and the like, having specific binding to one or more target polypeptide, including the protein of interest (sIL-6R and CTGF).

The binding protein that selectively and/or specifically binds sIL-6R can comprise one or more CDRs as set forth in SEQ ID NOs: 2-4, SEQ ID NOs: 6-8, SEQ ID NOS: 10-12, SEQ ID NOs: 14-16, SEQ ID NOs: 18-20, SEQ ID NOs: 22-24, SEQ ID NOs: 26-28, SEQ ID NOs: 30-32, SEQ ID NOs: 34-36, SEQ ID NOs: 38-40, SEQ ID NOs: 42-44, SEQ ID NOs: 46-48, SEQ ID NOs: 50-52, SEQ ID NOs: 54-56, SEQ ID NOs: 58-60, SEQ ID NOs: 62-64, SEQ ID NOs: 66-68, or

SEQ ID NO: 138-194 or a binding protein having CDRs with 80% or greater identity to the CDRs of the binding protein comprising SEQ ID NOs: 2-4, SEQ ID NOs: 6-8, SEQ ID NOs: 10-12, SEQ ID NOs: 14-16, SEQ ID NOs: 18-20, SEQ ID NOs: 22-24, SEQ ID NOs: 26-28, SEQ ID NOs: 30-32, SEQ ID NOs: 34-36, SEQ ID NOs: 38-40, SEQ ID NOs: 42-44, SEQ ID NOs: 46-48, SEQ ID NOs: 50-52, SEQ ID NOs: 54-56, SEQ ID NOs: 58-60, SEQ ID NOs: 62-64, SEQ ID NOs: 66-68, or SEQ ID NO: 138-194 and the antigen binding specificity thereof.

The binding protein that selectively and/or specifically binds CTGF can comprise one or more CDRs as set forth in SEQ ID NOs: 70-72, SEQ ID NOs: 74-76, SEQ ID NOs: 78-80, SEQ ID NOs: 82-84, SEQ ID NOS: 86-88, SEQ ID NOs: 90-92, SEQ ID NOs: 233-286, or SEQ ID NOs: 291-293 or a binding protein having CDRs with 80% or greater identity to the CDRs of the binding protein comprising SEQ ID NOs: 70-72, SEQ ID NOs: 74-76, SEQ ID NOs: 78-80, SEQ ID NOs: 82-84, SEQ ID NOs: 86-88, SEQ ID NOs: 90-92, SEQ ID NOs: 233-286, or SEQ ID NOs: 291-293 and the antigen binding specificity thereof.

The terms “sequence identity” or “percent identity” are used interchangeably herein. To determine the percent identity of two polypeptide molecules or two polynucleotide sequences, the sequences are aligned for optimal comparison purposes (e.g., gaps can be introduced in the sequence of a first polypeptide or polynucleotide for optimal alignment with a second polypeptide or polynucleotide sequence). The amino acids or nucleotides at corresponding amino acid or nucleotide positions are then compared. When a position in the first sequence is occupied by the same amino acid or nucleotide as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). In some embodiments the length of reference sequences (e.g., SEQ ID NOs: 1-92) aligned for comparison purposes is at least 80% of the length of the comparison sequence, and in some embodiments is at least 90% or 100%. In an embodiment, the two sequences are the same length.

Ranges of desired degrees of sequence identity are approximately 75% to 100% and integer values in between. Percent identities between a disclosed sequence and a claimed sequence can be at least 75%, at least 80%, at least 85%, at least 90%, at least 95%, at least 96%, at least 97%, at least 98%, at least 99%, at least 99.5%, or at least 99.9%. In general, an exact match indicates 100% identity over the length of the reference sequences (e.g., SEQ ID NOs: 1-92). Polypeptides and polynucleotides that are about 85, 86, 87, 88, 89, 90, 91, 92, 93, 94, 95, 96, 97, 98, 99 99.5% or more identical to polypeptides and polynucleotides described herein are embodied within the disclosure. For example, a polypeptide can have 80%, 85%, 90%, 91%, 92%, 93%, 94%, 95%, 96%, 97%, 98%, 99% or more identity to SEQ ID NOs: 1-92.

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions. A conservative substitution for an amino acid in a binding protein may be selected from other members of the class to which the amino acid belongs. For example, it is well-known in the art of protein biochemistry that an amino acid belonging to a grouping of amino acids having a particular size or characteristic (such as charge, hydrophobicity and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr so that a free-OH is maintained; and Gln for Asn to maintain a free-NH2. Likewise, biologically active analogues of a protein containing deletions or additions of one or more contiguous or noncontiguous amino acids that do not eliminate a functional activity of the protein are also contemplated.

Variants of the disclosed sequences also include proteins, or full-length protein, that contain substitutions, deletions, or insertions into the protein backbone, that would still leave at least about 70% homology to the original protein over the corresponding portion. A yet greater degree of departure from homology is allowed if like-amino acids, i.e., conservative amino acid substitutions, do not count as a change in the sequence. Examples of conservative substitutions can involve amino acids that have the same or similar properties. Illustrative amino acid conservative substitutions include the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine to leucine.

The antibody can have a better binding affinity to soluble IL-6R as compared to a binding affinity to membrane bound IL-6R.

“Binding affinity” can be defined at the strength of the binding interaction between a binding protein (e.g., the binding proteins described herein) to its ligand/binding partner (e.g., its binding target, such as sIL-6R). It can be defined as the concentration of ligand required to occupy 50% of the targets at equilibrium. That is a binding protein that selectively and/or specifically binds sIL-6R can bind both soluble and membrane bound IL-6R, but can have a greater affinity for the soluble form of the receptor as compared to the membrane bound form. In such an example, a lower concentration of the binding protein would be needed to occupy 50% of a sIL-6R binding protein, as compared to the concentration of the binding protein that would be necessary to occupy 50% of a membrane bound IL-6R. The binding protein can an antibody or an antigen-binding fragment thereof.

The term “antibody” or “binding protein” generally refers to immunoglobulin molecules and immunologically active portions of immunoglobulin molecules, i.e., molecules that contain an antigen binding site that immunospecifically binds an antigen. “Native antibodies” and “intact immunoglobulins”, or the like, are usually heterotetrameric glycoproteins of about 150,000 daltons, composed of two identical light (L) chains and two identical heavy (H) chains. The light chains from any vertebrate species can be assigned to one of two clearly distinct types, called kappa (κ) and lambda (A), based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of immunoglobulins are called α, δ, ε, γ, and μ, respectively. The subunit structures and three-dimensional configurations of different classes of immunoglobulins are well known. In a typical antibody molecule, each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains. Each variable region includes three segments called CDRs or hypervariable regions and a more highly conserved portions of variable domains are called the framework region (FR). The variable domains of heavy and light chains each includes four FR regions, largely adopting a β-sheet configuration, connected by three CDRs, which form loops connecting, and in some cases forming part of the β-sheet structure. The CDRs in each chain are held together in close proximity by the FRs and, with the CDRs from the other chain, contribute to the formation of the antigen-binding domain or targeting domain of antibodies (see Kabat et al., NIH Publ. No. 91-3242, Vol. I, pages 647-669 [1991]). The constant domains are not involved directly in binding an antibody to an antigen, but exhibit various effector functions, such as participation of the antibody in antibody dependent cellular cytotoxicity.

The term “antigen-binding domain” refers to the part of an antibody molecule that comprises the area specifically binding to or complementary to a part or all of an antigen. Where an antigen is large, an antibody may only bind to a particular part of the antigen. The “epitope” or “antigenic determinant” is a portion of an antigen molecule that is responsible for interactions with the antigen-binding domain of an antibody. An antigen-binding domain may be provided by one or more antibody variable domains (e.g., a so-called Fd antibody fragment consisting of a VH domain). An antigen-binding domain may comprise an antibody light chain variable region (VL) and an antibody heavy chain variable region (VH) or be defined by the CDRs of the VH and VL regions.

Antibodies can be cleaved experimentally with the proteolytic enzyme papain, which causes each of the heavy chains to break, producing three separate antibody fragments. The two units that consist of a light chain and a fragment of the heavy chain approximately equal in mass to the light chain are called the Fab fragments (i.e., the “antigen binding” fragments). The third unit, consisting of two equal segments of the heavy chain, is called the Fc fragment. The Fc fragment is typically not involved in antigen-antibody binding but is important in later processes involved in ridding the body of the antigen. As used herein, “antibody fragments” include a portion of an intact antibody including the antigen binding or variable region of the intact antibody. Examples of antibody fragments include Fab, Fab′ and F(ab′)2, Fc fragments or Fc-fusion products, single-chain Fvs (scFv), disulfide-linked Fvs (sdfv) and fragments including either a VL or VH domain; diabodies, tribodies and the like (Zapata et al. Protein Eng. 8 (10): 1057-1062).

A Fab fragment contains a constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F (ab′)2 antibody fragments originally were produced as pairs of Fab′ fragments which have hinge cysteines between them. Other chemical couplings of antibody fragments are also known.

The Fc region of an antibody is the tail region of an antibody that interacts with cell surface receptors and some proteins of the complement system. This property allows antibodies to activate the immune system. In IgG, IgA and IgD antibody isotypes, the Fc region is composed of two identical protein fragments, derived from the second and third constant domains of the antibody's two heavy chains; IgM and IgE Fc regions contain three heavy chain constant domains (CH domains 2-4) in each polypeptide chain. The Fc regions of IgGs bear a highly conserved N-glycosylation site. Glycosylation of the Fc fragment is essential for Fc receptor-mediated activity. The N-glycans attached to this site are predominantly core-fucosylated diantennary structures of the complex type. In addition, small amounts of these N-glycans also bear bisecting GlcNAc and α-2,6 linked sialic acid residues.

Fc-Fusion proteins (also known as Fc chimeric fusion protein, Fc-lg, Ig-based Chimeric Fusion protein and Fc-tag protein) are composed of the Fc domain of IgG genetically linked to a peptide or protein of interest, such as a binding protein or a multispecific binding protein described herein. Fc-Fusion proteins have become valuable reagents for in vivo and in vitro research. The Fc-fused binding partner can range from a single peptide, a ligand that activates upon binding with a cell surface receptor, signaling molecules, the extracellular domain of a receptor that is activated upon dimerization or as a bait protein that is used to identify binding partners in a protein microarray. One of the most valuable features of the Fc domain in vivo, is it can dramatically prolong the plasma half-life of the protein of interest, which for bio-therapeutic drugs, results in an improved therapeutic efficacy; an attribute that has made Fc-Fusion proteins attractive bio-therapeutic agents. The Fc fusion protein may be part of a pharmaceutical composition including an Fc fusion protein and a pharmaceutically acceptable carrier excipients or carrier. Pharmaceutically acceptable carriers, excipients or stabilizers are well known in the art (Remington's Pharmaceutical Sciences, 16th edition, Osol, A. Ed. (1980)). Acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG).

In an aspect, a binding protein or multispecific binding protein can be linked or fused to one or more Fc domains. In certain aspects, an Fc domain can be engineered to pair or heterodimerize with a first and second binding protein. For example, a multispecific binding protein can comprise a first polypeptide comprising a fusion of a binding protein specific for sIL-6R and an Fc domain and a second polypeptide comprising a fusion of a binding protein specific for CTGF and a second Fc domain capable of dimerizing with the first Fc domain.

“Fv” is the minimum antibody fragment which contains a complete antigen-recognition and -binding site. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

“Single-chain Fv” or “sFv” antibody fragments comprise the VH and VL domains of an antibody, wherein these domains are present in a single polypeptide chain. The Fv polypeptide can further comprise a polypeptide linker between the VH and VL domains which enables the sFv to form the desired structure for antigen binding. For a review of sFv see Pluckthun in The Pharmacology of Monoclonal Antibodies, vol. 113, Rosenburg and Moore eds., Springer-Verlag, New York, pp. 269-315 (1994).

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan

et al., Science, 229:81 [1985]). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F (ab′2 fragments (Carter et al., Bio/Technology 10:163-167 [1992]). According to another approach, F (ab′)2 fragments can be isolated directly from recombinant host cell culture. Other techniques for the production of antibody fragments will be apparent to the skilled practitioner. In other embodiments, the antibody of choice is a single chain Fv fragment (scFv). See WO 93/16185.

In one aspect, the antigen-binding fragment described herein is a Fab fragment, Fab′ fragment, F (ab)′2 fragment, variable fragment (Fv) fragment, single chain antibody, single chain variable fragment (scFv), or a disulfide stabilized variable fragment (dsFv).

The antibody can be a monoclonal antibody.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies and is not to be construed as requiring production of the antibody by any particular method. For example, the monoclonal antibodies can be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The antibody can be a chimeric antibody, a humanized antibody or a camelid antibody. The antibody can be a camelid antibody.

Monoclonal antibodies can include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 [1984]). Chimeric antibody of interest can include “primatized” antibodies including variable domain antigen-binding sequences derived from a non-human primate (e.g., Old World Monkey, Ape etc.) and human constant region sequences; or “humanized” antibodies.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementarity determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies may comprise residues which are found neither in the recipient antibody nor in the imported CDR or framework sequences. These modifications are made to further refine and maximize antibody performance. In general, the humanized antibody will comprise substantially all of at least one, and typically two, variable domains, in which all or substantially all of the CDRs correspond to those of a non-human immunoglobulin and all or substantially all of the FRs are those of a human immunoglobulin sequence. The humanized antibody optimally also will comprise at least a portion of an immunoglobulin constant region (Fc), typically that of a human immunoglobulin. For further details, see Jones et al., Nature, 321:522-525 (1986); Reichmann et al., Nature, 332:323-329 (1988); and Presta, Curr. Op. Struct. Biol., 2:593-596 (1992). The humanized antibody includes a PRIMATIZED™ antibody wherein the antigen-binding region of the antibody is derived from an antibody produced by immunizing macaque monkeys with the antigen of interest. Methods for humanizing non-human antibodies are well known in the art.

Camelid, single-domain antibody (sdAb) or nanobodies (VHH fragment antibodies) are binding proteins having single, variable heavy chain (VHH) immunoglobulin (lg) domains that can be derived by antigen stimulation of camelids such as camels, llamas, and alpacas. Following immunization, the camelids can produce, among the normal Ig response, special heavy-chain only antibodies (hcAb) harboring the VHH. VHH fragment antibodies, single Ig domain proteins, are small prolate-shaped molecules (<15 kDa that retain the epitope-recognizing function of an antibody. VHH fragment antibodies may be selected to contain an extended and more flexible CDR3 loop than the regular VH domains, partly contributing to their high epitope affinity and their ability to better access smaller and cryptic epitopes. Moreover, VHH domains are amenable to cloning and protein modifications, and can be produced in bacterial expression systems in scalable amounts. VHH fragment antibodies also display superior solubility, stability, in vivo half-lives and pharmacodynamics compared to conventional antibodies. Single-domain camelid antibodies have been shown to be just as specific as a regular antibody and in some cases are more robust. VHH can easily be isolated using a phage panning procedure as used for traditional antibodies, allowing in vitro culture in large concentrations. The smaller size and single domain make these antibodies easier to transform into bacterial cells for bulk production.

A VHH fragment antibody can comprise the amino acid sequence of SEQ ID NO: 1, SEQ ID NO: 5, SEQ ID NO:9, SEQ ID NO: 13, SEQ ID NO: 17, SEQ ID NO:21, SEQ ID NO:25, SEQ ID NO: 29, SEQ ID NO: 33, SEQ ID NO: 37, SEQ ID NO:41, SEQ ID NO: 45, SEQ ID NO:49, SEQ ID NO: 53, SEQ ID NO:57, SEQ ID NO: 61, SEQ ID NO: 65, SEQ ID NO:69, SEQ ID NO:73, SEQ ID NO: 77, SEQ ID NO:81, SEQ ID NO:85, SEQ ID NO:89, SEQ ID NOS: 119-137, SEQ ID NOS: 233-286, or SEQ ID NOs: 291-293.

An embodiment provides an isolated amino acid sequence as set forth in any one of SEQ ID NOs: 1-92 or SEQ ID NOs: 100-286.

An embodiment provides an single domain antibody isolated variable domain of a single heavy chain antibody (VHH) Ig (VHH fragment antibody) that selectively and/or specifically binds to human sIL-6R.

A single domain antibody or VHH fragment antibody can comprise CDRs as set forth in SEQ ID NOs: 2-4, SEQ ID NOs: 6-8, SEQ ID NOs: 10-12, SEQ ID NOs: 14-16, SEQ ID NOs: 18-20, SEQ ID NOS: 22-24, SEQ ID NOs: 26-28, SEQ ID NOs: 30-32, SEQ ID NOs: 34-36, SEQ ID NOs: 38-40, SEQ ID NOs: 42-44, SEQ ID NOs: 46-48, SEQ ID NOs: 50-52, SEQ ID NOs: 54-56, SEQ ID NOs: 58-60, SEQ ID NOs: 62-64, SEQ ID NOs: 66-68, or SEQ ID NO: 138-194 or a binding protein having CDRs with 80% or greater identity to the CDRs of the binding protein comprising SEQ ID NOs: 2-4, SEQ ID NOs: 6-8, SEQ ID NOs: 10-12, SEQ ID NOS: 14-16, SEQ ID NOs: 18-20, SEQ ID NOS: 22-24, SEQ ID NOs: 26-28, SEQ ID NOs: 30-32, SEQ ID NOs: 34-36, SEQ ID NOs: 38-40, SEQ ID NOs: 42-44, SEQ ID NOs: 46-48, SEQ ID NOs: 50-52, SEQ ID NOs: 54-56, SEQ ID NOs: 58-60, SEQ ID NOS: 62-64, SEQ ID NOs: 66-68, or SEQ ID NO: 138-194 and the antigen binding specificity thereof.

An embodiment provides a single domain antibody or an isolated variable domain of a single heavy chain antibody (VHH) that selectively and/or specifically binds to human CTGF. A VHH can comprise CDRs as set forth in SEQ ID NOs: 70-72, SEQ ID NOs: 74-76, SEQ ID NOs: 78-80, SEQ ID NOs: 82-84, SEQ ID NOs: 86-88, SEQ ID NOs: 90-92, SEQ ID NOs: 233-286, or SEQ ID NOs: 291-293 or a binding protein having CDRs with 80% or greater identity to the CDRs of the binding protein comprising SEQ ID NOs: 70-72, SEQ ID NOs: 74-76, SEQ ID NOs: 78-80, SEQ ID NOs: 82-84, SEQ ID NOs: 86-88 or SEQ ID NOs: 90-92, SEQ ID NOs: 233-286, or SEQ ID NOs: 291-293 and the antigen binding specificity thereof.

Multispecific binding proteins

The disclosure provides binding proteins that binds to more than one target proteins, i.e., multispecific binding proteins. The multispecific binding proteins can include any one of the binding proteins described herein.

An embodiment provides a multispecific binding protein that selectively and/or specifically binds to human sIL-6R and to human CTGF.

The terms “multispecific compound” or multispecific binding protein” refers to a “fusion molecule” or “fusion protein” and refer to a biologically active polypeptide, including two or more binding protein portions, with or without a further effector molecule, covalently linked (i.e., fused) by recombinant, chemical or other suitable method. For example, the one or more binding proteins of a multispecific compound can be linked to one another through a peptide linker sequence. Alternatively, the peptide linker may be used to assist in the construction of the fusion molecule. Fusion molecules can be fusion proteins. Generally, a fusion molecule can also include conjugate molecules.

A multispecific binding protein can include a binding protein that selectively and/or specifically binds sIL-6R and a binding protein that selectively and/or specifically binds CTGF. The binding protein that selectively and/or specifically binds sIL-6R can be any of the sIL-6R binding proteins described herein, and the binding protein that selectively and/or specifically binds CTGF can be any of the CTGF binding proteins described herein. That is the multispecific binding proteins can include any combination CDRs sequences that define the binding proteins that target SIL-6R and CTGF.

For example, the multispecific binding protein can comprise CDRs as set forth in:
(i) SEQ ID NOs: 2-4 and SEQ ID NOs: 70-72, SEQ ID NOs: 6-8 and SEQ ID NOs: 70-72, SEQ ID NOs: 10-12 and SEQ ID NOs: 70-72, SEQ ID NOs: 14-16 and SEQ ID NOs: 70-72, SEQ ID NOs: 18-20 and SEQ ID NOs: 70-72, SEQ ID NOs: 22-24 and SEQ ID NOs: 70-72, SEQ ID NOs: 26-28 and SEQ ID NOs: 70-72, SEQ ID NOs: 30-32 and SEQ ID NOs: 70-72, SEQ ID NOs: 34-36 and SEQ ID NOs: 70-72, SEQ ID NOs: 38-40 and SEQ ID NOs: 70-72, SEQ ID NOs: 42-44 and SEQ ID NOs: 70-72, SEQ ID NOs: 46-48 and SEQ ID NOs: 70-72, SEQ ID NOs: 50-52 and SEQ ID NOs: 70-72, SEQ ID NOs: 54-56 and SEQ ID NOs: 70-72, SEQ ID NOs: 58-60 and SEQ ID NOs: 70-72, SEQ ID NOs: 62-64 and SEQ ID NOs: 70-72 or SEQ ID NOs: 66-68 and SEQ ID NOs: 70-72 (furthermore any of the sIL-6R CDR sets shown in SEQ ID NOs: 138-194 can similarly be combined with SEQ ID NO: 70-72 and any of the CTGF CDR sets as set forth in SEQ ID NOs: 233-286 can be used in place of SEQ ID NOs: 70-72);
(ii) SEQ ID NOs: 2-4 and SEQ ID NOs: 74-76, SEQ ID NOs: 6-8 and SEQ ID NOs: 74-76, SEQ ID NOs: 10-12 and SEQ ID NOs: 74-76, SEQ ID NOs: 14-16 and SEQ ID NOs: 74-76, SEQ ID NOs: 18-20 and SEQ ID NOs: 74-76, SEQ ID NOs: 22-24 and SEQ ID NOs: 74-76, SEQ ID NOs: 26-28 and SEQ ID NOs: 74-76, SEQ ID NOs: 30-32 and SEQ ID NOs: 74-76, SEQ ID NOs: 34-36 and SEQ ID NOs: 74-76, SEQ ID NOs: 38-40 and SEQ ID NOs: 74-76, SEQ ID NOs: 42-44 and SEQ ID NOs: 74-76, SEQ ID NOs: 46-48 and SEQ ID NOs: 74-76, SEQ ID NOs: 50-52 and SEQ ID NOs: 74-76, SEQ ID NOs: 54-56 and SEQ ID NOs: 74-76, SEQ ID NOs: 58-60 and SEQ ID NOs: 74-76, SEQ ID NOs: 62-64 and SEQ ID NOs: 74-76 or SEQ ID NOs: 66-68 and SEQ ID NOs: 74-76; (furthermore any of the sIL-6R CDR sets shown in SEQ ID NOs: 138-194 can similarly be combined with SEQ ID NO:74-76 and any of the CTGF CDR sets as set forth in SEQ ID NOs: 233-286 can be used in place of SEQ ID NOs: 74-76);
(iii) SEQ ID NOs: 2-4 and SEQ ID NOs: 78-80, SEQ ID NOs: 6-8 and SEQ ID NOs: 78-80, SEQ ID NOs: 10-12 and SEQ ID NOs: 78-80, SEQ ID NOs: 14-16 and SEQ ID NOs: 78-80, SEQ ID NOs: 18-20 and SEQ ID NOs: 78-80, SEQ ID NOs: 22-24 and SEQ ID NOs: 78-80, SEQ ID NOs: 26-28 and SEQ ID NOs: 78-80, SEQ ID NOs: 30-32 and SEQ ID NOs: 78-80, SEQ ID NOs: 34-36 and SEQ ID NOs: 78-80, SEQ ID NOs: 38-40 and SEQ ID NOs: 78-80, SEQ ID NOs: 42-44 and SEQ ID NOs: 78-80, SEQ ID NOs: 46-48 and SEQ ID NOs: 78-80, SEQ ID NOs: 50-52 and SEQ ID NOs: 78-80, SEQ ID NOs: 54-56 and SEQ ID NOs: 78-80, SEQ ID NOs: 58-60 and SEQ ID NOs: 78-80, SEQ ID NOs: 62-64 and SEQ ID NOs: 78-80 or SEQ ID NOs: 66-68 and SEQ ID NOs: 78-80 (furthermore any of the sIL-6R CDR sets shown in SEQ ID NOs: 138-194 can similarly be combined with SEQ ID NO: 78-80 and any of the CTGF CDR sets as set forth in SEQ ID NOs: 233-286 can be used in place of SEQ ID NOs: 78-80);
(iv) SEQ ID NOs: 2-4 and SEQ ID NOs: 82-84, SEQ ID NOs: 6-8 and SEQ ID NOs: 82-84, SEQ ID NOs: 10-12 and SEQ ID NOs: 82-84, SEQ ID NOs: 14-16 and SEQ ID NOs: 82-84, SEQ ID NOs: 18-20 and SEQ ID NOs: 82-84, SEQ ID NOs: 22-24 and SEQ ID NOs: 82-84, SEQ ID NOs: 26-28 and SEQ ID NOs: 82-84, SEQ ID NOs: 30-32 and SEQ ID NOs: 82-84, SEQ ID NOs: 34-36 and SEQ ID NOs: 82-84, SEQ ID NOs: 38-40 and SEQ ID NOs: 82-84, SEQ ID NOs: 42-44 and SEQ ID NOs: 82-84, SEQ ID NOs: 46-48 and SEQ ID NOs: 82-84, SEQ ID NOs: 50-52 and SEQ ID NOs: 82-84, SEQ ID NOs: 54-56 and SEQ ID NOs: 82-84, SEQ ID NOs: 58-60 and SEQ ID NOs: 82-84, SEQ ID NOs: 62-64 and SEQ ID NOs: 82-84 or SEQ ID NOs: 66-68 and SEQ ID NOs: 82-84 (furthermore any of the sIL-6R CDR sets shown in SEQ ID NOs: 138-194 can similarly be combined with SEQ ID NO: 82-84 and any of the CTGF CDR sets as set forth in SEQ ID NOs: 233-286 can be used in place of SEQ ID NOs: 82-84);
(v) SEQ ID NOs: 2-4 and SEQ ID NOs: 86-88, SEQ ID NOs: 6-8 and SEQ ID NOs: 86-88, SEQ ID NOs: 10-12 and SEQ ID NOs: 86-88, SEQ ID NOs: 14-16 and SEQ ID NOs: 86-88, SEQ ID NOs: 18-20 and SEQ ID NOs: 86-88, SEQ ID NOs: 22-24 and SEQ ID NOs: 86-88, SEQ ID NOs: 26-28 and SEQ ID NOs: 86-88, SEQ ID NOs: 30-32 and SEQ ID NOs: 86-88, SEQ ID NOs: 34-36 and SEQ ID NOs: 86-88, SEQ ID NOs: 38-40 and SEQ ID NOs: 86-88, SEQ ID NOs: 42-44 and SEQ ID NOs: 86-88, SEQ ID NOs: 46-48 and SEQ ID NOs: 86-88, SEQ ID NOs: 50-52 and SEQ ID NOs: 86-88, SEQ ID NOs: 54-56 and SEQ ID NOs: 86-88, SEQ ID NOs: 58-60 and SEQ ID NOs: 86-88, SEQ ID NOs: 62-64 and SEQ ID NOs: 86-88 or SEQ ID NOs: 66-68 and SEQ ID NOs: 86-88 (furthermore any of the sIL-6R CDR sets shown in SEQ ID NOs: 138-194 can similarly be combined with SEQ ID NO: 86-88 and any of the CTGF CDR sets as set forth in SEQ ID NOs: 233-286 can be used in place of SEQ ID NOs: 86-88); or
(vi) SEQ ID NOs: 2-4 and SEQ ID NOs: 90-92, SEQ ID NOs: 6-8 and SEQ ID NOs: 90-92, SEQ ID NOs: 10-12 and SEQ ID NOs: 90-92, SEQ ID NOs: 14-16 and SEQ ID NOs: 90-92, SEQ ID NOs: 18-20 and SEQ ID NOs: 90-92, SEQ ID NOs: 22-24 and SEQ ID NOs: 90-92, SEQ ID NOs: 26-28 and SEQ ID NOs: 90-92, SEQ ID NOs: 30-32 and SEQ ID NOs: 90-92, SEQ ID NOs: 34-36 and SEQ ID NOs: 90-92, SEQ ID NOs: 38-40 and SEQ ID NOs: 90-92, SEQ ID NOs: 42-44 and SEQ ID NOs: 90-92, SEQ ID NOs: 46-48 and SEQ ID NOs: 90-92, SEQ ID NOs: 50-52 and SEQ ID NOs: 90-92, SEQ ID NOs: 54-56 and SEQ ID NOs: 90-92, SEQ ID NOs: 58-60 and SEQ ID NOs: 90-92, SEQ ID NOs: 62-64 and SEQ ID NOs: 90-92 or SEQ ID NOs: 66-68 and SEQ ID NOs: 90-92 (furthermore any of the sIL-6R CDR sets shown in SEQ ID NOs: 138-194 can similarly be combined with SEQ ID NO: 90-92 and any of the CTGF CDR sets as set forth in SEQ ID NOs: 233-286 can be used in place of SEQ ID NOs: 90-92).

A multispecific binding protein can comprise the amino acid sequence of:
(i) SEQ ID NO: 1 and SEQ ID NO:69, SEQ ID NO:5 and SEQ ID NO:69, SEQ ID NO: 9 and SEQ ID NO:69, SEQ ID NO: 13 and SEQ ID NO:69, SEQ ID NO: 17 and SEQ ID NO:69, SEQ ID NO: 21 and SEQ ID NO: 69, SEQ ID NO:25 and SEQ ID NO: 69, SEQ ID NO:29 and SEQ ID NO:69, SEQ ID NO:33 and SEQ ID NO:69, SEQ ID NO:37 and SEQ ID NO:69, SEQ ID NO:41 and SEQ ID NO: 69, SEQ ID NO: 45 and SEQ ID NO:69, SEQ ID NO:49 and SEQ ID NO:69, SEQ ID NO:53 and SEQ ID NO:69, SEQ ID NO:57 and SEQ ID NO:69, SEQ ID NO:61 and SEQ ID NO:69, or SEQ ID NO:65 and SEQ ID NO:69 (furthermore any of the sIL-6R VHHs shown in SEQ ID NOs: 119-137 can similarly be combined with SEQ ID NO:69 and any of SEQ ID NO:214-232 can be used in place of SEQ ID NO:69);
(ii) SEQ ID NO:1 and SEQ ID NO:73, SEQ ID NO:5 and SEQ ID NO:73, SEQ ID NO:9 and SEQ ID NO:73, SEQ ID NO: 13 and SEQ ID NO:73, SEQ ID NO: 17 and SEQ ID NO:73, SEQ ID NO: 21 and SEQ ID NO:73, SEQ ID NO:25 and SEQ ID NO:73, SEQ ID NO:29 and SEQ ID NO: 73, SEQ ID NO:33 and SEQ ID NO:73, SEQ ID NO:37 and SEQ ID NO:73, SEQ ID NO:41 and SEQ ID NO:73, SEQ ID NO:45 and SEQ ID NO:73, SEQ ID NO:49 and SEQ ID NO:73, SEQ ID NO: 53 and SEQ ID NO:73, SEQ ID NO:57 and SEQ ID NO:73, SEQ ID NO:61 and SEQ ID NO: 73, or SEQ ID NO: 65 and SEQ ID NO: 73 (furthermore any of the sIL-6R VHHs shown in SEQ ID NOs: 119-137 can similarly be combined with SEQ ID NO:73 and any of SEQ ID NO:214-232 can be used in place of SEQ ID NO:73);
(iii) SEQ ID NO:1 and SEQ ID NO:77, SEQ ID NO:5 and SEQ ID NO:77, SEQ ID NO:9 and SEQ ID NO:77, SEQ ID NO: 13 and SEQ ID NO:77, SEQ ID NO: 17 and SEQ ID NO:77, SEQ ID NO: 21 and SEQ ID NO:77, SEQ ID NO:25 and SEQ ID NO:77, SEQ ID NO:29 and SEQ ID NO: 77, SEQ ID NO:33 and SEQ ID NO:77, SEQ ID NO:37 and SEQ ID NO:77, SEQ ID NO:41 and SEQ ID NO:77, SEQ ID NO:45 and SEQ ID NO:77, SEQ ID NO:49 and SEQ ID NO:77, SEQ ID NO: 53 and SEQ ID NO:77, SEQ ID NO:57 and SEQ ID NO:77, SEQ ID NO:61 and SEQ ID NO: 77, or SEQ ID NO: 65 and SEQ ID NO:77 (furthermore any of the sIL-6R VHHs shown in SEQ ID NOs: 119-137 can similarly be combined with SEQ ID NO:77 and any of SEQ ID NO:214-232 can be used in place of SEQ ID NO:77);
(iv) SEQ ID NO: 1 and SEQ ID NO:81, SEQ ID NO:5 and SEQ ID NO:81, SEQ ID NO:9 and SEQ ID NO:81, SEQ ID NO: 13 and SEQ ID NO:81, SEQ ID NO: 17 and SEQ ID NO:81, SEQ ID NO: 21 and SEQ ID NO:81, SEQ ID NO:25 and SEQ ID NO:81, SEQ ID NO:29 and SEQ ID NO: 81, SEQ ID NO:33 and SEQ ID NO:81, SEQ ID NO:37 and SEQ ID NO:81, SEQ ID NO:41 and SEQ ID NO: 81, SEQ ID NO:45 and SEQ ID NO:81, SEQ ID NO:49 and SEQ ID NO:81, SEQ ID NO: 53 and SEQ ID NO:81, SEQ ID NO:57 and SEQ ID NO:81, SEQ ID NO:61 and SEQ ID NO: 81, or SEQ ID NO: 65 and SEQ ID NO: 81 (furthermore any of the sIL-6R VHHs shown in SEQ ID NOs: 119-137 can similarly be combined with SEQ ID NO:81 and any of SEQ ID NO:214-232 can be used in place of SEQ ID NO:81);
(v) SEQ ID NO:1 and SEQ ID NO:85, SEQ ID NO:5 and SEQ ID NO:85, SEQ ID NO:9 and SEQ ID NO:85, SEQ ID NO: 13 and SEQ ID NO:85, SEQ ID NO: 17 and SEQ ID NO:85, SEQ ID NO: 21 and SEQ ID NO:85, SEQ ID NO:25 and SEQ ID NO:85, SEQ ID NO:29 and SEQ ID NO: 85, SEQ ID NO:33 and SEQ ID NO:85, SEQ ID NO:37 and SEQ ID NO:85, SEQ ID NO:41 and SEQ ID NO: 85, SEQ ID NO:45 and SEQ ID NO:85, SEQ ID NO:49 and SEQ ID NO:85, SEQ ID NO: 53 and SEQ ID NO:85, SEQ ID NO:57 and SEQ ID NO:85, SEQ ID NO:61 and SEQ ID NO: 85, or SEQ ID NO: 65 and SEQ ID NO: 85 (furthermore any of the sIL-6R VHHs shown in SEQ ID NOs: 119-137 can similarly be combined with SEQ ID NO:85 and any of SEQ ID NO:214-232 can be used in place of SEQ ID NO:85); or
(vi) SEQ ID NO:1 and SEQ ID NO:89, SEQ ID NO:5 and SEQ ID NO:89, SEQ ID NO:9 and SEQ ID NO: 89, SEQ ID NO: 13 and SEQ ID NO:89, SEQ ID NO: 17 and SEQ ID NO:89, SEQ ID NO: 21 and SEQ ID NO:89, SEQ ID NO:25 and SEQ ID NO:89, SEQ ID NO:29 and SEQ ID NO: 89, SEQ ID NO:33 and SEQ ID NO:89, SEQ ID NO:37 and SEQ ID NO:89, SEQ ID NO:41 and SEQ ID NO: 89, SEQ ID NO:45 and SEQ ID NO:89, SEQ ID NO:49 and SEQ ID NO:89, SEQ ID NO: 53 and SEQ ID NO:89, SEQ ID NO:57 and SEQ ID NO:89, SEQ ID NO:61 and SEQ ID NO: 89, or SEQ ID NO: 65 and SEQ ID NO: 89 (furthermore any of the sIL-6R VHHs shown in SEQ ID NOs: 119-137 can similarly be combined with SEQ ID NO:89 and any of SEQ ID NO:214-232 can be used in place of SEQ ID NO:89).

A multispecific binding protein can have a better binding affinity to soluble IL-6R as compared to a binding affinity to membrane bound IL-6R.

### Fusion Proteins

An embodiment provides a fusion protein including a first binding protein that selectively and/or specifically binds to human sIL-6R linked to a second binding protein that selectively and/or specifically binds to human CTGF, and optionally an additional binding protein.

For example, a fusion protein can include any of the binding proteins described herein, or any of the multispecific binding proteins described herein, each of which can further include an additional binding protein.

The first binding protein and the second binding protein can be VHH antibodies The VHH antibodies can comprise CDRs as set forth in:
(i) SEQ ID NOs: 2-4 and SEQ ID NOs: 70-72, SEQ ID NOs: 6-8 and SEQ ID NOs: 70-72, SEQ ID NOs: 10-12 and SEQ ID NOs: 70-72, SEQ ID NOs: 14-16 and SEQ ID NOs: 70-72, SEQ ID NOs: 18-20 and SEQ ID NOs: 70-72, SEQ ID NOs: 22-24 and SEQ ID NOs: 70-72, SEQ ID NOs: 26-28 and SEQ ID NOs: 70-72, SEQ ID NOs: 30-32 and SEQ ID NOs: 70-72, SEQ ID NOs: 34-36 and SEQ ID NOs: 70-72, SEQ ID NOs: 38-40 and SEQ ID NOs: 70-72, SEQ ID NOs: 42-44 and SEQ ID NOs: 70-72, SEQ ID NOs: 46-48 and SEQ ID NOs: 70-72, SEQ ID NOs: 50-52 and SEQ ID NOs: 70-72, SEQ ID NOs: 54-56 and SEQ ID NOs: 70-72, SEQ ID NOs: 58-60 and SEQ ID NOs: 70-72, SEQ ID NOs: 62-64 and SEQ ID NOs: 70-72 or SEQ ID NOs: 66-68 and SEQ ID NOs: 70-72 (furthermore any of the sIL-6R CDR sets shown in SEQ ID NOs: 138-194 can similarly be combined with SEQ ID NO: 70-72 and any of the CTGF CDR sets as set forth in SEQ ID NOs: 233-286 can be used in place of SEQ ID NOs: 70-72);
(ii) SEQ ID NOs: 2-4 and SEQ ID NOs: 74-76, SEQ ID NOs: 6-8 and SEQ ID NOs: 74-76, SEQ ID NOs: 10-12 and SEQ ID NOs: 74-76, SEQ ID NOs: 14-16 and SEQ ID NOs: 74-76, SEQ ID NOs: 18-20 and SEQ ID NOs: 74-76, SEQ ID NOs: 22-24 and SEQ ID NOs: 74-76, SEQ ID NOS: 26-28 and SEQ ID NOs: 74-76, SEQ ID NOs: 30-32 and SEQ ID NOs: 74-76, SEQ ID NOs: 34-36 and SEQ ID NOs: 74-76, SEQ ID NOs: 38-40 and SEQ ID NOs: 74-76, SEQ ID NOs: 42-44 and SEQ ID NOs: 74-76, SEQ ID NOs: 46-48 and SEQ ID NOs: 74-76, SEQ ID NOs: 50-52 and SEQ ID NOs: 74-76, SEQ ID NOs: 54-56 and SEQ ID NOs: 74-76, SEQ ID NOs: 58-60 and SEQ ID NOs: 74-76, SEQ ID NOs: 62-64 and SEQ ID NOs: 74-76 or SEQ ID NOs: 66-68 and SEQ ID NOs: 74-76 (furthermore any of the sIL-6R CDR sets shown in SEQ ID NOs: 138-194 can similarly be combined with SEQ ID NO: 74-76 and any of the CTGF CDR sets as set forth in SEQ ID NOs: 233-286 can be used in place of SEQ ID NOs: 74-76);
(iii) SEQ ID NOs: 2-4 and SEQ ID NOs: 78-80, SEQ ID NOs: 6-8 and SEQ ID NOs: 78-80, SEQ ID NOs: 10-12 and SEQ ID NOs: 78-80, SEQ ID NOs: 14-16 and SEQ ID NOs: 78-80, SEQ ID NOs: 18-20 and SEQ ID NOs: 78-80, SEQ ID NOs: 22-24 and SEQ ID NOs: 78-80, SEQ ID NOS: 26-28 and SEQ ID NOs: 78-80, SEQ ID NOs: 30-32 and SEQ ID NOs: 78-80, SEQ ID NOs: 34-36 and SEQ ID NOs: 78-80, SEQ ID NOs: 38-40 and SEQ ID NOs: 78-80, SEQ ID NOs: 42-44 and SEQ ID NOs: 78-80, SEQ ID NOs: 46-48 and SEQ ID NOs: 78-80, SEQ ID NOs: 50-52 and SEQ ID NOs: 78-80, SEQ ID NOs: 54-56 and SEQ ID NOs: 78-80, SEQ ID NOs: 58-60 and SEQ ID NOs: 78-80, SEQ ID NOs: 62-64 and SEQ ID NOs: 78-80 or SEQ ID NOs: 66-68 and SEQ ID NOs: 78-80 (furthermore any of the sIL-6R CDR sets shown in SEQ ID NOs: 138-194 can similarly be combined with SEQ ID NO: 78-80 and any of the CTGF CDR sets as set forth in SEQ ID NOs: 233-286 can be used in place of SEQ ID NOs: 78-80);
(iv) SEQ ID NOs: 2-4 and SEQ ID NOs: 82-84, SEQ ID NOs: 6-8 and SEQ ID NOs: 82-84, SEQ ID NOs: 10-12 and SEQ ID NOs: 82-84, SEQ ID NOs: 14-16 and SEQ ID NOs: 82-84, SEQ ID NOs: 18-20 and SEQ ID NOs: 82-84, SEQ ID NOs: 22-24 and SEQ ID NOs: 82-84, SEQ ID NOs: 26-28 and SEQ ID NOs: 82-84, SEQ ID NOs: 30-32 and SEQ ID NOs: 82-84, SEQ ID NOs: 34-36 and SEQ ID NOs: 82-84, SEQ ID NOs: 38-40 and SEQ ID NOs: 82-84, SEQ ID NOs: 42-44 and SEQ ID NOs: 82-84, SEQ ID NOs: 46-48 and SEQ ID NOs: 82-84, SEQ ID NOs: 50-52 and SEQ ID NOs: 82-84, SEQ ID NOs: 54-56 and SEQ ID NOs: 82-84, SEQ ID NOs: 58-60 and SEQ ID NOs: 82-84, SEQ ID NOs: 62-64 and SEQ ID NOs: 82-84 or SEQ ID NOs: 66-68 and SEQ ID NOs: 82-84 (furthermore any of the sIL-6R CDR sets shown in SEQ ID NOs: 138-194 can similarly be combined with SEQ ID NO: 82-84 and any of the CTGF CDR sets as set forth in SEQ ID NOs: 233-286 can be used in place of SEQ ID NOs: 82-84);
(v) SEQ ID NOs: 2-4 and SEQ ID NOs: 86-88, SEQ ID NOs: 6-8 and SEQ ID NOs: 86-88, SEQ ID NOs: 10-12 and SEQ ID NOs: 86-88, SEQ ID NOs: 14-16 and SEQ ID NOs: 86-88, SEQ ID NOs: 18-20 and SEQ ID NOs: 86-88, SEQ ID NOs: 22-24 and SEQ ID NOs: 86-88, SEQ ID NOs: 26-28 and SEQ ID NOs: 86-88, SEQ ID NOs: 30-32 and SEQ ID NOs: 86-88, SEQ ID NOs: 34-36 and SEQ ID NOs: 86-88, SEQ ID NOs: 38-40 and SEQ ID NOs: 86-88, SEQ ID NOs: 42-44 and SEQ ID NOs: 86-88, SEQ ID NOs: 46-48 and SEQ ID NOs: 86-88, SEQ ID NOs: 50-52 and SEQ ID NOs: 86-88, SEQ ID NOs: 54-56 and SEQ ID NOs: 86-88, SEQ ID NOs: 58-60 and SEQ ID NOs: 86-88, SEQ ID NOs: 62-64 and SEQ ID NOs: 86-88 or SEQ ID NOs: 66-68 and SEQ ID NOs: 86-88 (furthermore any of the sIL-6R CDR sets shown in SEQ ID NOs: 138-194 can similarly be combined with SEQ ID NO: 86-88 and any of the CTGF CDR sets as set forth in SEQ ID NOs: 233-286 can be used in place of SEQ ID NOs: 86-88); or
(vi) SEQ ID NOs: 2-4 and SEQ ID NOs: 90-92, SEQ ID NOs: 6-8 and SEQ ID NOs: 90-92, SEQ ID NOs: 10-12 and SEQ ID NOs: 90-92, SEQ ID NOs: 14-16 and SEQ ID NOs: 90-92, SEQ ID NOs: 18-20 and SEQ ID NOs: 90-92, SEQ ID NOs: 22-24 and SEQ ID NOs: 90-92, SEQ ID NOs: 26-28 and SEQ ID NOs: 90-92, SEQ ID NOs: 30-32 and SEQ ID NOs: 90-92, SEQ ID NOs: 34-36 and SEQ ID NOs: 90-92, SEQ ID NOs: 38-40 and SEQ ID NOs: 90-92, SEQ ID NOs: 42-44 and SEQ ID NOs: 90-92, SEQ ID NOs: 46-48 and SEQ ID NOs: 90-92, SEQ ID NOs: 50-52 and SEQ ID NOs: 90-92, SEQ ID NOs: 54-56 and SEQ ID NOs: 90-92, SEQ ID NOs: 58-60 and SEQ ID NOs: 90-92, SEQ ID NOs: 62-64 and SEQ ID NOs: 90-92 or SEQ ID NOs: 66-68 and SEQ ID NOs: 90-92 (furthermore any of the sIL-6R CDR sets shown in SEQ ID NOs: 138-194 can similarly be combined with SEQ ID NO: 90-92 and any of the CTGF CDR sets as set forth in SEQ ID NOs: 233-286 can be used in place of SEQ ID NOs: 90-92.

A fusion protein can comprise the amino acid sequence of:
(i) SEQ ID NO: 1 and SEQ ID NO:69, SEQ ID NO:5 and SEQ ID NO:69, SEQ ID NO:9 and SEQ ID NO:69, SEQ ID NO: 13 and SEQ ID NO:69, SEQ ID NO: 17 and SEQ ID NO:69, SEQ ID NO: 21 and SEQ ID NO: 69, SEQ ID NO:25 and SEQ ID NO: 69, SEQ ID NO:29 and SEQ ID NO:69, SEQ ID NO:33 and SEQ ID NO:69, SEQ ID NO:37 and SEQ ID NO:69, SEQ ID NO:41 and SEQ ID NO: 69, SEQ ID NO:45 and SEQ ID NO:69, SEQ ID NO:49 and SEQ ID NO:69, SEQ ID NO:53 and SEQ ID NO:69, SEQ ID NO:57 and SEQ ID NO:69, SEQ ID NO:61 and SEQ ID NO:69, or SEQ ID NO:65 and SEQ ID NO:69 (furthermore any of the sIL-6R VHHs shown in SEQ ID NOs: 119-137 can similarly be combined with SEQ ID NO:69 and any of SEQ ID NO:214-232 can be used in place of SEQ ID NO:69);
(ii) SEQ ID NO:1 and SEQ ID NO:73, SEQ ID NO:5 and SEQ ID NO:73, SEQ ID NO:9 and SEQ ID NO: 73, SEQ ID NO: 13 and SEQ ID NO:73, SEQ ID NO: 17 and SEQ ID NO:73, SEQ ID NO: 21 and SEQ ID NO:73, SEQ ID NO:25 and SEQ ID NO:73, SEQ ID NO:29 and SEQ ID NO: 73, SEQ ID NO:33 and SEQ ID NO:73, SEQ ID NO:37 and SEQ ID NO:73, SEQ ID NO:41 and SEQ ID NO:73, SEQ ID NO:45 and SEQ ID NO:73, SEQ ID NO:49 and SEQ ID NO:73, SEQ ID NO: 53 and SEQ ID NO:73, SEQ ID NO:57 and SEQ ID NO:73, SEQ ID NO:61 and SEQ ID NO: 73, or SEQ ID NO: 65 and SEQ ID NO: 73 (furthermore any of the sIL-6R VHHs shown in SEQ ID NOs: 119-137 can similarly be combined with SEQ ID NO:73 and any of SEQ ID NO:214-232 can be used in place of SEQ ID NO:73);
(iii) SEQ ID NO:1 and SEQ ID NO:77, SEQ ID NO:5 and SEQ ID NO:77, SEQ ID NO:9 and SEQ ID NO:77, SEQ ID NO: 13 and SEQ ID NO:77, SEQ ID NO: 17 and SEQ ID NO:77, SEQ ID NO: 21 and SEQ ID NO:77, SEQ ID NO:25 and SEQ ID NO:77, SEQ ID NO:29 and SEQ ID NO: 77, SEQ ID NO:33 and SEQ ID NO:77, SEQ ID NO:37 and SEQ ID NO:77, SEQ ID NO:41 and SEQ ID NO:77, SEQ ID NO:45 and SEQ ID NO:77, SEQ ID NO:49 and SEQ ID NO:77, SEQ ID NO: 53 and SEQ ID NO:77, SEQ ID NO:57 and SEQ ID NO:77, SEQ ID NO:61 and SEQ ID NO: 77, or SEQ ID NO: 65 and SEQ ID NO: 77 (furthermore any of the sIL-6R VHHs shown in SEQ ID NOs: 119-137 can similarly be combined with SEQ ID NO:77 and any of SEQ ID NO:214-232 can be used in place of SEQ ID NO:77);
(iv) SEQ ID NO:1 and SEQ ID NO:81, SEQ ID NO:5 and SEQ ID NO:81, SEQ ID NO:9 and SEQ ID NO:81, SEQ ID NO: 13 and SEQ ID NO:81, SEQ ID NO: 17 and SEQ ID NO:81, SEQ ID NO: 21 and SEQ ID NO:81, SEQ ID NO:25 and SEQ ID NO:81, SEQ ID NO:29 and SEQ ID NO: 81, SEQ ID NO:33 and SEQ ID NO:81, SEQ ID NO:37 and SEQ ID NO:81, SEQ ID NO:41 and SEQ ID NO:81, SEQ ID NO: 45 and SEQ ID NO:81, SEQ ID NO:49 and SEQ ID NO:81, SEQ ID NO: 53 and SEQ ID NO:81, SEQ ID NO:57 and SEQ ID NO:81, SEQ ID NO:61 and SEQ ID NO: 81, or SEQ ID NO: 65 and SEQ ID NO: 81 (furthermore any of the sIL-6R VHHs shown in SEQ ID NOs: 119-137 can similarly be combined with SEQ ID NO:81 and any of SEQ ID NO:214-232 can be used in place of SEQ ID NO:81);
(v) SEQ ID NO:1 and SEQ ID NO:85, SEQ ID NO:5 and SEQ ID NO:85, SEQ ID NO:9 and SEQ ID NO:85, SEQ ID NO: 13 and SEQ ID NO:85, SEQ ID NO: 17 and SEQ ID NO:85, SEQ ID NO: 21 and SEQ ID NO:85, SEQ ID NO:25 and SEQ ID NO:85, SEQ ID NO:29 and SEQ ID NO: 85, SEQ ID NO:33 and SEQ ID NO:85, SEQ ID NO:37 and SEQ ID NO:85, SEQ ID NO:41 and SEQ ID NO: 85, SEQ ID NO: 45 and SEQ ID NO:85, SEQ ID NO:49 and SEQ ID NO:85, SEQ ID NO: 53 and SEQ ID NO:85, SEQ ID NO:57 and SEQ ID NO:85, SEQ ID NO:61 and SEQ ID NO: 85, or SEQ ID NO: 65 and SEQ ID NO: 85 (furthermore any of the sIL-6R VHHs shown in SEQ ID NOs: 119-137 can similarly be combined with SEQ ID NO:85 and any of SEQ ID NO:214-232 can be used in place of SEQ ID NO:85);
(vi) SEQ ID NO:1 and SEQ ID NO:89, SEQ ID NO:5 and SEQ ID NO:89, SEQ ID NO:9 and SEQ ID NO: 89, SEQ ID NO: 13 and SEQ ID NO: 89, SEQ ID NO: 17 and SEQ ID NO: 89, SEQ ID NO: 21 and SEQ ID NO: 89, SEQ ID NO:25 and SEQ ID NO:89, SEQ ID NO:29 and SEQ ID NO: 89, SEQ ID NO:33 and SEQ ID NO:89, SEQ ID NO:37 and SEQ ID NO:89, SEQ ID NO:41 and SEQ ID NO: 89, SEQ ID NO:45 and SEQ ID NO:89, SEQ ID NO:49 and SEQ ID NO:89, SEQ ID NO: 53 and SEQ ID NO:89, SEQ ID NO:57 and SEQ ID NO:89, SEQ ID NO:61 and SEQ ID NO: 89, or SEQ ID NO: 65 and SEQ ID NO: 89 (furthermore any of the sIL-6R VHHs shown in SEQ ID NOs: 119-137 can similarly be combined with SEQ ID NO:89 and any of SEQ ID NO:214-232 can be used in place of SEQ ID NO:89).

The fusion proteins can have a better binding affinity to soluble IL-6R as compared to a binding affinity to membrane bound IL-6R.

In one aspect, the binding proteins described herein, the multispecific binding proteins, and the fusion proteins described herein can include at least one additional binding protein domain.

The at least one additional binding protein domain can be a binding protein domain or “targeting moiety” that specifically and/or selectively binds to an additional target of interest, such as a protein that can facilitate or enhance blood brain barrier crossing of the binding protein and/or multispecific protein. As used herein, “targeting moiety” refers to a molecule that has the ability to localize and bind to a specific molecule or cellular component. The targeting moiety can be an antibody, antibody fragment, scFv, Fc-containing polypeptide, fusion antibody, polypeptide, peptide, aptamer, ligand, nucleic acid, or any combination thereof. In one embodiment, a targeting moiety can bind to a molecule present in a cell or tissue. In one aspect, the targeting molecule can bind a normal cell or tissue.

In such example, the binding multiprotein can be: (i) a bispecific binding protein that can recognize and selectively and/or specifically bind to a sIL-6R and/or to a cell that expresses the additional target protein; (ii) a bispecific binding protein that can recognize and selectively and/or specifically bind to a cell that expresses CTGF and/or to a cell that expresses the additional target protein; (iii) a multispecific binding protein that can recognize and selectively and/or specifically bind to a sIL-6R, a cell that expresses CTGF and/or to a cell that expresses the additional target protein; or (iv) a fusion protein that can include a sIL-6R binding protein, a CTGF binding protein and/or an additional target protein.

A transferrin receptor (TfR) is highly expressed by brain capillary endothelial cells (BCECs) forming the blood-brain barrier (BBB) and is therefore considered as a potential target for brain drug delivery. The brain has remained inaccessible to immunotherapies as a result of the barrier that separates it from the systemic circulation; the blood: brain barrier (BBB). Specialized tight junctions between the endothelial cells that make up the front line of the BBB mean that while a small amount of most antibodies will gain access to the brain after peripheral injection, this is reported to be as low as 0.1% of injected dose. Therefore, most antibodies would have to be administered at very high levels in order to produce a therapeutic response, which could lead to potentially toxic side effects.

Iron is an important molecule for brain function, but must be imported across the BBB for use by the cells of the brain, and transferrin receptor is the primary iron transporting protein. Iron-loaded transferrin receptor (holo-transferrin) binds to transferrin receptors on the surface of microvascular endothelial cells and is transported across the BBB to the brain. The transferrin receptor must transcytose from the blood to the brain side of the barrier to release its cargo.

An antibody is that binds to an endogenous receptor on the luminal face of the BBB endothelial cells (i.e., an anti-transferrin receptor antibody) can be internalized and transported through the cells, the antibody is transported alongside and then released on the abluminal side. Once the antibody has been established as a BBB-crossing technology, it can be used to shuttle therapeutic molecules into the brain to treat otherwise unreachable conditions. Monoclonal antibodies binding to the TfR can internalize into BCECs in vivo.

The transferrin receptor 1 (TfR1), also known as CD71, is a target for anti-transferrin receptor antibody-based therapy due to its high expression levels on the surface of BCECs and its ability to internalize. Any anti-CD71 antibody and any specific binding fragments thereof can be used herein. Non-limiting examples of commercially available anti-CD71 antibodies include monoclonal antibody clones H68.4, OKT9, R17217, 236-15375, 10F11, 3B82A1, MEM-189, MRC OX-26, MRQ-48, DF1513, ICO-92, RM384, MEM-75, YTA74.4, T56/14, TFRC 2898R, 3C11F11, 1B6, TFRC 3630, TRFC 1059, TRFC 1839, OTI6H9, 1E6H68.4, BGX.24, 66IG10 and JF0956. The additional target protein can be an anti-transferrin receptor antibody.

Any one of the binding proteins described herein can be conjugated to an anti-transferrin receptor antibody.

For example, any one of the sIL-6R binding protein as described herein, for example including CDRs as set forth in SEQ ID NOs: 2-4, SEQ ID NOS: 6-8, SEQ ID NOs: 10-12, SEQ ID NOs: 14-16, SEQ ID NOs: 18-20, SEQ ID NOs: 22-24, SEQ ID NOs: 26-28, SEQ ID NOs: 30-32, SEQ ID NOs: 34-36, SEQ ID NOs: 38-40, SEQ ID NOs: 42-44, SEQ ID NOs: 46-48, SEQ ID NOs: 50-52, SEQ ID NOs: 54-56, SEQ ID NOs: 58-60, SEQ ID NOS: 62-64, SEQ ID NOs: 66-68, or SEQ ID NO: 138-194 can further include an anti-transferrin receptor antibody.

In another example, any of the sIL-6R binding protein as described herein, for example including the amino acid sequence of SEQ ID NO:1, SEQ ID NO:5, SEQ ID NO:9, SEQ ID NO: 13, SEQ ID NO: 17, SEQ ID NO:21, SEQ ID NO:25, SEQ ID NO:29, SEQ ID NO:33, SEQ ID NO:37, SEQ ID NO:41, SEQ ID NO:45, SEQ ID NO:49, SEQ ID NO:53, SEQ ID NO:57, SEQ ID NO:61, SEQ ID NO:65, or SEQ ID NO: 119-137 can further include an anti-transferrin receptor antibody.

In an additional example, any CTGF binding protein as described herein, for example including CDRs as set forth in NOs: 70-72, SEQ ID NOs: 74-76, SEQ ID NOs: 78-80, SEQ ID NOs: 82-84, SEQ ID NOs: 86-88, SEQ ID NOs: 90-92, SEQ ID NOs: 233-286, or SEQ ID NOS: 291-293 can further include an anti-transferrin receptor antibody.

In a further example, any CTGF binding protein as described herein, for example including the amino acid sequence of SEQ ID NO:73, SEQ ID NO:77, SEQ ID NO:81, SEQ ID NO:85, SEQ ID NO: 89, or SEQ ID NO:214-232 can further include an anti-transferrin receptor antibody.

Any one of the multispecific binding proteins described herein can be conjugated to an anti-transferrin receptor antibody.

For example, any multispecific binding protein that selectively and/or specifically binds to human sIL-6R and to human CTGF as described herein, for example including CDRs as set forth in SEQ ID NOs: 2-4 and SEQ ID NOs: 70-72, SEQ ID NOs: 6-8 and SEQ ID NOs: 70-72, SEQ ID NOs: 10-12 and SEQ ID NOs: 70-72, SEQ ID NOs: 14-16 and SEQ ID NOs: 70-72, SEQ ID NOs: 18-20 and SEQ ID NOs: 70-72, SEQ ID NOs: 22-24 and SEQ ID NOs: 70-72, SEQ ID NOs: 26-28 and SEQ ID NOs: 70-72, SEQ ID NOs: 30-32 and SEQ ID NOs: 70-72, SEQ ID NOs: 34-36 and SEQ ID NOs: 70-72, SEQ ID NOs: 38-40 and SEQ ID NOs: 70-72, SEQ ID NOs: 42-44 and SEQ ID NOs: 70-72, SEQ ID NOs: 46-48 and SEQ ID NOs: 70-72, SEQ ID NOs: 50-52 and SEQ ID NOs: 70-72, SEQ ID NOs: 54-56 and SEQ ID NOs: 70-72, SEQ ID NOs: 58-60 and SEQ ID NOs: 70-72, SEQ ID NOs: 62-64 and SEQ ID NOs: 70-72; SEQ ID NOs: 66-68 and SEQ ID NOs: 70-72 (furthermore any of the sIL-6R CDR sets shown in SEQ ID NOs: 138-194 can similarly be combined with SEQ ID NO: 70-72 and any of the CTGF CDR sets as set forth in SEQ ID NOs: 233-286 can be used in place of SEQ ID NOs: 70-72); SEQ ID NOs: 2-4 and SEQ ID NOs: 74-76, SEQ ID NOs: 6-8 and SEQ ID NOs: 74-76, SEQ ID NOs: 10-12 and SEQ ID NOs: 74-76, SEQ ID NOs: 14-16 and SEQ ID NOs: 74-76, SEQ ID NOs: 18-20 and SEQ ID NOs: 74-76, SEQ ID NOs: 22-24 and SEQ ID NOs: 74-76, SEQ ID NOs: 26-28 and SEQ ID NOs: 74-76, SEQ ID NOs: 30-32 and SEQ ID NOs: 74-76, SEQ ID NOs: 34-36 and SEQ ID NOs: 74-76, SEQ ID NOs: 38-40 and SEQ ID NOs: 74-76, SEQ ID NOs: 42-44 and SEQ ID NOs: 74-76, SEQ ID NOs: 46-48 and SEQ ID NOs: 74-76, SEQ ID NOs: 50-52 and SEQ ID NOs: 74-76, SEQ ID NOs: 54-56 and SEQ ID NOs: 74-76, SEQ ID NOs: 58-60 and SEQ ID NOs: 74-76, SEQ ID NOs: 62-64 and SEQ ID NOs: 74-76; SEQ ID NOs: 66-68 and SEQ ID NOs: 74-76 (furthermore any of the sIL-6R CDR sets shown in SEQ ID NOs: 138-194 can similarly be combined with SEQ ID NO:74-76 and any of the CTGF CDR sets as set forth in SEQ ID NOS: 233-286 can be used in place of SEQ ID NOs: 74-76); SEQ ID NOs: 2-4 and SEQ ID NOs: 78-80, SEQ ID NOs: 6-8 and SEQ ID NOs: 78-80, SEQ ID NOs: 10-12 and SEQ ID NOs: 78-80, SEQ ID NOs: 14-16 and SEQ ID NOs: 78-80, SEQ ID NOs: 18-20 and SEQ ID NOs: 78-80, SEQ ID NOs: 22-24 and SEQ ID NOs: 78-80, SEQ ID NOs: 26-28 and SEQ ID NOs: 78-80, SEQ ID NOs: 30-32 and SEQ ID NOs: 78-80, SEQ ID NOs: 34-36 and SEQ ID NOs: 78-80, SEQ ID NOs: 38-40 and SEQ ID NOs: 78-80, SEQ ID NOs: 42-44 and SEQ ID NOs: 78-80, SEQ ID NOs: 46-48 and SEQ ID NOs: 78-80, SEQ ID NOs: 50-52 and SEQ ID NOs: 78-80, SEQ ID NOs: 54-56 and SEQ ID NOs: 78-80, SEQ

ID NOs: 58-60 and SEQ ID NOs: 78-80, SEQ ID NOs: 62-64 and SEQ ID NOs: 78-80; SEQ ID NOs: 66-68 and SEQ ID NOs: 78-80 (furthermore any of the sIL-6R CDR sets shown in SEQ ID NOs: 138-194 can similarly be combined with SEQ ID NO:78-80 and any of the CTGF CDR sets as set forth in SEQ ID NOs: 233-286 can be used in place of SEQ ID NOs: 78-80); SEQ ID NOs: 2-4 and SEQ ID NOs: 82-84, SEQ ID NOs: 6-8 and SEQ ID NOs: 82-84, SEQ ID NOs: 10-12 and SEQ ID NOs: 82-84, SEQ ID NOs: 14-16 and SEQ ID NOs: 82-84, SEQ ID NOs: 18-20 and SEQ ID NOs: 82-84, SEQ ID NOs: 22-24 and SEQ ID NOs: 82-84, SEQ ID NOs: 26-28 and SEQ ID NOs: 82-84, SEQ ID NOs: 30-32 and SEQ ID NOs: 82-84, SEQ ID NOs: 34-36 and SEQ ID NOs: 82-84, SEQ ID NOs: 38-40 and SEQ ID NOs: 82-84, SEQ ID NOs: 42-44 and SEQ ID NOs: 82-84, SEQ ID NOs: 46-48 and SEQ ID NOs: 82-84, SEQ ID NOs: 50-52 and SEQ ID NOs: 82-84, SEQ ID NOs: 54-56 and SEQ ID NOs: 82-84, SEQ ID NOs: 58-60 and SEQ ID NOs: 82-84, SEQ ID NOs: 62-64 and SEQ ID NOs: 82-84; SEQ ID NOs: 66-68 and SEQ ID NOs: 82-84 (furthermore any of the sIL-6R CDR sets shown in SEQ ID NOs: 138-194 can similarly be combined with SEQ ID NO:82-84 and any of the CTGF CDR sets as set forth in SEQ ID NOs: 233-286 can be used in place of SEQ ID NOs: 82-84); SEQ ID NOs: 2-4 and SEQ ID NOs: 86-88, SEQ ID NOs: 6-8 and SEQ ID NOs: 86-88, SEQ ID NOs: 10-12 and SEQ ID NOs: 86-88, SEQ ID NOs: 14-16 and SEQ ID NOs: 86-88, SEQ ID NOs: 18-20 and SEQ ID NOs: 86-88, SEQ ID NOs: 22-24 and SEQ ID NOs: 86-88, SEQ ID NOs: 26-28 and SEQ ID NOs: 86-88, SEQ ID NOs: 30-32 and SEQ ID NOs: 86-88, SEQ ID NOs: 34-36 and SEQ ID NOs: 86-88, SEQ ID NOs: 38-40 and SEQ ID NOs: 86-88, SEQ ID NOs: 42-44 and SEQ ID NOs: 86-88, SEQ ID NOs: 46-48 and SEQ ID NOs: 86-88, SEQ ID NOs: 50-52 and SEQ ID NOs: 86-88, SEQ ID NOs: 54-56 and SEQ ID NOs: 86-88, SEQ ID NOs: 58-60 and SEQ ID NOs: 86-88, SEQ ID NOs: 62-64 and SEQ ID NOs: 86-88; SEQ ID NOs: 66-68 and SEQ ID NOs: 86-88 (furthermore any of the sIL-6R CDR sets shown in SEQ ID NOs: 138-194 can similarly be combined with SEQ ID NO: 86-88 and any of the CTGF CDR sets as set forth in SEQ ID NOs: 233-286 can be used in place of SEQ ID NOs: 86-88); SEQ ID NOs: 2-4 and SEQ ID NOs: 90-92, SEQ ID NOs: 6-8 and SEQ ID NOs: 90-92, SEQ ID NOs: 10-12 and SEQ ID NOs: 90-92, SEQ ID NOs: 14-16 and SEQ ID NOs: 90-92, SEQ ID NOs: 18-20 and SEQ ID NOs: 90-92, SEQ ID NOs: 22-24 and SEQ ID NOs: 90-92, SEQ ID NOs: 26-28 and SEQ ID NOs: 90-92, SEQ ID NOs: 30-32 and SEQ ID NOs: 90-92, SEQ ID NOs: 34-36 and SEQ ID NOs: 90-92, SEQ ID NOs: 38-40 and SEQ ID NOs: 90-92, SEQ ID NOs: 42-44 and SEQ ID NOs: 90-92, SEQ ID NOs: 46-48 and SEQ ID NOs: 90-92, SEQ ID NOs: 50-52 and SEQ ID NOs: 90-92, SEQ ID NOs: 54-56 and SEQ ID NOs: 90-92, SEQ ID NOs: 58-60 and SEQ ID NOs: 90-92, SEQ ID NOs: 62-64 and SEQ ID NOs: 90-92 or SEQ ID NOs: 66-68 and SEQ ID NOs: 90-92 (furthermore any of the sIL-6R CDR sets shown in SEQ ID NOs: 138-194 can similarly be combined with SEQ ID NO: 90-92 and any of the CTGF CDR sets as set forth in SEQ ID NOs: 233-286 can be used in place of SEQ ID NOs: 90-92); can further include an anti-transferrin receptor antibody.

Anyone of the fusion proteins described herein can be conjugated to an anti-transferrin receptor antibody.

For example, any multispecific binding protein that selectively and/or specifically binds to human sIL-6R and to human CTGF as described herein, for example including the amino acid sequence of: SEQ ID NO: 1 and SEQ ID NO:69, SEQ ID NO:5 and SEQ ID NO:69, SEQ ID NO:9 and SEQ ID NO:69, SEQ ID NO: 13 and SEQ ID NO:69, SEQ ID NO: 17 and SEQ ID NO:69, SEQ ID NO: 21 and SEQ ID NO:69, SEQ ID NO:25 and SEQ ID NO:69, SEQ ID NO:29 and SEQ ID NO: 69, SEQ ID NO:33 and SEQ ID NO:69, SEQ ID NO:37 and SEQ ID NO:69, SEQ ID NO:41 and SEQ ID NO: 69, SEQ ID NO:45 and SEQ ID NO:69, SEQ ID NO:49 and SEQ ID NO:69, SEQ ID NO: 53 and SEQ ID NO:69, SEQ ID NO:57 and SEQ ID NO:69, SEQ ID NO:61 and SEQ ID NO: 69, SEQ ID NO: 65 and SEQ ID NO: 69 (furthermore any of the sIL-6R VHHs shown in SEQ ID NOs: 119-137 can similarly be combined with SEQ ID NO:69 and any of SEQ ID NO:214-232 can be used in place of SEQ ID NO:69); SEQ ID NO:1 and SEQ ID NO:73, SEQ ID NO:5 and SEQ ID NO:73, SEQ ID NO:9 and SEQ ID NO:73, SEQ ID NO: 13 and SEQ ID NO:73, SEQ ID NO: 17 and SEQ ID NO: 73, SEQ ID NO: 21 and SEQ ID NO: 73, SEQ ID NO:25 and SEQ ID NO:73, SEQ ID NO:29 and SEQ ID NO:73, SEQ ID NO:33 and SEQ ID NO:73, SEQ ID NO:37 and SEQ ID NO: 73, SEQ ID NO:41 and SEQ ID NO:73, SEQ ID NO: 45 and SEQ ID NO:73, SEQ ID NO:49 and SEQ ID NO:73, SEQ ID NO:53 and SEQ ID NO:73, SEQ ID NO:57 and SEQ ID NO:73, SEQ ID NO: 61 and SEQ ID NO:73, SEQ ID NO:65 and SEQ ID NO:73 (furthermore any of the sIL-6R VHHs shown in SEQ ID NOs: 119-137 can similarly be combined with SEQ ID NO:73 and any of SEQ ID NO:214-232 can be used in place of SEQ ID NO:73); SEQ ID NO:1 and SEQ ID NO:77, SEQ ID NO:5 and SEQ ID NO:77, SEQ ID NO:9 and SEQ ID NO:77, SEQ ID NO: 13 and SEQ ID NO: 77, SEQ ID NO: 17 and SEQ ID NO:77, SEQ ID NO:21 and SEQ ID NO:77, SEQ ID NO:25 and SEQ ID NO:77, SEQ ID NO:29 and SEQ ID NO:77, SEQ ID NO:33 and SEQ ID NO:77, SEQ ID NO: 37 and SEQ ID NO:77, SEQ ID NO:41 and SEQ ID NO:77, SEQ ID NO:45 and SEQ ID NO: 77, SEQ ID NO:49 and SEQ ID NO:77, SEQ ID NO:53 and SEQ ID NO:77, SEQ ID NO:57 and SEQ ID NO:77, SEQ ID NO:61 and SEQ ID NO:77, SEQ ID NO:65 and SEQ ID NO:77 (furthermore any of the sIL-6R VHHs shown in SEQ ID NOs: 119-137 can similarly be combined with SEQ ID NO:77 and any of SEQ ID NO:214-232 can be used in place of SEQ ID NO:77); SEQ ID NO: 1 and SEQ ID NO:81, SEQ ID NO:5 and SEQ ID NO:81, SEQ ID NO: 9 and SEQ ID NO:81, SEQ ID NO: 13 and SEQ ID NO:81, SEQ ID NO: 17 and SEQ ID NO:81, SEQ ID NO:21 and SEQ ID NO: 81, SEQ ID NO:25 and SEQ ID NO:81, SEQ ID NO:29 and SEQ ID NO:81, SEQ ID NO:33 and SEQ ID NO:81, SEQ ID NO:37 and SEQ ID NO:81, SEQ ID NO:41 and SEQ ID NO:81, SEQ ID NO: 45 and SEQ ID NO:81, SEQ ID NO:49 and SEQ ID NO:81, SEQ ID NO:53 and SEQ ID NO: 81, SEQ ID NO:57 and SEQ ID NO:81, SEQ ID NO:61 and SEQ ID NO:81, SEQ ID NO:65 and SEQ ID NO:81 (furthermore any of the sIL-6R VHHs shown in SEQ ID NOs: 119-137 can similarly be combined with SEQ ID NO:81 and any of SEQ ID NO:214-232 can be used in place of SEQ ID NO:81); SEQ ID NO: 1 and SEQ ID NO: 85, SEQ ID NO:5 and SEQ ID NO:85, SEQ ID NO: 9 and SEQ ID NO: 85, SEQ ID NO: 13 and SEQ ID NO:85, SEQ ID NO: 17 and SEQ ID NO:85, SEQ ID NO:21 and SEQ ID NO:85, SEQ ID NO:25 and SEQ ID NO:85, SEQ ID NO:29 and SEQ ID NO: 85, SEQ ID NO:33 and SEQ ID NO: 85, SEQ ID NO:37 and SEQ ID NO:85, SEQ ID NO:41 and SEQ ID NO: 85, SEQ ID NO:45 and SEQ ID NO:85, SEQ ID NO:49 and SEQ ID NO:85, SEQ ID NO: 53 and SEQ ID NO:85, SEQ ID NO:57 and SEQ ID NO:85, SEQ ID NO:61 and SEQ ID NO: 85, SEQ ID NO: 65 and SEQ ID NO: 85 (furthermore any of the sIL-6R VHHs shown in SEQ ID NOs: 119-137 can similarly be combined with SEQ ID NO:85 and any of SEQ ID NO:214-232 can be used in place of SEQ ID NO:85); SEQ ID NO: 1 and SEQ ID NO:89, SEQ ID NO:5 and SEQ ID NO:89, SEQ ID NO:9 and SEQ ID NO:89, SEQ ID NO: 13 and SEQ ID NO:89, SEQ ID NO: 17 and SEQ ID NO: 89, SEQ ID NO:21 and SEQ ID NO: 89, SEQ ID NO:25 and SEQ ID NO: 89, SEQ ID NO:29 and SEQ ID NO:89, SEQ ID NO:33 and SEQ ID NO:89, SEQ ID NO:37 and SEQ ID NO: 89, SEQ ID NO:41 and SEQ ID NO:89, SEQ ID NO: 45 and SEQ ID NO:89, SEQ ID NO:49 and SEQ ID NO: 89, SEQ ID NO:53 and SEQ ID NO:89, SEQ ID NO:57 and SEQ ID NO:89, SEQ ID NO: 61 and SEQ ID NO:89, or SEQ ID NO: 65 and SEQ ID NO:89 (furthermore any of the sIL-6R VHHs shown in SEQ ID NOs: 119-137 can similarly be combined with SEQ ID NO:89 and any of SEQ ID NO:214-232 can be used in place of SEQ ID NO:89) can further include an anti-transferrin receptor antibody.

Proteins offer unique opportunities as therapeutics and basic research tools. However, one major challenge to the broader use of proteins in biomedical applications is their general inability to efficiently cross the lipid bilayer of mammalian cells and access the cytosol. Thus, most current protein drugs and basic research tools target disease-relevant receptors that reside on the surface of the cell or the extracellular matrix. Efforts to unlock the full potential of proteins in biomedical applications by enabling potent and functional cell penetration have been a major focus of modern biologics research. Incorporation of polycationic linkages-such as polyarginine can be a means to enable cell penetration of various cargo, including proteins. “Protein engineering” to generate polycationic features on the protein surface, or “polycationic resurfacing” can include for example (i) “arginine grafting”, the mutagenesis of clustered solvent exposed amino acids to arginine, which can enable cellular uptake; (ii) protein “supercharging”, which is the extensive mutagenesis of a large number of solvent-exposed residues to positively charged lysine or arginine, and can result in potent penetration of mammalian cells.

Any one of the binding proteins, multispecific binding proteins and fusion proteins described herein can be mutated by polycationic resurfacing.

### Nucleic Acid Molecules

An embodiment provides an isolated nucleic acid molecule encoding any one of the binding proteins, the multispecific binding proteins, or the fusion proteins described herein.

As used herein, the term “nucleic acid molecule” or “oligonucleotide” refers to polynucleotides such as deoxyribonucleic acid (DNA) or ribonucleic acid (RNA). Nucleic acid molecules include but are not limited to genomic DNA, cDNA, mRNA, IRNA, miRNA, tRNA, ncRNA, rRNA, and recombinantly produced and chemically synthesized molecules such as aptamers, plasmids, anti-sense DNA strands, shRNA, ribozymes, nucleic acids conjugated and oligonucleotides. A nucleic acid molecule may be present as a single-stranded or double-stranded and linear or covalently circularly closed molecule. A nucleic acid molecule can be isolated. The term “isolated nucleic acid molecule” means, that the nucleic acid molecule (i) was amplified in vitro, for example via polymerase chain reaction (PCR), (ii) was produced recombinantly by cloning, (iii) was purified, for example, by cleavage and separation by gel electrophoresis, (iv) was synthesized, for example, by chemical synthesis, or (vi) extracted from a sample. A nucleic acid molecule can be employed for introduction into, i.e., transfection of, cells, in particular, in the form of RNA which can be prepared by in vitro transcription from a DNA template. The RNA can moreover be modified before application by stabilizing sequences, capping, and polyadenylation.

As used herein “amplified DNA” or “PCR product” refers to an amplified fragment of DNA of defined size. Various techniques are available and well known in the art to detect PCR products. PCR product detection methods include, but are not restricted to, gel electrophoresis using agarose or polyacrylamide gel and adding ethidium bromide staining (a DNA intercalant), labeled probes (radioactive or non-radioactive labels, southern blotting), labeled deoxyribonucleotides (for the direct incorporation of radioactive or non-radioactive labels) or silver staining for the direct visualization of the amplified PCR products; restriction endonuclease digestion, that relies agarose or polyacrylamide gel or High-performance liquid chromatography (HPLC); dot blots, using the hybridization of the amplified DNA on specific labeled probes (radioactive or non-radioactive labels); high-pressure liquid chromatography using ultraviolet detection; electro-chemiluminescence coupled with voltage-initiated chemical reaction/photon detection; and direct sequencing using radioactive or fluorescently labeled deoxyribonucleotides for the determination of the precise order of nucleotides with a DNA fragment of interest, oligo ligation assay (OLA), PCR, qPCR, DNA sequencing, fluorescence, gel electrophoresis, magnetic beads, allele specific primer extension (ASPE) and/or direct hybridization.

Generally, nucleic acid molecules can be extracted, isolated, amplified, or analyzed by a variety of techniques such as those described by Green and Sambrook, Molecular Cloning: A Laboratory Manual (Fourth Edition), Cold Spring Harbor Laboratory Press, Woodbury, NY 2,028 pages (2012); or as described in U.S. Pat. Nos. 7,957,913; 7,776,616; 5,234,809; U.S. Pub. 2010/0285578; and U.S. Pub. 2002/0190663. Examples of nucleic acid analysis include, but are not limited to, sequencing and DNA-protein interaction. Sequencing may be by any method known in the art. DNA sequencing techniques include classic dideoxy sequencing reactions (Sanger method) using labeled terminators or primers and gel separation in slab or capillary, and next generation sequencing methods such as sequencing by synthesis using reversibly terminated labeled nucleotides, pyrosequencing, 454 sequencing, Illumina/Solexa sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Separated molecules may be sequenced by sequential or single extension reactions using polymerases or ligases as well as by single or sequential differential hybridizations with libraries of probes.

For example, provided herein with this disclosure are nucleic acid molecules encoding:
(i) a sIL-6R binding protein as described herein, for example including CDRs as set forth in SEQ ID NOs: 2-4, SEQ ID NOs: 6-8, SEQ ID NOs: 10-12, SEQ ID NOs: 14-16, SEQ ID NOs: 18-20, SEQ ID NOs: 22-24, SEQ ID NOs: 26-28, SEQ ID NOs: 30-32, SEQ ID NOs: 34-36, SEQ ID NOs: 38-40, SEQ ID NOs: 42-44, SEQ ID NOs: 46-48, SEQ ID NOs: 50-52, SEQ ID NOs: 54-56, SEQ ID NOs: 58-60, SEQ ID NOs: 62-64, SEQ ID NOs: 66-68, or SEQ ID NO: 138-194;
(ii) a sIL-6R binding protein as described herein, for example including the amino acid sequence of SEQ ID NO:1, SEQ ID NO:5, SEQ ID NO:9, SEQ ID NO: 13, SEQ ID NO: 17, SEQ ID NO: 21, SEQ ID NO:25, SEQ ID NO:29, SEQ ID NO:33, SEQ ID NO:37, SEQ ID NO:41, SEQ ID NO: 45, SEQ ID NO:49, SEQ ID NO:53, SEQ ID NO:57, SEQ ID NO:61, SEQ ID NO:65, or SEQ ID NOs: 119-137;
(iii) a CTGF binding protein as described herein, for example including CDRs as set forth in NOs: 70-72, SEQ ID NOs: 74-76, SEQ ID NOs: 78-80, SEQ ID NOS: 82-84, SEQ ID NOS: 86-88, SEQ ID NOs: 90-92, SEQ ID NOs: 233-286, or SEQ ID NOs: 291-293;
(iv) a CTGF binding protein as described herein, for example including the amino acid sequence of SEQ ID NO:73, SEQ ID NO:77, SEQ ID NO:81, SEQ ID NO:85 or SEQ ID NO:89 or SEQ ID NOs: 214-232;
(v) a multispecific binding protein that selectively and/or specifically binds to human sIL-6R and to human CTGF as described herein, for example including CDRs as set forth in SEQ ID NOs: 2-4 and SEQ ID NOs: 70-72, SEQ ID NOs: 6-8 and SEQ ID NOs: 70-72, SEQ ID NOs: 10-12 and SEQ ID NOs: 70-72, SEQ ID NOs: 14-16 and SEQ ID NOs: 70-72, SEQ ID NOs: 18-20 and SEQ ID NOs: 70-72, SEQ ID NOs: 22-24 and SEQ ID NOs: 70-72, SEQ ID NOs: 26-28 and SEQ ID NOs: 70-72, SEQ ID NOs: 30-32 and SEQ ID NOs: 70-72, SEQ ID NOs: 34-36 and SEQ ID NOs: 70-72, SEQ ID NOs: 38-40 and SEQ ID NOs: 70-72, SEQ ID NOs: 42-44 and SEQ ID NOs: 70-72, SEQ ID NOs: 46-48 and SEQ ID NOs: 70-72, SEQ ID NOs: 50-52 and SEQ ID NOs: 70-72, SEQ ID NOs: 54-56 and SEQ ID NOs: 70-72, SEQ ID NOs: 58-60 and SEQ ID NOs: 70-72, SEQ ID NOs: 62-64 and SEQ ID NOs: 70-72; SEQ ID NOs: 66-68 and SEQ ID NOs: 70-72 (furthermore any of the SIL-6R CDR sets shown in SEQ ID NOs: 138-194 can similarly be combined with SEQ ID NO:70-72 and any of the CTGF CDR sets as set forth in SEQ ID NOs: 233-286 can be used in place of SEQ ID NOs: 70-72); SEQ ID NOs: 2-4 and SEQ ID NOs: 74-76, SEQ ID NOs: 6-8 and SEQ ID NOs: 74-76, SEQ ID NOs: 10-12 and SEQ ID NOs: 74-76, SEQ ID NOs: 14-16 and SEQ ID NOs: 74-76, SEQ ID NOs: 18-20 and SEQ ID NOs: 74-76, SEQ ID NOs: 22-24 and SEQ ID NOs: 74-76, SEQ ID NOs: 26-28 and SEQ ID NOs: 74-76, SEQ ID NOs: 30-32 and SEQ ID NOs: 74-76, SEQ ID NOs: 34-36 and SEQ ID NOs: 74-76, SEQ ID NOs: 38-40 and SEQ ID NOs: 74-76, SEQ ID NOs: 42-44 and SEQ ID NOs: 74-76, SEQ ID NOs: 46-48 and SEQ ID NOs: 74-76, SEQ ID NOs: 50-52 and SEQ ID NOs: 74-76, SEQ ID NOs: 54-56 and SEQ ID NOs: 74-76, SEQ ID NOs: 58-60 and SEQ ID NOs: 74-76, SEQ ID NOs: 62-64 and SEQ ID NOs: 74-76; SEQ ID NOs: 66-68 and SEQ ID NOs: 74-76 (furthermore any of the sIL-6R CDR sets shown in SEQ ID NOs: 138-194 can similarly be combined with SEQ ID NO:74-76 and any of the CTGF CDR sets as set forth in SEQ ID NOs: 233-286 can be used in place of SEQ ID NOs: 74-76); SEQ ID NOs: 2-4 and SEQ ID NOs: 78-80, SEQ ID NOs: 6-8 and SEQ ID NOs: 78-80, SEQ ID NOs: 10-12 and SEQ ID NOs: 78-80, SEQ ID NOs: 14-16 and SEQ ID NOs: 78-80, SEQ ID NOs: 18-20 and SEQ ID NOs: 78-80, SEQ ID NOs: 22-24 and SEQ ID NOs: 78-80, SEQ ID NOs: 26-28 and SEQ ID NOs: 78-80, SEQ ID NOs: 30-32 and SEQ ID NOs: 78-80, SEQ ID NOs: 34-36 and SEQ ID NOs: 78-80, SEQ ID NOs: 38-40 and SEQ ID NOs: 78-80, SEQ ID NOs: 42-44 and SEQ ID NOs: 78-80, SEQ ID NOs: 46-48 and SEQ ID NOs: 78-80, SEQ ID NOs: 50-52 and SEQ ID NOs: 78-80, SEQ ID NOs: 54-56 and SEQ ID NOs: 78-80, SEQ ID NOs: 58-60 and SEQ ID NOs: 78-80, SEQ ID NOs: 62-64 and SEQ ID NOs: 78-80; SEQ ID NOs: 66-68 and SEQ ID NOs: 78-80 (furthermore any of the sIL-6R CDR sets shown in SEQ ID NOs: 138-194 can similarly be combined with SEQ ID NO:78-80 and any of the CTGF CDR sets as set forth in SEQ ID NOs: 233-286 can be used in place of SEQ ID NOs: 78-80); SEQ ID NOs: 2-4 and SEQ ID NOs: 82-84, SEQ ID NOs: 6-8 and SEQ ID NOs: 82-84, SEQ ID NOs: 10-12 and SEQ ID NOs: 82-84, SEQ ID NOs: 14-16 and SEQ ID NOs: 82-84, SEQ ID NOs: 18-20 and SEQ ID NOs: 82-84, SEQ ID NOs: 22-24 and SEQ ID NOs: 82-84, SEQ ID NOs: 26-28 and SEQ ID NOs: 82-84, SEQ ID NOs: 30-32 and SEQ ID NOs: 82-84, SEQ ID NOs: 34-36 and SEQ ID NOs: 82-84, SEQ ID NOs: 38-40 and SEQ ID NOs: 82-84, SEQ ID NOs: 42-44 and SEQ ID NOs: 82-84, SEQ ID NOs: 46-48 and SEQ ID NOs: 82-84, SEQ ID NOs: 50-52 and SEQ ID NOs: 82-84, SEQ ID NOs: 54-56 and SEQ ID NOs: 82-84, SEQ ID NOs: 58-60 and SEQ ID NOs: 82-84, SEQ ID NOs: 62-64 and SEQ ID NOs: 82-84; SEQ ID NOs: 66-68 and SEQ ID NOs: 82-84 (furthermore any of the sIL-6R CDR sets shown in SEQ ID NOs: 138-194 can similarly be combined with SEQ ID NO:82-84 and any of the CTGF CDR sets as set forth in SEQ ID NOs: 233-286 can be used in place of SEQ ID NOs: 82-84); SEQ ID NOs: 2-4 and SEQ ID NOs: 86-88, SEQ ID NOs: 6-8 and SEQ ID NOs: 86-88, SEQ ID NOs: 10-12 and SEQ ID NOs: 86-88, SEQ ID NOs: 14-16 and SEQ ID NOs: 86-88, SEQ ID NOs: 18-20 and SEQ ID NOs: 86-88, SEQ ID NOs: 22-24 and SEQ ID NOs: 86-88, SEQ ID NOs: 26-28 and SEQ ID NOs: 86-88, SEQ ID NOs: 30-32 and SEQ ID NOs: 86-88, SEQ ID NOs: 34-36 and SEQ ID NOs: 86-88, SEQ ID NOs: 38-40 and SEQ ID NOs: 86-88, SEQ ID NOs: 42-44 and SEQ ID NOs: 86-88, SEQ ID NOs: 46-48 and SEQ ID NOs: 86-88, SEQ ID NOs: 50-52 and SEQ ID NOs: 86-88, SEQ ID NOs: 54-56 and SEQ ID NOs: 86-88, SEQ ID NOs: 58-60 and SEQ ID NOs: 86-88, SEQ ID NOs: 62-64 and SEQ ID NOs: 86-88; SEQ ID NOs: 66-68 and SEQ ID NOs: 86-88 (furthermore any of the sIL-6R CDR sets shown in SEQ ID NOs: 138-194 can similarly be combined with SEQ ID NO: 86-88 and any of the CTGF CDR sets as set forth in SEQ ID NOs: 233-286 can be used in place of SEQ ID NOs: 86-88); SEQ ID NOs: 2-4 and SEQ ID NOs: 90-92, SEQ ID NOs: 6-8 and SEQ ID NOs: 90-92, SEQ ID NOs: 10-12 and SEQ ID NOs: 90-92, SEQ ID NOs: 14-16 and SEQ ID NOs: 90-92, SEQ ID NOs: 18-20 and SEQ ID NOs: 90-92, SEQ ID NOs: 22-24 and SEQ ID NOs: 90-92, SEQ ID NOs: 26-28 and SEQ ID NOs: 90-92, SEQ ID NOs: 30-32 and SEQ ID NOs: 90-92, SEQ ID NOs: 34-36 and SEQ ID NOs: 90-92, SEQ ID NOs: 38-40 and SEQ ID NOs: 90-92, SEQ ID NOs: 42-44 and SEQ ID NOs: 90-92, SEQ ID NOs: 46-48 and SEQ ID NOs: 90-92, SEQ ID NOs: 50-52 and SEQ ID NOs: 90-92, SEQ ID NOs: 54-56 and SEQ ID NOs: 90-92, SEQ ID NOs: 58-60 and SEQ ID NOs: 90-92, SEQ ID NOs: 62-64 and SEQ ID NOs: 90-92 or SEQ ID NOs: 66-68 and SEQ ID NOs: 90-92 (furthermore any of the sIL-6R CDR sets shown in SEQ ID NOs: 138-194 can similarly be combined with SEQ ID NO: 90-92 and any of the CTGF CDR sets as set forth in SEQ ID NOs: 233-286 can be used in place of SEQ ID NOs: 90-92);
(vi) a multispecific binding protein that selectively and/or specifically binds to human sIL-6R and to human CTGF as described herein, for example including the amino acid sequence of: SEQ ID NO: 1 and SEQ ID NO:69, SEQ ID NO:5 and SEQ ID NO:69, SEQ ID NO:9 and SEQ ID NO: 69, SEQ ID NO: 13 and SEQ ID NO:69, SEQ ID NO: 17 and SEQ ID NO:69, SEQ ID NO:21 and SEQ ID NO:69, SEQ ID NO:25 and SEQ ID NO:69, SEQ ID NO:29 and SEQ ID NO:69, SEQ ID NO: 33 and SEQ ID NO:69, SEQ ID NO:37 and SEQ ID NO:69, SEQ ID NO:41 and SEQ ID NO: 69, SEQ ID NO:45 and SEQ ID NO:69, SEQ ID NO:49 and SEQ ID NO:69, SEQ ID NO:53 and SEQ ID NO: 69, SEQ ID NO:57 and SEQ ID NO:69, SEQ ID NO:61 and SEQ ID NO:69, SEQ ID NO: 65 and SEQ ID NO:69 (furthermore any of the sIL-6R VHHs shown in SEQ ID NOs: 119-137 can similarly be combined with SEQ ID NO:69 and any of SEQ ID NO:214-232 can be used in place of SEQ ID NO:69); SEQ ID NO: 1 and SEQ ID NO:73, SEQ ID NO:5 and SEQ ID NO:73, SEQ ID NO:9 and SEQ ID NO:73, SEQ ID NO: 13 and SEQ ID NO:73, SEQ ID NO: 17 and SEQ ID NO: 73, SEQ ID NO:21 and SEQ ID NO:73, SEQ ID NO:25 and SEQ ID NO:73, SEQ ID NO:29 and SEQ ID NO:73, SEQ ID NO:33 and SEQ ID NO:73, SEQ ID NO:37 and SEQ ID NO:73, SEQ ID NO: 41 and SEQ ID NO:73, SEQ ID NO:45 and SEQ ID NO:73, SEQ ID NO:49 and SEQ ID NO: 73, SEQ ID NO:53 and SEQ ID NO:73, SEQ ID NO:57 and SEQ ID NO:73, SEQ ID NO:61 and SEQ ID NO:73, SEQ ID NO:65 and SEQ ID NO:73 (furthermore any of the sIL-6R VHHs shown in SEQ ID NOs: 119-137 can similarly be combined with SEQ ID NO:73 and any of SEQ ID NO: 214-232 can be used in place of SEQ ID NO:73); SEQ ID NO: 1 and SEQ ID NO:77, SEQ ID NO: 5 and SEQ ID NO:77, SEQ ID NO:9 and SEQ ID NO:77, SEQ ID NO: 13 and SEQ ID NO: 77, SEQ ID NO: 17 and SEQ ID NO:77, SEQ ID NO:21 and SEQ ID NO:77, SEQ ID NO:25 and SEQ ID NO:77, SEQ ID NO:29 and SEQ ID NO:77, SEQ ID NO:33 and SEQ ID NO:77, SEQ ID NO: 37 and SEQ ID NO:77, SEQ ID NO:41 and SEQ ID NO:77, SEQ ID NO:45 and SEQ ID NO: 77, SEQ ID NO:49 and SEQ ID NO:77, SEQ ID NO:53 and SEQ ID NO:77, SEQ ID NO:57 and SEQ ID NO:77, SEQ ID NO:61 and SEQ ID NO:77, SEQ ID NO:65 and SEQ ID NO:77 (furthermore any of the sIL-6R VHHs shown in SEQ ID NOs: 119-137 can similarly be combined with SEQ ID NO: 77 and any of SEQ ID NO:214-232 can be used in place of SEQ ID NO:77); SEQ ID NO: 1 and SEQ ID NO:81, SEQ ID NO:5 and SEQ ID NO:81, SEQ ID NO: 9 and SEQ ID NO:81, SEQ ID NO: 13 and SEQ ID NO:81, SEQ ID NO: 17 and SEQ ID NO:81, SEQ ID NO:21 and SEQ ID NO: 81, SEQ ID NO:25 and SEQ ID NO:81, SEQ ID NO:29 and SEQ ID NO:81, SEQ ID NO:33 and SEQ ID NO:81, SEQ ID NO:37 and SEQ ID NO:81, SEQ ID NO:41 and SEQ ID NO:81, SEQ ID NO: 45 and SEQ ID NO:81, SEQ ID NO:49 and SEQ ID NO:81, SEQ ID NO:53 and SEQ ID NO: 81, SEQ ID NO:57 and SEQ ID NO:81, SEQ ID NO:61 and SEQ ID NO:81, SEQ ID NO:65 and SEQ ID NO:81 (furthermore any of the sIL-6R VHHs shown in SEQ ID NOs: 119-137 can similarly be combined with SEQ ID NO:81 and any of SEQ ID NO:214-232 can be used in place of SEQ ID NO:81); SEQ ID NO: 1 and SEQ ID NO:85, SEQ ID NO:5 and SEQ ID NO:85, SEQ ID NO: 9 and SEQ ID NO: 85, SEQ ID NO: 13 and SEQ ID NO:85, SEQ ID NO: 17 and SEQ ID NO:85, SEQ ID NO:21 and SEQ ID NO:85, SEQ ID NO:25 and SEQ ID NO:85, SEQ ID NO:29 and SEQ ID NO: 85, SEQ ID NO:33 and SEQ ID NO:85, SEQ ID NO:37 and SEQ ID NO:85, SEQ ID NO:41 and SEQ ID NO: 85, SEQ ID NO:45 and SEQ ID NO: 85, SEQ ID NO:49 and SEQ ID NO:85, SEQ ID NO: 53 and SEQ ID NO:85, SEQ ID NO:57 and SEQ ID NO:85, SEQ ID NO:61 and SEQ ID NO: 85, SEQ ID NO: 65 and SEQ ID NO: 85 (furthermore any of the sIL-6R VHHs shown in SEQ ID NOs: 119-137 can similarly be combined with SEQ ID NO:85 and any of SEQ ID NO:214-232 can be used in place of SEQ ID NO:85); SEQ ID NO: 1 and SEQ ID NO:89, SEQ ID NO:5 and SEQ ID NO:89, SEQ ID NO:9 and SEQ ID NO:89, SEQ ID NO: 13 and SEQ ID NO:89, SEQ ID NO: 17 and SEQ ID NO:89, SEQ ID NO:21 and SEQ ID NO: 89, SEQ ID NO:25 and SEQ ID NO:89, SEQ ID NO:29 and SEQ ID NO:89, SEQ ID NO:33 and SEQ ID NO:89, SEQ ID NO:37 and SEQ ID NO: 89, SEQ ID NO:41 and SEQ ID NO: 89, SEQ ID NO:45 and SEQ ID NO:89, SEQ ID NO:49 and SEQ ID NO: 89, SEQ ID NO:53 and SEQ ID NO:89, SEQ ID NO:57 and SEQ ID NO:89, SEQ ID NO: 61 and SEQ ID NO:89, or SEQ ID NO: 65 and SEQ ID NO:89 (furthermore any of the sIL-6R VHHs shown in SEQ ID NOs: 119-137 can similarly be combined with SEQ ID NO:89 and any of SEQ ID NO:214-232 can be used in place of SEQ ID NO:89);
(vii) a fusion protein including a sIL-6R binding protein as described herein and an anti-transferrin receptor antibody, for example including CDRs as set forth in SEQ ID NOs: 2-4, SEQ ID NOs: 6-8, SEQ ID NOs: 10-12, SEQ ID NOs: 14-16, SEQ ID NOs: 18-20, SEQ ID NOS: 22-24, SEQ ID NOs: 26-28, SEQ ID NOs: 30-32, SEQ ID NOs: 34-36, SEQ ID NOs: 38-40, SEQ ID NOs: 42-44, SEQ ID NOs: 46-48, SEQ ID NOs: 50-52, SEQ ID NOs: 54-56, SEQ ID NOs: 58-60, SEQ ID NOs: 62-64, SEQ ID NOs: 66-68, or SEQ ID NO: 138-194 each further including an amino acid sequence encoding an anti-transferrin receptor antibody;
(viii) a fusion protein including a sIL-6R binding protein as described herein and an anti-transferrin receptor antibody, for example including the amino acid sequence of SEQ ID NO:1, SEQ ID NO:5, SEQ ID NO:9, SEQ ID NO: 13, SEQ ID NO: 17, SEQ ID NO:21, SEQ ID NO:25, SEQ ID NO:29, SEQ ID NO:33, SEQ ID NO:37, SEQ ID NO:41, SEQ ID NO:45, SEQ ID NO:49, SEQ ID NO:53, SEQ ID NO:57, SEQ ID NO:61, SEQ ID NO:65, or SEQ ID NO: 119-137, each further including an amino acid sequence encoding an anti-transferrin receptor antibody;
(ix) a fusion protein including a CTGF binding protein as described herein and an anti-transferrin receptor antibody, for example including CDRs as set forth in NOs: 70-72, SEQ ID NOs: 74-76, SEQ ID NOs: 78-80, SEQ ID NOS: 82-84, SEQ ID NOS: 86-88, SEQ ID NOS: 90-92, SEQ ID NOs: 233-286, or SEQ ID NOs: 291-293 each further including an amino acid sequence encoding an anti-transferrin receptor antibody;
(x) a fusion protein including a CTGF binding protein as described herein and an anti-transferrin receptor antibody, for example including the amino acid sequence of SEQ ID NO:73, SEQ ID NO:77, SEQ ID NO:81, SEQ ID NO:85, SEQ ID NO:89, or SEQ ID NOs: 214-232 each further including an amino acid sequence encoding an anti-transferrin receptor antibody;
(xi) a fusion protein including a multispecific binding protein that selectively and/or specifically binds to human sIL-6R and to human CTGF as described herein and an anti-transferrin receptor antibody, for example including CDRs as set forth in SEQ ID NOs: 2-4 and SEQ ID NOs: 70-72, SEQ ID NOs: 6-8 and SEQ ID NOs: 70-72, SEQ ID NOs: 10-12 and SEQ ID NOs: 70-72, SEQ ID NOs: 14-16 and SEQ ID NOs: 70-72, SEQ ID NOs: 18-20 and SEQ ID NOs: 70-72, SEQ ID NOs: 22-24 and SEQ ID NOs: 70-72, SEQ ID NOs: 26-28 and SEQ ID NOs: 70-72, SEQ ID NOs: 30-32 and SEQ ID NOs: 70-72, SEQ ID NOs: 34-36 and SEQ ID NOs: 70-72, SEQ ID NOs: 38-40 and SEQ ID NOs: 70-72, SEQ ID NOs: 42-44 and SEQ ID NOs: 70-72, SEQ ID NOs: 46-48 and SEQ ID NOs: 70-72, SEQ ID NOs: 50-52 and SEQ ID NOs: 70-72, SEQ ID NOs: 54-56 and SEQ ID NOs: 70-72, SEQ ID NOs: 58-60 and SEQ ID NOs: 70-72, SEQ ID NOs: 62-64 and SEQ ID NOs: 70-72; SEQ ID NOs: 66-68 and SEQ ID NOs: 70-72 (furthermore any of the sIL-6R CDR sets shown in SEQ ID NOs: 138-194 can similarly be combined with SEQ ID NO:70-72 and any of the CTGF CDR sets as set forth in SEQ ID NOs: 233-286 can be used in place of SEQ ID NOs: 70-72); SEQ ID NOs: 2-4 and SEQ ID NOs: 74-76, SEQ ID NOs: 6-8 and SEQ ID NOs: 74-76, SEQ ID NOs: 10-12 and SEQ ID NOs: 74-76, SEQ ID NOs: 14-16 and SEQ ID NOs: 74-76, SEQ ID NOs: 18-20 and SEQ ID NOs: 74-76, SEQ ID NOs: 22-24 and SEQ ID NOs: 74-76, SEQ ID NOs: 26-28 and SEQ ID NOs: 74-76, SEQ ID NOs: 30-32 and SEQ ID NOs: 74-76, SEQ ID NOs: 34-36 and SEQ ID NOs: 74-76, SEQ ID NOs: 38-40 and SEQ ID NOs: 74-76, SEQ ID NOs: 42-44 and SEQ ID NOs: 74-76, SEQ ID NOs: 46-48 and SEQ ID NOs: 74-76, SEQ ID NOs: 50-52 and SEQ ID NOs: 74-76, SEQ ID NOs: 54-56 and SEQ ID NOs: 74-76, SEQ ID NOs: 58-60 and SEQ ID NOs: 74-76, SEQ ID NOs: 62-64 and SEQ ID NOs: 74-76; SEQ ID NOs: 66-68 and SEQ ID NOs: 74-76 (furthermore any of the sIL-6R CDR sets shown in SEQ ID NOs: 138-194 can similarly be combined with SEQ ID NO: 74-76 and any of the CTGF CDR sets as set forth in SEQ ID NOs: 233-286 can be used in place of SEQ ID NOs: 74-76); SEQ ID NOs: 2-4 and SEQ ID NOs: 78-80, SEQ ID NOs: 6-8 and SEQ ID NOs: 78-80, SEQ ID NOs: 10-12 and SEQ ID NOs: 78-80, SEQ ID NOs: 14-16 and SEQ ID NOs: 78-80, SEQ ID NOs: 18-20 and SEQ ID NOs: 78-80, SEQ ID NOs: 22-24 and SEQ ID NOs: 78-80, SEQ ID NOs: 26-28 and SEQ ID NOs: 78-80, SEQ ID NOs: 30-32 and SEQ ID NOs: 78-80, SEQ ID NOs: 34-36 and SEQ ID NOs: 78-80, SEQ ID NOs: 38-40 and SEQ ID NOs: 78-80, SEQ ID NOs: 42-44 and SEQ ID NOs: 78-80, SEQ ID NOs: 46-48 and SEQ ID NOs: 78-80, SEQ ID NOs: 50-52 and SEQ ID NOs: 78-80, SEQ ID NOs: 54-56 and SEQ ID NOs: 78-80, SEQ ID NOs: 58-60 and SEQ ID NOs: 78-80, SEQ ID NOs: 62-64 and SEQ ID NOs: 78-80; SEQ ID NOs: 66-68 and SEQ ID NOs: 78-80 (furthermore any of the sIL-6R CDR sets shown in SEQ ID NOs: 138-194 can similarly be combined with SEQ ID NO:78-80 and any of the CTGF CDR sets as set forth in SEQ ID NOs: 233-286 can be used in place of SEQ ID NOs: 78-80); SEQ ID NOs: 2-4 and SEQ ID NOs: 82-84, SEQ ID NOs: 6-8 and SEQ ID NOs: 82-84, SEQ ID NOs: 10-12 and SEQ ID NOs: 82-84, SEQ ID NOs: 14-16 and SEQ ID NOs: 82-84, SEQ ID NOs: 18-20 and SEQ ID NOs: 82-84, SEQ ID NOs: 22-24 and SEQ ID NOs: 82-84, SEQ ID NOs: 26-28 and SEQ ID NOs: 82-84, SEQ ID NOs: 30-32 and SEQ ID NOs: 82-84, SEQ ID NOs: 34-36 and SEQ ID NOs: 82-84, SEQ ID NOs: 38-40 and SEQ ID NOs: 82-84, SEQ ID NOs: 42-44 and SEQ ID NOs: 82-84, SEQ ID NOs: 46-48 and SEQ ID NOs: 82-84, SEQ ID NOs: 50-52 and SEQ ID NOs: 82-84, SEQ ID NOs: 54-56 and SEQ ID NOs: 82-84, SEQ ID NOs: 58-60 and SEQ ID NOs: 82-84, SEQ ID NOs: 62-64 and SEQ ID NOs: 82-84; SEQ ID NOs: 66-68 and SEQ ID NOs: 82-84 (furthermore any of the sIL-6R CDR sets shown in SEQ ID NOs: 138-194 can similarly be combined with SEQ ID NO:82-84 and any of the CTGF CDR sets as set forth in SEQ ID NOs: 233-286 can be used in place of SEQ ID NOs: 82-84); SEQ ID NOs: 2-4 and SEQ ID NOs: 86-88, SEQ ID NOs: 6-8 and SEQ ID NOs: 86-88, SEQ ID NOs: 10-12 and SEQ ID NOs: 86-88, SEQ ID NOs: 14-16 and SEQ ID NOs: 86-88, SEQ ID NOs: 18-20 and SEQ ID NOs: 86-88, SEQ ID NOs: 22-24 and SEQ ID NOs: 86-88, SEQ ID NOs: 26-28 and SEQ ID NOs: 86-88, SEQ ID NOs: 30-32 and SEQ ID NOs: 86-88, SEQ ID NOs: 34-36 and SEQ ID NOs: 86-88, SEQ ID NOs: 38-40 and SEQ ID NOs: 86-88, SEQ ID NOs: 42-44 and SEQ ID NOs: 86-88, SEQ ID NOs: 46-48 and SEQ ID NOs: 86-88, SEQ ID NOs: 50-52 and SEQ ID NOs: 86-88, SEQ ID NOs: 54-56 and SEQ ID NOs: 86-88, SEQ ID NOs: 58-60 and SEQ ID NOs: 86-88, SEQ ID NOs: 62-64 and SEQ ID NOs: 86-88; SEQ ID NOs: 66-68 and SEQ ID NOs: 86-88 (furthermore any of the sIL-6R CDR sets shown in SEQ ID NOs: 138-194 can similarly be combined with SEQ ID NO:86-88 and any of the CTGF CDR sets as set forth in SEQ ID NOs: 233-286 can be used in place of SEQ ID NOs: 86-88); SEQ ID NOs: 2-4 and SEQ ID NOs: 90-92, SEQ ID NOs: 6-8 and SEQ ID NOs: 90-92, SEQ ID NOs: 10-12 and SEQ ID NOs: 90-92, SEQ ID NOs: 14-16 and SEQ ID NOs: 90-92, SEQ ID NOs: 18-20 and SEQ ID NOs: 90-92, SEQ ID NOs: 22-24 and SEQ ID NOs: 90-92, SEQ ID NOs: 26-28 and SEQ ID NOs: 90-92, SEQ ID NOs: 30-32 and SEQ ID NOs: 90-92, SEQ ID NOs: 34-36 and SEQ ID NOs: 90-92, SEQ ID NOs: 38-40 and SEQ ID NOs: 90-92, SEQ ID NOs: 42-44 and SEQ ID NOs: 90-92, SEQ ID NOs: 46-48 and SEQ ID NOs: 90-92, SEQ ID NOs: 50-52 and SEQ ID NOs: 90-92, SEQ ID NOs: 54-56 and SEQ ID NOs: 90-92, SEQ ID NOs: 58-60 and SEQ ID NOs: 90-92, SEQ ID NOs: 62-64 and SEQ ID NOs: 90-92 or SEQ ID NOs: 66-68 and SEQ ID NOs: 90-92 (furthermore any of the sIL-6R CDR sets shown in SEQ ID NOs: 138-194 can similarly be combined with SEQ ID NO: 90-92 and any of the CTGF CDR sets as set forth in SEQ ID NOs: 233-286 can be used in place of SEQ ID NOs: 90-92), each further including an amino acid sequence encoding an anti-transferrin receptor antibody;
(xii) a fusion protein including a multispecific binding protein that selectively and/or specifically binds to human sIL-6R and to human CTGF as described herein and an anti-transferrin receptor antibody, for example including the amino acid sequence of: SEQ ID NO:1 and SEQ ID NO:69, SEQ ID NO:5 and SEQ ID NO:69, SEQ ID NO:9 and SEQ ID NO:69, SEQ ID NO: 13 and SEQ ID NO:69, SEQ ID NO: 17 and SEQ ID NO:69, SEQ ID NO:21 and SEQ ID NO: 69, SEQ ID NO:25 and SEQ ID NO:69, SEQ ID NO:29 and SEQ ID NO:69, SEQ ID NO:33 and SEQ ID NO:69, SEQ ID NO:37 and SEQ ID NO:69, SEQ ID NO:41 and SEQ ID NO:69, SEQ ID NO: 45 and SEQ ID NO:69, SEQ ID NO:49 and SEQ ID NO:69, SEQ ID NO:53 and SEQ ID NO: 69, SEQ ID NO:57 and SEQ ID NO:69, SEQ ID NO:61 and SEQ ID NO:69, SEQ ID NO:65 and SEQ ID NO:69 (furthermore any of the sIL-6R VHHs shown in SEQ ID NOs: 119-137 can similarly be combined with SEQ ID NO:69 and any of SEQ ID NO:214-232 can be used in place of SEQ ID NO: 69); SEQ ID NO: 1 and SEQ ID NO:73, SEQ ID NO:5 and SEQ ID NO:73, SEQ ID NO: 9 and SEQ ID NO:73, SEQ ID NO: 13 and SEQ ID NO:73, SEQ ID NO: 17 and SEQ ID NO:73, SEQ ID NO:21 and SEQ ID NO:73, SEQ ID NO:25 and SEQ ID NO:73, SEQ ID NO:29 and SEQ ID NO: 73, SEQ ID NO:33 and SEQ ID NO:73, SEQ ID NO:37 and SEQ ID NO:73, SEQ ID NO:41 and SEQ ID NO:73, SEQ ID NO:45 and SEQ ID NO:73, SEQ ID NO:49 and SEQ ID NO:73, SEQ ID NO: 53 and SEQ ID NO:73, SEQ ID NO:57 and SEQ ID NO:73, SEQ ID NO:61 and SEQ ID NO: 73, SEQ ID NO: 65 and SEQ ID NO:73 (furthermore any of the sIL-6R VHHs shown in SEQ ID NOs: 119-137 can similarly be combined with SEQ ID NO:73 and any of SEQ ID NO:214-232 can be used in place of SEQ ID NO:73); SEQ ID NO: 1 and SEQ ID NO:77, SEQ ID NO:5 and SEQ ID NO:77, SEQ ID NO:9 and SEQ ID NO:77, SEQ ID NO: 13 and SEQ ID NO:77, SEQ ID NO: 17 and SEQ ID NO: 77, SEQ ID NO:21 and SEQ ID NO:77, SEQ ID NO:25 and SEQ ID NO:77, SEQ ID NO:29 and SEQ ID NO:77, SEQ ID NO:33 and SEQ ID NO:77, SEQ ID NO:37 and SEQ ID NO: 77, SEQ ID NO:41 and SEQ ID NO:77, SEQ ID NO:45 and SEQ ID NO:77, SEQ ID NO:49 and SEQ ID NO:77, SEQ ID NO:53 and SEQ ID NO:77, SEQ ID NO:57 and SEQ ID NO:77, SEQ ID NO: 61 and SEQ ID NO:77, SEQ ID NO:65 and SEQ ID NO:77 (furthermore any of the sIL-6R VHHs shown in SEQ ID NOs: 119-137 can similarly be combined with SEQ ID NO:77 and any of SEQ ID NO:214-232 can be used in place of SEQ ID NO:77); SEQ ID NO:1 and SEQ ID NO:81, SEQ ID NO:5 and SEQ ID NO:81, SEQ ID NO:9 and SEQ ID NO:81, SEQ ID NO: 13 and SEQ ID NO: 81, SEQ ID NO: 17 and SEQ ID NO:81, SEQ ID NO:21 and SEQ ID NO:81, SEQ ID NO:25 and SEQ ID NO:81, SEQ ID NO:29 and SEQ ID NO:81, SEQ ID NO:33 and SEQ ID NO:81, SEQ ID NO: 37 and SEQ ID NO:81, SEQ ID NO:41 and SEQ ID NO:81, SEQ ID NO:45 and SEQ ID NO: 81, SEQ ID NO:49 and SEQ ID NO:81, SEQ ID NO:53 and SEQ ID NO:81, SEQ ID NO:57 and SEQ ID NO:81, SEQ ID NO:61 and SEQ ID NO:81, SEQ ID NO:65 and SEQ ID NO:81 (furthermore any of the sIL-6R VHHs shown in SEQ ID NOs: 119-137 can similarly be combined with SEQ ID NO: 81 and any of SEQ ID NO:214-232 can be used in place of SEQ ID NO:81); SEQ ID NO: 1 and SEQ ID NO:85, SEQ ID NO:5 and SEQ ID NO:85, SEQ ID NO: 9 and SEQ ID NO:85, SEQ ID NO: 13 and SEQ ID NO:85, SEQ ID NO: 17 and SEQ ID NO:85, SEQ ID NO:21 and SEQ ID NO: 85, SEQ ID NO:25 and SEQ ID NO:85, SEQ ID NO:29 and SEQ ID NO:85, SEQ ID NO:33 and SEQ ID NO:85, SEQ ID NO:37 and SEQ ID NO:85, SEQ ID NO:41 and SEQ ID NO:85, SEQ ID NO: 45 and SEQ ID NO:85, SEQ ID NO:49 and SEQ ID NO:85, SEQ ID NO:53 and SEQ ID NO: 85, SEQ ID NO:57 and SEQ ID NO:85, SEQ ID NO:61 and SEQ ID NO:85, SEQ ID NO:65 and SEQ ID NO:85 (furthermore any of the sIL-6R VHHs shown in SEQ ID NOs: 119-137 can similarly be combined with SEQ ID NO:85 and any of SEQ ID NO:214-232 can be used in place of SEQ ID NO: 85); SEQ ID NO: 1 and SEQ ID NO:89, SEQ ID NO:5 and SEQ ID NO:89, SEQ ID NO: 9 and SEQ ID NO: 89, SEQ ID NO: 13 and SEQ ID NO:89, SEQ ID NO: 17 and SEQ ID NO:89, SEQ ID NO:21 and SEQ ID NO:89, SEQ ID NO:25 and SEQ ID NO:89, SEQ ID NO:29 and SEQ ID NO: 89, SEQ ID NO:33 and SEQ ID NO: 89, SEQ ID NO:37 and SEQ ID NO:89, SEQ ID NO:41 and SEQ ID NO:89, SEQ ID NO:45 and SEQ ID NO:89, SEQ ID NO:49 and SEQ ID NO:89, SEQ ID NO: 53 and SEQ ID NO:89, SEQ ID NO:57 and SEQ ID NO:89, SEQ ID NO:61 and SEQ ID NO: 89, or SEQ ID NO: 65 and SEQ ID NO: 89 (furthermore any of the sIL-6R VHHs shown in SEQ ID NOs: 119-137 can similarly be combined with SEQ ID NO:89 and any of SEQ ID NO:214-232 can be used in place of SEQ ID NO:89), each further including an amino acid sequence encoding an anti-transferrin receptor antibody.

### Expression Vectors

An embodiment provides an expression vector comprising any one of the isolated nucleic acid molecules described herein.

As used herein, the term “vector”, “expression vector”, or “plasmid DNA” is used herein to refer to a recombinant nucleic acid construct that is manipulated by human intervention. A recombinant nucleic acid construct can contain two or more nucleic acid molecules that are linked in a manner such that the product is not found in a cell in nature. In particular, the two or more nucleic acid molecules can be operatively linked, such as a gene encoding a protein of interest, one or more protein tags, functional domains and the like. In a specific embodiment the proteins can include sIL-6R binding protein, CTGF binding protein, sIL-6R and anti-transferrin receptor bispecific antibody, CTGF and anti-transferrin receptor bispecific antibody, sIL-6R and CTGF multispecific binding protein, sIL-6R and CTGF and anti-transferrin receptor fusion protein.

Any of the nucleic acid molecules described herein can be incorporated into an expression vector. Vectors suitable for use in preparation of proteins and/or protein conjugates include those selected from baculovirus, phage, plasmid, phagemid, cosmid, fosmid, bacterial artificial chromosome, viral DNA, PI-based artificial chromosome, yeast plasmid, and yeast artificial chromosome. For example, the viral DNA vector can be selected from vaccinia, adenovirus, foul pox virus, pseudorabies and a derivative of SV40. Suitable bacterial vectors for use in the methods described herein include bacterial plasmids. One type of vector is a genomic integrated vector, or “integrated vector,” which can become integrated into the chromosomal DNA of the host cell. Another type of vector is an episomal vector, e.g., a nucleic acid capable of extra-chromosomal replication. Vectors capable of directing the expression of genes to which they are operatively linked are referred to herein as “expression vectors.” Viral vectors include adenovirus, adeno-associated virus (AAV), retroviruses, lentiviruses, vaccinia virus, measles viruses, herpes viruses, and bovine papilloma virus vectors (see, Kay et al., Proc. Natl. Acad. Sci. USA 94:12744-12746 (1997) for a review of viral and non-viral vectors). Viral vectors are modified so the native tropism and pathogenicity of the virus has been altered or removed. The genome of a virus also can be modified to increase its infectivity and to accommodate packaging of the nucleic acid encoding the polypeptide of interest.

In the expression vectors, regulatory elements controlling transcription can be generally derived from mammalian, microbial, viral or insect genes. The ability to replicate in a host, usually conferred by an origin of replication, and a selection gene to facilitate recognition of transformants may additionally be incorporated. Those of skill in the art can select a suitable regulatory region to be included in such a vector.

For example, an expression vector usually comprises one or more promoters, operably linked to the nucleic acid molecules of interest. As used herein, a promoter is intended mean a polynucleotide molecule capable of facilitating transcription of genes in operable linkage with the promoter. Many types of promoters are suitable for use with the constructs described herein. The promoter can be constitutive or inducible. Non-limiting examples of constitutive promoters include cytomegalovirus (CMV) promoter and the Rous sarcoma virus promoter, that allows for unregulated expression in mammalian cells. As used herein, “inducible” refers to both up-regulation and down regulation. An inducible promoter is a promoter that is capable of directly or indirectly activating transcription of one or more DNA sequences or genes in response to an inducer. In the absence of an inducer, the DNA sequences or genes will not be transcribed.

Additional regulatory elements that may be useful in vectors, include, but are not limited to, polyadenylation sequences, translation control sequences (e.g., an internal ribosome entry segment, IRES), enhancers, or introns. Such elements may not be necessary, although they may increase expression by affecting transcription, stability of the mRNA, translational efficiency, or the like. Such elements can be included in a nucleic acid construct as desired to obtain optimal expression of the nucleic acids in the cell(s). Sufficient expression, however, may sometimes be obtained without such additional elements. Vectors also can include other elements. For example, a vector can include a nucleic acid that encodes a signal peptide such that the encoded polypeptide is directed to a particular cellular location (e.g., a signal secretion sequence to cause the protein to be secreted by the cell) or a nucleic acid that encodes a selectable marker. Non-limiting examples of selectable markers include puromycin, adenosine deaminase (ADA), aminoglycoside phosphotransferase (neo, G418, APH), dihydrofolate reductase (DHFR), hygromycin-β-phosphotransferase, thymidine kinase (TK), and xanthin-guanine phosphoribosyltransferase (XGPRT). Such markers are useful for selecting stable transformants in culture.

For example, provided herein with this disclosure are expression vectors including:
(i) one or more nucleic acid molecules encoding a sIL-6R binding protein as described herein, for example including CDRs as set forth in SEQ ID NOs: 2-4, SEQ ID NOS: 6-8, SEQ ID NOs: 10-12, SEQ ID NOs: 14-16, SEQ ID NOs: 18-20, SEQ ID NOs: 22-24, SEQ ID NOs: 26-28, SEQ ID NOs: 30-32, SEQ ID NOs: 34-36, SEQ ID NOs: 38-40, SEQ ID NOs: 42-44, SEQ ID NOs: 46-48, SEQ ID NOs: 50-52, SEQ ID NOs: 54-56, SEQ ID NOs: 58-60, SEQ ID NOs: 62-64, SEQ ID NOs: 66-68, or SEQ ID NO: 138-194;
(ii) one or more nucleic acid molecules encoding a sIL-6R binding protein as described herein, for example including the amino acid sequence of SEQ ID NO:1, SEQ ID NO:5, SEQ ID NO: 9, SEQ ID NO: 13, SEQ ID NO: 17, SEQ ID NO:21, SEQ ID NO:25, SEQ ID NO:29, SEQ ID NO: 33, SEQ ID NO:37, SEQ ID NO:41, SEQ ID NO: 45, SEQ ID NO:49, SEQ ID NO:53, SEQ ID NO: 57, SEQ ID NO:61, SEQ ID NO:65, or SEQ ID NOs: 119-137;
(iii) one or more nucleic acid molecules encoding a CTGF binding protein as described herein, for example including CDRs as set forth in NOs: 70-72, SEQ ID NOs: 74-76, SEQ ID NOs: 78-80, SEQ ID NOs: 82-84, SEQ ID NOs: 86-88, SEQ ID NOs: 90-92, SEQ ID NOS: 233-286, or SEQ ID NOs: 291-293;
(iv) one or more nucleic acid molecules encoding a CTGF binding protein as described herein, for example including the amino acid sequence of SEQ ID NO:73, SEQ ID NO:77, SEQ ID NO: 81, SEQ ID NO:85, SEQ ID NO:89, or SEQ ID NOs: 214-232;
(v) one or more nucleic acid molecules encoding a multispecific binding protein that selectively and/or specifically binds to human sIL-6R and to human CTGF as described herein, for example including CDRs as set forth in SEQ ID NOs: 2-4 and SEQ ID NOs: 70-72, SEQ ID NOs: 6-8 and SEQ ID NOs: 70-72, SEQ ID NOs: 10-12 and SEQ ID NOs: 70-72, SEQ ID NOs: 14-16 and SEQ ID NOs: 70-72, SEQ ID NOs: 18-20 and SEQ ID NOs: 70-72, SEQ ID NOs: 22-24 and SEQ ID NOs: 70-72, SEQ ID NOs: 26-28 and SEQ ID NOs: 70-72, SEQ ID NOs: 30-32 and SEQ ID NOs: 70-72, SEQ ID NOs: 34-36 and SEQ ID NOs: 70-72, SEQ ID NOs: 38-40 and SEQ ID NOs: 70-72, SEQ ID NOs: 42-44 and SEQ ID NOs: 70-72, SEQ ID NOs: 46-48 and SEQ ID NOs: 70-72, SEQ ID NOs: 50-52 and SEQ ID NOs: 70-72, SEQ ID NOs: 54-56 and SEQ ID NOs: 70-72, SEQ ID NOs: 58-60 and SEQ ID NOs: 70-72, SEQ ID NOs: 62-64 and SEQ ID NOs: 70-72; SEQ ID NOs: 66-68 and SEQ ID NOs: 70-72 (furthermore any of the sIL-6R CDR sets shown in SEQ ID NOs: 138-194 can similarly be combined with SEQ ID NO:70-72 and any of the CTGF CDR sets as set forth in SEQ ID NOs: 233-286 can be used in place of SEQ ID NOs: 70-72); SEQ ID NOs: 2-4 and SEQ ID NOs: 74-76, SEQ ID NOs: 6-8 and SEQ ID NOs: 74-76, SEQ ID NOs: 10-12 and SEQ ID NOs: 74-76, SEQ ID NOs: 14-16 and SEQ ID NOs: 74-76, SEQ ID NOs: 18-20 and SEQ ID NOs: 74-76, SEQ ID NOs: 22-24 and SEQ ID NOs: 74-76, SEQ ID NOs: 26-28 and SEQ ID NOs: 74-76, SEQ ID NOs: 30-32 and SEQ ID NOs: 74-76, SEQ ID NOs: 34-36 and SEQ ID NOs: 74-76, SEQ ID NOs: 38-40 and SEQ ID NOs: 74-76, SEQ ID NOs: 42-44 and SEQ ID NOs: 74-76, SEQ ID NOs: 46-48 and SEQ ID NOs: 74-76, SEQ ID NOs: 50-52 and SEQ ID NOs: 74-76, SEQ ID NOs: 54-56 and SEQ ID NOs: 74-76, SEQ ID NOs: 58-60 and SEQ ID NOs: 74-76, SEQ ID NOs: 62-64 and SEQ ID NOs: 74-76; SEQ ID NOs: 66-68 and SEQ ID NOs: 74-76 (furthermore any of the sIL-6R CDR sets shown in SEQ ID NOs: 138-194 can similarly be combined with SEQ ID NO:74-76 and any of the CTGF CDR sets as set forth in SEQ ID NOs: 233-286 can be used in place of SEQ ID NOs: 74-76); SEQ ID NOs: 2-4 and SEQ ID NOs: 78-80, SEQ ID NOs: 6-8 and SEQ ID NOs: 78-80, SEQ ID NOs: 10-12 and SEQ ID NOs: 78-80, SEQ ID NOs: 14-16 and SEQ ID NOs: 78-80, SEQ ID NOs: 18-20 and SEQ ID NOs: 78-80, SEQ ID NOs: 22-24 and SEQ ID NOs: 78-80, SEQ ID NOs: 26-28 and SEQ ID NOs: 78-80, SEQ ID NOs: 30-32 and SEQ ID NOs: 78-80, SEQ ID NOs: 34-36 and SEQ ID NOs: 78-80, SEQ ID NOs: 38-40 and SEQ ID NOs: 78-80, SEQ ID NOs: 42-44 and SEQ ID NOs: 78-80, SEQ ID NOs: 46-48 and SEQ ID NOs: 78-80, SEQ ID NOs: 50-52 and SEQ ID NOs: 78-80, SEQ ID NOs: 54-56 and SEQ ID NOs: 78-80, SEQ ID NOs: 58-60 and SEQ ID NOs: 78-80, SEQ ID NOs: 62-64 and SEQ ID NOs: 78-80; SEQ ID NOs: 66-68 and SEQ ID NOs: 78-80; (furthermore any of the SIL-6R CDR sets shown in SEQ ID NOs: 138-194 can similarly be combined with SEQ ID NO:78-80 and any of the CTGF CDR sets as set forth in SEQ ID NOs: 233-286 can be used in place of SEQ ID NOs: 78-80); SEQ ID NOs: 2-4 and SEQ ID NOs: 82-84, SEQ ID NOs: 6-8 and SEQ ID NOs: 82-84, SEQ ID NOs: 10-12 and SEQ ID NOs: 82-84, SEQ ID NOs: 14-16 and SEQ ID NOs: 82-84, SEQ ID NOs: 18-20 and SEQ ID NOs: 82-84, SEQ ID NOs: 22-24 and SEQ ID NOs: 82-84, SEQ ID NOs: 26-28 and SEQ ID NOs: 82-84, SEQ ID NOs: 30-32 and SEQ ID NOs: 82-84, SEQ ID NOs: 34-36 and SEQ ID NOs: 82-84, SEQ ID NOs: 38-40 and SEQ ID NOs: 82-84, SEQ ID NOs: 42-44 and SEQ ID NOs: 82-84, SEQ ID NOs: 46-48 and SEQ ID NOs: 82-84, SEQ ID NOs: 50-52 and SEQ ID NOs: 82-84, SEQ ID NOs: 54-56 and SEQ ID NOs: 82-84, SEQ ID NOs: 58-60 and SEQ ID NOs: 82-84, SEQ ID NOs: 62-64 and SEQ ID NOs: 82-84; SEQ ID NOs: 66-68 and SEQ ID NOs: 82-84 (furthermore any of the sIL-6R CDR sets shown in SEQ ID NOs: 138-194 can similarly be combined with SEQ ID NO:82-84 and any of the CTGF CDR sets as set forth in SEQ ID NOS: 233-286 can be used in place of SEQ ID NOs: 82-84); SEQ ID NOs: 2-4 and SEQ ID NOs: 86-88, SEQ ID NOs: 6-8 and SEQ ID NOs: 86-88, SEQ ID NOs: 10-12 and SEQ ID NOs: 86-88, SEQ ID NOs: 14-16 and SEQ ID NOs: 86-88, SEQ ID NOs: 18-20 and SEQ ID NOs: 86-88, SEQ ID NOs: 22-24 and SEQ ID NOs: 86-88, SEQ ID NOs: 26-28 and SEQ ID NOs: 86-88, SEQ ID NOs: 30-32 and SEQ ID NOs: 86-88, SEQ ID NOs: 34-36 and SEQ ID NOs: 86-88, SEQ ID NOs: 38-40 and SEQ ID NOs: 86-88, SEQ ID NOs: 42-44 and SEQ ID NOs: 86-88, SEQ ID NOs: 46-48 and SEQ ID NOs: 86-88, SEQ ID NOs: 50-52 and SEQ ID NOs: 86-88, SEQ ID NOs: 54-56 and SEQ ID NOs: 86-88, SEQ ID NOs: 58-60 and SEQ ID NOs: 86-88, SEQ ID NOs: 62-64 and SEQ ID NOs: 86-88; SEQ ID NOs: 66-68 and SEQ ID NOs: 86-88; (furthermore any of the sIL-6R CDR sets shown in SEQ ID NOs: 138-194 can similarly be combined with SEQ ID NO: 86-88 and any of the CTGF CDR sets as set forth in SEQ ID NOs: 233-286 can be used in place of SEQ ID NOs: 86-88); SEQ ID NOs: 2-4 and SEQ ID NOs: 90-92, SEQ ID NOs: 6-8 and SEQ ID NOs: 90-92, SEQ ID NOs: 10-12 and SEQ ID NOs: 90-92, SEQ ID NOs: 14-16 and SEQ ID NOs: 90-92, SEQ ID NOs: 18-20 and SEQ ID NOs: 90-92, SEQ ID NOs: 22-24 and SEQ ID NOs: 90-92, SEQ ID NOs: 26-28 and SEQ ID NOs: 90-92, SEQ ID NOs: 30-32 and SEQ ID NOs: 90-92, SEQ ID NOs: 34-36 and SEQ ID NOs: 90-92, SEQ ID NOs: 38-40 and SEQ ID NOs: 90-92, SEQ ID NOs: 42-44 and SEQ ID NOs: 90-92, SEQ ID NOs: 46-48 and SEQ ID NOs: 90-92, SEQ ID NOs: 50-52 and SEQ ID NOs: 90-92, SEQ ID NOs: 54-56 and SEQ ID NOs: 90-92, SEQ ID NOs: 58-60 and SEQ ID NOs: 90-92, SEQ ID NOs: 62-64 and SEQ ID NOs: 90-92 or SEQ ID NOs: 66-68 and SEQ ID NOs: 90-92 (furthermore any of the sIL-6R CDR sets shown in SEQ ID NOs: 138-194 can similarly be combined with SEQ ID NO:90-92 and any of the CTGF CDR sets as set forth in SEQ ID NOs: 233-286 can be used in place of SEQ ID NOs: 90-92);
(vi) one or more nucleic acid molecules encoding a multispecific binding protein that selectively and/or specifically binds to human sIL-6R and to human CTGF as described herein, for example including the amino acid sequence of: SEQ ID NO: 1 and SEQ ID NO:69, SEQ ID NO: 5 and SEQ ID NO: 69, SEQ ID NO: 9 and SEQ ID NO:69, SEQ ID NO: 13 and SEQ ID NO:69, SEQ ID NO: 17 and SEQ ID NO:69, SEQ ID NO:21 and SEQ ID NO:69, SEQ ID NO:25 and SEQ ID NO: 69, SEQ ID NO:29 and SEQ ID NO:69, SEQ ID NO:33 and SEQ ID NO:69, SEQ ID NO:37 and SEQ ID NO:69, SEQ ID NO:41 and SEQ ID NO:69, SEQ ID NO:45 and SEQ ID NO:69, SEQ ID NO: 49 and SEQ ID NO:69, SEQ ID NO:53 and SEQ ID NO:69, SEQ ID NO:57 and SEQ ID NO: 69, SEQ ID NO:61 and SEQ ID NO:69, SEQ ID NO:65 and SEQ ID NO:69 (furthermore any of the sIL-6R VHHs shown in SEQ ID NOs: 119-137 can similarly be combined with SEQ ID NO:69 and any of SEQ ID NO:214-232 can be used in place of SEQ ID NO:69); SEQ ID NO: 1 and SEQ ID NO: 73, SEQ ID NO:5 and SEQ ID NO:73, SEQ ID NO:9 and SEQ ID NO:73, SEQ ID NO: 13 and SEQ ID NO:73, SEQ ID NO: 17 and SEQ ID NO:73, SEQ ID NO:21 and SEQ ID NO:73, SEQ ID NO: 25 and SEQ ID NO:73, SEQ ID NO:29 and SEQ ID NO:73, SEQ ID NO:33 and SEQ ID NO: 73, SEQ ID NO:37 and SEQ ID NO:73, SEQ ID NO:41 and SEQ ID NO:73, SEQ ID NO:45 and SEQ ID NO:73, SEQ ID NO:49 and SEQ ID NO:73, SEQ ID NO:53 and SEQ ID NO:73, SEQ ID NO: 57 and SEQ ID NO:73, SEQ ID NO:61 and SEQ ID NO:73, SEQ ID NO:65 and SEQ ID NO: 73 (furthermore any of the sIL-6R VHHs shown in SEQ ID NOs: 119-137 can similarly be combined with SEQ ID NO:73 and any of SEQ ID NO:214-232 can be used in place of SEQ ID NO: 73); SEQ ID NO: 1 and SEQ ID NO:77, SEQ ID NO:5 and SEQ ID NO:77, SEQ ID NO:9 and SEQ ID NO:77, SEQ ID NO: 13 and SEQ ID NO:77, SEQ ID NO: 17 and SEQ ID NO:77, SEQ ID NO: 21 and SEQ ID NO: 77, SEQ ID NO:25 and SEQ ID NO:77, SEQ ID NO:29 and SEQ ID NO:77, SEQ ID NO:33 and SEQ ID NO:77, SEQ ID NO:37 and SEQ ID NO:77, SEQ ID NO:41 and SEQ ID NO: 77, SEQ ID NO:45 and SEQ ID NO:77, SEQ ID NO:49 and SEQ ID NO:77, SEQ ID NO:53 and SEQ ID NO:77, SEQ ID NO:57 and SEQ ID NO:77, SEQ ID NO:61 and SEQ ID NO:77, SEQ ID NO: 65 and SEQ ID NO:77 (furthermore any of the sIL-6R VHHs shown in SEQ ID NOs: 119-137 can similarly be combined with SEQ ID NO:77 and any of SEQ ID NO:214-232 can be used in place of SEQ ID NO:77); SEQ ID NO: 1 and SEQ ID NO:81, SEQ ID NO:5 and SEQ ID NO:81, SEQ ID NO:9 and SEQ ID NO:81, SEQ ID NO: 13 and SEQ ID NO:81, SEQ ID NO: 17 and SEQ ID NO: 81, SEQ ID NO:21 and SEQ ID NO:81, SEQ ID NO:25 and SEQ ID NO:81, SEQ ID NO:29 and SEQ ID NO:81, SEQ ID NO:33 and SEQ ID NO:81, SEQ ID NO:37 and SEQ ID NO:81, SEQ ID NO: 41 and SEQ ID NO:81, SEQ ID NO:45 and SEQ ID NO:81, SEQ ID NO:49 and SEQ ID NO: 81, SEQ ID NO:53 and SEQ ID NO:81, SEQ ID NO:57 and SEQ ID NO:81, SEQ ID NO:61 and SEQ ID NO:81, SEQ ID NO:65 and SEQ ID NO:81 (furthermore any of the sIL-6R VHHs shown in SEQ ID NOs: 119-137 can similarly be combined with SEQ ID NO:81 and any of SEQ ID NO: 214-232 can be used in place of SEQ ID NO:81); SEQ ID NO: 1 and SEQ ID NO:85, SEQ ID NO: 5 and SEQ ID NO:85, SEQ ID NO:9 and SEQ ID NO:85, SEQ ID NO: 13 and SEQ ID NO: 85, SEQ ID NO: 17 and SEQ ID NO:85, SEQ ID NO:21 and SEQ ID NO:85, SEQ ID NO:25 and SEQ ID NO: 85, SEQ ID NO:29 and SEQ ID NO:85, SEQ ID NO:33 and SEQ ID NO:85, SEQ ID NO: 37 and SEQ ID NO:85, SEQ ID NO:41 and SEQ ID NO:85, SEQ ID NO:45 and SEQ ID NO: 85, SEQ ID NO:49 and SEQ ID NO:85, SEQ ID NO:53 and SEQ ID NO:85, SEQ ID NO:57 and SEQ ID NO:85, SEQ ID NO:61 and SEQ ID NO:85, SEQ ID NO:65 and SEQ ID NO:85 (furthermore any of the sIL-6R VHHs shown in SEQ ID NOs: 119-137 can similarly be combined with SEQ ID NO: 85 and any of SEQ ID NO:214-232 can be used in place of SEQ ID NO: 85); SEQ ID NO: 1 and SEQ ID NO:89, SEQ ID NO:5 and SEQ ID NO: 89, SEQ ID NO:9 and SEQ ID NO: 89, SEQ ID NO: 13 and SEQ ID NO:89, SEQ ID NO: 17 and SEQ ID NO:89, SEQ ID NO:21 and SEQ ID NO: 89, SEQ ID NO:25 and SEQ ID NO:89, SEQ ID NO:29 and SEQ ID NO:89, SEQ ID NO:33 and SEQ ID NO:89, SEQ ID NO:37 and SEQ ID NO:89, SEQ ID NO:41 and SEQ ID NO:89, SEQ ID NO: 45 and SEQ ID NO:89, SEQ ID NO:49 and SEQ ID NO:89, SEQ ID NO:53 and SEQ ID NO: 89, SEQ ID NO:57 and SEQ ID NO: 89, SEQ ID NO:61 and SEQ ID NO:89, or SEQ ID NO:65 and SEQ ID NO:89 (furthermore any of the sIL-6R VHHs shown in SEQ ID NOs: 119-137 can similarly be combined with SEQ ID NO:89 and any of SEQ ID NO:214-232 can be used in place of SEQ ID NO:89);
(vii) one or more nucleic acid molecules encoding a fusion protein including a sIL-6R binding protein as described herein and an anti-transferrin receptor antibody, for example including CDRs as set forth in SEQ ID NOs: 2-4, SEQ ID NOS: 6-8, SEQ ID NOs: 10-12, SEQ ID NOs: 14-16, SEQ ID NOs: 18-20, SEQ ID NOs: 22-24, SEQ ID NOs: 26-28, SEQ ID NOs: 30-32, SEQ ID NOs: 34-36, SEQ ID NOs: 38-40, SEQ ID NOs: 42-44, SEQ ID NOs: 46-48, SEQ ID NOs: 50-52, SEQ ID NOs: 54-56, SEQ ID NOs: 58-60, SEQ ID NOs: 62-64, SEQ ID NOs: 66-68, or SEQ ID NO: 138-194 each further including a nucleic acid molecule encoding an anti-transferrin receptor antibody;
(viii) one or more nucleic acid molecules encoding a fusion protein including a sIL-6R binding protein as described herein and an anti-transferrin receptor antibody, for example including the amino acid sequence of SEQ ID NO:1, SEQ ID NO:5, SEQ ID NO:9, SEQ ID NO: 13, SEQ ID NO: 17, SEQ ID NO:21, SEQ ID NO:25, SEQ ID NO:29, SEQ ID NO:33, SEQ ID NO:37, SEQ ID NO:41, SEQ ID NO:45, SEQ ID NO:49, SEQ ID NO:53, SEQ ID NO:57, SEQ ID NO:61, SEQ ID NO:65, or SEQ ID NO: 119-137, each further including a nucleic acid molecule encoding an anti-transferrin receptor antibody;
(ix) one or more nucleic acid molecule encoding a fusion protein including a CTGF binding protein as described herein and an anti-transferrin receptor antibody, for example including CDRs as set forth in NOs: 70-72, SEQ ID NOs: 74-76, SEQ ID NOs: 78-80, SEQ ID NOS: 82-84, SEQ ID NOs: 86-88, SEQ ID NOs: 90-92, SEQ ID NOs: 233-286, or SEQ ID NOs: 291-293 each further including a nucleic acid molecule an anti-transferrin receptor antibody;
(x) one or more nucleic acid molecules encoding a fusion protein including a CTGF binding protein as described herein and an anti-transferrin receptor antibody, for example including the amino acid sequence of SEQ ID NO:73, SEQ ID NO:77, SEQ ID NO:81, SEQ ID NO:85, SEQ ID NO: 89, or SEQ ID NO:214-232 each further including a nucleic acid molecule encoding an anti-transferrin receptor antibody;
(xi) one or more nucleic acid molecule encoding a fusion protein including a multispecific binding protein that selectively and/or specifically binds to human sIL-6R and to human CTGF as described herein and an anti-transferrin receptor antibody, for example including CDRs as set forth in SEQ ID NOs: 2-4 and SEQ ID NOs: 70-72, SEQ ID NOs: 6-8 and SEQ ID NOs: 70-72, SEQ ID NOs: 10-12 and SEQ ID NOs: 70-72, SEQ ID NOs: 14-16 and SEQ ID NOs: 70-72, SEQ ID NOs: 18-20 and SEQ ID NOs: 70-72, SEQ ID NOs: 22-24 and SEQ ID NOs: 70-72, SEQ ID NOs: 26-28 and SEQ ID NOs: 70-72, SEQ ID NOs: 30-32 and SEQ ID NOs: 70-72, SEQ ID NOs: 34-36 and SEQ ID NOs: 70-72, SEQ ID NOs: 38-40 and SEQ ID NOs: 70-72, SEQ ID NOs: 42-44 and SEQ ID NOs: 70-72, SEQ ID NOs: 46-48 and SEQ ID NOs: 70-72, SEQ ID NOs: 50-52 and SEQ ID NOs: 70-72, SEQ ID NOs: 54-56 and SEQ ID NOs: 70-72, SEQ ID NOs: 58-60 and SEQ ID NOs: 70-72, SEQ ID NOs: 62-64 and SEQ ID NOs: 70-72; SEQ ID NOs: 66-68 and SEQ ID NOs: 70-72 (furthermore any of the sIL-6R CDR sets shown in SEQ ID NOs: 138-194 can similarly be combined with SEQ ID NO: 70-72 and any of the CTGF CDR sets as set forth in SEQ ID NOs: 233-286 can be used in place of SEQ ID NOs: 70-72); SEQ ID NOs: 2-4 and SEQ ID NOs: 74-76, SEQ ID NOs: 6-8 and SEQ ID NOs: 74-76, SEQ ID NOs: 10-12 and SEQ ID NOs: 74-76, SEQ ID NOs: 14-16 and SEQ ID NOs: 74-76, SEQ ID NOs: 18-20 and SEQ ID NOs: 74-76, SEQ ID NOs: 22-24 and SEQ ID NOs: 74-76, SEQ ID NOs: 26-28 and SEQ ID NOs: 74-76, SEQ ID NOs: 30-32 and SEQ ID NOs: 74-76, SEQ ID NOs: 34-36 and SEQ ID NOs: 74-76, SEQ ID NOs: 38-40 and SEQ ID NOs: 74-76, SEQ ID NOs: 42-44 and SEQ ID NOs: 74-76, SEQ ID NOs: 46-48 and SEQ ID NOs: 74-76, SEQ ID NOs: 50-52 and SEQ ID NOs: 74-76, SEQ ID NOs: 54-56 and SEQ ID NOs: 74-76, SEQ ID NOs: 58-60 and SEQ ID NOs: 74-76, SEQ ID NOs: 62-64 and SEQ ID NOs: 74-76; SEQ ID NOs: 66-68 and SEQ ID NOs: 74-76 (furthermore any of the sIL-6R CDR sets shown in SEQ ID NOs: 138-194 can similarly be combined with SEQ ID NO:74-76 and any of the CTGF CDR sets as set forth in SEQ ID NOs: 233-286 can be used in place of SEQ ID NOs: 74-76); SEQ ID NOs: 2-4 and SEQ ID NOs: 78-80, SEQ ID NOs: 6-8 and SEQ ID NOs: 78-80, SEQ ID NOs: 10-12 and SEQ ID NOs: 78-80, SEQ ID NOs: 14-16 and SEQ ID NOs: 78-80, SEQ ID NOs: 18-20 and SEQ ID NOs: 78-80, SEQ ID NOs: 22-24 and SEQ ID NOs: 78-80, SEQ ID NOs: 26-28 and SEQ ID NOs: 78-80, SEQ ID NOs: 30-32 and SEQ ID NOs: 78-80, SEQ ID NOs: 34-36 and SEQ ID NOs: 78-80, SEQ ID NOs: 38-40 and SEQ ID NOs: 78-80, SEQ ID NOs: 42-44 and SEQ ID NOs: 78-80, SEQ ID NOs: 46-48 and SEQ ID NOs: 78-80, SEQ ID NOs: 50-52 and SEQ ID NOs: 78-80, SEQ ID NOs: 54-56 and SEQ ID NOs: 78-80, SEQ ID NOs: 58-60 and SEQ ID NOs: 78-80, SEQ ID NOs: 62-64 and SEQ ID NOs: 78-80; SEQ ID NOs: 66-68 and SEQ ID NOs: 78-80 (furthermore any of the sIL-6R CDR sets shown in SEQ ID NOs: 138-194 can similarly be combined with SEQ ID NO:78-80 and any of the CTGF CDR sets as set forth in SEQ ID NOs: 233-286 can be used in place of SEQ ID NOs: 78-80); SEQ ID NOS: 2-4 and SEQ ID NOs: 82-84, SEQ ID NOs: 6-8 and SEQ ID NOs: 82-84, SEQ ID NOs: 10-12 and SEQ ID NOs: 82-84, SEQ ID NOs: 14-16 and SEQ ID NOs: 82-84, SEQ ID NOs: 18-20 and SEQ ID NOs: 82-84, SEQ ID NOs: 22-24 and SEQ ID NOs: 82-84, SEQ ID NOs: 26-28 and SEQ ID NOs: 82-84, SEQ ID NOs: 30-32 and SEQ ID NOs: 82-84, SEQ ID NOs: 34-36 and SEQ ID NOs: 82-84, SEQ ID NOs: 38-40 and SEQ ID NOs: 82-84, SEQ ID NOs: 42-44 and SEQ ID NOs: 82-84, SEQ ID NOs: 46-48 and SEQ ID NOs: 82-84, SEQ ID NOs: 50-52 and SEQ ID NOs: 82-84, SEQ ID NOs: 54-56 and SEQ ID NOs: 82-84, SEQ ID NOs: 58-60 and SEQ ID NOs: 82-84, SEQ ID NOs: 62-64 and SEQ ID NOs: 82-84; SEQ ID NOs: 66-68 and SEQ ID NOs: 82-84 (furthermore any of the sIL-6R CDR sets shown in SEQ ID NOs: 138-194 can similarly be combined with SEQ ID NO:82-84 and any of the CTGF CDR sets as set forth in SEQ ID NOs: 233-286 can be used in place of SEQ ID NOs: 82-84); SEQ ID NOs: 2-4 and SEQ ID NOs: 86-88, SEQ ID NOs: 6-8 and SEQ ID NOs: 86-88, SEQ ID NOs: 10-12 and SEQ ID NOs: 86-88, SEQ ID NOs: 14-16 and SEQ ID NOs: 86-88, SEQ ID NOs: 18-20 and SEQ ID NOs: 86-88, SEQ ID NOs: 22-24 and SEQ ID NOs: 86-88, SEQ ID NOS: 26-28 and SEQ ID NOs: 86-88, SEQ ID NOs: 30-32 and SEQ ID NOs: 86-88, SEQ ID NOs: 34-36 and SEQ ID NOs: 86-88, SEQ ID NOs: 38-40 and SEQ ID NOs: 86-88, SEQ ID NOs: 42-44 and SEQ ID NOs: 86-88, SEQ ID NOs: 46-48 and SEQ ID NOs: 86-88, SEQ ID NOs: 50-52 and SEQ ID NOs: 86-88, SEQ ID NOs: 54-56 and SEQ ID NOs: 86-88, SEQ ID NOs: 58-60 and SEQ ID NOs: 86-88, SEQ ID NOs: 62-64 and SEQ ID NOs: 86-88; SEQ ID NOs: 66-68 and SEQ ID NOs: 86-88 (furthermore any of the sIL-6R CDR sets shown in SEQ ID NOs: 138-194 can similarly be combined with SEQ ID NO: 86-88 and any of the CTGF CDR sets as set forth in SEQ ID NOs: 233-286 can be used in place of SEQ ID NOs: 86-88); SEQ ID NOs: 2-4 and SEQ ID NOs: 90-92, SEQ ID NOs: 6-8 and SEQ ID NOs: 90-92, SEQ ID NOs: 10-12 and SEQ ID NOs: 90-92, SEQ ID NOs: 14-16 and SEQ ID NOs: 90-92, SEQ ID NOs: 18-20 and SEQ ID NOs: 90-92, SEQ ID NOs: 22-24 and SEQ ID NOs: 90-92, SEQ ID NOs: 26-28 and SEQ ID NOs: 90-92, SEQ ID NOs: 30-32 and SEQ ID NOs: 90-92, SEQ ID NOs: 34-36 and SEQ ID NOs: 90-92, SEQ ID NOs: 38-40 and SEQ ID NOs: 90-92, SEQ ID NOs: 42-44 and SEQ ID NOs: 90-92, SEQ ID NOs: 46-48 and SEQ ID NOs: 90-92, SEQ ID NOs: 50-52 and SEQ ID NOs: 90-92, SEQ ID NOs: 54-56 and SEQ ID NOs: 90-92, SEQ ID NOs: 58-60 and SEQ ID NOs: 90-92, SEQ ID NOs: 62-64 and SEQ ID NOs: 90-92 or SEQ ID NOs: 66-68 and SEQ ID NOs: 90-92 (furthermore any of the sIL-6R CDR sets shown in SEQ ID NOs: 138-194 can similarly be combined with SEQ ID NO:90-92 and any of the CTGF CDR sets as set forth in SEQ ID NOs: 233-286 can be used in place of SEQ ID NOs: 90-92), each further including a nucleic acid molecule encoding an anti-transferrin receptor antibody; and
(xii) one or more nucleic acid molecule encoding a fusion protein including a multispecific binding protein that selectively and/or specifically binds to human sIL-6R and to human CTGF as described herein and an anti-transferrin receptor antibody, for example including the amino acid sequence of: SEQ ID NO: 1 and SEQ ID NO:69, SEQ ID NO:5 and SEQ ID NO:69, SEQ ID NO:9 and SEQ ID NO:69, SEQ ID NO: 13 and SEQ ID NO:69, SEQ ID NO: 17 and SEQ ID NO:69, SEQ ID NO: 21 and SEQ ID NO:69, SEQ ID NO:25 and SEQ ID NO:69, SEQ ID NO:29 and SEQ ID NO: 69, SEQ ID NO:33 and SEQ ID NO:69, SEQ ID NO:37 and SEQ ID NO:69, SEQ ID NO:41 and SEQ ID NO:69, SEQ ID NO:45 and SEQ ID NO:69, SEQ ID NO:49 and SEQ ID NO:69, SEQ ID NO: 53 and SEQ ID NO:69, SEQ ID NO:57 and SEQ ID NO:69, SEQ ID NO:61 and SEQ ID NO: 69, SEQ ID NO:65 and SEQ ID NO:69 (furthermore any of the sIL-6R VHHs shown in SEQ ID NOs: 119-137 can similarly be combined with SEQ ID NO:69 and any of SEQ ID NO:214-232 can be used in place of SEQ ID NO:69); SEQ ID NO:1 and SEQ ID NO:73, SEQ ID NO:5 and SEQ ID NO:73, SEQ ID NO:9 and SEQ ID NO:73, SEQ ID NO: 13 and SEQ ID NO:73, SEQ ID NO: 17 and SEQ ID NO:73, SEQ ID NO:21 and SEQ ID NO:73, SEQ ID NO:25 and SEQ ID NO:73, SEQ ID NO:29 and SEQ ID NO:73, SEQ ID NO:33 and SEQ ID NO:73, SEQ ID NO:37 and SEQ ID NO: 73, SEQ ID NO: 41 and SEQ ID NO:73, SEQ ID NO:45 and SEQ ID NO:73, SEQ ID NO:49 and SEQ ID NO:73, SEQ ID NO:53 and SEQ ID NO:73, SEQ ID NO:57 and SEQ ID NO:73, SEQ ID NO: 61 and SEQ ID NO:73, SEQ ID NO: 65 and SEQ ID NO:73 (furthermore any of the sIL-6R VHHs shown in SEQ ID NOs: 119-137 can similarly be combined with SEQ ID NO:73 and any of SEQ ID NO:214-232 can be used in place of SEQ ID NO:73); SEQ ID NO:1 and SEQ ID NO:77, SEQ ID NO:5 and SEQ ID NO:77, SEQ ID NO:9 and SEQ ID NO:77, SEQ ID NO: 13 and SEQ ID NO: 77, SEQ ID NO: 17 and SEQ ID NO:77, SEQ ID NO:21 and SEQ ID NO:77, SEQ ID NO:25 and SEQ ID NO:77, SEQ ID NO:29 and SEQ ID NO:77, SEQ ID NO:33 and SEQ ID NO:77, SEQ ID NO: 37 and SEQ ID NO:77, SEQ ID NO:41 and SEQ ID NO:77, SEQ ID NO:45 and SEQ ID NO: 77, SEQ ID NO:49 and SEQ ID NO:77, SEQ ID NO:53 and SEQ ID NO:77, SEQ ID NO:57 and SEQ ID NO:77, SEQ ID NO:61 and SEQ ID NO:77, SEQ ID NO:65 and SEQ ID NO:77 (furthermore any of the sIL-6R VHHs shown in SEQ ID NOs: 119-137 can similarly be combined with SEQ ID NO:77 and any of SEQ ID NO:214-232 can be used in place of SEQ ID NO:77); SEQ ID NO: 1 and SEQ ID NO:81, SEQ ID NO:5 and SEQ ID NO:81, SEQ ID NO:9 and SEQ ID NO:81, SEQ ID NO: 13 and SEQ ID NO:81, SEQ ID NO: 17 and SEQ ID NO:81, SEQ ID NO:21 and SEQ ID NO: 81, SEQ ID NO:25 and SEQ ID NO:81, SEQ ID NO:29 and SEQ ID NO:81, SEQ ID NO:33 and SEQ ID NO:81, SEQ ID NO:37 and SEQ ID NO:81, SEQ ID NO:41 and SEQ ID NO:81, SEQ ID NO: 45 and SEQ ID NO:81, SEQ ID NO:49 and SEQ ID NO:81, SEQ ID NO:53 and SEQ ID NO: 81, SEQ ID NO:57 and SEQ ID NO:81, SEQ ID NO:61 and SEQ ID NO:81, SEQ ID NO:65 and SEQ ID NO:81 (furthermore any of the sIL-6R VHHs shown in SEQ ID NOs: 119-137 can similarly be combined with SEQ ID NO:81 and any of SEQ ID NO:214-232 can be used in place of SEQ ID NO:81); SEQ ID NO:1 and SEQ ID NO:85, SEQ ID NO:5 and SEQ ID NO:85, SEQ ID NO: 9 and SEQ ID NO: 85, SEQ ID NO: 13 and SEQ ID NO:85, SEQ ID NO: 17 and SEQ ID NO:85, SEQ ID NO:21 and SEQ ID NO:85, SEQ ID NO:25 and SEQ ID NO:85, SEQ ID NO:29 and SEQ ID NO: 85, SEQ ID NO:33 and SEQ ID NO:85, SEQ ID NO:37 and SEQ ID NO:85, SEQ ID NO:41 and SEQ ID NO: 85, SEQ ID NO: 45 and SEQ ID NO:85, SEQ ID NO:49 and SEQ ID NO:85, SEQ ID NO: 53 and SEQ ID NO:85, SEQ ID NO:57 and SEQ ID NO:85, SEQ ID NO:61 and SEQ ID NO: 85, SEQ ID NO: 65 and SEQ ID NO:85 (furthermore any of the sIL-6R VHHs shown in SEQ ID NOs: 119-137 can similarly be combined with SEQ ID NO:85 and any of SEQ ID NO:214-232 can be used in place of SEQ ID NO:85); SEQ ID NO: 1 and SEQ ID NO:89, SEQ ID NO:5 and SEQ ID NO:89, SEQ ID NO:9 and SEQ ID NO:89, SEQ ID NO: 13 and SEQ ID NO:89, SEQ ID NO: 17 and SEQ ID NO: 89, SEQ ID NO:21 and SEQ ID NO: 89, SEQ ID NO:25 and SEQ ID NO:89, SEQ ID NO:29 and SEQ ID NO:89, SEQ ID NO:33 and SEQ ID NO:89, SEQ ID NO:37 and SEQ ID NO: 89, SEQ ID NO: 41 and SEQ ID NO: 89, SEQ ID NO:45 and SEQ ID NO: 89, SEQ ID NO:49 and SEQ ID NO:89, SEQ ID NO:53 and SEQ ID NO:89, SEQ ID NO:57 and SEQ ID NO:89, SEQ ID NO: 61 and SEQ ID NO:89, or SEQ ID NO:65 and SEQ ID NO:89 (furthermore any of the sIL-6R VHHs shown in SEQ ID NOs: 119-137 can similarly be combined with SEQ ID NO:89 and any of SEQ ID NO:214-232 can be used in place of SEQ ID NO:89), each further including a nucleic acid molecule encoding an anti-transferrin receptor antibody.

### Host Cells

An embodiment provides an isolated host cell comprising any one of the expression vectors described herein.

Nucleic acid constructs described herein may be introduced into a host cell to be altered thus allowing expression of the chimeric protein within the cell, thereby generating a genetically engineered cell. A variety of methods are suitable for introduction of nucleic acid molecules and/or constructs into a cell, including viral and non-viral mediated techniques. Examples of typical non-viral mediated techniques include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion. Other methods of transfection include proprietary transfection reagents such as Lipofectamine™, Dojindo Hilymax™, Fugene™, jetPEI™, Effectene™ and DreamFect™

Nucleic acid constructs can be introduced into a host cell to be altered thus allowing expression of the chimeric protein within the cell. A variety of host cells are known in the art and suitable for chimeric proteins expression. Examples of typical cell used for transfection include, but are not limited to, a bacterial cell, a eukaryotic cell, a yeast cell, an insect cell, an immune cell, or a plant cell. For example, E. coli, Bacillus, Streptomyces, Pichia pastoris, Salmonella typhimurium, Drosophila S2, Spodoptera SJ9, CHO, COS (e.g., COS-7),3T3-F442A, HeLa, HUVEC, HUAEC, NIH 3T3, Jurkat, 293, 293H, or 293F.

For example, provided herein with this disclosure are host cells including:
(i) one or more expression vectors including nucleic acid molecules encoding a sIL-6R binding protein as described herein, for example including CDRs as set forth in SEQ ID NOs: 2-4, SEQ ID NOs: 6-8, SEQ ID NOs: 10-12, SEQ ID NOs: 14-16, SEQ ID NOs: 18-20, SEQ ID NOS: 22-24, SEQ ID NOs: 26-28, SEQ ID NOs: 30-32, SEQ ID NOs: 34-36, SEQ ID NOs: 38-40, SEQ ID NOs: 42-44, SEQ ID NOs: 46-48, SEQ ID NOs: 50-52, SEQ ID NOs: 54-56, SEQ ID NOs: 58-60, SEQ ID NOs: 62-64, SEQ ID NOs: 66-68, or SEQ ID NO: 138-194;
(ii) one or more expression vectors including nucleic acid molecules encoding a sIL-6R binding protein as described herein, for example including the amino acid sequence of SEQ ID NO: 1, SEQ ID NO:5, SEQ ID NO:9, SEQ ID NO: 13, SEQ ID NO: 17, SEQ ID NO:21, SEQ ID NO: 25, SEQ ID NO:29, SEQ ID NO:33, SEQ ID NO:37, SEQ ID NO:41, SEQ ID NO:45, SEQ ID NO: 49, SEQ ID NO:53, SEQ ID NO:57, SEQ ID NO:61, SEQ ID NO:65, or SEQ ID NOs: 119-137;
(iii) one or more expression vectors including nucleic acid molecules encoding a CTGF binding protein as described herein, for example including CDRs as set forth in NOs: 70-72, SEQ ID NOs: 74-76, SEQ ID NOs: 78-80, SEQ ID NOs: 82-84, SEQ ID NOs: 86-88, SEQ ID NOs: 90-92, SEQ ID NOs: 233-286, or SEQ ID NOs: 291-293;
(iv) one or more expression vectors including nucleic acid molecules encoding a CTGF binding protein as described herein, for example including the amino acid sequence of SEQ ID NO: 73, SEQ ID NO: 77, SEQ ID NO: 81, SEQ ID NO: 85, SEQ ID NO:89 or SEQ ID NOs: 214-232;
(v) one or more expression vectors including nucleic acid molecules encoding a multispecific binding protein that selectively and/or specifically binds to human sIL-6R and to human CTGF as described herein, for example including CDRs as set forth in SEQ ID NOs: 2-4 and SEQ ID NOs: 70-72, SEQ ID NOs: 6-8 and SEQ ID NOs: 70-72, SEQ ID NOs: 10-12 and SEQ ID NOs: 70-72, SEQ ID NOs: 14-16 and SEQ ID NOs: 70-72, SEQ ID NOs: 18-20 and SEQ ID NOs: 70-72, SEQ ID NOs: 22-24 and SEQ ID NOs: 70-72, SEQ ID NOs: 26-28 and SEQ ID NOs: 70-72, SEQ ID NOs: 30-32 and SEQ ID NOs: 70-72, SEQ ID NOs: 34-36 and SEQ ID NOs: 70-72, SEQ ID NOs: 38-40 and SEQ ID NOs: 70-72, SEQ ID NOs: 42-44 and SEQ ID NOs: 70-72, SEQ ID NOs: 46-48 and SEQ ID NOs: 70-72, SEQ ID NOs: 50-52 and SEQ ID NOs: 70-72, SEQ ID NOs: 54-56 and SEQ ID NOs: 70-72, SEQ ID NOs: 58-60 and SEQ ID NOs: 70-72, SEQ ID NOs: 62-64 and SEQ ID NOs: 70-72; SEQ ID NOs: 66-68 and SEQ ID NOs: 70-72 (furthermore any of the sIL-6R CDR sets shown in SEQ ID NOs: 138-194 can similarly be combined with SEQ ID NO:70-72 and any of the CTGF CDR sets as set forth in SEQ ID NOs: 233-286 can be used in place of SEQ ID NOs: 70-72); SEQ ID NOs: 2-4 and SEQ ID NOs: 74-76, SEQ ID NOs: 6-8 and SEQ ID NOs: 74-76, SEQ ID NOs: 10-12 and SEQ ID NOs: 74-76, SEQ ID NOs: 14-16 and SEQ ID NOs: 74-76, SEQ ID NOs: 18-20 and SEQ ID NOs: 74-76, SEQ ID NOs: 22-24 and SEQ ID NOs: 74-76, SEQ ID NOS: 26-28 and SEQ ID NOs: 74-76, SEQ ID NOs: 30-32 and SEQ ID NOs: 74-76, SEQ ID NOs: 34-36 and SEQ ID NOs: 74-76, SEQ ID NOs: 38-40 and SEQ ID NOs: 74-76, SEQ ID NOs: 42-44 and SEQ ID NOs: 74-76, SEQ ID NOs: 46-48 and SEQ ID NOs: 74-76, SEQ ID NOs: 50-52 and SEQ ID NOs: 74-76, SEQ ID NOs: 54-56 and SEQ ID NOs: 74-76, SEQ ID NOs: 58-60 and SEQ ID NOs: 74-76, SEQ ID NOs: 62-64 and SEQ ID NOs: 74-76; SEQ ID NOs: 66-68 and SEQ ID NOs: 74-76 (furthermore any of the sIL-6R CDR sets shown in SEQ ID NOs: 138-194 can similarly be combined with SEQ ID NO: 74-76 and any of the CTGF CDR sets as set forth in SEQ ID NOs: 233-286 can be used in place of SEQ ID NOs: 74-76); SEQ ID NOs: 2-4 and SEQ ID NOs: 78-80, SEQ ID NOs: 6-8 and SEQ ID NOs: 78-80, SEQ ID NOs: 10-12 and SEQ ID NOs: 78-80, SEQ ID NOs: 14-16 and SEQ ID NOs: 78-80, SEQ ID NOs: 18-20 and SEQ ID NOs: 78-80, SEQ ID NOs: 22-24 and SEQ ID NOs: 78-80, SEQ ID NOs: 26-28 and SEQ ID NOs: 78-80, SEQ ID NOs: 30-32 and SEQ ID NOs: 78-80, SEQ ID NOs: 34-36 and SEQ ID NOs: 78-80, SEQ ID NOs: 38-40 and SEQ ID NOs: 78-80, SEQ ID NOs: 42-44 and SEQ ID NOs: 78-80, SEQ ID NOs: 46-48 and SEQ ID NOs: 78-80, SEQ ID NOs: 50-52 and SEQ ID NOs: 78-80, SEQ ID NOs: 54-56 and SEQ ID NOs: 78-80, SEQ ID NOs: 58-60 and SEQ ID NOs: 78-80, SEQ ID NOs: 62-64 and SEQ ID NOs: 78-80; SEQ ID NOs: 66-68 and SEQ ID NOs: 78-80 (furthermore any of the sIL-6R CDR sets shown in SEQ ID NOs: 138-194 can similarly be combined with SEQ ID NO:78-80 and any of the CTGF CDR sets as set forth in SEQ ID NOs: 233-286 can be used in place of SEQ ID NOs: 78-80); SEQ ID NOs: 2-4 and SEQ ID NOs: 82-84, SEQ ID NOs: 6-8 and SEQ ID Nos: 82-84, SEQ ID NOs: 10-12 and SEQ ID Nos: 82-84, SEQ ID NOs: 14-16 and SEQ ID Nos: 82-84, SEQ ID NOs: 18-20 and SEQ ID Nos: 82-84, SEQ ID NOs: 22-24 and SEQ ID Nos: 82-84, SEQ ID NOs: 26-28 and SEQ ID Nos: 82-84, SEQ ID NOs: 30-32 and SEQ ID Nos: 82-84, SEQ ID NOs: 34-36 and SEQ ID Nos: 82-84, SEQ ID NOs: 38-40 and SEQ ID Nos: 82-84, SEQ ID NOs: 42-44 and SEQ ID Nos: 82-84, SEQ ID NOs: 46-48 and SEQ ID Nos: 82-84, SEQ ID NOs: 50-52 and SEQ ID Nos: 82-84, SEQ ID NOs: 54-56 and SEQ ID Nos: 82-84, SEQ ID NOs: 58-60 and SEQ ID Nos: 82-84, SEQ ID NOs: 62-64 and SEQ ID Nos: 82-84; SEQ ID NOs: 66-68 and SEQ ID Nos: 82-84 (furthermore any of the sIL-6R CDR sets shown in SEQ ID NOs: 138-194 can similarly be combined with SEQ ID NO:82-84 and any of the CTGF CDR sets as set forth in SEQ ID NOs: 233-286 can be used in place of SEQ ID NOs: 82-84); SEQ ID NOs: 2-4 and SEQ ID NOs: 86-88, SEQ ID NOs: 6-8 and SEQ ID NOs: 86-88, SEQ ID NOs: 10-12 and SEQ ID NOs: 86-88, SEQ ID NOs: 14-16 and SEQ ID NOs: 86-88, SEQ ID NOs: 18-20 and SEQ ID NOs: 86-88, SEQ ID NOs: 22-24 and SEQ ID NOs: 86-88, SEQ ID NOs: 26-28 and SEQ ID NOs: 86-88, SEQ ID NOs: 30-32 and SEQ ID NOs: 86-88, SEQ ID NOs: 34-36 and SEQ ID NOs: 86-88, SEQ ID NOs: 38-40 and SEQ ID NOs: 86-88, SEQ ID NOs: 42-44 and SEQ ID NOs: 86-88, SEQ ID NOs: 46-48 and SEQ ID NOs: 86-88, SEQ ID NOs: 50-52 and SEQ ID NOs: 86-88, SEQ ID NOs: 54-56 and SEQ ID NOs: 86-88, SEQ ID NOs: 58-60 and SEQ ID NOs: 86-88, SEQ ID NOs: 62-64 and SEQ ID NOs: 86-88; SEQ ID NOs: 66-68 and SEQ ID NOs: 86-88 (furthermore any of the sIL-6R CDR sets shown in SEQ ID NOs: 138-194 can similarly be combined with SEQ ID NO:86-88 and any of the CTGF CDR sets as set forth in SEQ ID NOs: 233-286 can be used in place of SEQ ID NOs: 86-88); SEQ ID NOs: 2-4 and SEQ ID NOs: 90-92, SEQ ID NOs: 6-8 and SEQ ID NOs: 90-92, SEQ ID NOs: 10-12 and SEQ ID NOs: 90-92, SEQ ID NOs: 14-16 and SEQ ID NOs: 90-92, SEQ ID NOs: 18-20 and SEQ ID NOs: 90-92, SEQ ID NOs: 22-24 and SEQ ID NOs: 90-92, SEQ ID NOs: 26-28 and SEQ ID NOs: 90-92, SEQ ID NOs: 30-32 and SEQ ID NOs: 90-92, SEQ ID NOs: 34-36 and SEQ ID NOs: 90-92, SEQ ID NOs: 38-40 and SEQ ID NOs: 90-92, SEQ ID NOs: 42-44 and SEQ ID NOs: 90-92, SEQ ID NOs: 46-48 and SEQ ID NOs: 90-92, SEQ ID NOs: 50-52 and SEQ ID NOs: 90-92, SEQ ID NOs: 54-56 and SEQ ID NOs: 90-92, SEQ ID NOs: 58-60 and SEQ ID NOs: 90-92, SEQ ID NOs: 62-64 and SEQ ID NOs: 90-92 or SEQ ID NOs: 66-68 and SEQ ID NOs: 90-92 (furthermore any of the sIL-6R CDR sets shown in SEQ ID NOs: 138-194 can similarly be combined with SEQ ID NO: 90-92 and any of the CTGF CDR sets as set forth in SEQ ID NOs: 233-286 can be used in place of SEQ ID NOs: 90-92);
(vi) one or more expression vectors including nucleic acid molecules encoding a multispecific binding protein that selectively and/or specifically binds to human sIL-6R and to human CTGF as described herein, for example including the amino acid sequence of: SEQ ID NO: 1 and SEQ ID NO:69, SEQ ID NO:5 and SEQ ID NO:69, SEQ ID NO:9 and SEQ ID NO:69, SEQ ID NO: 13 and SEQ ID NO:69, SEQ ID NO: 17 and SEQ ID NO:69, SEQ ID NO:21 and SEQ ID NO: 69, SEQ ID NO:25 and SEQ ID NO:69, SEQ ID NO:29 and SEQ ID NO:69, SEQ ID NO:33 and SEQ ID NO:69, SEQ ID NO: 37 and SEQ ID NO:69, SEQ ID NO: 41 and SEQ ID NO:69, SEQ ID NO: 45 and SEQ ID NO:69, SEQ ID NO:49 and SEQ ID NO:69, SEQ ID NO:53 and SEQ ID NO: 69, SEQ ID NO:57 and SEQ ID NO:69, SEQ ID NO:61 and SEQ ID NO:69, SEQ ID NO:65 and SEQ ID NO:69 (furthermore any of the sIL-6R VHHs shown in SEQ ID NOs: 119-137 can similarly be combined with SEQ ID NO:69 and any of SEQ ID NO:214-232 can be used in place of SEQ ID NO:69); SEQ ID NO: 1 and SEQ ID NO:73, SEQ ID NO:5 and SEQ ID NO:73, SEQ ID NO: 9 and SEQ ID NO:73, SEQ ID NO: 13 and SEQ ID NO:73, SEQ ID NO: 17 and SEQ ID NO:73, SEQ ID NO:21 and SEQ ID NO:73, SEQ ID NO:25 and SEQ ID NO:73, SEQ ID NO:29 and SEQ ID NO: 73, SEQ ID NO:33 and SEQ ID NO:73, SEQ ID NO:37 and SEQ ID NO:73, SEQ ID NO:41 and SEQ ID NO: 73, SEQ ID NO: 45 and SEQ ID NO:73, SEQ ID NO:49 and SEQ ID NO:73, SEQ ID NO: 53 and SEQ ID NO:73, SEQ ID NO:57 and SEQ ID NO:73, SEQ ID NO:61 and SEQ ID NO: 73, SEQ ID NO: 65 and SEQ ID NO:73 (furthermore any of the sIL-6R VHHs shown in SEQ ID NOs: 119-137 can similarly be combined with SEQ ID NO:73 and any of SEQ ID NO:214-232 can be used in place of SEQ ID NO:73); SEQ ID NO: 1 and SEQ ID NO:77, SEQ ID NO:5 and SEQ ID NO:77, SEQ ID NO:9 and SEQ ID NO:77, SEQ ID NO: 13 and SEQ ID NO:77, SEQ ID NO: 17 and SEQ ID NO:77, SEQ ID NO:21 and SEQ ID NO:77, SEQ ID NO:25 and SEQ ID NO:77, SEQ ID NO:29 and SEQ ID NO:77, SEQ ID NO:33 and SEQ ID NO:77, SEQ ID NO:37 and SEQ ID NO: 77, SEQ ID NO:41 and SEQ ID NO:77, SEQ ID NO:45 and SEQ ID NO:77, SEQ ID NO:49 and SEQ ID NO:77, SEQ ID NO:53 and SEQ ID NO:77, SEQ ID NO:57 and SEQ ID NO:77, SEQ ID NO: 61 and SEQ ID NO:77, SEQ ID NO: 65 and SEQ ID NO:77 (furthermore any of the sIL-6R VHHs shown in SEQ ID NOs: 119-137 can similarly be combined with SEQ ID NO:77 and any of SEQ ID NO:214-232 can be used in place of SEQ ID NO:77); SEQ ID NO:1 and SEQ ID NO:81, SEQ ID NO:5 and SEQ ID NO:81, SEQ ID NO:9 and SEQ ID NO:81, SEQ ID NO: 13 and SEQ ID NO: 81, SEQ ID NO: 17 and SEQ ID NO:81, SEQ ID NO:21 and SEQ ID NO:81, SEQ ID NO:25 and SEQ ID NO:81, SEQ ID NO:29 and SEQ ID NO:81, SEQ ID NO:33 and SEQ ID NO:81, SEQ ID NO: 37 and SEQ ID NO:81, SEQ ID NO:41 and SEQ ID NO:81, SEQ ID NO:45 and SEQ ID NO: 81, SEQ ID NO:49 and SEQ ID NO:81, SEQ ID NO:53 and SEQ ID NO:81, SEQ ID NO:57 and SEQ ID NO:81, SEQ ID NO:61 and SEQ ID NO:81, SEQ ID NO:65 and SEQ ID NO:81 (furthermore any of the sIL-6R VHHs shown in SEQ ID NOs: 119-137 can similarly be combined with SEQ ID NO: 81 and any of SEQ ID NO:214-232 can be used in place of SEQ ID NO:81); SEQ ID NO: 1 and SEQ ID NO:85, SEQ ID NO:5 and SEQ ID NO:85, SEQ ID NO:9 and SEQ ID NO:85, SEQ ID NO: 13 and SEQ ID NO:85, SEQ ID NO: 17 and SEQ ID NO:85, SEQ ID NO:21 and SEQ ID NO: 85, SEQ ID NO:25 and SEQ ID NO:85, SEQ ID NO:29 and SEQ ID NO:85, SEQ ID NO:33 and SEQ ID NO: 85, SEQ ID NO:37 and SEQ ID NO: 85, SEQ ID NO:41 and SEQ ID NO:85, SEQ ID NO: 45 and SEQ ID NO:85, SEQ ID NO:49 and SEQ ID NO:85, SEQ ID NO:53 and SEQ ID NO: 85, SEQ ID NO:57 and SEQ ID NO:85, SEQ ID NO:61 and SEQ ID NO:85, SEQ ID NO:65 and SEQ ID NO:85 (furthermore any of the sIL-6R VHHs shown in SEQ ID NOs: 119-137 can similarly be combined with SEQ ID NO:85 and any of SEQ ID NO:214-232 can be used in place of SEQ ID NO:85); SEQ ID NO: 1 and SEQ ID NO:89, SEQ ID NO:5 and SEQ ID NO:89, SEQ ID NO: 9 and SEQ ID NO: 89, SEQ ID NO: 13 and SEQ ID NO: 89, SEQ ID NO: 17 and SEQ ID NO:89, SEQ ID NO:21 and SEQ ID NO: 89, SEQ ID NO:25 and SEQ ID NO: 89, SEQ ID NO:29 and SEQ ID NO: 89, SEQ ID NO:33 and SEQ ID NO:89, SEQ ID NO:37 and SEQ ID NO:89, SEQ ID NO:41 and SEQ ID NO: 89, SEQ ID NO:45 and SEQ ID NO:89, SEQ ID NO:49 and SEQ ID NO:89, SEQ ID NO: 53 and SEQ ID NO:89, SEQ ID NO:57 and SEQ ID NO:89, SEQ ID NO:61 and SEQ ID NO: 89, or SEQ ID NO: 65 and SEQ ID NO: 89 (furthermore any of the sIL-6R VHHs shown in SEQ ID NOs: 119-137 can similarly be combined with SEQ ID NO:89 and any of SEQ ID NO:214-232 can be used in place of SEQ ID NO:89);
(vii) one or more expression vectors including nucleic acid molecules encoding a fusion protein including a sIL-6R binding protein as described herein and an anti-transferrin receptor antibody, for example including CDRs as set forth in SEQ ID NOs: 2-4, SEQ ID NOS: 6-8, SEQ ID NOs: 10-12, SEQ ID NOs: 14-16, SEQ ID NOs: 18-20, SEQ ID NOS: 22-24, SEQ ID NOS: 26-28, SEQ ID NOs: 30-32, SEQ ID NOs: 34-36, SEQ ID NOs: 38-40, SEQ ID NOs: 42-44, SEQ ID NOs: 46-48, SEQ ID NOs: 50-52, SEQ ID NOs: 54-56, SEQ ID NOs: 58-60, SEQ ID NOS: 62-64, SEQ ID NOs: 66-68, or SEQ ID NO: 138-194 each further including a nucleic acid molecule encoding an anti-transferrin receptor antibody;
(viii) one or more expression vectors including nucleic acid molecules encoding a fusion protein including a sIL-6R binding protein as described herein and an anti-transferrin receptor antibody, for example including the amino acid sequence of SEQ ID NO:1, SEQ ID NO:5, SEQ ID NO: 9, SEQ ID NO: 13, SEQ ID NO: 17, SEQ ID NO:21, SEQ ID NO:25, SEQ ID NO:29, SEQ ID NO: 33, SEQ ID NO:37, SEQ ID NO:41, SEQ ID NO:45, SEQ ID NO:49, SEQ ID NO:53, SEQ ID NO: 57, SEQ ID NO:61, SEQ ID NO:65, or SEQ ID NOs: 119-137, each further including a nucleic acid molecule encoding an anti-transferrin receptor antibody;
(ix) one or more expression vectors including nucleic acid molecules encoding a fusion protein including a CTGF binding protein as described herein and an anti-transferrin receptor antibody, for example including CDRs as set forth in NOs: 70-72, SEQ ID NOs: 74-76, SEQ ID NOs: 78-80, SEQ ID NOs: 82-84, SEQ ID NOS: 86-88, SEQ ID NOs: 90-92, SEQ ID NOs: 233-286, or SEQ ID NOs: 291-293, each further including a nucleic acid molecule encoding an anti-transferrin receptor antibody;
(x) one or more expression vectors including nucleic acid molecules encoding a fusion protein including a CTGF binding protein as described herein and an anti-transferrin receptor antibody, for example including the amino acid sequence of SEQ ID NO:73, SEQ ID NO:77, SEQ ID NO: 81, SEQ ID NO: 85, SEQ ID NO: 89, or SEQ ID NO:214-232, each further including a nucleic acid molecule encoding an anti-transferrin receptor antibody;
(xi) one or more expression vectors including nucleic acid molecules encoding a fusion protein including a multispecific binding protein that selectively and/or specifically binds to human sIL-6R and to human CTGF as described herein and an anti-transferrin receptor antibody, for example including CDRs as set forth in SEQ ID NOs: 2-4 and SEQ ID NOs: 70-72, SEQ ID NOs: 6-8 and SEQ ID NOs: 70-72, SEQ ID NOs: 10-12 and SEQ ID NOs: 70-72, SEQ ID NOs: 14-16 and SEQ ID NOs: 70-72, SEQ ID NOs: 18-20 and SEQ ID NOs: 70-72, SEQ ID NOs: 22-24 and SEQ ID NOs: 70-72, SEQ ID NOs: 26-28 and SEQ ID NOs: 70-72, SEQ ID NOs: 30-32 and SEQ ID NOs: 70-72, SEQ ID NOs: 34-36 and SEQ ID NOs: 70-72, SEQ ID NOs: 38-40 and SEQ ID NOs: 70-72, SEQ ID NOs: 42-44 and SEQ ID NOs: 70-72, SEQ ID NOs: 46-48 and SEQ ID NOs: 70-72, SEQ ID NOs: 50-52 and SEQ ID NOs: 70-72, SEQ ID NOs: 54-56 and SEQ ID NOs: 70-72, SEQ ID NOs: 58-60 and SEQ ID NOs: 70-72, SEQ ID NOs: 62-64 and SEQ ID NOs: 70-72; SEQ ID NOs: 66-68 and SEQ ID NOs: 70-72 (furthermore any of the sIL-6R CDR sets shown in SEQ ID NOs: 138-194 can similarly be combined with SEQ ID NO:70-72 and any of the CTGF CDR sets as set forth in SEQ ID NOs: 233-286 can be used in place of SEQ ID NOs: 70-72); SEQ ID NOs: 2-4 and SEQ ID NOs: 74-76, SEQ ID NOs: 6-8 and SEQ ID NOs: 74-76, SEQ ID NOs: 10-12 and SEQ ID NOs: 74-76, SEQ ID NOs: 14-16 and SEQ ID NOs: 74-76, SEQ ID NOs: 18-20 and SEQ ID NOs: 74-76, SEQ ID NOs: 22-24 and SEQ ID NOs: 74-76, SEQ ID NOs: 26-28 and SEQ ID NOs: 74-76, SEQ ID NOs: 30-32 and SEQ ID NOs: 74-76, SEQ ID NOs: 34-36 and SEQ ID NOs: 74-76, SEQ ID NOs: 38-40 and SEQ ID NOs: 74-76, SEQ ID NOs: 42-44 and SEQ ID NOs: 74-76, SEQ ID NOs: 46-48 and SEQ ID NOs: 74-76, SEQ ID NOs: 50-52 and SEQ ID NOs: 74-76, SEQ ID NOs: 54-56 and SEQ ID NOs: 74-76, SEQ ID NOs: 58-60 and SEQ ID NOs: 74-76, SEQ ID NOs: 62-64 and SEQ ID NOs: 74-76; SEQ ID NOs: 66-68 and SEQ ID NOs: 74-76 (furthermore any of the sIL-6R CDR sets shown in SEQ ID NOs: 138-194 can similarly be combined with SEQ ID NO:74-76 and any of the CTGF CDR sets as set forth in SEQ ID NOs: 233-286 can be used in place of SEQ ID NOs: 74-76); SEQ ID NOs: 2-4 and SEQ ID NOs: 78-80, SEQ ID NOs: 6-8 and SEQ ID NOs: 78-80, SEQ ID NOs: 10-12 and SEQ ID NOs: 78-80, SEQ ID NOs: 14-16 and SEQ ID NOs: 78-80, SEQ ID NOs: 18-20 and SEQ ID NOs: 78-80, SEQ ID NOs: 22-24 and SEQ ID NOs: 78-80, SEQ ID NOs: 26-28 and SEQ ID NOs: 78-80, SEQ ID NOs: 30-32 and SEQ ID NOs: 78-80, SEQ ID NOs: 34-36 and SEQ ID NOs: 78-80, SEQ ID NOs: 38-40 and SEQ ID NOs: 78-80, SEQ ID NOs: 42-44 and SEQ ID NOs: 78-80, SEQ ID NOs: 46-48 and SEQ ID NOs: 78-80, SEQ ID NOs: 50-52 and SEQ ID NOs: 78-80, SEQ ID NOs: 54-56 and SEQ ID NOs: 78-80, SEQ ID NOs: 58-60 and SEQ ID NOs: 78-80, SEQ ID NOS: 62-64 and SEQ ID NOs: 78-80; SEQ ID NOs: 66-68 and SEQ ID NOs: 78-80 (furthermore any of the SIL-6R CDR sets shown in SEQ ID NOs: 138-194 can similarly be combined with SEQ ID NO:78-80 and any of the CTGF CDR sets as set forth in SEQ ID NOs: 233-286 can be used in place of SEQ ID NOs: 78-80); SEQ ID NOs: 2-4 and SEQ ID NOs: 82-84, SEQ ID NOs: 6-8 and SEQ ID NOs: 82-84, SEQ ID NOs: 10-12 and SEQ ID NOs: 82-84, SEQ ID NOs: 14-16 and SEQ ID NOs: 82-84, SEQ ID NOs: 18-20 and SEQ ID NOs: 82-84, SEQ ID NOs: 22-24 and SEQ ID NOs: 82-84, SEQ ID NOs: 26-28 and SEQ ID NOs: 82-84, SEQ ID NOs: 30-32 and SEQ ID NOs: 82-84, SEQ ID NOs: 34-36 and SEQ ID NOs: 82-84, SEQ ID NOs: 38-40 and SEQ ID NOs: 82-84, SEQ ID NOs: 42-44 and SEQ ID NOs: 82-84, SEQ ID NOs: 46-48 and SEQ ID NOs: 82-84, SEQ ID NOs: 50-52 and SEQ ID NOs: 82-84, SEQ ID NOs: 54-56 and SEQ ID NOs: 82-84, SEQ ID NOs: 58-60 and SEQ ID NOs: 82-84, SEQ ID NOs: 62-64 and SEQ ID NOs: 82-84; SEQ ID NOs: 66-68 and SEQ ID NOs: 82-84 (furthermore any of the sIL-6R CDR sets shown in SEQ ID NOs: 138-194 can similarly be combined with SEQ ID NO:82-84 and any of the CTGF CDR sets as set forth in SEQ ID NOS: 233-286 can be used in place of SEQ ID NOs: 82-84); SEQ ID NOs: 2-4 and SEQ ID NOs: 86-88, SEQ ID NOs: 6-8 and SEQ ID NOs: 86-88, SEQ ID NOs: 10-12 and SEQ ID NOs: 86-88, SEQ ID NOs: 14-16 and SEQ ID NOs: 86-88, SEQ ID NOs: 18-20 and SEQ ID NOs: 86-88, SEQ ID NOS: 22-24 and SEQ ID NOs: 86-88, SEQ ID NOs: 26-28 and SEQ ID NOs: 86-88, SEQ ID NOs: 30-32 and SEQ ID NOs: 86-88, SEQ ID NOs: 34-36 and SEQ ID NOs: 86-88, SEQ ID NOs: 38-40 and SEQ ID NOs: 86-88, SEQ ID NOs: 42-44 and SEQ ID NOs: 86-88, SEQ ID NOs: 46-48 and SEQ ID NOs: 86-88, SEQ ID NOs: 50-52 and SEQ ID NOs: 86-88, SEQ ID NOs: 54-56 and SEQ ID NOs: 86-88, SEQ ID NOs: 58-60 and SEQ ID NOs: 86-88, SEQ ID NOs: 62-64 and SEQ ID NOs: 86-88; SEQ ID NOs: 66-68 and SEQ ID NOs: 86-88 (furthermore any of the sIL-6R CDR sets shown in SEQ ID NOs: 138-194 can similarly be combined with SEQ ID NO: 86-88 and any of the CTGF CDR sets as set forth in SEQ ID NOs: 233-286 can be used in place of SEQ ID NOs: 86-88); SEQ ID NOs: 2-4 and SEQ ID NOs: 90-92, SEQ ID NOs: 6-8 and SEQ ID NOs: 90-92, SEQ ID NOs: 10-12 and SEQ ID NOs: 90-92, SEQ ID NOs: 14-16 and SEQ ID NOs: 90-92, SEQ ID NOs: 18-20 and SEQ ID NOs: 90-92, SEQ ID NOs: 22-24 and SEQ ID NOs: 90-92, SEQ ID NOs: 26-28 and SEQ ID NOs: 90-92, SEQ ID NOs: 30-32 and SEQ ID NOs: 90-92, SEQ ID NOs: 34-36 and SEQ ID NOs: 90-92, SEQ ID NOs: 38-40 and SEQ ID NOs: 90-92, SEQ ID NOs: 42-44 and SEQ ID NOs: 90-92, SEQ ID NOs: 46-48 and SEQ ID NOs: 90-92, SEQ ID NOs: 50-52 and SEQ ID NOs: 90-92, SEQ ID NOs: 54-56 and SEQ ID NOs: 90-92, SEQ ID NOs: 58-60 and SEQ ID NOs: 90-92, SEQ ID NOs: 62-64 and SEQ ID NOs: 90-92 or SEQ ID NOs: 66-68 and SEQ ID NOs: 90-92 (furthermore any of the sIL-6R CDR sets shown in SEQ ID NOs: 138-194 can similarly be combined with SEQ ID NO:90-92 and any of the CTGF CDR sets as set forth in SEQ ID NOs: 233-286 can be used in place of SEQ ID NOs: 90-92), each further including a nucleic acid molecule encoding an anti-transferrin receptor antibody; and
(xii) one or more expression vectors including nucleic acid molecules encoding a fusion protein including a multispecific binding protein that selectively and/or specifically binds to human sIL-6R and to human CTGF as described herein and an anti-transferrin receptor antibody, for example including the amino acid sequence of: SEQ ID NO: 1 and SEQ ID NO:69, SEQ ID NO:5 and SEQ ID NO:69, SEQ ID NO:9 and SEQ ID NO:69, SEQ ID NO: 13 and SEQ ID NO:69, SEQ ID NO: 17 and SEQ ID NO:69, SEQ ID NO:21 and SEQ ID NO:69, SEQ ID NO:25 and SEQ ID NO: 69, SEQ ID NO:29 and SEQ ID NO:69, SEQ ID NO:33 and SEQ ID NO:69, SEQ ID NO:37 and SEQ ID NO:69, SEQ ID NO:41 and SEQ ID NO:69, SEQ ID NO:45 and SEQ ID NO:69, SEQ ID NO: 49 and SEQ ID NO:69, SEQ ID NO:53 and SEQ ID NO:69, SEQ ID NO:57 and SEQ ID NO: 69, SEQ ID NO: 61 and SEQ ID NO: 69, SEQ ID NO:65 and SEQ ID NO:69 (furthermore any of the sIL-6R VHHs shown in SEQ ID NOs: 119-137 can similarly be combined with SEQ ID NO:69 and any of SEQ ID NO:214-232 can be used in place of SEQ ID NO:69); SEQ ID NO: 1 and SEQ ID NO: 73, SEQ ID NO:5 and SEQ ID NO:73, SEQ ID NO:9 and SEQ ID NO:73, SEQ ID NO: 13 and SEQ ID NO:73, SEQ ID NO: 17 and SEQ ID NO:73, SEQ ID NO:21 and SEQ ID NO:73, SEQ ID NO: 25 and SEQ ID NO:73, SEQ ID NO:29 and SEQ ID NO:73, SEQ ID NO:33 and SEQ ID NO: 73, SEQ ID NO: 37 and SEQ ID NO:73, SEQ ID NO:41 and SEQ ID NO:73, SEQ ID NO:45 and SEQ ID NO: 73, SEQ ID NO:49 and SEQ ID NO:73, SEQ ID NO:53 and SEQ ID NO:73, SEQ ID NO: 57 and SEQ ID NO:73, SEQ ID NO:61 and SEQ ID NO:73, SEQ ID NO:65 and SEQ ID NO: 73 (furthermore any of the sIL-6R VHHs shown in SEQ ID NOs: 119-137 can similarly be combined with SEQ ID NO:73 and any of SEQ ID NO:214-232 can be used in place of SEQ ID NO: 73); SEQ ID NO: 1 and SEQ ID NO:77, SEQ ID NO:5 and SEQ ID NO:77, SEQ ID NO:9 and SEQ ID NO:77, SEQ ID NO: 13 and SEQ ID NO:77, SEQ ID NO: 17 and SEQ ID NO:77, SEQ ID NO: 21 and SEQ ID NO: 77, SEQ ID NO:25 and SEQ ID NO:77, SEQ ID NO:29 and SEQ ID NO:77, SEQ ID NO:33 and SEQ ID NO:77, SEQ ID NO:37 and SEQ ID NO:77, SEQ ID NO:41 and SEQ ID NO: 77, SEQ ID NO:45 and SEQ ID NO:77, SEQ ID NO:49 and SEQ ID NO:77, SEQ ID NO:53 and SEQ ID NO:77, SEQ ID NO:57 and SEQ ID NO:77, SEQ ID NO:61 and SEQ ID NO:77, SEQ ID NO: 65 and SEQ ID NO:77 (furthermore any of the sIL-6R VHHs shown in SEQ ID NOs: 119-137 can similarly be combined with SEQ ID NO:77 and any of SEQ ID NO:214-232 can be used in place of SEQ ID NO:77); SEQ ID NO: 1 and SEQ ID NO:81, SEQ ID NO:5 and SEQ ID NO:81, SEQ ID NO:9 and SEQ ID NO:81, SEQ ID NO: 13 and SEQ ID NO:81, SEQ ID NO: 17 and SEQ ID NO: 81, SEQ ID NO:21 and SEQ ID NO:81, SEQ ID NO:25 and SEQ ID NO:81, SEQ ID NO:29 and SEQ ID NO:81, SEQ ID NO:33 and SEQ ID NO:81, SEQ ID NO:37 and SEQ ID NO:81, SEQ ID NO: 41 and SEQ ID NO:81, SEQ ID NO:45 and SEQ ID NO:81, SEQ ID NO:49 and SEQ ID NO: 81, SEQ ID NO:53 and SEQ ID NO:81, SEQ ID NO:57 and SEQ ID NO:81, SEQ ID NO:61 and SEQ ID NO:81, SEQ ID NO:65 and SEQ ID NO:81 (furthermore any of the sIL-6R VHHs shown in SEQ ID NOs: 119-137 can similarly be combined with SEQ ID NO:81 and any of SEQ ID NO: 214-232 can be used in place of SEQ ID NO:81); SEQ ID NO: 1 and SEQ ID NO:85, SEQ ID NO: 5 and SEQ ID NO:85, SEQ ID NO:9 and SEQ ID NO:85, SEQ ID NO: 13 and SEQ ID NO: 85, SEQ ID NO: 17 and SEQ ID NO:85, SEQ ID NO:21 and SEQ ID NO:85, SEQ ID NO:25 and SEQ ID NO: 85, SEQ ID NO:29 and SEQ ID NO:85, SEQ ID NO:33 and SEQ ID NO:85, SEQ ID NO: 37 and SEQ ID NO:85, SEQ ID NO:41 and SEQ ID NO:85, SEQ ID NO:45 and SEQ ID NO: 85, SEQ ID NO:49 and SEQ ID NO:85, SEQ ID NO:53 and SEQ ID NO:85, SEQ ID NO:57 and SEQ ID NO:85, SEQ ID NO:61 and SEQ ID NO:85, SEQ ID NO:65 and SEQ ID NO:85 (furthermore any of the sIL-6R VHHs shown in SEQ ID NOs: 119-137 can similarly be combined with SEQ ID NO: 85 and any of SEQ ID NO:214-232 can be used in place of SEQ ID NO:85); SEQ ID NO: 1 and SEQ ID NO:89, SEQ ID NO:5 and SEQ ID NO:89, SEQ ID NO:9 and SEQ ID NO:89, SEQ ID NO: 13 and SEQ ID NO:89, SEQ ID NO: 17 and SEQ ID NO:89, SEQ ID NO:21 and SEQ ID NO: 89, SEQ ID NO:25 and SEQ ID NO:89, SEQ ID NO:29 and SEQ ID NO:89, SEQ ID NO:33 and SEQ ID NO:89, SEQ ID NO:37 and SEQ ID NO:89, SEQ ID NO:41 and SEQ ID NO:89, SEQ ID NO: 45 and SEQ ID NO:89, SEQ ID NO:49 and SEQ ID NO:89, SEQ ID NO:53 and SEQ ID NO: 89, SEQ ID NO:57 and SEQ ID NO: 89, SEQ ID NO:61 and SEQ ID NO:89, or SEQ ID NO:65 and SEQ ID NO:89 (furthermore any of the sIL-6R VHHs shown in SEQ ID NOs: 119-137 can similarly be combined with SEQ ID NO:89 and any of SEQ ID NO:214-232 can be used in place of SEQ ID NO:89), each further including a nucleic acid molecule encoding an anti-transferrin receptor antibody.

### Pharmaceutical Compositions

An embodiment provides a pharmaceutical composition comprising one or more binding proteins, one or more multispecific binding proteins, and/or one or more of any one of the fusion proteins described herein, and a pharmaceutically acceptable carrier.

As used herein, “pharmaceutical composition” refers to a formulation comprising an active ingredient, and optionally a pharmaceutically acceptable carrier, diluent or excipient. The term “active ingredient” can interchangeably refer to an “effective ingredient” and is meant to refer to any agent that is capable of inducing a sought-after effect upon administration. In one embodiment, the active ingredient includes a biologically active molecule. As used herein, the phrase “biologically active molecule” refers to a molecule that has a biological effect in a cell. In certain embodiments the active molecule may be an inorganic molecule, an organic molecule, a small organic molecule, a drug compound, a peptide, a polypeptide, such as an enzyme or transcription factor, an antibody, an antibody fragment, a peptidomimetic, a lipid, a nucleic acid such as a DNA or RNA molecule, a ribozyme, hairpin RNA, siRNA (small interfering RNAs) of varying chemistries, miRNA, siRNA-protein conjugate, an siRNA-peptide conjugate, and siRNA-antibody conjugate, an antagomir, a PNA (peptide nucleic acid), an LNA (locked nucleic acids), or a morpholino.

By “pharmaceutically acceptable” it is meant that the carrier, diluent or excipient is compatible with the other ingredients of the formulation and not deleterious to the recipient thereof. Pharmaceutically acceptable carriers, excipients or stabilizers are described in, for example Remington's Pharmaceutical Sciences, 16th edition, Osol, A. Ed. (1980).

Pharmaceutically acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™ PLURONICS™ or polyethylene glycol (PEG).

For example, provided herein with this disclosure are pharmaceutical compositions including:
(i) one or more sIL-6R binding proteins as described herein, for example including CDRs as set forth in SEQ ID NOs: 2-4, SEQ ID NOS: 6-8, SEQ ID NOs: 10-12, SEQ ID NOS: 14-16, SEQ ID NOs: 18-20, SEQ ID NOs: 22-24, SEQ ID NOs: 26-28, SEQ ID NOs: 30-32, SEQ ID NOs: 34-36, SEQ ID NOs: 38-40, SEQ ID NOs: 42-44, SEQ ID NOs: 46-48, SEQ ID NOs: 50-52, SEQ ID NOs: 54-56, SEQ ID NOs: 58-60, SEQ ID NOs: 62-64, SEQ ID NOs: 66-68, or SEQ ID NO: 138-194 and a pharmaceutically acceptable carrier;
(ii) one or more sIL-6R binding proteins as described herein, for example including the amino acid sequence of SEQ ID NO:1, SEQ ID NO:5, SEQ ID NO:9, SEQ ID NO: 13, SEQ ID NO: 17, SEQ ID NO:21, SEQ ID NO:25, SEQ ID NO:29, SEQ ID NO:33, SEQ ID NO:37, SEQ ID NO: 41, SEQ ID NO:45, SEQ ID NO: 49, SEQ ID NO:53, SEQ ID NO:57, SEQ ID NO:61, SEQ ID NO: 65, or SEQ ID NOs: 119-137, and a pharmaceutically acceptable carrier;
(iii) one or more CTGF binding proteins as described herein, for example including CDRs as set forth in NOs: 70-72, SEQ ID NOs: 74-76, SEQ ID NOs: 78-80, SEQ ID NOS: 82-84, SEQ ID NOs: 86-88, SEQ ID NOs: 90-92, SEQ ID NOs: 233-286, or SEQ ID NOs: 291-293 and a pharmaceutically acceptable carrier;
(iv) one or more CTGF binding proteins as described herein, for example including the amino acid sequence of SEQ ID NO:73, SEQ ID NO:77, SEQ ID NO:81, SEQ ID NO:85, SEQ ID NO: 89, or SEQ ID NOs: 214-232 and a pharmaceutically acceptable carrier;
(v) one or more multispecific binding proteins that selectively and/or specifically binds to human sIL-6R and to human CTGF as described herein, for example including CDRs as set forth in SEQ ID NOs: 2-4 and SEQ ID NOs: 70-72, SEQ ID NOs: 6-8 and SEQ ID NOs: 70-72, SEQ ID NOs: 10-12 and SEQ ID NOs: 70-72, SEQ ID NOs: 14-16 and SEQ ID NOs: 70-72, SEQ ID NOs: 18-20 and SEQ ID NOs: 70-72, SEQ ID NOs: 22-24 and SEQ ID NOs: 70-72, SEQ ID NOs: 26-28 and SEQ ID NOs: 70-72, SEQ ID NOs: 30-32 and SEQ ID NOs: 70-72, SEQ ID NOs: 34-36 and SEQ ID NOs: 70-72, SEQ ID NOs: 38-40 and SEQ ID NOs: 70-72, SEQ ID NOs: 42-44 and SEQ ID NOs: 70-72, SEQ ID NOs: 46-48 and SEQ ID NOs: 70-72, SEQ ID NOs: 50-52 and SEQ ID NOs: 70-72, SEQ ID NOs: 54-56 and SEQ ID NOs: 70-72, SEQ ID NOs: 58-60 and SEQ ID NOs: 70-72, SEQ ID NOs: 62-64 and SEQ ID NOs: 70-72; SEQ ID NOs: 66-68 and SEQ ID NOs: 70-72 (furthermore any of the sIL-6R CDR sets shown in SEQ ID NOs: 138-194 can similarly be combined with SEQ ID NO: 70-72 and any of the CTGF CDR sets as set forth in SEQ ID NOs: 233-286 can be used in place of SEQ ID NOs: 70-72); SEQ ID NOs: 2-4 and SEQ ID NOs: 74-76, SEQ ID NOs: 6-8 and SEQ ID NOs: 74-76, SEQ ID NOs: 10-12 and SEQ ID NOs: 74-76, SEQ ID NOs: 14-16 and SEQ ID NOs: 74-76, SEQ ID NOs: 18-20 and SEQ ID NOs: 74-76, SEQ ID NOs: 22-24 and SEQ ID NOs: 74-76, SEQ ID NOs: 26-28 and SEQ ID NOs: 74-76, SEQ ID NOs: 30-32 and SEQ ID NOs: 74-76, SEQ ID NOs: 34-36 and SEQ ID NOs: 74-76, SEQ ID NOs: 38-40 and SEQ ID NOs: 74-76, SEQ ID NOs: 42-44 and SEQ ID NOs: 74-76, SEQ ID NOs: 46-48 and SEQ ID NOs: 74-76, SEQ ID NOs: 50-52 and SEQ ID NOs: 74-76, SEQ ID NOs: 54-56 and SEQ ID NOs: 74-76, SEQ ID NOs: 58-60 and SEQ ID NOs: 74-76, SEQ ID NOs: 62-64 and SEQ ID NOs: 74-76; SEQ ID NOs: 66-68 and SEQ ID NOs: 74-76 (furthermore any of the sIL-6R CDR sets shown in SEQ ID NOs: 138-194 can similarly be combined with SEQ ID NO:74-76 and any of the CTGF CDR sets as set forth in SEQ ID NOs: 233-286 can be used in place of SEQ ID NOs: 74-76); SEQ ID NOs: 2-4 and SEQ ID NOs: 78-80, SEQ ID NOs: 6-8 and SEQ ID NOs: 78-80, SEQ ID NOs: 10-12 and SEQ ID NOs: 78-80, SEQ ID NOs: 14-16 and SEQ ID NOs: 78-80, SEQ ID NOs: 18-20 and SEQ ID NOs: 78-80, SEQ ID NOs: 22-24 and SEQ ID NOs: 78-80, SEQ ID NOs: 26-28 and SEQ ID NOs: 78-80, SEQ ID NOs: 30-32 and SEQ ID NOs: 78-80, SEQ ID NOs: 34-36 and SEQ ID NOs: 78-80, SEQ ID NOs: 38-40 and SEQ ID NOs: 78-80, SEQ ID NOs: 42-44 and SEQ ID NOs: 78-80, SEQ ID NOs: 46-48 and SEQ ID NOs: 78-80, SEQ ID NOs: 50-52 and SEQ ID NOs: 78-80, SEQ ID NOs: 54-56 and SEQ ID NOs: 78-80, SEQ ID NOs: 58-60 and SEQ ID NOs: 78-80, SEQ ID NOs: 62-64 and SEQ ID NOs: 78-80; SEQ ID NOs: 66-68 and SEQ ID NOs: 78-80 (furthermore any of the sIL-6R CDR sets shown in SEQ ID NOs: 138-194 can similarly be combined with SEQ ID NO:78-80 and any of the CTGF CDR sets as set forth in SEQ ID NOs: 233-286 can be used in place of SEQ ID NOs: 78-80); SEQ ID NOs: 2-4 and SEQ ID NOs: 82-84, SEQ ID NOs: 6-8 and SEQ ID NOs: 82-84, SEQ ID NOs: 10-12 and SEQ ID NOs: 82-84, SEQ ID NOs: 14-16 and SEQ ID NOs: 82-84, SEQ ID NOs: 18-20 and SEQ ID NOs: 82-84, SEQ ID NOs: 22-24 and SEQ ID NOs: 82-84, SEQ ID NOs: 26-28 and SEQ ID NOs: 82-84, SEQ ID NOs: 30-32 and SEQ ID NOs: 82-84, SEQ ID NOs: 34-36 and SEQ ID NOs: 82-84, SEQ ID NOs: 38-40 and SEQ ID NOs: 82-84, SEQ ID NOs: 42-44 and SEQ ID NOs: 82-84, SEQ ID NOs: 46-48 and SEQ ID NOs: 82-84, SEQ ID NOs: 50-52 and SEQ ID NOs: 82-84, SEQ ID NOs: 54-56 and SEQ ID NOs: 82-84, SEQ ID NOs: 58-60 and SEQ ID NOs: 82-84, SEQ ID NOs: 62-64 and SEQ ID NOs: 82-84; SEQ ID NOs: 66-68 and SEQ ID NOs: 82-84 (furthermore any of the sIL-6R CDR sets shown in SEQ ID NOs: 138-194 can similarly be combined with SEQ ID NO:82-84 and any of the CTGF CDR sets as set forth in SEQ ID NOs: 233-286 can be used in place of SEQ ID NOs: 82-84); SEQ ID NOs: 2-4 and SEQ ID NOs: 86-88, SEQ ID NOs: 6-8 and SEQ ID NOs: 86-88, SEQ ID NOs: 10-12 and SEQ ID NOs: 86-88, SEQ ID NOs: 14-16 and SEQ ID NOs: 86-88, SEQ ID NOs: 18-20 and SEQ ID NOs: 86-88, SEQ ID NOs: 22-24 and SEQ ID NOs: 86-88, SEQ ID NOs: 26-28 and SEQ ID NOs: 86-88, SEQ ID NOs: 30-32 and SEQ ID NOs: 86-88, SEQ ID NOs: 34-36 and SEQ ID NOs: 86-88, SEQ ID NOs: 38-40 and SEQ ID NOs: 86-88, SEQ ID NOs: 42-44 and SEQ ID NOs: 86-88, SEQ ID NOs: 46-48 and SEQ ID NOs: 86-88, SEQ ID NOs: 50-52 and SEQ ID NOs: 86-88, SEQ ID NOs: 54-56 and SEQ ID NOs: 86-88, SEQ ID NOs: 58-60 and SEQ ID NOs: 86-88, SEQ ID NOs: 62-64 and SEQ ID NOs: 86-88; SEQ ID NOs: 66-68 and SEQ ID NOs: 86-88 (furthermore any of the sIL-6R CDR sets shown in SEQ ID NOs: 138-194 can similarly be combined with SEQ ID NO: 86-88 and any of the CTGF CDR sets as set forth in SEQ ID NOs: 233-286 can be used in place of SEQ ID NOs: 86-88); SEQ ID NOs: 2-4 and SEQ ID NOs: 90-92, SEQ ID NOs: 6-8 and SEQ ID NOs: 90-92, SEQ ID NOs: 10-12 and SEQ ID NOs: 90-92, SEQ ID NOs: 14-16 and SEQ ID NOs: 90-92, SEQ ID NOs: 18-20 and SEQ ID NOs: 90-92, SEQ ID NOs: 22-24 and SEQ ID NOs: 90-92, SEQ ID NOs: 26-28 and SEQ ID NOs: 90-92, SEQ ID NOs: 30-32 and SEQ ID NOs: 90-92, SEQ ID NOs: 34-36 and SEQ ID NOs: 90-92, SEQ ID NOs: 38-40 and SEQ ID NOs: 90-92, SEQ ID NOs: 42-44 and SEQ ID NOs: 90-92, SEQ ID NOs: 46-48 and SEQ ID NOs: 90-92, SEQ ID NOs: 50-52 and SEQ ID NOs: 90-92, SEQ ID NOs: 54-56 and SEQ ID NOs: 90-92, SEQ ID NOs: 58-60 and SEQ ID NOs: 90-92, SEQ ID NOs: 62-64 and SEQ ID NOs: 90-92 or SEQ ID NOs: 66-68 and SEQ ID NOs: 90-92 (furthermore any of the sIL-6R CDR sets shown in SEQ ID NOs: 138-194 can similarly be combined with SEQ ID NO:90-92 and any of the CTGF CDR sets as set forth in SEQ ID NOs: 233-286 can be used in place of SEQ ID NOs: 90-92), and a pharmaceutically acceptable carrier;
(vi) one or more multispecific binding proteins that selectively and/or specifically binds to human sIL-6R and to human CTGF as described herein, for example including the amino acid sequence of: SEQ ID NO: 1 and SEQ ID NO:69, SEQ ID NO:5 and SEQ ID NO:69, SEQ ID NO:9 and SEQ ID NO: 69, SEQ ID NO: 13 and SEQ ID NO:69, SEQ ID NO: 17 and SEQ ID NO:69, SEQ ID NO: 21 and SEQ ID NO:69, SEQ ID NO:25 and SEQ ID NO:69, SEQ ID NO:29 and SEQ ID NO: 69, SEQ ID NO:33 and SEQ ID NO:69, SEQ ID NO:37 and SEQ ID NO:69, SEQ ID NO:41 and SEQ ID NO:69, SEQ ID NO: 45 and SEQ ID NO:69, SEQ ID NO:49 and SEQ ID NO:69, SEQ ID NO: 53 and SEQ ID NO:69, SEQ ID NO:57 and SEQ ID NO:69, SEQ ID NO:61 and SEQ ID NO: 69, SEQ ID NO: 65 and SEQ ID NO: 69 (furthermore any of the sIL-6R VHHs shown in SEQ ID NOs: 119-137 can similarly be combined with SEQ ID NO:69 and any of SEQ ID NO:214-232 can be used in place of SEQ ID NO:69); SEQ ID NO: 1 and SEQ ID NO:73, SEQ ID NO:5 and SEQ ID NO:73, SEQ ID NO:9 and SEQ ID NO:73, SEQ ID NO: 13 and SEQ ID NO:73, SEQ ID NO: 17 and SEQ ID NO:73, SEQ ID NO:21 and SEQ ID NO:73, SEQ ID NO:25 and SEQ ID NO:73, SEQ ID NO:29 and SEQ ID NO:73, SEQ ID NO:33 and SEQ ID NO:73, SEQ ID NO:37 and SEQ ID NO: 73, SEQ ID NO:41 and SEQ ID NO:73, SEQ ID NO: 45 and SEQ ID NO:73, SEQ ID NO:49 and SEQ ID NO:73, SEQ ID NO:53 and SEQ ID NO:73, SEQ ID NO:57 and SEQ ID NO:73, SEQ ID NO: 61 and SEQ ID NO:73, SEQ ID NO:65 and SEQ ID NO:73 (furthermore any of the sIL-6R VHHs shown in SEQ ID NOs: 119-137 can similarly be combined with SEQ ID NO:73 and any of SEQ ID NO:214-232 can be used in place of SEQ ID NO:73); SEQ ID NO: 1 and SEQ ID NO:77, SEQ ID NO:5 and SEQ ID NO:77, SEQ ID NO:9 and SEQ ID NO:77, SEQ ID NO: 13 and SEQ ID NO: 77, SEQ ID NO: 17 and SEQ ID NO:77, SEQ ID NO:21 and SEQ ID NO:77, SEQ ID NO:25 and SEQ ID NO:77, SEQ ID NO:29 and SEQ ID NO:77, SEQ ID NO:33 and SEQ ID NO:77, SEQ ID NO: 37 and SEQ ID NO:77, SEQ ID NO:41 and SEQ ID NO:77, SEQ ID NO:45 and SEQ ID NO: 77, SEQ ID NO:49 and SEQ ID NO:77, SEQ ID NO:53 and SEQ ID NO:77, SEQ ID NO:57 and SEQ ID NO:77, SEQ ID NO:61 and SEQ ID NO:77, SEQ ID NO:65 and SEQ ID NO:77 (furthermore any of the sIL-6R VHHs shown in SEQ ID NOs: 119-137 can similarly be combine with SEQ ID NO:77 and any of SEQ ID NO:214-232 can be used in place of SEQ ID NO:77); SEQ ID NO: 1 and SEQ ID NO:81, SEQ ID NO:5 and SEQ ID NO: 81, SEQ ID NO: 9 and SEQ ID NO:81, SEQ ID NO: 13 and SEQ ID NO:81, SEQ ID NO: 17 and SEQ ID NO:81, SEQ ID NO:21 and SEQ ID NO: 81, SEQ ID NO:25 and SEQ ID NO:81, SEQ ID NO:29 and SEQ ID NO:81, SEQ ID NO:33 and SEQ ID NO:81, SEQ ID NO:37 and SEQ ID NO:81, SEQ ID NO: 41 and SEQ ID NO:81, SEQ ID NO: 45 and SEQ ID NO:81, SEQ ID NO:49 and SEQ ID NO:81, SEQ ID NO:53 and SEQ ID NO: 81, SEQ ID NO:57 and SEQ ID NO:81, SEQ ID NO:61 and SEQ ID NO:81, SEQ ID NO:65 and SEQ ID NO:81 (furthermore any of the sIL-6R VHHs shown in SEQ ID NOs: 119-137 can similarly be combined with SEQ ID NO:81 and any of SEQ ID NO:214-232 can be used in place of SEQ ID NO:81); SEQ ID NO: 1 and SEQ ID NO:85, SEQ ID NO:5 and SEQ ID NO:85, SEQ ID NO: 9 and SEQ ID NO: 85, SEQ ID NO: 13 and SEQ ID NO:85, SEQ ID NO: 17 and SEQ ID NO:85, SEQ ID NO:21 and SEQ ID NO:85, SEQ ID NO:25 and SEQ ID NO:85, SEQ ID NO:29 and SEQ ID NO: 85, SEQ ID NO:33 and SEQ ID NO:85, SEQ ID NO:37 and SEQ ID NO:85, SEQ ID NO:41 and SEQ ID NO: 85, SEQ ID NO:45 and SEQ ID NO:85, SEQ ID NO:49 and SEQ ID NO:85, SEQ ID NO: 53 and SEQ ID NO:85, SEQ ID NO:57 and SEQ ID NO:85, SEQ ID NO:61 and SEQ ID NO: 85, SEQ ID NO: 65 and SEQ ID NO: 85 (furthermore any of the sIL-6R VHHs shown in SEQ ID NOs: 119-137 can similarly be combined with SEQ ID NO:85 and any of SEQ ID NO:214-232 can be used in place of SEQ ID NO:85); SEQ ID NO: 1 and SEQ ID NO:89, SEQ ID NO:5 and SEQ ID NO:89, SEQ ID NO:9 and SEQ ID NO:89, SEQ ID NO: 13 and SEQ ID NO:89, SEQ ID NO: 17 and SEQ ID NO: 89, SEQ ID NO:21 and SEQ ID NO:89, SEQ ID NO:25 and SEQ ID NO:89, SEQ ID NO:29 and SEQ ID NO: 89, SEQ ID NO:33 and SEQ ID NO:89, SEQ ID NO:37 and SEQ ID NO: 89, SEQ ID NO:41 and SEQ ID NO:89, SEQ ID NO:45 and SEQ ID NO:89, SEQ ID NO:49 and SEQ ID NO: 89, SEQ ID NO:53 and SEQ ID NO:89, SEQ ID NO:57 and SEQ ID NO:89, SEQ ID NO: 61 and SEQ ID NO:89, or SEQ ID NO:65 and SEQ ID NO:89 (furthermore any of the sIL-6R VHHs shown in SEQ ID NOs: 119-137 can similarly be combined with SEQ ID NO:89 and any of SEQ ID NO:214-232 can be used in place of SEQ ID NO:89), and a pharmaceutically acceptable carrier;
(vii) one or more fusion proteins including a sIL-6R binding protein as described herein and an anti-transferrin receptor antibody, for example including CDRs as set forth in SEQ ID NOs: 2-4, SEQ ID NOs: 6-8, SEQ ID NOs: 10-12, SEQ ID NOs: 14-16, SEQ ID NOs: 18-20, SEQ ID NOs: 22-24, SEQ ID NOs: 26-28, SEQ ID NOs: 30-32, SEQ ID NOs: 34-36, SEQ ID NOs: 38-40, SEQ ID NOs: 42-44, SEQ ID NOs: 46-48, SEQ ID NOs: 50-52, SEQ ID NOs: 54-56, SEQ ID NOs: 58-60, SEQ ID NOs: 62-64, SEQ ID NOs: 66-68, or SEQ ID NO: 138-194 each further including an amino acid sequence encoding an anti-transferrin receptor antibody, and a pharmaceutically acceptable carrier;
(viii) one or more fusion proteins including a sIL-6R binding protein as described herein and an anti-transferrin receptor antibody, for example including the amino acid sequence of SEQ ID NO: 1, SEQ ID NO:5, SEQ ID NO:9, SEQ ID NO: 13, SEQ ID NO: 17, SEQ ID NO:21, SEQ ID NO: 25, SEQ ID NO:29, SEQ ID NO:33, SEQ ID NO:37, SEQ ID NO:41, SEQ ID NO:45, SEQ ID NO: 49, SEQ ID NO:53, SEQ ID NO:57, SEQ ID NO:61, SEQ ID NO:65, or SEQ ID NOs: 119-137, each further including an amino acid sequence encoding an anti-transferrin receptor antibody, and a pharmaceutically acceptable carrier;
(ix) one or more fusion proteins including a CTGF binding protein as described herein and an anti-transferrin receptor antibody, for example including CDRs as set forth in NOs: 70-72, SEQ ID NOs: 74-76, SEQ ID NOs: 78-80, SEQ ID NOs: 82-84, SEQ ID NOS: 86-88, SEQ ID NOS: 90-92, SEQ ID NOs: 233-286, or SEQ ID NOs: 291-293 each further including an amino acid sequence encoding an anti-transferrin receptor antibody, and a pharmaceutically acceptable carrier;
(x) one or more fusion proteins including a CTGF binding protein as described herein and an anti-transferrin receptor antibody, for example including the amino acid sequence of SEQ ID NO: 73, SEQ ID NO:77, SEQ ID NO:81, SEQ ID NO:85, SEQ ID NO:89, or SEQ ID NOs: 214-232, each further including an amino acid sequence encoding an anti-transferrin receptor antibody, and a pharmaceutically acceptable carrier;
(xi) one or more fusion proteins including a multispecific binding protein that selectively and/or specifically binds to human sIL-6R and to human CTGF as described herein and an anti-transferrin receptor antibody, for example including CDRs as set forth in SEQ ID NOs: 2-4 and SEQ ID NOs: 70-72, SEQ ID NOs: 6-8 and SEQ ID NOs: 70-72, SEQ ID NOs: 10-12 and SEQ ID NOs: 70-72, SEQ ID NOs: 14-16 and SEQ ID NOs: 70-72, SEQ ID NOs: 18-20 and SEQ ID NOs: 70-72, SEQ ID NOs: 22-24 and SEQ ID NOs: 70-72, SEQ ID NOs: 26-28 and SEQ ID NOs: 70-72, SEQ ID NOs: 30-32 and SEQ ID NOs: 70-72, SEQ ID NOs: 34-36 and SEQ ID NOs: 70-72, SEQ ID NOs: 38-40 and SEQ ID NOs: 70-72, SEQ ID NOs: 42-44 and SEQ ID NOs: 70-72, SEQ ID NOs: 46-48 and SEQ ID NOs: 70-72, SEQ ID NOs: 50-52 and SEQ ID NOs: 70-72, SEQ ID NOs: 54-56 and SEQ ID NOs: 70-72, SEQ ID NOs: 58-60 and SEQ ID NOs: 70-72, SEQ ID NOs: 62-64 and SEQ ID NOs: 70-72; SEQ ID NOs: 66-68 and SEQ ID NOs: 70-72 (furthermore any of the sIL-6R CDR sets shown in SEQ ID NOs: 138-194 can similarly be combined with SEQ ID NO:70-72 and any of the CTGF CDR sets as set forth in SEQ ID NOs: 233-286 can be used in place of SEQ ID NOs: 70-72); SEQ ID NOs: 2-4 and SEQ ID NOs: 74-76, SEQ ID NOs: 6-8 and SEQ ID NOs: 74-76, SEQ ID NOs: 10-12 and SEQ ID NOs: 74-76, SEQ ID NOs: 14-16 and SEQ ID NOs: 74-76, SEQ ID NOs: 18-20 and SEQ ID NOs: 74-76, SEQ ID NOs: 22-24 and SEQ ID NOs: 74-76, SEQ ID NOs: 26-28 and SEQ ID NOs: 74-76, SEQ ID NOs: 30-32 and SEQ ID NOs: 74-76, SEQ ID NOs: 34-36 and SEQ ID NOs: 74-76, SEQ ID NOs: 38-40 and SEQ ID NOs: 74-76, SEQ ID NOs: 42-44 and SEQ ID NOs: 74-76, SEQ ID NOs: 46-48 and SEQ ID NOs: 74-76, SEQ ID NOs: 50-52 and SEQ ID NOs: 74-76, SEQ ID NOs: 54-56 and SEQ ID NOs: 74-76, SEQ ID NOs: 58-60 and SEQ ID NOs: 74-76, SEQ ID NOs: 62-64 and SEQ ID NOs: 74-76; SEQ ID NOs: 66-68 and SEQ ID NOs: 74-76 (furthermore any of the sIL-6R CDR sets shown in SEQ ID NOs: 138-194 can similarly be combined with SEQ ID NO: 74-76 and any of the CTGF CDR sets as set forth in SEQ ID NOs: 233-286 can be used in place of SEQ ID NOs: 74-76); SEQ ID NOs: 2-4 and SEQ ID NOs: 78-80, SEQ ID NOs: 6-8 and SEQ ID NOs: 78-80, SEQ ID NOs: 10-12 and SEQ ID NOs: 78-80, SEQ ID NOs: 14-16 and SEQ ID NOs: 78-80, SEQ ID NOs: 18-20 and SEQ ID NOs: 78-80, SEQ ID NOs: 22-24 and SEQ ID NOs: 78-80, SEQ ID NOs: 26-28 and SEQ ID NOs: 78-80, SEQ ID NOs: 30-32 and SEQ ID NOs: 78-80, SEQ ID NOs: 34-36 and SEQ ID NOs: 78-80, SEQ ID NOs: 38-40 and SEQ ID NOs: 78-80, SEQ ID NOs: 42-44 and SEQ ID NOs: 78-80, SEQ ID NOs: 46-48 and SEQ ID NOs: 78-80, SEQ ID NOs: 50-52 and SEQ ID NOs: 78-80, SEQ ID NOs: 54-56 and SEQ ID NOs: 78-80, SEQ ID NOs: 58-60 and SEQ ID NOs: 78-80, SEQ ID NOs: 62-64 and SEQ ID NOs: 78-80; SEQ ID NOs: 66-68 and SEQ ID NOs: 78-80 (furthermore any of the sIL-6R CDR sets shown in SEQ ID NOs: 138-194 can similarly be combined with SEQ ID NO:78-80 and any of the CTGF CDR sets as set forth in SEQ ID NOs: 233-286 can be used in place of SEQ ID NOs: 78-80); SEQ ID NOs: 2-4 and SEQ ID NOs: 82-84, SEQ ID NOs: 6-8 and SEQ ID NOs: 82-84, SEQ ID NOs: 10-12 and SEQ ID NOs: 82-84, SEQ ID NOs: 14-16 and SEQ ID NOs: 82-84, SEQ ID NOs: 18-20 and SEQ ID NOs: 82-84, SEQ ID NOs: 22-24 and SEQ ID NOs: 82-84, SEQ ID NOs: 26-28 and SEQ ID NOs: 82-84, SEQ ID NOs: 30-32 and SEQ ID NOs: 82-84, SEQ ID NOs: 34-36 and SEQ ID NOs: 82-84, SEQ ID NOs: 38-40 and SEQ ID NOs: 82-84, SEQ ID NOs: 42-44 and SEQ ID NOs: 82-84, SEQ ID NOs: 46-48 and SEQ ID NOs: 82-84, SEQ ID NOs: 50-52 and SEQ ID NOs: 82-84, SEQ ID NOs: 54-56 and SEQ ID NOs: 82-84, SEQ ID NOs: 58-60 and SEQ ID NOs: 82-84, SEQ ID NOs: 62-64 and SEQ ID NOs: 82-84; SEQ ID NOs: 66-68 and SEQ ID NOs: 82-84 (furthermore any of the sIL-6R CDR sets shown in SEQ ID NOs: 138-194 can similarly be combined with SEQ ID NO: 82-84 and any of the CTGF CDR sets as set forth in SEQ ID NOs: 233-286 can be used in place of SEQ ID NOs: 82-84); SEQ ID NOS: 2-4 and SEQ ID NOs: 86-88, SEQ ID NOs: 6-8 and SEQ ID NOs: 86-88, SEQ ID NOs: 10-12 and SEQ ID NOs: 86-88, SEQ ID NOs: 14-16 and SEQ ID NOs: 86-88, SEQ ID NOs: 18-20 and SEQ ID NOs: 86-88, SEQ ID NOs: 22-24 and SEQ ID NOs: 86-88, SEQ ID NOs: 26-28 and SEQ ID NOs: 86-88, SEQ ID NOs: 30-32 and SEQ ID NOs: 86-88, SEQ ID NOs: 34-36 and SEQ ID NOs: 86-88, SEQ ID NOs: 38-40 and SEQ ID NOs: 86-88, SEQ ID NOs: 42-44 and SEQ ID NOs: 86-88, SEQ ID NOs: 46-48 and SEQ ID NOs: 86-88, SEQ ID NOs: 50-52 and SEQ ID NOs: 86-88, SEQ ID NOs: 54-56 and SEQ ID NOs: 86-88, SEQ ID NOs: 58-60 and SEQ ID NOs: 86-88, SEQ ID NOs: 62-64 and SEQ ID NOs: 86-88; SEQ ID NOs: 66-68 and SEQ ID NOs: 86-88 (furthermore any of the sIL-6R CDR sets shown in SEQ ID NOs: 138-194 can similarly be combined with SEQ ID NO:86-88 and any of the CTGF CDR sets as set forth in SEQ ID NOs: 233-286 can be used in place of SEQ ID NOs: 86-88); SEQ ID NOs: 2-4 and SEQ ID NOs: 90-92, SEQ ID NOs: 6-8 and SEQ ID NOs: 90-92, SEQ ID NOs: 10-12 and SEQ ID NOs: 90-92, SEQ ID NOs: 14-16 and SEQ ID NOs: 90-92, SEQ ID NOs: 18-20 and SEQ ID NOs: 90-92, SEQ ID NOs: 22-24 and SEQ ID NOs: 90-92, SEQ ID NOs: 26-28 and SEQ ID NOs: 90-92, SEQ ID NOs: 30-32 and SEQ ID NOs: 90-92, SEQ ID NOs: 34-36 and SEQ ID NOs: 90-92, SEQ ID NOs: 38-40 and SEQ ID NOs: 90-92, SEQ ID NOs: 42-44 and SEQ ID NOs: 90-92, SEQ ID NOs: 46-48 and SEQ ID NOs: 90-92, SEQ ID NOs: 50-52 and SEQ ID NOs: 90-92, SEQ ID NOs: 54-56 and SEQ ID NOs: 90-92, SEQ ID NOs: 58-60 and SEQ ID NOs: 90-92, SEQ ID NOs: 62-64 and SEQ ID NOs: 90-92 or SEQ ID NOs: 66-68 and SEQ ID NOs: 90-92 (furthermore any of the sIL-6R CDR sets shown in SEQ ID NOs: 138-194 can similarly be combined with SEQ ID NO: 90-92 and any of the CTGF CDR sets as set forth in SEQ ID NOs: 233-286 can be used in place of SEQ ID NOs: 90-92), each further including an amino acid sequence encoding an anti-transferrin receptor antibody, and a pharmaceutically acceptable carrier; and
(xii) one or more fusion proteins including a multispecific binding protein that selectively and/or specifically binds to human sIL-6R and to human CTGF as described herein and an anti-transferrin receptor antibody, for example including the amino acid sequence of: SEQ ID NO:1 and SEQ ID NO:69, SEQ ID NO:5 and SEQ ID NO:69, SEQ ID NO:9 and SEQ ID NO:69, SEQ ID NO: 13 and SEQ ID NO:69, SEQ ID NO: 17 and SEQ ID NO:69, SEQ ID NO:21 and SEQ ID NO: 69, SEQ ID NO:25 and SEQ ID NO:69, SEQ ID NO:29 and SEQ ID NO:69, SEQ ID NO:33 and SEQ ID NO:69, SEQ ID NO:37 and SEQ ID NO:69, SEQ ID NO:41 and SEQ ID NO:69, SEQ ID NO: 45 and SEQ ID NO:69, SEQ ID NO:49 and SEQ ID NO:69, SEQ ID NO:53 and SEQ ID NO: 69, SEQ ID NO:57 and SEQ ID NO:69, SEQ ID NO:61 and SEQ ID NO:69, SEQ ID NO:65 and SEQ ID NO:69 (furthermore any of the sIL-6R VHHs shown in SEQ ID NOs: 119-137 can similarly be combined with SEQ ID NO:69 and any of SEQ ID NO:214-232 can be used in place of SEQ ID NO:69); SEQ ID NO: 1 and SEQ ID NO:73, SEQ ID NO:5 and SEQ ID NO:73, SEQ ID NO: 9 and SEQ ID NO:73, SEQ ID NO: 13 and SEQ ID NO:73, SEQ ID NO: 17 and SEQ ID NO:73, SEQ ID NO:21 and SEQ ID NO:73, SEQ ID NO:25 and SEQ ID NO:73, SEQ ID NO:29 and SEQ ID NO: 73, SEQ ID NO:33 and SEQ ID NO:73, SEQ ID NO:37 and SEQ ID NO:73, SEQ ID NO:41 and SEQ ID NO:73, SEQ ID NO:45 and SEQ ID NO:73, SEQ ID NO:49 and SEQ ID NO:73, SEQ ID NO: 53 and SEQ ID NO:73, SEQ ID NO:57 and SEQ ID NO:73, SEQ ID NO:61 and SEQ ID NO: 73, SEQ ID NO: 65 and SEQ ID NO:73 (furthermore any of the sIL-6R VHHs shown in SEQ ID NOs: 119-137 can similarly be combined with SEQ ID NO:73 and any of SEQ ID NO:214-232 can be used in place of SEQ ID NO:73); SEQ ID NO:1 and SEQ ID NO:77, SEQ ID NO:5 and SEQ ID NO:77, SEQ ID NO:9 and SEQ ID NO:77, SEQ ID NO: 13 and SEQ ID NO:77, SEQ ID NO: 17 and SEQ ID NO: 77, SEQ ID NO:21 and SEQ ID NO:77, SEQ ID NO:25 and SEQ ID NO:77, SEQ ID NO:29 and SEQ ID NO:77, SEQ ID NO:33 and SEQ ID NO:77, SEQ ID NO:37 and SEQ ID NO: 77, SEQ ID NO:41 and SEQ ID NO:77, SEQ ID NO:45 and SEQ ID NO:77, SEQ ID NO:49 and SEQ ID NO:77, SEQ ID NO:53 and SEQ ID NO:77, SEQ ID NO:57 and SEQ ID NO:77, SEQ ID NO: 61 and SEQ ID NO:77, SEQ ID NO:65 and SEQ ID NO:77 (furthermore any of the sIL-6R VHHs shown in SEQ ID NOs: 119-137 can similarly be combined with SEQ ID NO:77 and any of SEQ ID NO:214-232 can be used in place of SEQ ID NO:77); SEQ ID NO:1 and SEQ ID NO:81, SEQ ID NO:5 and SEQ ID NO:81, SEQ ID NO:9 and SEQ ID NO:81, SEQ ID NO: 13 and SEQ ID NO: 81, SEQ ID NO: 17 and SEQ ID NO:81, SEQ ID NO:21 and SEQ ID NO:81, SEQ ID NO:25 and SEQ ID NO:81, SEQ ID NO:29 and SEQ ID NO:81, SEQ ID NO:33 and SEQ ID NO:81, SEQ ID NO: 37 and SEQ ID NO:81, SEQ ID NO:41 and SEQ ID NO:81, SEQ ID NO:45 and SEQ ID NO: 81, SEQ ID NO:49 and SEQ ID NO:81, SEQ ID NO:53 and SEQ ID NO:81, SEQ ID NO:57 and SEQ ID NO:81, SEQ ID NO:61 and SEQ ID NO:81, SEQ ID NO:65 and SEQ ID NO:81 (furthermore any of the sIL-6R VHHs shown in SEQ ID NOs: 119-137 can similarly be combined with SEQ ID NO: 81 and any of SEQ ID NO:214-232 can be used in place of SEQ ID NO:81); SEQ ID NO: 1 and SEQ ID NO:85, SEQ ID NO:5 and SEQ ID NO:85, SEQ ID NO:9 and SEQ ID NO:85, SEQ ID NO: 13 and SEQ ID NO:85, SEQ ID NO: 17 and SEQ ID NO:85, SEQ ID NO:21 and SEQ ID NO: 85, SEQ ID NO:25 and SEQ ID NO:85, SEQ ID NO:29 and SEQ ID NO:85, SEQ ID NO:33 and SEQ ID NO:85, SEQ ID NO:37 and SEQ ID NO:85, SEQ ID NO:41 and SEQ ID NO:85, SEQ ID NO: 45 and SEQ ID NO:85, SEQ ID NO:49 and SEQ ID NO:85, SEQ ID NO:53 and SEQ ID NO: 85, SEQ ID NO:57 and SEQ ID NO:85, SEQ ID NO:61 and SEQ ID NO:85, SEQ ID NO:65 and SEQ ID NO:85 (furthermore any of the sIL-6R VHHs shown in SEQ ID NOs: 119-137 can similarly be combined with SEQ ID NO:85 and any of SEQ ID NO:214-232 can be used in place of SEQ ID NO: 85); SEQ ID NO: 1 and SEQ ID NO:89, SEQ ID NO:5 and SEQ ID NO:89, SEQ ID NO: 9 and SEQ ID NO: 89, SEQ ID NO: 13 and SEQ ID NO: 89, SEQ ID NO: 17 and SEQ ID NO:89, SEQ ID NO:21 and SEQ ID NO: 89, SEQ ID NO:25 and SEQ ID NO:89, SEQ ID NO:29 and SEQ ID NO: 89, SEQ ID NO:33 and SEQ ID NO:89, SEQ ID NO:37 and SEQ ID NO:89, SEQ ID NO:41 and SEQ ID NO: 89, SEQ ID NO: 45 and SEQ ID NO: 89, SEQ ID NO:49 and SEQ ID NO:89, SEQ ID NO: 53 and SEQ ID NO:89, SEQ ID NO:57 and SEQ ID NO:89, SEQ ID NO:61 and SEQ ID NO: 89, or SEQ ID NO: 65 and SEQ ID NO: 89 (furthermore any of the sIL-6R VHHs shown in SEQ ID NOs: 119-137 can similarly be combined with SEQ ID NO:89 and any of SEQ ID NO:214-232 can be used in place of SEQ ID NO:89), each further including an amino acid sequence encoding an anti-transferrin receptor antibody, and a pharmaceutically acceptable carrier.

### Methods of Use

An embodiment provides a method of treating a neurological disease or condition in a subject comprising administering to the subject a therapeutically effective amount of any one of the binding peptides, any one of the multispecific binding protein, any one of the fusion proteins described herein, or any one of the pharmaceutical compositions described herein, thereby treating the neurological disease or condition in the subject.

The term “subject” as used herein refers to any individual or patient to which the subject methods are performed. Generally, the subject is human, although as will be appreciated by those in the art, the subject may be an animal. Thus, other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

The term “treatment” is used interchangeably herein with the term “therapeutic method” and refers to both 1) therapeutic treatments or measures that cure, slow down, lessen symptoms of, and/or halt progression of a diagnosed pathologic conditions or disorder, and 2) and prophylactic/preventative measures. Those in need of treatment may include individuals already having a particular medical disorder as well as those who may ultimately acquire the disorder (i.e., those needing preventive measures).

The terms “therapeutically effective amount”, “effective dose,” “therapeutically effective dose”, “effective amount,” or the like refer to that amount of the subject compound that will elicit the biological or medical response of a tissue, system, animal, or human that is being sought by the researcher, veterinarian, medical doctor or other clinician. Generally, the response is either amelioration of symptoms in a patient or a desired biological outcome (e.g., treating the neurological disease or condition in the subject). Such amount should be sufficient to treat the neurological disease or condition. The effective amount can be determined as described herein.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, as well infusion, inhalation, and nebulization. The phrases “parenteral administration” and “administered parenterally” as used herein means modes of administration other than enteral and topical administration.

Administering can comprise intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal, intrasternal, oral, sublingual buccal or nasal administrations, infusion, inhalation, or nebulization. For example, administering can comprise crossing blood brain barrier and delivering to intracellular space enter the brain hippocampal region.

The binding peptides, multispecific binding proteins, fusion proteins, or pharmaceutical compositions can be any of the ones described herein.

For example, the binding peptide can be:
(i) a sIL-6R binding protein as described herein, for example including CDRs as set forth in SEQ ID NOs: 2-4, SEQ ID NOs: 6-8, SEQ ID NOs: 10-12, SEQ ID NOs: 14-16, SEQ ID NOs: 18-20, SEQ ID NOs: 22-24, SEQ ID NOs: 26-28, SEQ ID NOs: 30-32, SEQ ID NOs: 34-36, SEQ ID NOs: 38-40, SEQ ID NOs: 42-44, SEQ ID NOs: 46-48, SEQ ID NOs: 50-52, SEQ ID NOs: 54-56, SEQ ID NOs: 58-60, SEQ ID NOs: 62-64, SEQ ID NOs: 66-68, or SEQ ID NO: 138-194;
(ii) a sIL-6R binding protein as described herein, for example including the amino acid sequence of SEQ ID NO:1, SEQ ID NO:5, SEQ ID NO:9, SEQ ID NO: 13, SEQ ID NO: 17, SEQ ID NO: 21, SEQ ID NO:25, SEQ ID NO:29, SEQ ID NO:33, SEQ ID NO:37, SEQ ID NO:41, SEQ ID NO: 45, SEQ ID NO:49, SEQ ID NO:53, SEQ ID NO:57, SEQ ID NO:61, SEQ ID NO:65, or SEQ ID Nos: 119-137;
(iii) a CTGF binding protein as described herein, for example including CDRs as set forth in NOs: 70-72, SEQ ID NOs: 74-76, SEQ ID NOs: 78-80, SEQ ID NOs: 82-84, SEQ ID NOs: 86-88, SEQ ID NOs: 90-92, SEQ ID NOs: 233-286, or SEQ ID NOs: 291-293; or
(iv) a CTGF binding protein as described herein, for example including the amino acid sequence of SEQ ID NO:73, SEQ ID NO:77, SEQ ID NO:81, SEQ ID NO:85, SEQ ID NO:89 or SEQ ID NOs: 214-232.

For example, the multispecific binding protein can be:
(i) a multispecific binding protein that selectively and/or specifically binds to human sIL-6R and to human CTGF as described herein, for example including CDRs as set forth in SEQ ID NOs: 2-4 and SEQ ID NOs: 70-72, SEQ ID NOs: 6-8 and SEQ ID NOs: 70-72, SEQ ID NOs: 10-12 and SEQ ID NOs: 70-72, SEQ ID NOs: 14-16 and SEQ ID NOs: 70-72, SEQ ID NOs: 18-20 and SEQ ID NOs: 70-72, SEQ ID NOs: 22-24 and SEQ ID NOs: 70-72, SEQ ID NOs: 26-28 and SEQ ID NOs: 70-72, SEQ ID NOs: 30-32 and SEQ ID NOs: 70-72, SEQ ID NOs: 34-36 and SEQ ID NOs: 70-72, SEQ ID NOs: 38-40 and SEQ ID NOs: 70-72, SEQ ID NOs: 42-44 and SEQ ID NOs: 70-72, SEQ ID NOs: 46-48 and SEQ ID NOs: 70-72, SEQ ID NOs: 50-52 and SEQ ID NOs: 70-72, SEQ ID NOs: 54-56 and SEQ ID NOs: 70-72, SEQ ID NOs: 58-60 and SEQ ID NOs: 70-72, SEQ ID NOs: 62-64 and SEQ ID NOs: 70-72; SEQ ID NOs: 66-68 and SEQ ID NOs: 70-72 (furthermore any of the SIL-6R CDR sets shown in SEQ ID NOs: 138-194 can similarly be combined with SEQ ID NO:70-72 and any of the CTGF CDR sets as set forth in SEQ ID NOs: 233-286 can be used in place of SEQ ID NOs: 70-72); SEQ ID NOs: 2-4 and SEQ ID NOs: 74-76, SEQ ID NOs: 6-8 and SEQ ID NOs: 74-76, SEQ ID NOs: 10-12 and SEQ ID NOs: 74-76, SEQ ID NOs: 14-16 and SEQ ID NOs: 74-76, SEQ ID NOs: 18-20 and SEQ ID NOs: 74-76, SEQ ID NOs: 22-24 and SEQ ID NOs: 74-76, SEQ ID NOs: 26-28 and SEQ ID NOs: 74-76, SEQ ID NOs: 30-32 and SEQ ID NOs: 74-76, SEQ ID NOs: 34-36 and SEQ ID NOs: 74-76, SEQ ID NOs: 38-40 and SEQ ID NOs: 74-76, SEQ ID NOs: 42-44 and SEQ ID NOs: 74-76, SEQ ID NOs: 46-48 and SEQ ID NOs: 74-76, SEQ ID NOs: 50-52 and SEQ ID NOs: 74-76, SEQ ID NOs: 54-56 and SEQ ID NOs: 74-76, SEQ ID NOs: 58-60 and SEQ ID NOs: 74-76, SEQ ID NOs: 62-64 and SEQ ID NOs: 74-76; SEQ ID NOs: 66-68 and SEQ ID NOs: 74-76 (furthermore any of the sIL-6R CDR sets shown in SEQ ID NOs: 138-194 can similarly be combined with SEQ ID NO:74-76 and any of the CTGF CDR sets as set forth in SEQ ID NOs: 233-286 can be used in place of SEQ ID NOs: 74-76); SEQ ID NOs: 2-4 and SEQ ID NOs: 78-80, SEQ ID NOs: 6-8 and SEQ ID NOs: 78-80, SEQ ID NOs: 10-12 and SEQ ID NOs: 78-80, SEQ ID NOs: 14-16 and SEQ ID NOs: 78-80, SEQ ID NOs: 18-20 and SEQ ID NOs: 78-80, SEQ ID NOs: 22-24 and SEQ ID NOs: 78-80, SEQ ID NOs: 26-28 and SEQ ID NOs: 78-80, SEQ ID NOs: 30-32 and SEQ ID NOs: 78-80, SEQ ID NOs: 34-36 and SEQ ID NOs: 78-80, SEQ ID NOs: 38-40 and SEQ ID NOs: 78-80, SEQ ID NOs: 42-44 and SEQ ID NOs: 78-80, SEQ ID NOs: 46-48 and SEQ ID NOs: 78-80, SEQ ID NOs: 50-52 and SEQ ID NOs: 78-80, SEQ ID NOs: 54-56 and SEQ ID NOs: 78-80, SEQ ID NOs: 58-60 and SEQ ID NOs: 78-80, SEQ ID NOs: 62-64 and SEQ ID NOs: 78-80; SEQ ID NOs: 66-68 and SEQ ID NOs: 78-80 (furthermore any of the sIL-6R CDR sets shown in SEQ ID NOs: 138-194 can similarly be combined with SEQ ID NO:78-80 and any of the CTGF CDR sets as set forth in SEQ ID NOs: 233-286 can be used in place of SEQ ID NOs: 78-80); SEQ ID NOs: 2-4 and SEQ ID NOs: 82-84, SEQ ID NOs: 6-8 and SEQ ID NOs: 82-84, SEQ ID NOs: 10-12 and SEQ ID NOs: 82-84, SEQ ID NOs: 14-16 and SEQ ID NOs: 82-84, SEQ ID NOs: 18-20 and SEQ ID NOs: 82-84, SEQ ID NOs: 22-24 and SEQ ID NOs: 82-84, SEQ ID NOs: 26-28 and SEQ ID NOs: 82-84, SEQ ID NOs: 30-32 and SEQ ID NOs: 82-84, SEQ ID NOs: 34-36 and SEQ ID NOs: 82-84, SEQ ID NOs: 38-40 and SEQ ID NOs: 82-84, SEQ ID NOs: 42-44 and SEQ ID NOs: 82-84, SEQ ID NOs: 46-48 and SEQ ID NOs: 82-84, SEQ ID NOs: 50-52 and SEQ ID NOs: 82-84, SEQ ID NOs: 54-56 and SEQ ID NOs: 82-84, SEQ ID NOs: 58-60 and SEQ ID NOs: 82-84, SEQ ID NOs: 62-64 and SEQ ID NOs: 82-84; SEQ ID NOs: 66-68 and SEQ ID NOs: 82-84 (furthermore any of the sIL-6R CDR sets shown in SEQ ID NOs: 138-194 can similarly be combined with SEQ ID NO:82-84 and any of the CTGF CDR sets as set forth in SEQ ID NOs: 233-286 can be used in place of SEQ ID NOs: 82-84); SEQ ID NOs: 2-4 and SEQ ID NOs: 86-88, SEQ ID NOs: 6-8 and SEQ ID NOs: 86-88, SEQ ID NOs: 10-12 and SEQ ID NOs: 86-88, SEQ ID NOs: 14-16 and SEQ ID NOs: 86-88, SEQ ID NOs: 18-20 and SEQ ID NOs: 86-88, SEQ ID NOs: 22-24 and SEQ ID Nos: 86-88, SEQ ID NOs: 26-28 and SEQ ID NOs: 86-88, SEQ ID NOs: 30-32 and SEQ ID NOs: 86-88, SEQ ID NOs: 34-36 and SEQ ID NOs: 86-88, SEQ ID NOs: 38-40 and SEQ ID NOs: 86-88, SEQ ID NOs: 42-44 and SEQ ID NOs: 86-88, SEQ ID NOs: 46-48 and SEQ ID NOs: 86-88, SEQ ID NOs: 50-52 and SEQ ID NOs: 86-88, SEQ ID NOs: 54-56 and SEQ ID NOs: 86-88, SEQ ID NOs: 58-60 and SEQ ID NOs: 86-88, SEQ ID NOs: 62-64 and SEQ ID NOs: 86-88; SEQ ID NOs: 66-68 and SEQ ID NOs: 86-88 (furthermore any of the sIL-6R CDR sets shown in SEQ ID NOs: 138-194 can similarly be combined with SEQ ID NO: 86-88 and any of the CTGF CDR sets as set forth in SEQ ID NOs: 233-286 can be used in place of SEQ ID NOs: 86-88); SEQ ID NOs: 2-4 and SEQ ID NOs: 90-92, SEQ ID NOs: 6-8 and SEQ ID NOs: 90-92, SEQ ID NOs: 10-12 and SEQ ID NOs: 90-92, SEQ ID NOs: 14-16 and SEQ ID NOs: 90-92, SEQ ID NOs: 18-20 and SEQ ID NOs: 90-92, SEQ ID NOs: 22-24 and SEQ ID NOs: 90-92, SEQ ID NOs: 26-28 and SEQ ID NOs: 90-92, SEQ ID NOs: 30-32 and SEQ ID NOs: 90-92, SEQ ID NOs: 34-36 and SEQ ID NOs: 90-92, SEQ ID NOs: 38-40 and SEQ ID NOs: 90-92, SEQ ID NOs: 42-44 and SEQ ID NOs: 90-92, SEQ ID NOs: 46-48 and SEQ ID NOs: 90-92, SEQ ID NOs: 50-52 and SEQ ID NOs: 90-92, SEQ ID NOs: 54-56 and SEQ ID NOs: 90-92, SEQ ID NOs: 58-60 and SEQ ID NOs: 90-92, SEQ ID NOs: 62-64 and SEQ ID NOs: 90-92 or SEQ ID NOs: 66-68 and SEQ ID NOs: 90-92 (furthermore any of the sIL-6R CDR sets shown in SEQ ID NOs: 138-194 can similarly be combined with SEQ ID NO: 90-92 and any of the CTGF CDR sets as set forth in SEQ ID NOs: 233-286 can be used in place of SEQ ID NOs: 90-92); or
(ii) a multispecific binding protein that selectively and/or specifically binds to human sIL-6R and to human CTGF as described herein, for example including the amino acid sequence of: SEQ ID NO: 1 and SEQ ID NO:69, SEQ ID NO:5 and SEQ ID NO:69, SEQ ID NO:9 and SEQ ID NO: 69, SEQ ID NO: 13 and SEQ ID NO:69, SEQ ID NO: 17 and SEQ ID NO:69, SEQ ID NO:21 and SEQ ID NO:69, SEQ ID NO:25 and SEQ ID NO:69, SEQ ID NO:29 and SEQ ID NO:69, SEQ ID NO: 33 and SEQ ID NO:69, SEQ ID NO:37 and SEQ ID NO:69, SEQ ID NO:41 and SEQ ID NO: 69, SEQ ID NO:45 and SEQ ID NO:69, SEQ ID NO:49 and SEQ ID NO:69, SEQ ID NO:53 and SEQ ID NO:69, SEQ ID NO:57 and SEQ ID NO:69, SEQ ID NO:61 and SEQ ID NO:69, SEQ ID NO: 65 and SEQ ID NO:69 (furthermore any of the sIL-6R VHHs shown in SEQ ID NOs: 119-137 can similarly be combined with SEQ ID NO:69 and any of SEQ ID NO:214-232 can be used in place of SEQ ID NO:69); SEQ ID NO: 1 and SEQ ID NO:73, SEQ ID NO:5 and SEQ ID NO:73, SEQ ID NO:9 and SEQ ID NO:73, SEQ ID NO: 13 and SEQ ID NO:73, SEQ ID NO: 17 and SEQ ID NO: 73, SEQ ID NO:21 and SEQ ID NO:73, SEQ ID NO:25 and SEQ ID NO:73, SEQ ID NO:29 and SEQ ID NO:73, SEQ ID NO:33 and SEQ ID NO:73, SEQ ID NO:37 and SEQ ID NO:73, SEQ ID NO: 41 and SEQ ID NO:73, SEQ ID NO:45 and SEQ ID NO:73, SEQ ID NO:49 and SEQ ID NO: 73, SEQ ID NO:53 and SEQ ID NO:73, SEQ ID NO:57 and SEQ ID NO:73, SEQ ID NO:61 and SEQ ID NO:73, SEQ ID NO:65 and SEQ ID NO:73 (furthermore any of the sIL-6R VHHs shown in SEQ ID NOs: 119-137 can similarly be combined with SEQ ID NO:73 and any of SEQ ID NO: 214-232 can be used in place of SEQ ID NO:73); SEQ ID NO: 1 and SEQ ID NO:77, SEQ ID NO: 5 and SEQ ID NO:77, SEQ ID NO:9 and SEQ ID NO:77, SEQ ID NO: 13 and SEQ ID NO: 77, SEQ ID NO: 17 and SEQ ID NO:77, SEQ ID NO:21 and SEQ ID NO:77, SEQ ID NO:25 and SEQ ID NO:77, SEQ ID NO:29 and SEQ ID NO:77, SEQ ID NO:33 and SEQ ID NO:77, SEQ ID NO: 37 and SEQ ID NO:77, SEQ ID NO:41 and SEQ ID NO:77, SEQ ID NO:45 and SEQ ID NO: 77, SEQ ID NO:49 and SEQ ID NO:77, SEQ ID NO:53 and SEQ ID NO:77, SEQ ID NO:57 and SEQ ID NO:77, SEQ ID NO:61 and SEQ ID NO:77, SEQ ID NO:65 and SEQ ID NO:77 (furthermore any of the sIL-6R VHHs shown in SEQ ID NOs: 119-137 can similarly be combined with SEQ ID NO: 77 and any of SEQ ID NO:214-232 can be used in place of SEQ ID NO:77); SEQ ID NO: 1 and SEQ ID NO:81, SEQ ID NO:5 and SEQ ID NO:81, SEQ ID NO:9 and SEQ ID NO:81, SEQ ID NO: 13 and SEQ ID NO:81, SEQ ID NO: 17 and SEQ ID NO:81, SEQ ID NO:21 and SEQ ID NO: 81, SEQ ID NO:25 and SEQ ID NO:81, SEQ ID NO:29 and SEQ ID NO:81, SEQ ID NO:33 and SEQ ID NO: 81, SEQ ID NO:37 and SEQ ID NO:81, SEQ ID NO:41 and SEQ ID NO:81, SEQ ID NO: 45 and SEQ ID NO:81, SEQ ID NO:49 and SEQ ID NO:81, SEQ ID NO:53 and SEQ ID NO: 81, SEQ ID NO:57 and SEQ ID NO:81, SEQ ID NO:61 and SEQ ID NO:81, SEQ ID NO:65 and SEQ ID NO:81 (furthermore any of the sIL-6R VHHs shown in SEQ ID NOs: 119-137 can similarly be combined with SEQ ID NO:81 and any of SEQ ID NO:214-232 can be used in place of SEQ ID NO:81); SEQ ID NO: 1 and SEQ ID NO:85, SEQ ID NO:5 and SEQ ID NO:85, SEQ ID NO: 9 and SEQ ID NO: 85, SEQ ID NO: 13 and SEQ ID NO:85, SEQ ID NO: 17 and SEQ ID NO:85, SEQ ID NO:21 and SEQ ID NO:85, SEQ ID NO:25 and SEQ ID NO:85, SEQ ID NO:29 and SEQ ID NO: 85, SEQ ID NO:33 and SEQ ID NO: 85, SEQ ID NO:37 and SEQ ID NO:85, SEQ ID NO:41 and SEQ ID NO: 85, SEQ ID NO: 45 and SEQ ID NO:85, SEQ ID NO:49 and SEQ ID NO:85, SEQ ID NO: 53 and SEQ ID NO:85, SEQ ID NO:57 and SEQ ID NO:85, SEQ ID NO:61 and SEQ ID NO: 85, SEQ ID NO: 65 and SEQ ID NO: 85 (furthermore any of the sIL-6R VHHs shown in SEQ ID NOs: 119-137 can similarly be combined with SEQ ID NO:85 and any of SEQ ID NO:214-232 can be used in place of SEQ ID NO:85); SEQ ID NO: 1 and SEQ ID NO:89, SEQ ID NO:5 and SEQ ID NO:89, SEQ ID NO:9 and SEQ ID NO:89, SEQ ID NO: 13 and SEQ ID NO:89, SEQ ID NO: 17 and SEQ ID NO: 89, SEQ ID NO:21 and SEQ ID NO: 89, SEQ ID NO:25 and SEQ ID NO:89, SEQ ID NO:29 and SEQ ID NO:89, SEQ ID NO:33 and SEQ ID NO:89, SEQ ID NO:37 and SEQ ID NO: 89, SEQ ID NO: 41 and SEQ ID NO: 89, SEQ ID NO:45 and SEQ ID NO:89, SEQ ID NO:49 and SEQ ID NO: 89, SEQ ID NO:53 and SEQ ID NO:89, SEQ ID NO:57 and SEQ ID NO:89, SEQ ID NO: 61 and SEQ ID NO:89, or SEQ ID NO:65 and SEQ ID NO:89 (furthermore any of the sIL-6R VHHs shown in SEQ ID NOs: 119-137 can similarly be combined with SEQ ID NO:89 and any of SEQ ID NO:214-232 can be used in place of SEQ ID NO:89).

For example, the fusion protein can be:
(i) a fusion protein including a sIL-6R binding protein as described herein and an anti-transferrin receptor antibody, for example including CDRs as set forth in SEQ ID NOs: 2-4, SEQ ID NOs: 6-8, SEQ ID NOs: 10-12, SEQ ID NOs: 14-16, SEQ ID NOs: 18-20, SEQ ID NOs: 22-24, SEQ ID NOs: 26-28, SEQ ID NOs: 30-32, SEQ ID NOs: 34-36, SEQ ID NOs: 38-40, SEQ ID NOs: 42-44, SEQ ID NOs: 46-48, SEQ ID NOs: 50-52, SEQ ID NOs: 54-56, SEQ ID NOs: 58-60, SEQ ID NOs: 62-64, SEQ ID NOs: 66-68, or SEQ ID NO: 138-194 each further including an amino acid sequence encoding an anti-transferrin receptor antibody;
(ii) a fusion protein including a sIL-6R binding protein as described herein and an anti-transferrin receptor antibody, for example including the amino acid sequence of SEQ ID NO:1, SEQ ID NO:5, SEQ ID NO:9, SEQ ID NO: 13, SEQ ID NO: 17, SEQ ID NO:21, SEQ ID NO:25, SEQ ID NO:29, SEQ ID NO:33, SEQ ID NO:37, SEQ ID NO:41, SEQ ID NO:45, SEQ ID NO:49, SEQ ID NO:53, SEQ ID NO:57, SEQ ID NO:61, SEQ ID NO:65, or SEQ ID NOs: 119-137, each further including an amino acid sequence encoding an anti-transferrin receptor antibody;
(iii) a fusion protein including a CTGF binding protein as described herein and an anti-transferrin receptor antibody, for example including CDRs as set forth in NOs: 70-72, SEQ ID NOs: 74-76, SEQ ID NOs: 78-80, SEQ ID NOs: 82-84, SEQ ID NOS: 86-88, SEQ ID NOs: 90-92, SEQ ID NOs: 233-286, or SEQ ID NOs: 291-293 each further including an amino acid sequence encoding an anti-transferrin receptor antibody;
(iv) a fusion protein including a CTGF binding protein as described herein and an anti-transferrin receptor antibody, for example including the amino acid sequence of SEQ ID NO:73, SEQ ID NO:77, SEQ ID NO:81, SEQ ID NO:85, SEQ ID NO:89, or SEQ ID NOs: 214-232, each further including an amino acid sequence encoding an anti-transferrin receptor antibody;
(v) a fusion protein including a multispecific binding protein that selectively and/or specifically binds to human sIL-6R and to human CTGF as described herein and an anti-transferrin receptor antibody, for example including CDRs as set forth in SEQ ID NOs: 2-4 and SEQ ID NOs: 70-72, SEQ ID NOs: 6-8 and SEQ ID NOs: 70-72, SEQ ID NOs: 10-12 and SEQ ID NOs: 70-72, SEQ ID NOs: 14-16 and SEQ ID NOs: 70-72, SEQ ID NOs: 18-20 and SEQ ID NOs: 70-72, SEQ ID NOs: 22-24 and SEQ ID NOs: 70-72, SEQ ID NOs: 26-28 and SEQ ID NOs: 70-72, SEQ ID NOs: 30-32 and SEQ ID NOs: 70-72, SEQ ID NOs: 34-36 and SEQ ID NOs: 70-72, SEQ ID NOs: 38-40 and SEQ ID NOs: 70-72, SEQ ID NOs: 42-44 and SEQ ID NOs: 70-72, SEQ ID NOs: 46-48 and SEQ ID NOs: 70-72, SEQ ID NOs: 50-52 and SEQ ID NOs: 70-72, SEQ ID NOs: 54-56 and SEQ ID NOs: 70-72, SEQ ID NOs: 58-60 and SEQ ID NOs: 70-72, SEQ ID NOs: 62-64 and SEQ ID NOs: 70-72; SEQ ID NOs: 66-68 and SEQ ID NOs: 70-72 (furthermore any of the sIL-6R CDR sets shown in SEQ ID NOs: 138-194 can similarly be combined with SEQ ID NO:70-72 and any of the CTGF CDR sets as set forth in SEQ ID NOs: 233-286 can be used in place of SEQ ID NOs: 70-72); SEQ ID NOs: 2-4 and SEQ ID NOs: 74-76, SEQ ID NOs: 6-8 and SEQ ID NOs: 74-76, SEQ ID NOs: 10-12 and SEQ ID NOs: 74-76, SEQ ID NOs: 14-16 and SEQ ID NOs: 74-76, SEQ ID NOs: 18-20 and SEQ ID NOs: 74-76, SEQ ID NOs: 22-24 and SEQ ID NOs: 74-76, SEQ ID NOs: 26-28 and SEQ ID NOs: 74-76, SEQ ID NOs: 30-32 and SEQ ID NOs: 74-76, SEQ ID NOs: 34-36 and SEQ ID NOs: 74-76, SEQ ID NOs: 38-40 and SEQ ID NOs: 74-76, SEQ ID NOs: 42-44 and SEQ ID NOs: 74-76, SEQ ID NOs: 46-48 and SEQ ID NOs: 74-76, SEQ ID NOs: 50-52 and SEQ ID NOs: 74-76, SEQ ID NOs: 54-56 and SEQ ID NOs: 74-76, SEQ ID NOs: 58-60 and SEQ ID NOs: 74-76, SEQ ID NOs: 62-64 and SEQ ID NOs: 74-76; SEQ ID NOs: 66-68 and SEQ ID NOs: 74-76 (furthermore any of the sIL-6R CDR sets shown in SEQ ID NOs: 138-194 can similarly be combined with SEQ ID NO: 74-76 and any of the CTGF CDR sets as set forth in SEQ ID NOs: 233-286 can be used in place of SEQ ID NOs: 74-76); SEQ ID NOs: 2-4 and SEQ ID NOs: 78-80, SEQ ID NOs: 6-8 and SEQ ID NOs: 78-80, SEQ ID NOs: 10-12 and SEQ ID NOs: 78-80, SEQ ID NOs: 14-16 and SEQ ID NOs: 78-80, SEQ ID NOs: 18-20 and SEQ ID NOs: 78-80, SEQ ID NOs: 22-24 and SEQ ID NOs: 78-80, SEQ ID NOs: 26-28 and SEQ ID NOs: 78-80, SEQ ID NOs: 30-32 and SEQ ID NOs: 78-80, SEQ ID NOs: 34-36 and SEQ ID NOs: 78-80, SEQ ID NOs: 38-40 and SEQ ID NOs: 78-80, SEQ ID NOs: 42-44 and SEQ ID NOs: 78-80, SEQ ID NOs: 46-48 and SEQ ID NOs: 78-80, SEQ ID NOs: 50-52 and SEQ ID NOs: 78-80, SEQ ID NOs: 54-56 and SEQ ID NOs: 78-80, SEQ ID NOs: 58-60 and SEQ ID NOs: 78-80, SEQ ID NOs: 62-64 and SEQ ID NOs: 78-80; SEQ ID NOs: 66-68 and SEQ ID NOs: 78-80 (furthermore any of the sIL-6R CDR sets shown in SEQ ID NOs: 138-194 can similarly be combined with SEQ ID NO:78-80 and any of the CTGF CDR sets as set forth in SEQ ID NOs: 233-286 can be used in place of SEQ ID NOs: 78-80; SEQ ID NOs: 2-4 and SEQ ID NOs: 82-84, SEQ ID NOs: 6-8 and SEQ ID NOs: 82-84, SEQ ID NOs: 10-12 and SEQ ID NOs: 82-84, SEQ ID NOs: 14-16 and SEQ ID NOs: 82-84, SEQ ID NOs: 18-20 and SEQ ID NOs: 82-84, SEQ ID NOS: 22-24 and SEQ ID NOs: 82-84, SEQ ID NOs: 26-28 and SEQ ID NOs: 82-84, SEQ ID NOs: 30-32 and SEQ ID NOs: 82-84, SEQ ID NOs: 34-36 and SEQ ID NOs: 82-84, SEQ ID NOs: 38-40 and SEQ ID NOs: 82-84, SEQ ID NOs: 42-44 and SEQ ID NOs: 82-84, SEQ ID NOs: 46-48 and SEQ ID NOs: 82-84, SEQ ID NOs: 50-52 and SEQ ID NOs: 82-84, SEQ ID NOs: 54-56 and SEQ ID NOs: 82-84, SEQ ID NOs: 58-60 and SEQ ID NOs: 82-84, SEQ ID NOs: 62-64 and SEQ ID NOs: 82-84; SEQ ID NOs: 66-68 and SEQ ID NOs: 82-84 (furthermore any of the sIL-6R CDR sets shown in SEQ ID NOs: 138-194 can similarly be combined with SEQ ID NO:82-84 and any of the CTGF CDR sets as set forth in SEQ ID NOs: 233-286 can be used in place of SEQ ID NOs: 82-84); SEQ ID NOS: 2-4 and SEQ ID NOs: 86-88, SEQ ID NOs: 6-8 and SEQ ID NOs: 86-88, SEQ ID NOs: 10-12 and SEQ ID NOs: 86-88, SEQ ID NOs: 14-16 and SEQ ID NOs: 86-88, SEQ ID NOs: 18-20 and SEQ ID NOs: 86-88, SEQ ID NOs: 22-24 and SEQ ID NOs: 86-88, SEQ ID NOs: 26-28 and SEQ ID NOs: 86-88, SEQ ID NOs: 30-32 and SEQ ID NOs: 86-88, SEQ ID NOs: 34-36 and SEQ ID NOs: 86-88, SEQ ID NOs: 38-40 and SEQ ID NOs: 86-88, SEQ ID NOs: 42-44 and SEQ ID NOs: 86-88, SEQ ID NOs: 46-48 and SEQ ID NOs: 86-88, SEQ ID NOs: 50-52 and SEQ ID NOs: 86-88, SEQ ID NOs: 54-56 and SEQ ID NOs: 86-88, SEQ ID NOs: 58-60 and SEQ ID NOs: 86-88, SEQ ID NOs: 62-64 and SEQ ID NOs: 86-88; SEQ ID NOs: 66-68 and SEQ ID NOs: 86-88 (furthermore any of the sIL-6R CDR sets shown in SEQ ID NOs: 138-194 can similarly be combined with SEQ ID NO:86-88 and any of the CTGF CDR sets as set forth in SEQ ID NOs: 233-286 can be used in place of SEQ ID NOs: 86-88); SEQ ID NOs: 2-4 and SEQ ID NOs: 90-92, SEQ ID NOs: 6-8 and SEQ ID NOs: 90-92, SEQ ID NOs: 10-12 and SEQ ID NOs: 90-92, SEQ ID NOs: 14-16 and SEQ ID NOs: 90-92, SEQ ID NOs: 18-20 and SEQ ID NOs: 90-92, SEQ ID NOs: 22-24 and SEQ ID NOs: 90-92, SEQ ID NOs: 26-28 and SEQ ID NOs: 90-92, SEQ ID NOs: 30-32 and SEQ ID NOs: 90-92, SEQ ID NOs: 34-36 and SEQ ID NOs: 90-92, SEQ ID NOs: 38-40 and SEQ ID NOs: 90-92, SEQ ID NOs: 42-44 and SEQ ID NOs: 90-92, SEQ ID NOs: 46-48 and SEQ ID NOs: 90-92, SEQ ID NOs: 50-52 and SEQ ID NOs: 90-92, SEQ ID NOs: 54-56 and SEQ ID NOs: 90-92, SEQ ID NOs: 58-60 and SEQ ID NOs: 90-92, SEQ ID NOs: 62-64 and SEQ ID NOs: 90-92 or SEQ ID NOs: 66-68 and SEQ ID NOs: 90-92 (furthermore any of the sIL-6R CDR sets shown in SEQ ID NOs: 138-194 can similarly be combined with SEQ ID NO: 90-92 and any of the CTGF CDR sets as set forth in SEQ ID NOs: 233-286 can be used in place of SEQ ID NOs: 90-92), each further including an amino acid sequence encoding an anti-transferrin receptor antibody; or
(vi) a fusion protein including a multispecific binding protein that selectively and/or specifically binds to human sIL-6R and to human CTGF and an anti-transferrin receptor antibody as described herein, for example including the amino acid sequence of: SEQ ID NO: 1 and SEQ ID NO: 69, SEQ ID NO:5 and SEQ ID NO:69, SEQ ID NO:9 and SEQ ID NO:69, SEQ ID NO: 13 and SEQ ID NO:69, SEQ ID NO: 17 and SEQ ID NO:69, SEQ ID NO:21 and SEQ ID NO:69, SEQ ID NO: 25 and SEQ ID NO:69, SEQ ID NO:29 and SEQ ID NO:69, SEQ ID NO:33 and SEQ ID NO: 69, SEQ ID NO:37 and SEQ ID NO:69, SEQ ID NO:41 and SEQ ID NO:69, SEQ ID NO:45 and SEQ ID NO: 69, SEQ ID NO: 49 and SEQ ID NO:69, SEQ ID NO:53 and SEQ ID NO:69, SEQ ID NO: 57 and SEQ ID NO:69, SEQ ID NO:61 and SEQ ID NO:69, SEQ ID NO:65 and SEQ ID NO: 69 (furthermore any of the sIL-6R VHHs shown in SEQ ID NOs: 119-137 can similarly be combined with SEQ ID NO:69 and any of SEQ ID NO:214-232 can be used in place of SEQ ID NO: 69); SEQ ID NO: 1 and SEQ ID NO:73, SEQ ID NO:5 and SEQ ID NO:73, SEQ ID NO:9 and SEQ ID NO:73, SEQ ID NO: 13 and SEQ ID NO:73, SEQ ID NO: 17 and SEQ ID NO:73, SEQ ID NO: 21 and SEQ ID NO:73, SEQ ID NO:25 and SEQ ID NO:73, SEQ ID NO:29 and SEQ ID NO:73, SEQ ID NO:33 and SEQ ID NO:73, SEQ ID NO:37 and SEQ ID NO:73, SEQ ID NO:41 and SEQ ID NO: 73, SEQ ID NO:45 and SEQ ID NO:73, SEQ ID NO:49 and SEQ ID NO:73, SEQ ID NO:53 and SEQ ID NO:73, SEQ ID NO:57 and SEQ ID NO:73, SEQ ID NO:61 and SEQ ID NO:73, SEQ ID NO: 65 and SEQ ID NO:73 (furthermore any of the sIL-6R VHHs shown in SEQ ID NOs: 119-137 can similarly be combined with SEQ ID NO:73 and any of SEQ ID NO:214-232 can be used in place of SEQ ID NO:73); SEQ ID NO:1 and SEQ ID NO:77, SEQ ID NO:5 and SEQ ID NO:77, SEQ ID NO:9 and SEQ ID NO:77, SEQ ID NO: 13 and SEQ ID NO:77, SEQ ID NO: 17 and SEQ ID NO: 77, SEQ ID NO:21 and SEQ ID NO:77, SEQ ID NO:25 and SEQ ID NO:77, SEQ ID NO:29 and SEQ ID NO:77, SEQ ID NO:33 and SEQ ID NO:77, SEQ ID NO:37 and SEQ ID NO:77, SEQ ID NO: 41 and SEQ ID NO:77, SEQ ID NO:45 and SEQ ID NO:77, SEQ ID NO:49 and SEQ ID NO: 77, SEQ ID NO:53 and SEQ ID NO:77, SEQ ID NO:57 and SEQ ID NO:77, SEQ ID NO:61 and SEQ ID NO:77, SEQ ID NO:65 and SEQ ID NO:77 (furthermore any of the sIL-6R VHHs shown in SEQ ID NOs: 119-137 can similarly be combined with SEQ ID NO:77 and any of SEQ ID NO: 214-232 can be used in place of SEQ ID NO:77); SEQ ID NO: 1 and SEQ ID NO:81, SEQ ID NO: 5 and SEQ ID NO:81, SEQ ID NO:9 and SEQ ID NO:81, SEQ ID NO: 13 and SEQ ID NO: 81, SEQ ID NO: 17 and SEQ ID NO:81, SEQ ID NO:21 and SEQ ID NO:81, SEQ ID NO:25 and SEQ ID NO:81, SEQ ID NO:29 and SEQ ID NO:81, SEQ ID NO:33 and SEQ ID NO:81, SEQ ID NO: 37 and SEQ ID NO:81, SEQ ID NO:41 and SEQ ID NO:81, SEQ ID NO:45 and SEQ ID NO: 81, SEQ ID NO:49 and SEQ ID NO:81, SEQ ID NO:53 and SEQ ID NO:81, SEQ ID NO:57 and SEQ ID NO:81, SEQ ID NO:61 and SEQ ID NO:81, SEQ ID NO:65 and SEQ ID NO:81 (furthermore any of the sIL-6R VHHs shown in SEQ ID NOs: 119-137 can similarly be combined with SEQ ID NO: 81 and any of SEQ ID NO:214-232 can be used in place of SEQ ID NO:81); SEQ ID NO: 1 and SEQ ID NO:85, SEQ ID NO:5 and SEQ ID NO:85, SEQ ID NO:9 and SEQ ID NO:85, SEQ ID NO: 13 and SEQ ID NO:85, SEQ ID NO: 17 and SEQ ID NO:85, SEQ ID NO:21 and SEQ ID NO: 85, SEQ ID NO:25 and SEQ ID NO:85, SEQ ID NO:29 and SEQ ID NO:85, SEQ ID NO:33 and SEQ ID NO:85, SEQ ID NO:37 and SEQ ID NO:85, SEQ ID NO:41 and SEQ ID NO:85, SEQ ID NO: 45 and SEQ ID NO:85, SEQ ID NO:49 and SEQ ID NO:85, SEQ ID NO:53 and SEQ ID NO: 85, SEQ ID NO:57 and SEQ ID NO:85, SEQ ID NO:61 and SEQ ID NO:85, SEQ ID NO:65 and SEQ ID NO:85 (furthermore any of the sIL-6R VHHs shown in SEQ ID NOs: 119-137 can similarly be combined with SEQ ID NO:85 and any of SEQ ID NO:214-232 can be used in place of SEQ ID NO:85); SEQ ID NO: 1 and SEQ ID NO:89, SEQ ID NO:5 and SEQ ID NO:89, SEQ ID NO: 9 and SEQ ID NO: 89, SEQ ID NO: 13 and SEQ ID NO:89, SEQ ID NO: 17 and SEQ ID NO:89, SEQ ID NO:21 and SEQ ID NO:89, SEQ ID NO:25 and SEQ ID NO:89, SEQ ID NO:29 and SEQ ID NO: 89, SEQ ID NO:33 and SEQ ID NO: 89, SEQ ID NO:37 and SEQ ID NO:89, SEQ ID NO:41 and SEQ ID NO: 89, SEQ ID NO:45 and SEQ ID NO:89, SEQ ID NO:49 and SEQ ID NO:89, SEQ ID NO: 53 and SEQ ID NO:89, SEQ ID NO:57 and SEQ ID NO:89, SEQ ID NO:61 and SEQ ID NO: 89, or SEQ ID NO: 65 and SEQ ID NO: 89 (furthermore any of the sIL-6R VHHs shown in SEQ ID NOs: 119-137 can similarly be combined with SEQ ID NO:89 and any of SEQ ID NO:214-232 can be used in place of SEQ ID NO:89), each further including an amino acid sequence encoding an anti-transferrin receptor antibody.

The pharmaceutical composition can be a composition including any of the binding protein, fusion proteins, and multispecific binding protein described above, further including a pharmaceutically acceptable carrier.

The neurological disease or condition can be an inflammatory neurological disease or condition. The neurological disease or condition can be ALS, Parkinson disease, multiple sclerosis, Alzheimer's disease, or Huntington's disease.

ALS, also known as motor neuron disease (MND) or Lou Gehrig's disease, is a neurodegenerative disease that results in the progressive loss of motor neurons that control voluntary muscles. ALS is the most common type of motor neuron disease. Early symptoms of ALS include stiff muscles, muscle twitches, and gradual increasing weakness and muscle wasting. Limb-onset ALS begins with weakness in the arms or legs, while bulbar-onset ALS begins with difficulty speaking or swallowing. Half of the people with ALS develop at least mild difficulties with thinking and behavior, and about 15% develop frontotemporal dementia. Most people experience pain. The affected muscles are responsible for chewing food, speaking, and walking. Motor neuron loss continues until the ability to eat, speak, move, and finally the ability to breathe is lost. ALS eventually causes paralysis and early death, usually from respiratory failure. Most cases of ALS (about 90% to 95%) have no known cause and are known as sporadic ALS. However, both genetic and environmental factors are believed to be involved. The remaining 5% to 10% of cases have a genetic cause linked to a history of the disease in the family, and these are known as familial ALS. About half of these genetic cases are due to one of two specific genes. ALS and frontotemporal dementia (FTD) are considered to be part of a common disease spectrum (ALS-FTD) because of genetic, clinical, and pathological similarities. The underlying mechanism involves damage to both upper and lower motor neurons; in ALS-FTD, neurons in the frontal and temporal lobes of the brain die as well. The diagnosis is based on a person's signs and symptoms, with testing done to rule out other potential causes. The defining feature of ALS is the death of both upper motor neurons (located in the motor cortex of the brain) and lower motor neurons (located in the brainstem and spinal cord). In ALS with frontotemporal dementia, neurons throughout the frontal and temporal lobes of the brain die as well. The pathological hallmark of ALS is the presence of inclusion bodies (abnormal aggregations of protein) known as Bunina bodies in the cytoplasm of motor neurons. In about 97% of people with ALS, the main component of the inclusion bodies is TDP-43 protein; however, in those with SOD1 or FUS mutations, the main component of the inclusion bodies is SOD1 protein or FUS protein, respectively. The gross pathology of ALS, which are features of the disease that can be seen with the naked eye, include skeletal muscle atrophy, motor cortex atrophy, sclerosis of the corticospinal and corticobulbar tracts, thinning of the hypoglossal nerves (which control the tongue), and thinning of the anterior roots of the spinal cord. Aside from the death of motor neurons, two other characteristics common to most ALS variants are focal initial pathology, meaning that symptoms start in a single spinal cord region, and progressive continuous spread, meaning that symptoms spread to additional regions over time. Prion-like propagation of misfolded proteins from cell to cell may explain why ALS starts in one area and spreads to others. The glymphatic system may also be involved in the pathogenesis of ALS.

ALS is an incurable, fatal neurological condition that has few effective treatment options. The disease is marked by selective vulnerability and progressive loss of discrete neuronal populations, with significant number of non-neuronal cells contributing to neuronal loss. Inflammation, characterized by the appearance of ‘reactive’ non-neuronal cells (astrocytes, microglia, macrophages, T-lymphocytes), initiated by epigenetic alterations in innate immunity, is part of the disease progression. The pathogenicity of these astroglial cells resides in their proinflammatory and profibrotic properties. Early in the disease changes in innate immunity leads to increased production and release of significant levels of cytokines. Available treatment modalities for ALS have focused on: 1. replacement of astroglial cells that target the inflammation-mediated neurodegeneration, and 2. Quenching of free-radicals using small molecule drugs or intrabodies (intracellular antibodies) to inhibit enzymes that generate free radicals.

Parkinson's disease (PD), or simply Parkinson's, is a long-term degenerative disorder of the central nervous system that mainly affects the motor system. The symptoms usually emerge slowly, and as the disease worsens, non-motor symptoms become more common. The most obvious early symptoms are tremor, rigidity, slowness of movement, and difficulty with walking. Cognitive and behavioral problems may also occur with depression, anxiety, and apathy occurring in many people with PD. Parkinson's disease dementia becomes common in the advanced stages of the disease. Those with Parkinson's can also have problems with their sleep and sensory systems. The motor symptoms of the disease result from the death of cells in the substantia nigra, a region of the midbrain, leading to a dopamine deficit. The cause of this cell death is poorly understood but involves the build-up of misfolded proteins into Lewy bodies in the neurons. Collectively, the main motor symptoms are also known as parkinsonism or a parkinsonian syndrome. No cure for PD is known; treatment aims to reduce the effects of the symptoms. Initial treatment is typically with the medications levodopa (L-DOPA), MAO-B inhibitors, or dopamine agonists. As the disease progresses, these medications become less effective, while at the same time producing a side effect marked by involuntary muscle movements. At that time, medications may be used in combination and doses may be increased. Diet and certain forms of rehabilitation have shown some effectiveness at improving symptoms. Surgery to place microelectrodes for deep brain stimulation has been used to reduce motor symptoms in severe cases where drugs are ineffective. The main pathological characteristics of PD are cell death in the brain's basal ganglia (affecting up to 70% of the dopamine-secreting neurons in the substantia nigra pars compacta by the end of life). In Parkinson's disease, alpha-synuclein becomes misfolded and clump together with other alpha-synuclein. Cells are unable to remove these clumps, and the alpha-synuclein becomes cytotoxic, damaging the cells. These clumps can be seen in neurons under a microscope and are called Lewy bodies. Loss of neurons is accompanied by the death of astrocytes (star-shaped glial cells) and a significant increase in the number of microglia (another type of glial cell) in the substantia nigra. Braak staging is a way to explain the progression of the parts of the brain affected by PD. According to this staging, PD starts in the medulla and the olfactory bulb before moving to the substantia nigra pars compacta and the rest of the midbrain/basal forebrain. Movement symptom onset is associated when the disease begins to affect the substantia nigra pars compacta. Brain cells could be lost by several proposed mechanisms. One mechanism consists of an abnormal accumulation of the protein alpha-synuclein bound to ubiquitin in the damaged cells. This insoluble protein accumulates inside neurons forming inclusions called Lewy bodies. According to the Braak staging, a classification of the disease based on pathological findings proposed by Heiko Braak, Lewy bodies first appear in the olfactory bulb, medulla oblongata, and pontine tegmentum; individuals at this stage may be asymptomatic or may have early nonmotor symptoms (such as loss of sense of smell, or some sleep or automatic dysfunction). As the disease progresses, Lewy bodies develop in the substantia nigra, areas of the midbrain and basal forebrain, and finally, the neocortex. These brain sites are the main places of neuronal degeneration in PD, but Lewy bodies may not cause cell death and they may be protective (with the abnormal protein sequestered or walled off).

Multiple sclerosis (MS), also known as encephalomyelitis disseminata, is the most common demyelinating disease, in which the insulating covers of nerve cells in the brain and spinal cord are damaged. This damage disrupts the ability of parts of the nervous system to transmit signals, resulting in a range of signs and symptoms, including physical, mental, and sometimes psychiatric problems. Specific symptoms can include double vision, blindness in one eye, muscle weakness, and trouble with sensation or coordination. MS takes several forms, with new symptoms either occurring in isolated attacks (relapsing forms) or building up over time (progressive forms). Between attacks, symptoms may disappear completely, although permanent neurological problems often remain, especially as the disease advances. While the cause is unclear, the underlying mechanism is thought to be either destruction by the immune system or failure of the myelin-producing cells. Proposed causes for this include genetics and environmental factors, such as viral infections. MS is usually diagnosed based on the presenting signs and symptoms and the results of supporting medical tests. Multiple sclerosis is the most common immune-mediated disorder affecting the central nervous system. In 2015, about 2.3 million people were affected globally, with rates varying widely in different regions and among different populations. In that year, about 18,900 people died from MS, up from 12,000 in 1990. The disease usually begins between the ages of twenty and fifty and is twice as common in women as in men. MS was first described in 1868 by French neurologist Jean-Martin Charcot. The name multiple sclerosis refers to the numerous glial scars (or sclerae-essentially plaques or lesions) that develop on the white matter of the brain and spinal cord. As of 2009 a number of new treatments and diagnostic methods are under development. The three main characteristics of MS are the formation of lesions in the central nervous system (also called plaques), inflammation and the destruction of myelin sheaths of neurons. These features interact in a complex and not yet fully understood manner to produce the breakdown of nerve tissue and in turn the signs and symptoms of the disease. Cholesterol crystals are believed both to impair myelin repair and to aggravate inflammation. MS is believed to be an immune-mediated disorder that develops from an interaction of the individual's genetics and as yet unidentified environmental causes. Damage is believed to be caused, at least in part, by attack on the nervous system by a person's own immune system.

Alzheimer's disease (AD) is a neurodegenerative disease that usually starts slowly and progressively worsens. It is the cause of 60-70% of cases of dementia. The most common early symptom is difficulty in remembering recent events. As the disease advances, symptoms can include problems with language, disorientation (including easily getting lost), mood swings, loss of motivation, self-neglect, and behavioral issues. Gradually, bodily functions are lost, ultimately leading to death. Although the speed of progression can vary, the typical life expectancy following diagnosis is three to nine years. The disease process is largely associated with amyloid plaques, neurofibrillary tangles, and loss of neuronal connections in the brain. A probable diagnosis is based on the history of the illness and cognitive testing with medical imaging and blood tests to rule out other possible causes. Initial symptoms are often mistaken for normal aging. Examination of brain tissue is needed for a definite diagnosis, but this can only take place after death. Alzheimer's disease is characterized by loss of neurons and synapses in the cerebral cortex and certain subcortical regions. This loss results in gross atrophy of the affected regions, including degeneration in the temporal lobe and parietal lobe, and parts of the frontal cortex and cingulate gyrus. Degeneration is also present in brainstem nuclei particularly the locus coeruleus in the pons. Studies using MRI and PET have documented reductions in the size of specific brain regions in people with Alzheimer's disease as they progressed from mild cognitive impairment to Alzheimer's disease, and in comparison, with similar images from healthy older adults. Both AB plaques and neurofibrillary tangles are clearly visible by microscopy in brains of those with Alzheimer's disease, especially in the hippocampus. However, Alzheimer's disease may occur without neurofibrillary tangles in the neocortex. Plaques are dense, mostly insoluble deposits of beta-amyloid peptide and cellular material outside and around neurons. Tangles (neurofibrillary tangles) are aggregates of the microtubule-associated protein tau which has become hyperphosphorylated and accumulate inside the cells themselves. Although many older individuals develop some plaques and tangles as a consequence of aging, the brains of people with Alzheimer's disease have a greater number of them in specific brain regions such as the temporal lobe. Lewy bodies are not rare in the brains of people with Alzheimer's disease.

Huntington's disease (HD), also known as Huntington's chorea, is a neurodegenerative disease that is mostly inherited. The earliest symptoms are often subtle problems with mood or mental abilities. A general lack of coordination and an unsteady gait often follow. It is also a basal ganglia disease causing a hyperkinetic movement disorder known as chorea. As the disease advances, uncoordinated, involuntary body movements of chorea become more apparent.

Physical abilities gradually worsen until coordinated movement becomes difficult and the person is unable to talk. Mental abilities generally decline into dementia. Symptoms usually begin between 30 and 50 years of age but can start at any age. HD is typically inherited from an affected parent, who carries a mutation in the huntingtin gene (HTT). However, up to 10% of cases are due to a new mutation. The huntingtin gene provides the genetic information for huntingtin protein (Htt) Expansion of CAG repeats of cytosine-adenine-guanine (known as a trinucleotide repeat expansion) in the gene coding for the huntingtin protein results in an abnormal mutant protein (mHtt), which gradually damages brain cells through a number of possible mechanisms. Diagnosis is by genetic testing, which can be carried out at any time, regardless of whether or not symptoms are present. No cure for HD is known, and full-time care is required in the later stages. Treatments can relieve some symptoms, and in some, improve quality of life. The best evidence for treatment of the movement problems is with tetrabenazine. HD affects about 4 to 15 in 100,000 people of European descent. It is rare among Japanese, while the occurrence rate in Africa is unknown. The disease affects men and women equally. Huntingtin protein interacts with over 100 other proteins and appears to have multiple functions. The behavior of the mutated protein (mHtt) is not completely understood, but it is toxic to certain cell types, particularly brain cells. Early damage is most evident in the subcortical basal ganglia, initially in the striatum, but as the disease progresses, other areas of the brain are also affected, including regions of the cerebral cortex. Early symptoms are attributable to functions of the striatum and its cortical connections-namely control over movement, mood, and higher cognitive function. DNA methylation also appears to be changed in HD. Htt is expressed in all cells, with the highest concentrations found in the brain and testes, and moderate amounts in the liver, heart, and lungs. Its functions are unclear, but it does interact with proteins involved in transcription, cell signaling, and intracellular transporting. The toxic action of mHtt may manifest and produce the HD pathology through multiple cellular changes. In its mutant (polyglutamine expanded) form, the protein is more prone to cleavage that creates shorter fragments containing the polyglutamine expansion. These protein fragments have a propensity to undergo misfolding and aggregation, yielding fibrillar aggregates in which non-native polyglutamine β-strands from multiple proteins are bonded together by hydrogen bonds. These aggregates share the same fundamental cross-beta amyloid architecture seen in other protein deposition diseases. Over time, the aggregates accumulate to form inclusion bodies within cells, ultimately interfering with neuronal function.

Treating the neurological disease can comprise improving glial-neuronal communication and retarding neurodegenerative process.

Treating the neurological disease can comprise reducing cerebral inflammation, inhibiting can cerebral fibrosis and/or improving cerebral vascular function.

Reducing inflammation can comprise inhibiting reactive cells production of inflammatory cytokines and/or free radicals.

An inflammatory cytokine or proinflammatory cytokine is a type of signaling molecule (a cytokine) that is secreted from immune cells like helper T cells (Th) and macrophages, and certain other cell types that promote inflammation. They can include interleukin-1 (IL-1), IL-6, IL-12, and IL-18, tumor necrosis factor alpha (TNF-α), interferon gamma (IFNγ), and granulocyte-macrophage colony stimulating factor (GM-CSF) and play an important role in mediating the innate immune response. Inflammatory cytokines are predominantly produced by and involved in the upregulation of inflammatory reactions. Excessive chronic production of inflammatory cytokines can contribute to inflammatory diseases. Dysregulation has also been linked to depression and other neurological diseases. A balance between proinflammatory and anti-inflammatory cytokines is necessary to maintain health. Aging and exercise also play a role in the amount of inflammation from the release of proinflammatory cytokines. Therapies to treat inflammatory diseases include monoclonal antibodies that either neutralize inflammatory cytokines or their receptors.

Inflammatory cytokines can comprise IL-6 and monocyte chemoattractant protein-1 (MCP-1).

Reducing inflammation can comprise reducing IL-6 levels.

Reducing IL-6 levels can comprise suppressing pSTAT3 expression in healthy astroglial cells.

Inhibiting cerebral fibrosis can comprise inhibiting reactive cells production of serum amyloid. Reactive cells can comprise non neuronal cells selected from the group consisting of astrocytes, microglia, macrophages and T lymphocytes.

Improving cerebral vascular function can comprise inhibiting cytokine-induced vasoconstriction, inducing vasodilation and/or increasing cerebral blood flow.

Inhibiting cytokine-induced vasoconstriction can comprise inhibiting IL-6/AT1R mediated vasoconstriction of cerebral micro vessels.

An embodiment provides a method of treating a neurological disease or condition in a subject comprising administering to the subject a therapeutically effective amount of a fusion protein comprising a first binding protein that selectively and/or specifically binds to human sIL-6R linked to a second binding protein that selectively and/or specifically binds to human CTGF, thereby treating the neurological disease.

The first binding protein and the second binding protein can be VHH antibodies or nanobodies. The fusion protein can further comprise an anti-transferrin receptor binding protein.

The fusion protein can be any one of the fusion proteins including a first binding protein that selectively and/or specifically binds to human sIL-6R second binding protein that selectively and/or specifically binds to human CTGF, and optionally, an additional binding protein, such as an anti-transferrin receptor binding protein, as described herein.

For example, the fusion protein can include:
(i) a fusion protein including a sIL-6R binding protein as described herein and an anti-transferrin receptor antibody, for example including CDRs as set forth in SEQ ID NOs: 2-4, SEQ ID NOs: 6-8, SEQ ID NOs: 10-12, SEQ ID NOs: 14-16, SEQ ID NOs: 18-20, SEQ ID NOs: 22-24, SEQ ID NOs: 26-28, SEQ ID NOs: 30-32, SEQ ID NOs: 34-36, SEQ ID NOs: 38-40, SEQ ID NOs: 42-44, SEQ ID NOs: 46-48, SEQ ID NOs: 50-52, SEQ ID NOs: 54-56, SEQ ID NOs: 58-60, SEQ ID NOs: 62-64, SEQ ID NOs: 66-68, or SEQ ID NO: 138-194 each further including an amino acid sequence encoding an anti-transferrin receptor antibody;
(ii) a fusion protein including a sIL-6R binding protein as described herein and an anti-transferrin receptor antibody, for example including the amino acid sequence of SEQ ID NO:1, SEQ ID NO:5, SEQ ID NO:9, SEQ ID NO: 13, SEQ ID NO: 17, SEQ ID NO:21, SEQ ID NO:25, SEQ ID NO:29, SEQ ID NO:33, SEQ ID NO:37, SEQ ID NO:41, SEQ ID NO:45, SEQ ID NO:49, SEQ ID NO:53, SEQ ID NO:57, SEQ ID NO:61, SEQ ID NO:65, or SEQ ID NOs: 119-137, each further including an amino acid sequence encoding an anti-transferrin receptor antibody;
(iii) a fusion protein including a CTGF binding protein as described herein and an anti-transferrin receptor antibody, for example including CDRs as set forth in NOs: 70-72, SEQ ID NOs: 74-76, SEQ ID NOs: 78-80, SEQ ID NOs: 82-84, SEQ ID NOs: 86-88, SEQ ID NOs: 90-92, SEQ ID NOs: 233-286, or SEQ ID NOs: 291-293, each further including an amino acid sequence encoding an anti-transferrin receptor antibody;
(iv) a fusion protein including a CTGF binding protein as described herein and an anti-transferrin receptor antibody, for example including the amino acid sequence of SEQ ID NO:73, SEQ ID NO:77, SEQ ID NO:81, SEQ ID NO:85, SEQ ID NO:89, or SEQ ID NOs: 214-232, each further including an amino acid sequence encoding an anti-transferrin receptor antibody;
(v) a fusion protein including a multispecific binding protein that selectively and/or specifically binds to human sIL-6R and to human CTGF as described herein and an anti-transferrin receptor antibody, for example including CDRs as set forth in SEQ ID NOs: 2-4 and SEQ ID NOs: 70-72, SEQ ID NOs: 6-8 and SEQ ID NOs: 70-72, SEQ ID NOs: 10-12 and SEQ ID NOs: 70-72, SEQ ID NOs: 14-16 and SEQ ID NOs: 70-72, SEQ ID NOs: 18-20 and SEQ ID NOs: 70-72, SEQ ID NOs: 22-24 and SEQ ID NOs: 70-72, SEQ ID NOs: 26-28 and SEQ ID NOs: 70-72, SEQ ID NOs: 30-32 and SEQ ID NOs: 70-72, SEQ ID NOs: 34-36 and SEQ ID NOs: 70-72, SEQ ID NOs: 38-40 and SEQ ID NOs: 70-72, SEQ ID NOs: 42-44 and SEQ ID NOs: 70-72, SEQ ID NOs: 46-48 and SEQ ID NOs: 70-72, SEQ ID NOs: 50-52 and SEQ ID NOs: 70-72, SEQ ID NOs: 54-56 and SEQ ID NOs: 70-72, SEQ ID NOs: 58-60 and SEQ ID NOs: 70-72, SEQ ID NOs: 62-64 and SEQ ID NOs: 70-72; SEQ ID NOs: 66-68 and SEQ ID NOs: 70-72 (furthermore any of the sIL-6R CDR sets shown in SEQ ID NOs: 138-194 can similarly be combined with SEQ ID NO: 70-72 and any of the CTGF CDR sets as set forth in SEQ ID NOs: 233-286 can be used in place of SEQ ID NOs: 70-72); SEQ ID NOs: 2-4 and SEQ ID NOs: 74-76, SEQ ID NOs: 6-8 and SEQ ID NOs: 74-76, SEQ ID NOs: 10-12 and SEQ ID NOs: 74-76, SEQ ID NOs: 14-16 and SEQ ID NOs: 74-76, SEQ ID NOs: 18-20 and SEQ ID NOs: 74-76, SEQ ID NOs: 22-24 and SEQ ID NOs: 74-76, SEQ ID NOs: 26-28 and SEQ ID NOs: 74-76, SEQ ID NOs: 30-32 and SEQ ID NOs: 74-76, SEQ ID NOs: 34-36 and SEQ ID NOs: 74-76, SEQ ID NOs: 38-40 and SEQ ID NOs: 74-76, SEQ ID NOs: 42-44 and SEQ ID NOs: 74-76, SEQ ID NOs: 46-48 and SEQ ID NOs: 74-76, SEQ ID NOs: 50-52 and SEQ ID NOs: 74-76, SEQ ID NOs: 54-56 and SEQ ID NOs: 74-76, SEQ ID NOs: 58-60 and SEQ ID NOs: 74-76, SEQ ID NOs: 62-64 and SEQ ID NOs: 74-76; SEQ ID NOs: 66-68 and SEQ ID NOs: 74-76 (furthermore any of the sIL-6R CDR sets shown in SEQ ID NOs: 138-194 can similarly be combined with SEQ ID NO: 74-76 and any of the CTGF CDR sets as set forth in SEQ ID NOs: 233-286 can be used in place of SEQ ID NOs: 74-76); SEQ ID NOs: 2-4 and SEQ ID NOs: 78-80, SEQ ID NOs: 6-8 and SEQ ID NOs: 78-80, SEQ ID NOs: 10-12 and SEQ ID NOs: 78-80, SEQ ID NOs: 14-16 and SEQ ID NOs: 78-80, SEQ ID NOs: 18-20 and SEQ ID NOs: 78-80, SEQ ID NOs: 22-24 and SEQ ID NOs: 78-80, SEQ ID NOs: 26-28 and SEQ ID NOs: 78-80, SEQ ID NOs: 30-32 and SEQ ID NOs: 78-80, SEQ ID NOs: 34-36 and SEQ ID NOs: 78-80, SEQ ID NOs: 38-40 and SEQ ID NOs: 78-80, SEQ ID NOs: 42-44 and SEQ ID NOs: 78-80, SEQ ID NOs: 46-48 and SEQ ID NOs: 78-80, SEQ ID NOs: 50-52 and SEQ ID NOs: 78-80, SEQ ID NOs: 54-56 and SEQ ID NOs: 78-80, SEQ ID NOs: 58-60 and SEQ ID NOs: 78-80, SEQ ID NOs: 62-64 and SEQ ID NOs: 78-80; SEQ ID NOs: 66-68 and SEQ ID NOs: 78-80 (furthermore any of the sIL-6R CDR sets shown in SEQ ID NOs: 138-194 can similarly be combined with SEQ ID NO:78-80 and any of the CTGF CDR sets as set forth in SEQ ID NOs: 233-286 can be used in place of SEQ ID NOs: 78-80); SEQ ID NOs: 2-4 and SEQ ID NOs: 82-84, SEQ ID NOs: 6-8 and SEQ ID NOs: 82-84, SEQ ID NOs: 10-12 and SEQ ID NOs: 82-84, SEQ ID NOs: 14-16 and SEQ ID NOs: 82-84, SEQ ID NOs: 18-20 and SEQ ID NOs: 82-84, SEQ ID NOs: 22-24 and SEQ ID NOs: 82-84, SEQ ID NOs: 26-28 and SEQ ID NOs: 82-84, SEQ ID NOs: 30-32 and SEQ ID NOs: 82-84, SEQ ID NOs: 34-36 and SEQ ID NOs: 82-84, SEQ ID NOs: 38-40 and SEQ ID NOs: 82-84, SEQ ID NOs: 42-44 and SEQ ID NOs: 82-84, SEQ ID NOs: 46-48 and SEQ ID NOs: 82-84, SEQ ID NOs: 50-52 and SEQ ID NOs: 82-84, SEQ ID NOs: 54-56 and SEQ ID NOs: 82-84, SEQ ID NOs: 58-60 and SEQ ID NOs: 82-84, SEQ ID NOs: 62-64 and SEQ ID NOs: 82-84; SEQ ID NOs: 66-68 and SEQ ID NOs: 82-84 (furthermore any of the sIL-6R CDR sets shown in SEQ ID NOs: 138-194 can similarly be combined with SEQ ID NO:82-84 and any of the CTGF CDR sets as set forth in SEQ ID NOs: 233-286 can be used in place of SEQ ID NOs: 82-84); SEQ ID NOs: 2-4 and SEQ ID NOs: 86-88, SEQ ID NOs: 6-8 and SEQ ID NOs: 86-88, SEQ ID NOs: 10-12 and SEQ ID NOs: 86-88, SEQ ID NOs: 14-16 and SEQ ID NOs: 86-88, SEQ ID NOs: 18-20 and SEQ ID NOs: 86-88, SEQ ID NOs: 22-24 and SEQ ID NOs: 86-88, SEQ ID NOs: 26-28 and SEQ ID NOs: 86-88, SEQ ID NOs: 30-32 and SEQ ID NOs: 86-88, SEQ ID NOs: 34-36 and SEQ ID NOs: 86-88, SEQ ID NOs: 38-40 and SEQ ID NOs: 86-88, SEQ ID NOs: 42-44 and SEQ ID NOs: 86-88, SEQ ID NOs: 46-48 and SEQ ID NOs: 86-88, SEQ ID NOs: 50-52 and SEQ ID NOs: 86-88, SEQ ID NOs: 54-56 and SEQ ID NOs: 86-88, SEQ ID NOs: 58-60 and SEQ ID NOs: 86-88, SEQ ID NOs: 62-64 and SEQ ID NOs: 86-88; SEQ ID NOs: 66-68 and SEQ ID NOs: 86-88 (furthermore any of the sIL-6R CDR sets shown in SEQ ID NOs: 138-194 can similarly be combined with SEQ ID NO:86-88 and any of the CTGF CDR sets as set forth in SEQ ID NOs: 233-286 can be used in place of SEQ ID NOs: 86-88); SEQ ID NOs: 2-4 and SEQ ID NOs: 90-92, SEQ ID NOs: 6-8 and SEQ ID NOs: 90-92, SEQ ID NOs: 10-12 and SEQ ID NOs: 90-92, SEQ ID NOs: 14-16 and SEQ ID NOs: 90-92, SEQ ID NOs: 18-20 and SEQ ID NOs: 90-92, SEQ ID NOs: 22-24 and SEQ ID NOs: 90-92, SEQ ID NOs: 26-28 and SEQ ID NOs: 90-92, SEQ ID NOs: 30-32 and SEQ ID NOs: 90-92, SEQ ID NOs: 34-36 and SEQ ID NOs: 90-92, SEQ ID NOs: 38-40 and SEQ ID NOs: 90-92, SEQ ID NOs: 42-44 and SEQ ID NOs: 90-92, SEQ ID NOs: 46-48 and SEQ ID NOs: 90-92, SEQ ID NOs: 50-52 and SEQ ID NOs: 90-92, SEQ ID NOs: 54-56 and SEQ ID NOs: 90-92, SEQ ID NOs: 58-60 and SEQ ID NOs: 90-92, SEQ ID NOs: 62-64 and SEQ ID NOs: 90-92 or SEQ ID NOs: 66-68 and SEQ ID NOs: 90-92 (furthermore any of the sIL-6R CDR sets shown in SEQ ID NOs: 138-194 can similarly be combined with SEQ ID NO: 90-92 and any of the CTGF CDR sets as set forth in SEQ ID NOs: 233-286 can be used in place of SEQ ID NOs: 90-92), each further including an amino acid sequence encoding an anti-transferrin receptor antibody; or
(vi) a fusion protein including a multispecific binding protein that selectively and/or specifically binds to human sIL-6R and to human CTGF and an anti-transferrin receptor antibody as described herein, for example including the amino acid sequence of: SEQ ID NO: 1 and SEQ ID NO: 69, SEQ ID NO:5 and SEQ ID NO:69, SEQ ID NO:9 and SEQ ID NO:69, SEQ ID NO: 13 and SEQ ID NO:69, SEQ ID NO: 17 and SEQ ID NO:69, SEQ ID NO:21 and SEQ ID NO:69, SEQ ID NO: 25 and SEQ ID NO:69, SEQ ID NO:29 and SEQ ID NO:69, SEQ ID NO:33 and SEQ ID NO: 69, SEQ ID NO:37 and SEQ ID NO:69, SEQ ID NO:41 and SEQ ID NO:69, SEQ ID NO:45 and SEQ ID NO:69, SEQ ID NO:49 and SEQ ID NO:69, SEQ ID NO:53 and SEQ ID NO:69, SEQ ID NO: 57 and SEQ ID NO:69, SEQ ID NO:61 and SEQ ID NO:69, SEQ ID NO:65 and SEQ ID NO: 69 (furthermore any of the sIL-6R VHHs shown in SEQ ID NOs: 119-137 can similarly be combined with SEQ ID NO:69 and any of SEQ ID NO:214-232 can be used in place of SEQ ID NO: 69); SEQ ID NO: 1 and SEQ ID NO:73, SEQ ID NO:5 and SEQ ID NO:73, SEQ ID NO:9 and SEQ ID NO:73, SEQ ID NO: 13 and SEQ ID NO:73, SEQ ID NO: 17 and SEQ ID NO:73, SEQ ID NO: 21 and SEQ ID NO:73, SEQ ID NO:25 and SEQ ID NO:73, SEQ ID NO:29 and SEQ ID NO:73, SEQ ID NO:33 and SEQ ID NO:73, SEQ ID NO:37 and SEQ ID NO:73, SEQ ID NO:41 and SEQ ID NO: 73, SEQ ID NO:45 and SEQ ID NO:73, SEQ ID NO:49 and SEQ ID NO:73, SEQ ID NO:53 and SEQ ID NO:73, SEQ ID NO:57 and SEQ ID NO:73, SEQ ID NO:61 and SEQ ID NO:73, SEQ ID NO: 65 and SEQ ID NO:73 (furthermore any of the sIL-6R VHHs shown in SEQ ID NOs: 119-137 can similarly be combined with SEQ ID NO:73 and any of SEQ ID NO:214-232 can be used in place of SEQ ID NO:73); SEQ ID NO: 1 and SEQ ID NO:77, SEQ ID NO:5 and SEQ ID NO:77, SEQ ID NO:9 and SEQ ID NO:77, SEQ ID NO: 13 and SEQ ID NO:77, SEQ ID NO: 17 and SEQ ID NO: 77, SEQ ID NO:21 and SEQ ID NO:77, SEQ ID NO:25 and SEQ ID NO:77, SEQ ID NO:29 and SEQ ID NO:77, SEQ ID NO:33 and SEQ ID NO:77, SEQ ID NO:37 and SEQ ID NO:77, SEQ ID NO: 41 and SEQ ID NO:77, SEQ ID NO:45 and SEQ ID NO:77, SEQ ID NO:49 and SEQ ID NO: 77, SEQ ID NO:53 and SEQ ID NO:77, SEQ ID NO:57 and SEQ ID NO:77, SEQ ID NO:61 and SEQ ID NO:77, SEQ ID NO:65 and SEQ ID NO:77 (furthermore any of the sIL-6R VHHs shown in SEQ ID NOs: 119-137 can similarly be combined with SEQ ID NO:77 and any of SEQ ID NO: 214-232 can be used in place of SEQ ID NO:77); SEQ ID NO: 1 and SEQ ID NO:81, SEQ ID NO: 5 and SEQ ID NO:81, SEQ ID NO:9 and SEQ ID NO:81, SEQ ID NO: 13 and SEQ ID NO: 81, SEQ ID NO: 17 and SEQ ID NO:81, SEQ ID NO:21 and SEQ ID NO:81, SEQ ID NO:25 and SEQ ID NO:81, SEQ ID NO:29 and SEQ ID NO:81, SEQ ID NO:33 and SEQ ID NO:81, SEQ ID NO: 37 and SEQ ID NO:81, SEQ ID NO:41 and SEQ ID NO:81, SEQ ID NO:45 and SEQ ID NO: 81, SEQ ID NO:49 and SEQ ID NO:81, SEQ ID NO:53 and SEQ ID NO:81, SEQ ID NO:57 and SEQ ID NO:81, SEQ ID NO:61 and SEQ ID NO:81, SEQ ID NO:65 and SEQ ID NO:81 (furthermore any of the sIL-6R VHHs shown in SEQ ID NOs: 119-137 can similarly be combined with SEQ ID NO: 81 and any of SEQ ID NO:214-232 can be used in place of SEQ ID NO:81); SEQ ID NO: 1 and SEQ ID NO:85, SEQ ID NO:5 and SEQ ID NO:85, SEQ ID NO:9 and SEQ ID NO:85, SEQ ID NO: 13 and SEQ ID NO: 85, SEQ ID NO: 17 and SEQ ID NO:85, SEQ ID NO:21 and SEQ ID NO: 85, SEQ ID NO:25 and SEQ ID NO:85, SEQ ID NO:29 and SEQ ID NO:85, SEQ ID NO:33 and SEQ ID NO:85, SEQ ID NO:37 and SEQ ID NO:85, SEQ ID NO:41 and SEQ ID NO:85, SEQ ID NO: 45 and SEQ ID NO:85, SEQ ID NO:49 and SEQ ID NO:85, SEQ ID NO:53 and SEQ ID NO: 85, SEQ ID NO:57 and SEQ ID NO:85, SEQ ID NO:61 and SEQ ID NO:85, SEQ ID NO:65 and SEQ ID NO:85 (furthermore any of the sIL-6R VHHs shown in SEQ ID NOs: 119-137 can similarly be combined with SEQ ID NO:85 and any of SEQ ID NO:214-232 can be used in place of SEQ ID NO:85); SEQ ID NO: 1 and SEQ ID NO:89, SEQ ID NO:5 and SEQ ID NO:89, SEQ ID NO: 9 and SEQ ID NO: 89, SEQ ID NO: 13 and SEQ ID NO:89, SEQ ID NO: 17 and SEQ ID NO:89, SEQ ID NO:21 and SEQ ID NO:89, SEQ ID NO:25 and SEQ ID NO:89, SEQ ID NO:29 and SEQ ID NO: 89, SEQ ID NO:33 and SEQ ID NO: 89, SEQ ID NO:37 and SEQ ID NO:89, SEQ ID NO:41 and SEQ ID NO: 89, SEQ ID NO:45 and SEQ ID NO:89, SEQ ID NO:49 and SEQ ID NO:89, SEQ ID NO: 53 and SEQ ID NO:89, SEQ ID NO:57 and SEQ ID NO:89, SEQ ID NO:61 and SEQ ID NO: 89, or SEQ ID NO: 65 and SEQ ID NO: 89 (furthermore any of the sIL-6R VHHs shown in SEQ ID NOs: 119-137 can similarly be combined with SEQ ID NO:89 and any of SEQ ID NO:214-232 can be used in place of SEQ ID NO:89), each further including an amino acid sequence encoding an anti-transferrin receptor antibody.

The compositions and methods are more particularly described below, and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

The following are provided for exemplification purposes only and are not intended to limit the scope of the embodiments described in broad terms above.

### EXAMPLES

### Example 1: Immunogen

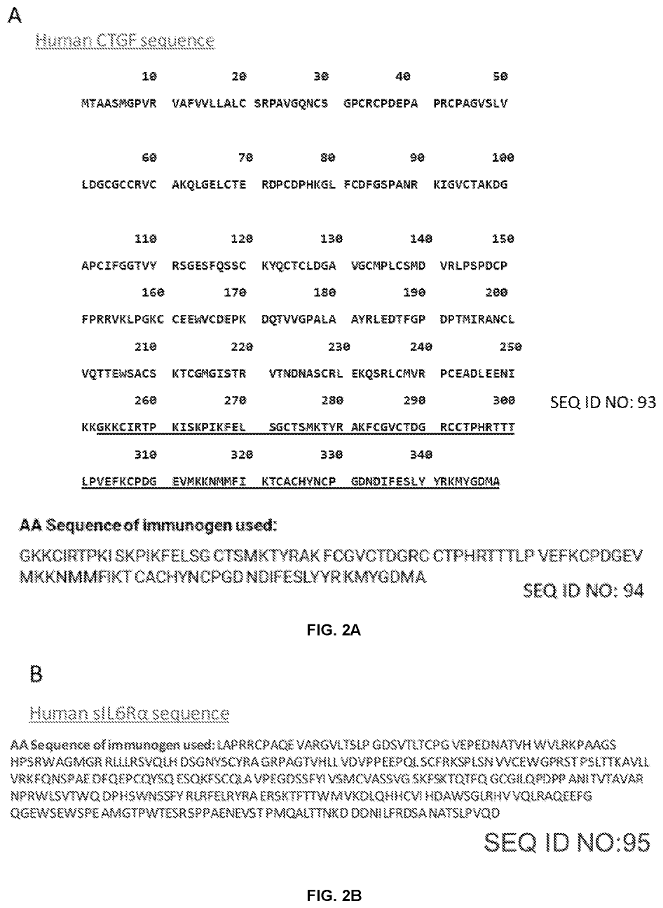

CTGF: The full length human CTGF is consisting of 349 amino acids (SEQ ID NO:93). Its amino acid sequence is shown in FIG. 2A. A truncated form of recombinant CTGF, consisting of 98 amino acids from the C-terminal end was used to immunize the camel. The molecular weight of this peptide is 11 kDa and the sequence of the peptide is show in FIG. 2A (SEQ ID NO:94). The immunogen used in this study were shown to exert full heparin binding activity, cell adhesion, and mitogenic activity.

sIL6Ra: IL-6 receptor consists of two chains, IL-6Ra and gp130. Recombinant Human sIL-6Ra is a 37.6 kDa glycoprotein corresponding to 339 amino acid residues of the extracellular domain of IL-6Ra. The sequence of the peptide with which camel was immunized is show in FIG. 2B (SEQ ID NO:95).

### Example 2: Induction of a Humoral Immune Response in Camel

Camel was immunized with 4 doses of CTGF or sIL6R along with Freunds complete Adjuvant (FCA) or incomplete adjuvant (FIA). While first dose was mixed with FCA, remining doses were mixed with FIA.

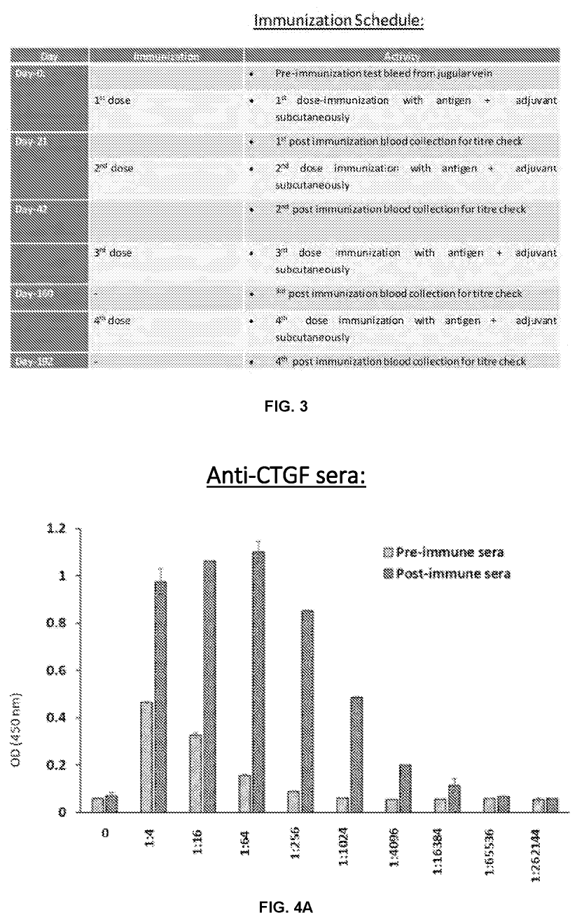

A total of 4 immunizations were made. The first 3 doses are given at 3-weekly intervals and the last dose was given about 4 months after the 3rd dose. About 600 μl of FCA was mixed with 600 μl of immunogen solution (containing 200 μg of protein), mixed thoroughly until a thick emulsion was formed. This antigen mix is subcutaneously injected into a young adult male camel (Camelus dromedarius), one camel for each antigen, at multiple sites (200 μl/site, total 1.2 ml). Immunization schedule is shown in FIG. 3.

### Example 3: Evaluation of Immune Response

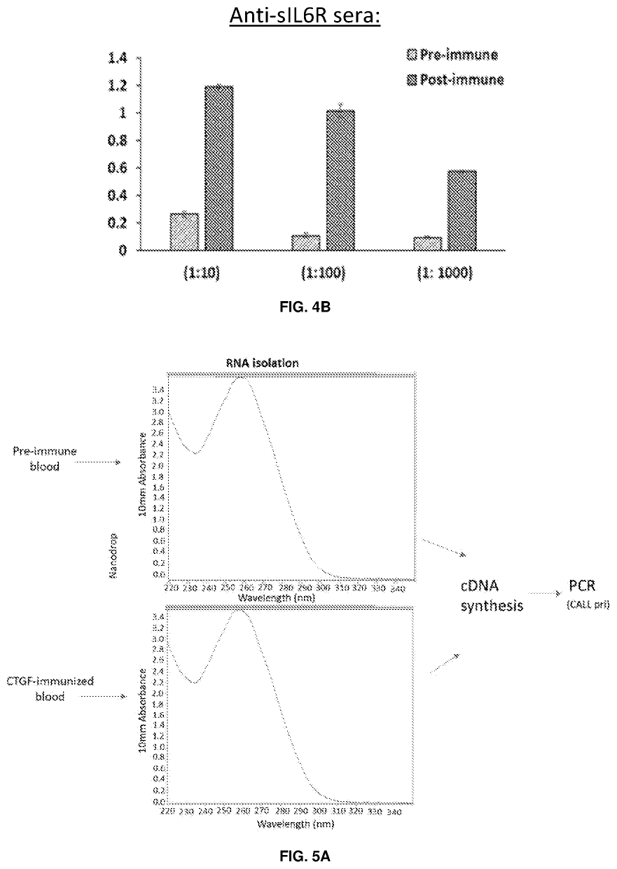

At day 0, 21, 42, 160 and 192, 10 ml of pre-immune/immune blood was collected, sera separated and was used to evaluate the induction of the immune responses in the immunized camels. For this, recombinant human CTGF or sIL6R at 1-5 μg/ml in coating buffer, were immobilized overnight at 4° C. in a 96 well Maxisorp plate (Nunc). Wells were blocked with 3% milk solution prepared in PBST buffer. After addition of various serum dilutions, specifically bound immunoglobulins were detected using an HRP-conjugated goat anti-camel IgG antibody. In ELISA, there is a significant reactivity with post CTGF-immunized sera compared to pre-immune sera across several dilutions (FIG. 4A). A clear positive response is seen up to a dilution of 1:16000. Similarly, a clear response is seen with 1:1000 dilution of anti-sIL6R sera.

### Example 4: Library Construction

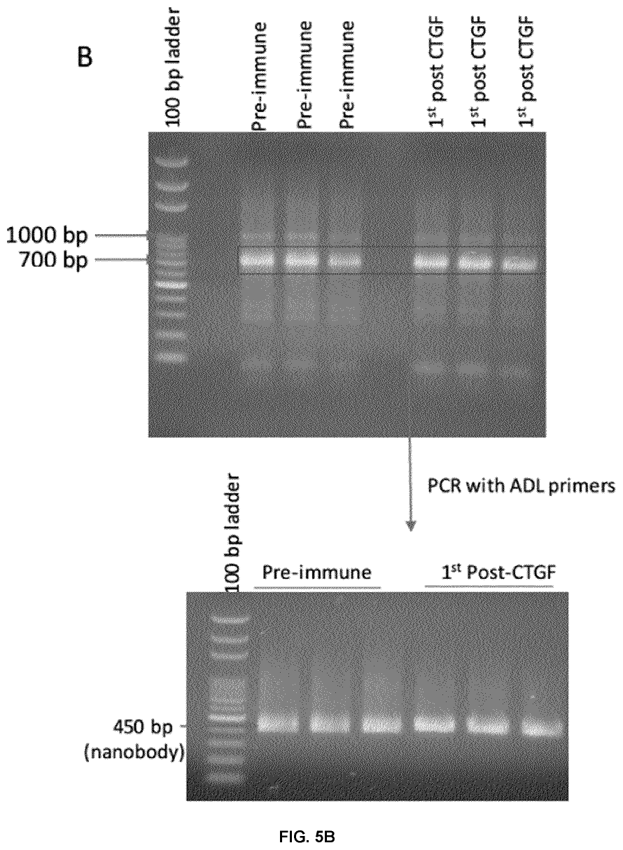

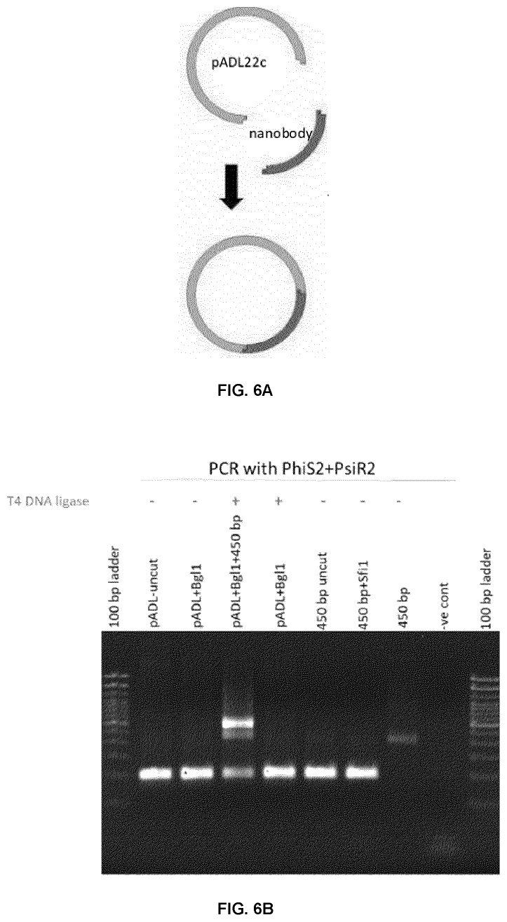

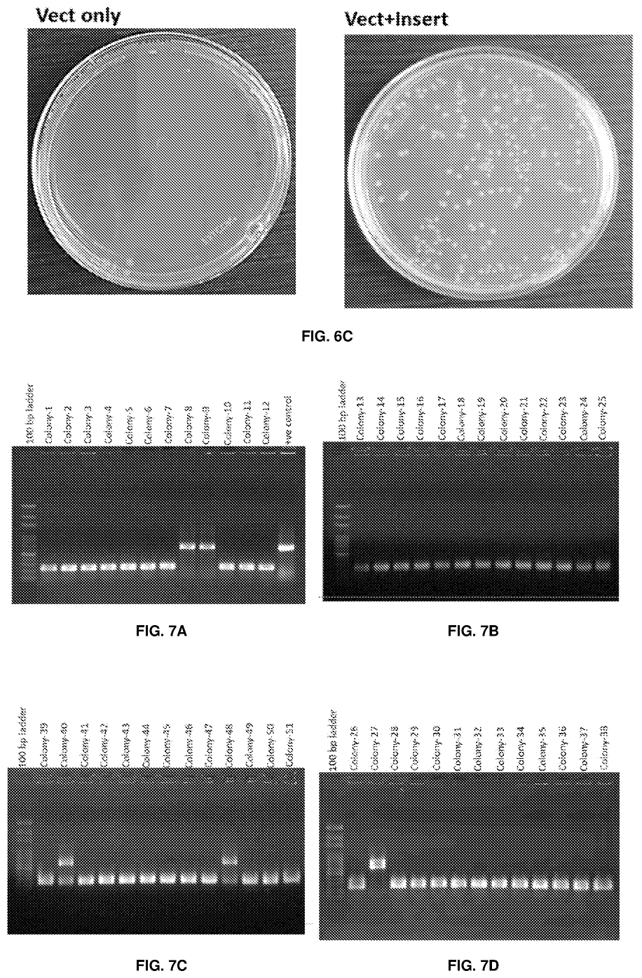

The blood of the pre-immune and post immunized (CTGF and sIL6R) animal were collected in RNA PAX tubes (PreAnalytix) and total RNA from peripheral blood cells extracted according to the manufacturer's instruction. RNA quantity was estimated using Nanodrop and representative example is shown for CTGF (FIG. 5A). RNA was retro-transcribed to cDNA using the High-Capacity cDNA Reverse Transcription Kit (Thermo Fisher). Over the years, several PCR strategies have been developed to amplify VHH gene fragments from lymphocyte cDNA. A two-step nested PCR approach have been used. One pair of primers, CALL001 (5′-GTCCTGGCTGCTCTTCTACAAGG-3′ SEQ ID NO:96) and CALL002 (5′-GGTACGTGCTGTTGAACTGTTCC-3′ SEQ ID NO:97) has been designed for the first PCR by using the cDNA as the template. The CALL002 primer anneals in a region of the second constant heavy-chain domain (CH2) that is conserved among all IgG isotypes of all camelids, whereas the CALL001 primer anneals in a well-conserved region of the leader signal sequence of all V elements of family III (by far the most abundant V family in camelids). Two prominent bands were observed: 1000 bp band and 700 bp fragment (FIG. 5B). While the 1000 bp fragment is the Variable domains of HC of IgG1, the 700 bp fragment represents the VHH-single domain Ab.

The 700 bp fragment was excised from agarose gel and was subjected to a second round of PCR using the primers ADL2-sense primer (GTG CAG GCC CAG CCG GCC ATG GCC CAT GGC CGA KGT SCA GCT; where K is G or T, S is C or G SEQ ID NO:98) and ADL2-antisense primer (G TGA TTG GCC TCC CGG GCC TGA GGA GAC GGT GAC CTG GGT SEQ ID NO:99) to amplify the VHH repertoire (FIG. 5B). After confirming the amplification product of approximately 450 bp fragment on gel electrophoresis, the PCR mix was cleaned up with the Qiagen PCR purification kit, according to the manufacturer's instructions.

### Example 5: Cloning of VHH fragments into PAL22c phagemid vector

The 450 bp purified fragments as obtained above were digested with Sfi1 restriction enzyme and the pADL22c phagemid vector was digested with Bgl1 restriction enzyme. The digested products were purified by Qiagen PCR purification kit. A ligation reaction was performed by mixing digested pADL22c vector and digested VHH fragments at a ratio of 1:3, followed by purification of ligation mix with a Qiagen kit. To confirm if the ligation worked, a PCR was performed using vector specific primers PhiS2 and PsiR2 (that flank the multicloning site in PADL22c vector). Appearance of a clear band around 600 bp confirmed the presence of VHH fragments inserted into pADL22 vector. The ligated products were introduced into TG1 cells by electroporation (Biorad) and the efficiency of transformation or size of the library was found to be 3.1×10⁷/ug of DNA.

### Example 6: Rescue of the Library and Phage Preparation

The library (Six OD600 units) was grown at 37° C. in 10 ml 2xTY medium containing 2% glucose, and 100 μg/ml ampicillin, until the OD600 nm reached 0.5. M13K07 phages in 10 times excess of TG1 bacteria (Lucigen) were added and the mixture was incubated at 37° C. for 2×30 minutes, first without shaking, then with shaking at 100 rpm. Cells were centrifuged for 10 minutes at 4500 rpm at room temperature. The bacterial pellet was resuspended in 50 ml of 2xTY medium containing 100 μg/ml ampicillin and 25 μg/ml kanamycin and incubated overnight at 37° C. with vigorously shaking at 250 rpm. The overnight cultures were centrifuged for 15 minutes at 4000 rpm at 4° C. Phages were precipitated using PEG (20% poly-ethylene-glycol and 1.5 M NaCl) and centrifuged for 30 minutes at 13000 rpm. The pellet was resuspended in 20 ml PBS. Phages were again PEG precipitated and centrifuged for 30 minutes at 13000 rpm and 4° C. The pellet was dissolved in 1 ml PBS. Phages were titrated by infection of TG1 cells at OD600 nm=0.5 and plating on LB agar plates containing 100 μg/ml ampicillin and 2% glucose. The number of transformants indicates the number of phages (=pfu). The phages were stored at −80° C. with 20% glycerol.

### Example 7: Antigen Presentation and Phage Selection by Panning

CTGF or sIL6R coated plate was incubated (1011 phages/well) with phages as obtained above and unbound phages were washed off with PBST. Bound phages were eluted with 0.25% trypsin and the phage count was made. TG1 E coli were infected with the eluted phages and rescued. The resulting bacteria were spread on LB-agar plates at various dilutions and grown overnight at 37° C.

### Example 8: Immune Library Colony Screening for the Presence of VHH (VHH Fragment Antibody) Gene

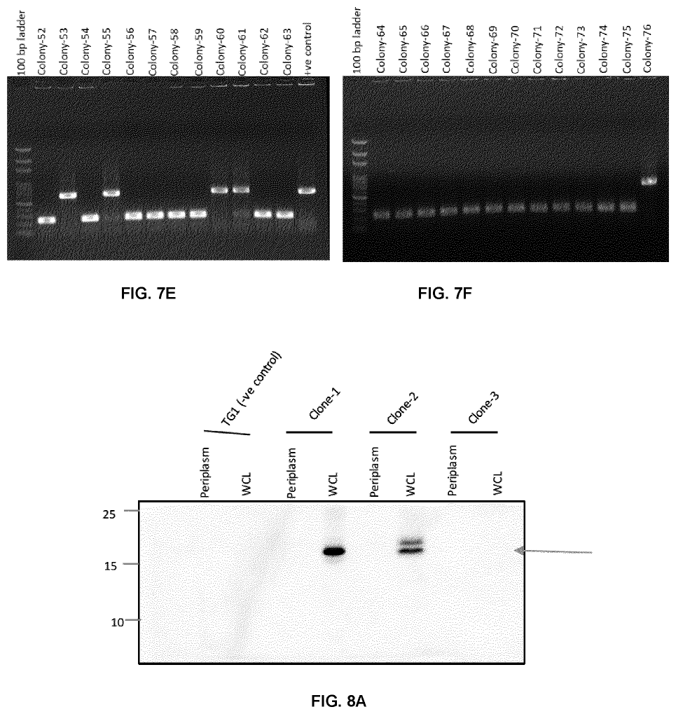

100 colonies from Vector+insert plate of CTGF and sIL6R and 3 colonies from Vector only plate were picked, and a colony PCR performed to check if they have VHH fragment antibody genes using vector-specific primers that flank the VHH genes. 15 out of 100 colonies in vector+insert displayed VHH fragment antibody genes, whereas no amplification of 600 bp fragment is seen in the colonies transformed with vector only. Representative example of screening of CTGF clones is shown FIG. 7. Similarly, no amplification is seen with empty phage display pADL22c vector.

### Example 9: Expression and Purification of Selected Sequences

Selected clones, that were having VHH fragment, were grown and induced with 1 mM IPTG to express VHH fragment antibody. Periplasmic extracts as well as whole cell lysates were prepared from these clones and subjected to western blot screening using HA tag-specific Abs. Out 15 clones, 13 displayed the presence of VHH fragment antibody protein expression, although the level of expression in each clone varies (FIG. 8). In most clones the VHH fragment antibody is localized to inclusion bodies and much less in soluble form as evident from this western blot analysis.

### Example 10: Recognition of CTGF by VHH Fragment Antibody

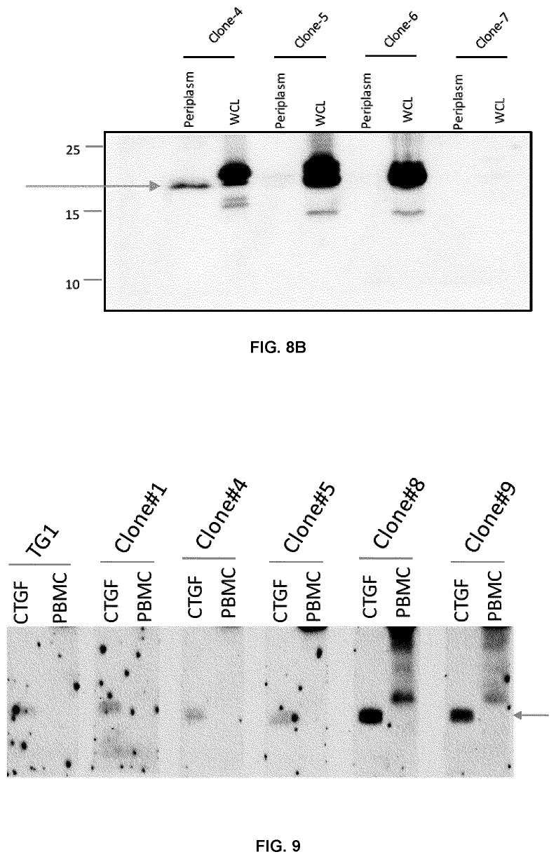

About 1 μg/well CTGF and 30 μg/well human control PBMC lysates were western blotted, and the blots were incubated with the periplasmic lysate collected as above, followed by incubation with anti-HA antibodies and anti-Ms-IgG-HRP. Clear recognition of CTGF by VHH fragment antibody present in the periplasmic extract was noticed (FIG. 9). These results suggest that the obtained VHH fragment antibodies are able to bind the CTGF.

### Example 11: Sequencing of the Selected Clones

The cDNA encoding for the selected clones were sequenced using a Big Die Terminator Cycle Sequencing Ready Reaction Kit (Applied Biosystems) in an ABI-Prism 377 DNA automatic sequencer (Perkin Elmer, Applied Biosystems) (FIG. 7).

All biochemical and molecular biology reagents were chemical grade purchased from various companies. Unless stated otherwise, the bacterial media were prepared as described (Sambrook et al., Molecular cloning: A Laboratory Manual (2nd Ed.). Cold Spring Harbor Laboratory, Cold Spring Harbor, NY (1989)). Phosphate-buffered saline (PBS) was prepared as described (Sambrook et al., supra). Induction medium was the same as Terrific Broth except that it contained no salts. Agarose top was prepared by combining the following reagents in a total volume of 1 liter: 10 g bacto-tryptone, 5 g yeast extract, 10 g NaCl, 1 g MgCl₂·6H₂O, and 7 g agarose. The mixture was autoclaved and stored solid at room temperature. The oligonucleotides were synthesized using the Applied Biosystems 394 DNA/RNA synthesizer. DNA sequencing was performed by the dideoxy method (Sanger et al., Biotechnology, 104-108 (1992)) using the AmpliTaq DNA Polymerase FS kit and 373A DNA Sequencer Stretch (PE Applied Biosystems, Mississauga, ON, Canada). The host bacteria used for cloning was TG1: supE hsdo thi (lac-proAB) F [fraD36 proAB+lacP/acZM15]. All the cloning steps were performed as described (Sambrook et al., supra).

TABLE 1
sIL-6Receptor specific VHH
CDRs are bold underlined.

| SIL6R VHH clone | | |
|---|---|---|
| ID | SIL6R-VHH seq | SEQ ID NO |
| SIL6R-G-A1 | MAESGGGSVQ AGGSLTLSCV GYGSKCMGWF | SEQ ID NO: 1 |
| | RQAPGKEREG VAGITTDSGI TYYADSVKGR | |
| | FIISQDKVKN TVYLEMSSLK PEDTAMYYCA | |
| | AHQNKLGSCG NWARYNEWGQ GTQVTVSSGP | |
| | GGQHHHHHHG AYPYDVPDYAS | |
| SIL6R-G-A1- | GWFRQ | SEQ ID NO: 2 |
| CDR1 | | |
| SIL6R-G-A1-CDR2 | DSGITYYADSVKGRFI | SEQ ID NO: 3 |
| SIL6R-G-A1-CDR3 | AHQNKLGSCGNWARYNEWGQG | SEQ ID NO: 4 |
| SIL6R-P-A1 | MAEVQLVESG GGSVQAGGSL TLSCVGYGSK | SEQ ID NO: 5 |
| | CMGWFRQAPG KEREGVAGIT TDSGITYYAD | |
| | SVKGRFIISQ DKVKNTVYLE MSSLKPEDTA | |
| | MYYCAAHQNK LGSCGNWARY NEWGQGTQVT | |
| | VSSGPGGQHH HHHHGAYPYD VPDYAS | |
| SIL6R-P-A1-CDR1 | GSKCM | SEQ ID NO: 6 |
| SIL6R-P-A1-CDR2 | AGITTDSGITYYADSV | SEQ ID NO: 7 |
| SIL6R-P-A1-CDR3 | YCAAHQNKLGSCGNWARYN | SEQ ID NO: 8 |
| SIL6R-R-A1 | MAESGGGSVL AGASLRLSCA ASGFPSSGKC | SEQ ID NO: 9 |
| | MGWFRQTPGK EREGVATINS RSGAIYYADS | |
| | VKGRFTVSQD NASNTVYLQM NSLEPEDTAV | |
| | YYCAARPTYV DYACSATPNY MDWGQGTQVT | |
| | VSSGPGGQHH HHHHGAYPYD VPDYAS | |
| SIL6R-R-A1-CDR1 | GKCMG | SEQ ID NO: 10 |
| SIL6R-R-A1-CDR2 | TINSRSGAIYYADSVK | SEQ ID NO: 11 |
| SIL6R-R-A1-CDR3 | CAARPTYVDYACSATPNYM | SEQ ID NO: 12 |
| SIL6R-Y-C2 | MAEVQLVESG GGLVQPGGSL RLSCAATGFS | SEQ ID NO: 13 |
| | FSSSYMNWVR QAPGKGLEWV SGINIEGSQT | |
| | YYSDSAKGRF TISRDNAKST VFLQMNSLKS | |
| | EDTALYYCAP IWYCTNVVSC TYWGQGTQVT | |
| | VCGPGGQHHH HHHGAYPYDV PDYAS | |
| SIL6R-Y-C2-CDR1 | GFSFS | SEQ ID NO: 14 |
| SIL6R-Y-C2-CDR2 | LEWVSGINIEGSQTYY | SEQ ID NO: 15 |
| SIL6R-Y-C2-CDR3 | TALYYCAPIWYCTNVVSCT | SEQ ID NO: 16 |
| SIL6R-Y-E2 | MXEVQLVESG GGLVQPGGSL RLSCAASGFA | SEQ ID NO: 17 |
| | FSSYYMSWVR QAPGKGLEWV SGIWHDGSNT | |
| | YYAGSVKGRF TISGDNAKNT VYLQMNNLKP | |
| | EDTAVYYCAT APWSSYGDNY WGQGTQVTVC | |
| | GPGGQHHHHH HGAYPYDVPD YAS | |
| SIL6R-Y-E2-CDR1 | GFAFS | SEQ ID NO: 18 |
| SIL6R-Y-E2-CDR2 | LEWVSGIWHDGSNTYY | SEQ ID NO: 19 |
| SIL6R-Y-E2-CDR3 | TAVYYCATAPWSSYGDNYWG | SEQ ID NO: 20 |
| SIL6R-Y-F1 | XXXXXXXX MAEVQPGGSL RLSCAASGFT FSDYDMSWVR | SEQ ID NO: 21 |
| | QAPGKGLEWV SNVGSGGGST | |
| | YYTDSVAGRF AISRDDAENT LYLQMNNLKP | |
| | EDTAVYYCAT GRNPATPGWV PPALYEYNYW | |
| | GQGTQVTVSS GPGGQHHHHH HGAYPYDVPD YAS | |
| SIL6R-Y-F1-CDR1 | GFTES | SEQ ID NO: 22 |
| SIL6R-Y-F1-CDR2 | LEWVSNVGSGGGSTYY | SEQ ID NO: 23 |
| SIL6R-Y-F1-CDR3 | TAVYYCATGRNPATPGWV | SEQ ID NO: 24 |
| SIL6R-Y-B6 | MXXVQLVESG GGLVQPGGSL RLSCAASGFT | SEQ ID NO: 25 |
| | FSIDAMSWVR QAPGKGLEWV SAINAGGSTY | |
| | YADSVKGRFT ISRDNAKNTL YLQLNSLKTE | |
| | DTAMYYCTNY NYGLNTLAGQ GTQVTVSSGP | |
| | GGQHHHHHHG AYPYDVPDYAS* | |
| SIL6R-Y-B6-CDR1 | GFTES | SEQ ID NO: 26 |
| SIL6R-Y-B6-CDR2 | LEWVSAINAGGSTYY | SEQ ID NO: 27 |
| SIL6R-Y-B6-CDR3 | TAMYYCTNYNYGLNTLAGQ | SEQ ID NO: 28 |
| SIL6R-Y-D6 | MAEVQLVESG GGLVQPGGSL SVSCAASGFT | SEQ ID NO: 29 |
| | FSAYYMSWVR QAPGKGLEWV SGINMDGSNT | |
| | YYADSVKGRF TISRDNAKNT LYLQMNSLKS | |
| | EDTARYYCAA PYGGNWYPTT SYNYWGQGTQ | |
| | VTVCGPGGQH HHHHHGAYPY DVPDYAS* | |
| SIL6R-Y-D6-CDR1 | GFTES | SEQ ID NO: 30 |
| SIL6R-Y-D6-CDR2 | LEWVSGINMDGSNTYY | SEQ ID NO: 31 |
| SIL6R-Y-D6-CDR3 | TARYYCAAPYGGNWYPTTS | SEQ ID NO: 32 |
| SIL6R-Y-E5 | MAEVQLVESG GDLVQPEESL SVSCAAAGFT | SEQ ID NO: 33 |
| | FSGYFMSWVR QAPGKGLQWV SGINTDGSKT | |
| | YYTDSVKGRF TISRDNAKNT LYLQMNSLKS | |
| | EDTALYYCVV DRYGGGYWGQ GTQVTVSSGP | |
| | GGQHHHHHHG AYPYDVPDYAS* | |
| SIL6R-Y-E5-CDR1 | GFTES | SEQ ID NO: 34 |
| SIL6R-Y-E5-CDR2 | LQWVSGINTDGSKTYY | SEQ ID NO: 35 |
| SIL6R-Y-E5-CDR3 | TALYYCVVDRYGGGYWGQGT | SEQ ID NO: 36 |
| SIL6R-Y-C9 | XADVQLVESG GGLVQPGGSL TLSCAASGFT | SEQ ID NO: 37 |
| | FSTYDMTWVR QAPGKGLEWV SRINSGGDTT | |
| | YYADSVKGRF AVSRDIAKNT LYLQMNSLKP | |
| | EDTAIYYCAA SSVSGLLYRS LVSNDYNYWG | |
| | QGTQVTVCGP GGQHHHHHH GAYPYDVPDY AS* | |
| SIL6R-Y-C9-CDR1 | GFTFS | SEQ ID NO: 38 |
| SIL6R-Y-C9-CDR2 | LEWVSRINSGGDTTYY | SEQ ID NO: 39 |
| SIL6R-Y-C9-CDR3 | TAIYYCAASSVSGLLY | SEQ ID NO: 40 |
| SIL6R-Y-D9 | PXADVQLVES GGGLVQPGGS LTLSCAASGF | SEQ ID NO: 41 |
| | TFSTYDMTWV XQAPGKGLEWV SRINSGGDTT | |
| | YYADSVKGQF TISRDNAKNMLYLQM NSLKPDDTAV | |
| | YYCAPVWNWA FWGQGTQVTV XGPGGXHHXH | |
| | HHGAXPXXXX XXXX* | |
| SIL6R-Y-D9-CDR1 | GFTFS | SEQ ID NO: 42 |
| SIL6R-Y-D9-CDR2 | LEWVSRINSGGDTTYY | SEQ ID NO: 43 |
| SIL6R-Y-D9-CDR3 | TAVYYCAPVWNWAFWGQGTQV | SEQ ID NO: 44 |
| SIL6R-Y-F4 | MAEVQLVESG GGLVQPGGSL RLSCAASGFT | SEQ ID NO: 45 |
| | FSSYDMSWVR QAPGKGLEWV SAINSGGGST | |
| | YYADSVKGRF TISRDNAKNT LYLQMNSLKP | |
| | EDTAVYYCAT DDGGSWYPTS SFGYWGQGTQ | |
| | VTVSSGPGGQ HHHHHHGAYP YDVPDYAS* | |
| SIL6R-Y-F4-CDR1 | GFTFS | SEQ ID NO: 46 |
| SIL6R-Y-F4-CDR2 | LEWVSAINSGGGSTYY | SEQ ID NO: 47 |
| SIL6R-Y-F4-CDR3 | TAVYYCATDDGGSWYPTSS | SEQ ID NO: 48 |
| SIL6R-Y-H4 | MAEVQLVESG GGLVQPGGSL RLSCAASGGF | SEQ ID NO: 49 |
| | TFSNYWMHWV RQAPRRGLEW VCGINSDGSN | |
| | TYYADSVKGR FTISRDSAKN TLYVQMNSLK | |
| | SEDTALYYCA RRPPYGYAYN YWGQGTQVTV | |
| | CGPGGQHHHH HHGAYPYDVP DYAS* | |
| SIL6R-Y-H4-CDR1 | GFTFS | SEQ ID NO: 50 |
| SIL6R-Y-H4-CDR2 | LEWVCGINSDGSNTYY | SEQ ID NO: 51 |
| SIL6R-Y-H4-CDR3 | TALYYCARRPPYGYAYNYWGQG | SEQ ID NO: 52 |
| SIL6R-Y-C7 | LAEVQLVESG GGLVQPGGSL RLSCAASGFT | SEQ ID NO: 53 |
| | FSNYYMTWVC QAPGKGLEWV SGINSDGTNT | |
| | YYTDSVKGRF TISRDNARNT LYLQMNSLKS | |
| | EDTALYYCAT DPLFYAWGQG TQVTVSSGPG | |
| | GQHHHHHHGA YPYDVPDYAS* | |
| SIL6R-Y-C7-CDR1 | GFTFS | SEQ ID NO: 54 |
| SIL6R-Y-C7-CDR2 | LEWVSGINSDGTNTYY | SEQ ID NO: 55 |
| SIL6R-Y-C7-CDR3 | ALYYCATDPLFYAWGQGT | SEQ ID NO: 56 |
| SIL6R-Y-D7 | XAEVQLVESG GGLVQPGGSL RLSCAASGFT | SEQ ID NO: 57 |
| | FSTYYMSWVR QAPGKGLEWV SGIRSDGSTT | |
| | WYGDSVKGRF TISRDNAENM LYLQMNSLKL | |
| | EDTAVYYCAG DPIFTMSTMG YWGQGTQVTV | |
| | CGPGGQHHHH HHGAYPYDVPDYAS* | |
| SIL6R-Y-D7-CDR1 | GFTFS | SEQ ID NO: 58 |
| SIL6R-Y-D7-CDR2 | LEWVSGIRSDGSTTWY | SEQ ID NO: 59 |
| SIL6R-Y-D7-CDR3 | AVYYCAGDPI FTMSTMGY | SEQ ID NO: 60 |
| SIL6R-Y-D8 | XAEVQLVESG GGLVQPGGSL RLSCAASGFT | SEQ ID NO: 61 |
| | FSTYYMSWVR QAPGKGLEWV SGIRSDGSTT | |
| | WYGDSVKGRF TISRDNAENM LYLQMNSLKL | |
| | EDTAVYYCAG DPIFTMSTMG YWGQGTQVTV | |
| | CGPGGQHHHH HHGAYPYDVP DYAS* | |
| SIL6R-Y-D8-CDR1 | GFTES | SEQ ID NO: 62 |
| SIL6R-Y-D8-CDR2 | LEWVSGIRSDGSTTWY | SEQ ID NO: 63 |
| SIL6R-Y-D8-CDR3 | AVYYCAGDPI FTMSTMGY | SEQ ID NO: 64 |
| SIL6R-Y-E8 | MADVQLVESG GGLVQPGGSL TLSCAASGFT | SEQ ID NO: 65 |
| | FSTYDMTWVR QAPGKGLEWV SRINSGGDTT | |
| | YYADSVKGQF TISRDNAKNM LYLQMNSLKP | |
| | DDTAVYYCAP VWNWAFWGQG TQVTVCGPGG | |
| | QHHHHHHGAY PYDVPDYAS* | |
| SIL6R-Y-E8-CDR1 | GFTFS | SEQ ID NO: 66 |
| SIL6R-Y-E8-CDR2 | LEWVSRINSGGDTTYY | SEQ ID NO: 67 |
| SIL6R-Y-E8-CDR3 | AVYYCAPVWNWAFWGQGT | SEQ ID NO: 68 |

Table 2 shows additional sIL6R VHH sequences. The ATG start codons for the VHH construct are bolded in the DNA Seq column. The end (last codons) of the VHH is GTCTCCTCA in the DNA sequences. The TAG stop codon is bolded in the DNA Seq column. The GGCCCGGGAGGCCAA (SEQ ID NO:288) in the DNA sequences is extra sequence generated when the VHH fragment is cloned into pADL22c vector using Bgl1 and Sfi1 restriction enzyme sites. The CACCATCACCACCATCAT (SEQ ID NO:289) in the DNA sequences is a HIS tag. The TATCCGTATGATGTGCCGGACTATGCT (SEQ ID NO:290) in the DNA sequence is a HA tag. In the protein sequences, the CDRs are bolded (CDR1, CDR2, and CDR3).

TABLE 2

| Clone ID | DNA Seq | Protein seq |
|---|---|---|
| SIL6R-1A2 | ATACCTATTGCCTACGGCGGCCGCTGGATTGTTATTACTCGCGGCCCAGCCGGCCATGGCCCAGGTGCAGCTGCAG | MAQVQLQESGGGSVQAGGSLRLSCAASEY |
| | GAGTCTGGAGGAGGCTCGGTGCAGGCTGGAGGGTCTCTGAGACTCTCCTGTGCAGCCTCTGAATATACCGTGACTA | TVTRAWFRQAPGKEREWVAGINSGGGST |
| | GGGCCTGGTTCCGCCAGGCTCCAGGAAAGGAGCGTGAGTGGGTCGCAGGCATTAATAGTGGTGGTGGTAGCACAT | YYTDSVKGRFTISQDKARNTVFLQMNSLKP |
| | ACTACACCGACTCCGTGAAGGGCCGATTCACCATCTCCCAAGACAAGGCCAGGAACACGGTGTTTCTGCAAATGAA | EDTAIYTCAAPSSSGDYKYWGQGTQVTVSS |
| | CAGCCTGAAACCTGAGGACACGGCCATCTACACCTGTGCGGCGCCATCAAGTTCCGGCGACTATAAGTACTGGGGC | GPGGQ SEQ ID NO: 119 |
| | CAGGGGACCCAGGTCACCGTCTCCTCAGGCCCGGGAGGCCAACACCATCACCACCATCATGGCGCATATCCGTATG | CDR-1: SEQ ID NO: 138 |
| | ATGTGCCGGACTATGCTTCTTAGGCCGAAACTGTTGAAAGTTGTTTAGCAAAACCTCATACAGAAAATTCATTTACTA | CDR-2: SEQ ID NO: 139 |
| | ACGTCTGGAAAGACGACAAACTTTAGATCGTTACGCTAACTATGAGGGCTGTCTGTGAATGCTACAGCGTTGTGTTT | CDR-3: SEQ ID NO: 140 |
| | GTACTGATGACGAACTCAGTGTACGTACATGGTCTATGGGCTGCTATCTTGAAATGAGGTGTGCCTTGAAGTGCGTC | |
| | TGAGTGCCGTCTGAGTTGCCGTTACATAACCCTCCTGA SEQ ID NO: 100 | |
| SIL6R-1A4 | ATACCCTAATTGCCTACGGCGGCCGCTGGATTGTTATTACTCGCGGCCCAGCCGGCCATGGCCCAGGTGCAGCTGCA | MAQVQLQESGGGLVQPGGSLRLSCAASVF |
| | GGAGTCTGGAGGAGGCTTGGTGCAGCCTGGGGGGTCTCTGAGACTCTCCTGTGCAGCCTCTGTATTCACATTCAGT | TFSGYHMSWVRQAPGKGLEWVSAIDSGG |
| | GGCTACCACATGAGCTGGGTCCGCCAGGCTCCAGGGAAGGGGCTCGAGTGGGTCTCAGCTATAGATAGTGGTGGT | GSTYYADSVKGRFTISRDNAKNSLYLQMNS |
| | GGTAGCACATACTATGCAGACTCCGTGAAGGGCCGATTCACCATCTCCAGAGACAACGCCAAGAACTCGCTGTATCT | LKPEDTAVYYCATAPDIYEVETGLPPRRDFG |
| | GCAAATGAACAGTTTGAAACCTGAGGACACTGCCGTGTATTACTGCGCCACAGCGCCCGACATCTATGAGGTGGAA | YWGQGTQVTVSS GPGGQ SEQ ID |
| | ACCGGTCTTCCCCCTAGAAGAGACTTTGGTTACTGGGGCCAGGGGACCCAGGTCACCGTCTCCTCAGGCCCGGGA | NO: 120 |
| | GGCCAACACCATCACCACCATCATGGCGCATATCCGTATGATGTGCCGGACTATGCTTCTTAGGCCGAAACTGTTGAA | CDR-1: SEQ ID NO: 141 |
| | AGTTGTTTAGCAAAACCTCATACAGAAAATTCATTTACTAACGTCTGGAAAGACGACAAAACTTTAGATCGTTACGCT | CDR-2: SEQ ID NO: 142 |
| | AACTATGAGGGCTGTCTGTGGAATGCTACAGGCGTTGTGGTTTGTACTGGTGACGAAACTCAGTGTACGGTACATG | CDR-3: SEQ ID NO: 143 |
| | GGTTCCTATTGGGCCTGGCTATCCTGAATTGAGGTTGTGCTCTGAGGGTGGCGATTCTGGAGGATGGCCGGCTCTC | |
| | TGA SEQ ID NO: 101 | |
| SIL6R- | ATACCTATTGCCTACGGCGGCCGCTGGATTGTTATTACTCGCGGCCCAGCCGGCCATGGCCCAGGTGCAGCTGCAG | MAQVQLQESGGGSVQAGGSLRLSCLVSGY |
| 1A8 | GAGTCTGGGGGAGGCTCGGTGCAGGCTGGAGGGTCTCTGAGACTCTCCTGTTTAGTCTCTGGATACACCTACACTA | TYTNVYMGYFRQAPGKGREGVASIHRGLG |
| | ACGTCTACATGGGCTACTTCCGCCAGGCTCCAGGGAAGGGGCGTGAGGGGGTCGCAAGTATTCATCGTGGTCTTGG | TTDYADSVKGRFAIYQDTAKNTVYLQMNSL |
| | AACCACAGACTACGCCGACTCCGTGAAGGGCCGATTCGCCATTTACCAGGACACCGCCAAAAACACGGTGTATCTA | KPEDTDIYYCALGPIANLAYAADYEGQGTQ |
| | CAAATGAACAGCCTGAAACCTGAGGACACGGACATCTATTACTGTGCGCTTGGGCCGATCGCTAACTTAGCCTATG | VTVSS GPGGQ SEQ ID NO: 121 |
| | CAGCTGACTATGAGGGCCAGGGGACCCAGGTCACCGTCTCCTCAGGCCCGGGAGGCCAACACCATCACCACCATCA | CDR-1: SEQ ID NO: 144 |
| | TGGCGCATATCCGTATGATGTGCCGGACTATGCTTCTTAGGCCGAAACTGTTGAAAGTTGTTTAGCAAAACCTCAT | CDR-2: SEQ ID NO: 145 |
| | ACAGAAAATTCATTTACTAACGTCTGGAAAGACGACAAAACTTTAGATCGTTACGCTAACTATGAGGGCTGTCTGT | CDR-3: SEQ ID NO: 146 |
| | GGAATGCTACAGGCGTTGTGTTTGTACTGATGACGAAACTCAGTGTACGTACATGGTCTATGGCTGGCTATCCTGA | |
| | AATGAGTGTACTCTGAAGTGCGTCCTGAAGTTGCCGTCTGAGGGTGGCGGTAACTAACT SEQ ID NO: 102 | |
| SIL6R- | TACCTATTGCCTACGGCGGCCGCTGGATTGTTATTACTCGCGGCCCAGCCGGCCATGGCCCAGGTGCAGCTGCAG | MAQVQLQESGGGSVQAGGSLRLSCAASGY |
| 1A9 | GAGTCTGGAGGAGGCTCGGTGCAGGCTGGAGGGTCTCTGAGACTCTCCTGTGCAGCCTCTGGATACATCGGCAG | IGSAGCIAWFRQAPGKEREGVAAVGAAGS |
| | TGCAGGCTGCATAGCCTGGTTCCGCCAGGCTCCAGGGAAGGAGCGCGAGGGGGTCGCAGCAGTCGGCGCTGCA | SSSSTYYADSVKGRFTISQDSAKNTIYLQ |
| | GGTAGTAGCAGTAGTAGCACATACTACGCCGACTCCGTGAAGGGCCGATTCACCATCTCCCAAGACAGCGCCAA | MNILKPEDTAMYYCAAGPLWSNGGRCSNH |
| | GAACACGATATATCTACAAATGAACATCCTGAAACCTGAGGACACTGCCATGTATTATTGTGCAGCAGGCCCACT | IFDRSWYYYWGQGTQVTVSS GPGGQ |
| | GTGGTCGAATGGGGGGCGTTGCTCGAACCACATTTTCGACCGCTCGTGGTACTACTACTGGGGCCAGGGGACCC | SEQ ID NO: 122 |
| | AGGTCACCGTCTCCTCAGGCCCGGGAGGCCAACACCATCACCACCATCATGGCGCATATCCGTATGATGTGCCGG | CDR-1: SEQ ID NO: 147 |
| | ACTATGCTTCTTAGGCCGAAACTGTTGAAAGTTGTTTAGCAAAACCTCATACAGAAAATTCATTTACTAACGTCTG | CDR-2: SEQ ID NO: 148 |
| | GAAAGACGACAAAACTTTAGATCGTTACGCTAACTATGAGGGCTGTCTGTGGAATGCTACAGGCGTTGTGATTTG | CDR-3: SEQ ID NO: 149 |
| | TACTGATGACGAAACTCAGTGTACGGTACATGGATCTATGGGCTGGCCTATCCCTTGAAATGAAGTTGTTGCTCG | |
| | GAGGGTGGCGGCTTCTG SEQ ID NO: 103 | |
| SIL6R- | ATCCTAATTGCCTACGGCGGCCGCTGGATTGTTATTACTCGCGGCCCAGCCGGCCATGGCCCAGGTGCAGCTGCAG | MAQVQLQESGGGSVQTGGSLRLSCAASGY |
| 1A12 | GAGTCTGGAGGAGGCTCGGTGCAGACTGGAGGGTCTCTGAGACTCTCCTGTGCAGCCTCTGGATACCGCTTCAGTC | RFSRYCKGWFRQAPGKEREAVAHISGGGY |
| | GTTATTGCAAGGGCTGGTTCCGCCAGGCTCCAGGAAAGGAGCGTGAGGCGGTCGCACATATCTCTGGTGGTGGATA | ETYYLDSVKGRFTISQDSADNTLYLQMNS |
| | TGAAACATACTACCTCGACTCCGTGAAGGGCCGATTCACCATCTCCCAGGACAGCGCCGACAACACGCTATATCTA | LKSEDTAIYYCAAGIVVSNRCEITDFSYW |
| | CAAATGAACAGCCTGAAATCTGAGGACACGGCCATCTATTACTGTGCGGCTGGTATCGTAGTGAGTAACCGGTGCG | GQGTQVTVSS GPGGQ |
| | AAATTACCGACTTTAGTTACTGGGGCCAGGGGACCCAGGTCACCGTCTCCTCAGGCCCGGGAGGCCAACACCATCA | SEQ ID NO: 123 |
| | CCACCATCATGGCGCATATCCGTATGATGTGCCGGACTATGCTTCTTAGGCCGAAACTGTTGAAAGTTGTTTAGCA | CDR-1: SEQ ID NO: 150 |
| | AAACCTCATACAGAAAATTCATTTACTAACGTCTGGAAAGACGACAAAACTTTAGATCGTTACGCTAACTATGAGG | CDR-2: SEQ ID NO: 151 |
| | GCTGTCTGTGGAATGCTACAGGCGTTGTGTTTGTACTGTGACGAAACTCAGTGTACGTACATGGCTCTATGGGCTG | CDR-3: SEQ ID NO: 152 |
| | CTATCTGAATGAGGTGGTGCTCTGAGGTTGCGTCTGAGGGTGGCGGTTTCTGGAGGGGGGCCGA SEQ ID | |
| | NO: 104 | |
| SIL6R- | AAACCCTATTGCCTACGGCGGCCGCTGGATTGTTATTACTCGCGGCCCAGCCGGCCATGGCCCAGGTGCAGCTGCA | MAQVQLQESGGGSVQAGGSLRLSCAASEY |
| 1B3 | GGAGTCTGGGGGAGGCTCGGTGCAGGCTGGAGGGTCTCTGAGACTCTCCTGTGCAGCCTCTGAATATACCATCACT | TITRAWFRQAPGKAREWVAGINSGGGSTF |
| | AGGGCCTGGTTCCGCCAGGCTCCAGGGAAGGCGCGTGAGTGGGTCGCAGGTATTAATAGTGGTGGTGGTAGCACA | YADSVKGRFTISQDNARNTVFLQMTSLKPE |
| | TTCTACGCCGACTCCGTGAAGGGCCGATTCACCATCTCCCAAGACAACGCCAGGAACACGGTGTTTCTGCAAATGA | DTAIYTCAAPSSSGDYKYWGLGTQVTVSS |
| | CCAGCCTGAAACCTGAGGACACGGCCATCTATACCTGTGCGGCGCCATCAAGCTCCGGCGACTATAAGTACTGGGG | GPGGQ SEQ ID NO: 124 |
| | CCTTGGGACCCAGGTCACCGTCTCCTCAGGCCCGGGAGGCCAACACCATCACCACCATCATGGCGCATATCCGTATG | CDR-1: SEQ ID NO: 153 |
| | ATGTGCCGGACTATGCTTCTTAGGCCGAAACTGTTGAAAGTTGTTTAGCAAAACCTCATACAGAAAATTCATTTACTA | CDR-2: SEQ ID NO: 154 |
| | ACGTCTGGAAAGACGACAAAACTTTAGATCGTTACGCTAACTATGAGGGCTGTCTGTGATGCTACAGGCGTTGTGTT | CDR-3: SEQ ID NO: 155 |
| | TGTACTGGTGACGAACTCAGTGTACGTACATGGGTTCTATGGGCTGGCTATCCCTGAAATGAAGTTGTGCCTCTGAA | |
| | GTGCGTCTGAGTGGCGTCTGAAGTGCGTACTAACCTCCTGAGTTACGGTG SEQ ID NO: 105 | |
| SIL6R- | ATAACCTAATTGCCTACGGCGGCCGCTGGATTGTTATTACTCGCGGCCCAGCCGGCCATGGCCCAGGTGCAGCTGC | MAQVQLQESGGGLVQPGGSLRLSCAASGF |
| 1C4 | AGGAGTCTGGGGGAGGCTTGGTGCAGCCCGGGGGGTCTCTGAGACTCTCCTGTGCAGCCTCTGGATTCACCTTCAG | TFSDYAMSWVRQAPGKGLEWVSSINSGG |
| | TGACTATGCCATGAGCTGGGTCCGCCAGGCTCCAGGGAAGGGGCTCGAGTGGGTCTCATCTATTAATAGTGGTGGT | DGTAVAASLEGRFTISRDNAKNTLYLQLNSL |
| | GATGGCACAGCCGTTGCAGCGTCCCTAGAGGGCCGATTCACCATCTCCAGAGACAACGCCAAGAACACGCTATATC | KTEDTAMYYCAKTGRVHIALLQEAVRGQGT |
| | TGCAATTGAATAGCCTGAAAACTGAAGACACGGCCATGTATTACTGTGCAAAAACGGGGAGGGTTCATATAGCGCT | QVTVSS GPGGQ SEQ ID NO: 125 |
| | GTTGCAAGAGGCCGTCCGGGGCCAGGGGACCCAGGTCACCGTCTCCTCAGGCCCGGGAGGCCAACACCATCACCAC | CDR-1: SEQ ID NO: 156 |
| | CATCATGGCGCATATCCGTATGATGTGCCGGACTATGCTTCTTAGGCCGAAACTGTTGAAAGTTGTTTAGCAAAAC | CDR-2: SEQ ID NO: 157 |
| | CTCATACAGAAAATTCATTTACTAACGTCTGGAAAGACGACAAAACTTTAGATCGTTACGCTAACTATGAGGGCTG | CDR-3: SEQ ID NO: 158 |
| | TCTGTGGAATGCTACAGGCGTTGTGTTTGTACTGTGACGAAACTCAGTGTTACGGTACATGGGTCTATGGCTGCTA | |
| | TCCCTGAAAATGAAGTGGTGCTCTGAGTTGCCGTCTGAGGTGGCGGTTCTGAGGGGTGGCGGTAACTTA SEQ ID | |
| | NO: 106 | |
| SIL6R- | TACCCTATGGCCTTACGGCGGCCGCTGGATTGTTATTACTCGCGGCCCAGCCGGCCATGGCCCAGGTGCAGCTGCA | MAQVQLQESGGGAVQAGGSLRLSCAASG |
| 1D6 | GGAGTCTGGGGGAGGCGCGGTGCAGGCTGGAGGGTCTCTGAGACTCTCCTGTGCAGCCTCTGGATACACCTACAG | YTYSSSCMGWFRQAPGKEREAVAAIYTDN |
| | TAGCTCCTGCATGGGCTGGTTCCGCCAGGCTCCAGGGAAGGAGCGCGAGGCAGTCGCAGCTATTTATACTGATAAT | GSTYYADSVKGRFTISQDNAKNTVYLQMNS |
| | GGTAGCACATACTATGCCGACTCCGTGAAGGGCCGATTCACCATCTCCCAAGACAACGCCAAGAATACGGTGTATCT | LKPEDTAMYYCAAAAVTLRMGGECIAAIQ |
| | GCAAATGAACAGCCTGAAACCTGAGGACACTGCCATGTACTACTGTGCAGCAGCGGCGGTAACCCTCCGGATGGG | ALGVMTFGADFGYWGQGTQVTVSS |
| | GGGGGAGTGCATAGCTGCTATTCAGGCTCTTGGTGTTATGACGTTCGGAGCTGACTTTGGTTACTGGGGCCAGGGG | GPGGQ SEQ ID NO: 126 |
| | ACCCAGGTCACCGTCTCCTCAGGCCCGGGAGGCCAACACCATCACCACCATCATGGCGCATATCCGTATGATGTGCC | CDR-1: SEQ ID NO: 159 |
| | GGACTATGCTTCTTAGGCCGAAACTGTTGAAAGTTGTTTAGCAAAACCTCATACAGAAAATTCATTTACTAACGTCTG | CDR-2: SEQ ID NO: 160 |
| | GAAAGACGACAAAACTTTAGATCGTTACGCTAACTATGAGGGCTGTCTGTGGAATGCTACAGGCGTTGTGGTTGTAC | CDR-3: SEQ ID NO: 161 |
| | TGGGTGACGAAACTCATGTACCGTACATTGGTCTATGGGCTTGCTATCCTGAAATGGAGGGTGGGTGGCCTCTTGAA | |
| | GGTG SEQ ID NO: 107 | |
| SIL6R- | AACCCTATTGCCTACGGCGGCCGCTGGATTGTTATTACTCGCGGCCCAGCCGGCCATGGCCCAGGAGCAGCTGCA | MAQEQLQESGGGSVQAGGSLRLSCAASGY |
| 1D12 | GGAGTCTGGGGGAGGCTCGGTGCAGGCTGGAGGGTCTCTGAGACTCTCCTGTGCAGCCTCTGGATACATCTACA | IYSSNCMGWFRQAPGKEREGVAAICTGGG |
| | GTAGCAACTGCATGGGGTGGTTCCGCCAGGCTCCAGGGAAGGAGCGCGAGGGGGTCGCAGCTATTTGTACTGG | STYYADSVKGRFTVSQDNAKNTVYLQMSSL |
| | TGGTGGTAGCACATACTATGCCGACTCCGTGAAGGGCCGATTCACCGTCTCCCAAGACAACGCCAAGAACACGGT | KPEDTAMYYCATGGAYAWRYYSDYDPAS |
| | GTATCTGCAAATGAGCAGCCTGAAACCTGAGGACACTGCCATGTACTACTGCGCAACAGGGGGAGCATATGCTT | WWGSLRYNMDYWGKGTRVTVSS |
| | GGAGATACTATAGCGACTATGACCCAGCCTCGTGGTGGGGCAGTTTAAGGTATAACATGGACTACTGGGGCAAA | GPGGQ SEQ ID NO: 127 |
| | GGGACCCGGGTCACCGTCTCCTCAGGCCCGGGAGGCCAACACCATCACCACCATCATGGCGCATATCCGTATGAT | CDR-1: SEQ ID NO: 162 |
| | GTGCCGGACTATGCTTCTTAGGCCGAAACTGCTGAAAGATGTTCAGCTAAACCTCATACGAAAGATCCATTTACTA | CDR-2: SEQ ID NO: 163 |
| | TCGTCTGGTAACGACAGACAACTTAGATTCGTTACCGCA SEQ ID NO: 108 | CDR-3: SEQ ID NO: 164 |
| SIL6R- | AACCTATTGCCTACGGCGGCCGCTGGATTGTTATTACTCGCGGCCCAGCCGGCCATGGCCCAGGTGCAGCTGCAG | MAQVQLQESGGGSVQAGGSLRLSCTASGF |
| 1E7 | GAGTCTGGGGGAGGCTCGGTGCAGGCTGGAGGGTCTCTGAGACTCTCCTGTACAGCCTCTGGATTCACTTTTGAT | TFDDSDMGWYHQAPGNECELVSGIISDGS |
| | GATTCTGACATGGGCTGGTACCACCAGGCTCCAGGGAATGAGTGCGAGTTGGTCTCAGGTATTATTAGTGATGG | TYYADSVKGRFTISRDNAKNTVYLKMNSLK |
| | TAGCACATACTATGCAGACTCCGTGAAGGGCCGATTCACCATCTCCAGAGACAACGCCAAAAACACGGTGTATCT | PEDTANYYCAAYPPGWPGCSLQKFHYDH |
| | GAAAATGAACAGCCTGAAACCTGAGGACACGGCCAACTATTATTGTGCGGCCTACCCCCCCGGGTGGCCCGGAT | WGQGTQVTVSS GPGGQ SEQ ID NO: 128 |
| | GTTCTCTCCAAAAATTTCATTATGATCACTGGGGCCAGGGGACCCAGGTCACCGTCTCCTCAGGCCCGGGAGGCC | CDR-1: SEQ ID NO: 165 |
| | AACACCATCACCACCATCATGGCGCATATCCTTATGATGTGCCGGACTATGCTTCTTAGGCCGAAGCTGTTGAAAG | CDR-2: SEQ ID NO: 166 |
| | TTGTTTAGCAAAACCTCATACAGAATATTCATTTACTAACGTCTGGAAAGACGACAAAACTTTAGATCGCTACGCT | CDR-3: SEQ ID NO: 167 |
| | AACTATGAGGGCTGTCTGTGGAATGCTACAGGCGTTGGTGTTTTGTACTGATGACAATACTCAGTGCTGCCACTC | |
| | GTGGGCTTCTAGTGGTGCTGCTATCCATGATATGGAGGTGGTGCTCATGGAGGGTGGGCGCTTTCAGGAGGGG | |
| | GCGGCGTCTTTCTGGGAGGGGGTGGGGCGGCTACTACA SEQ ID NO: 109 | |
| SIL6R- | ATACCTAATTGCCTACGGCGGCCGCTGGATTGTTATTACTCGCGGCCCAGCCGGCCATGGCCCAGGTGCAGCTGC | MAQVQLQESGGGSVQAGGSLRLSCLVSGY |
| 1E9 | AGGAGTCTGGGGGAGGCTCGGTGCAGGCTGGAGGGTCTCTGAGACTCTCCTGTTTAGTCTCTGGATACACCTAC | TYSNVYMGYFRQAPGKGREAVASIHRGLG |
| | AGTAACGTCTACATGGGCTACTTCCGCCAGGCTCCAGGGAAGGGGCGTGAGGCGGTCGCAAGTATTCATCGTGG | TADYADSVKGRFAIYQDTAKNTVYLQMNSL |
| | TCTTGGAACCGCAGACTACGCCGACTCCGTGAAGGGCCGATTCGCCATTTACCAGGACACCGCCAAAAACACGGT | KPEDTAIYYCALGPIANLAWASDYEGQGTQ |
| | GTATCTACAAATGAACAGCCTAAAACCTGAGGACACGGCCATCTATTACTGTGCGCTTGGGCCGATCGCTAACTT | VTVSS GPGGQ SEQ ID NO: 129 |
| | GGCCTGGGCATCTGACTATGAGGGCCAGGGGACCCAGGTCACCGTCTCCTCAGGCCCGGGAGGCCAACACCATC | CDR-1: SEQ ID NO: 168 |
| | ACCACCATCATGGCGCATATCCGTATGATGTGCCGGACTATGCTTCTTAGGCCGAAACTGTTGAAAGTTGTTTAGC | CDR-2: SEQ ID NO: 169 |
| | AAAACCTCATACAGAAAATTCATTTACTAACGTCTGGAAAGACGACAAAACTTTAGATCGTTACGCTAACTATGAG | CDR-3: SEQ ID NO: 170 |
| | GGCTGTCTGTGGAATGCTACAGGCGTTGTGGTTTGTACTGGTGACGAAACTCAGTGTTACGGTACATGGGTCTAT | |
| | TGGCTGCTATCCTGAAAATGAAGTGGTGCTCTGAGTTGCCGTTCTGAGGGTGCGATCTGAGGGTTGGGCGGGTA | |
| | CTTAAGCTC SEQ ID NO: 110 | |
| SIL6R- | ATACCCTATTGCCTACGGCGGCCGCTGGATTGTTATTACTCGCGGCCCAGCCGGCCATGGCCCAGGAGCAGCTGC | MAQEQLQESGGGLVQPGGSLRLSCAASGF |
| 1F1 | AGGAGTCTGGGGGAGGCTTGGTGCAGCCCGGGGGGTCTCTGAGACTCTCCTGTGCAGCCTCCGGATTCACCTCT | TSGFTFNDYDMSWVRQAPGKGLEWVSGI |
| | GGATTCACATTCAATGACTACGACATGAGCTGGGTCCGCCAGGCTCCAGGGAAGGGGCTCGAGTGGGTCTCAGG | NTADYTDYIDSVKGRFTISRDNAKNTLYLQ |
| | TATTAATACTGCTGATTACACAGACTATATAGACTCCGTGAAGGGCCGATTCACCATCTCCAGAGACAACGCCAA | MNNLKPEDTAVYFCATDWNPLMPTGFLP |
| | GAACACGCTGTATCTGCAAATGAACAATTTGAAACCTGAGGACACTGCCGTGTATTTCTGCGCCACAGATTGGAA | RGQGTQVTVSS GPGGQ SEQ ID NO: 130 |
| | TCCATTGATGCCTACGGGTTTTTTGCCCAGGGGCCAGGGGACCCAGGTCACCGTCTCCTCAGGCCCGGGAGGCCA | CDR-1: SEQ ID NO: 171 |
| | ACACCATCACCACCATCATGGCGCATATCCGTATGATGTGCCGGACTATGCTTCTTAGGCCGAAACTGTTGAAAGT | CDR-2: SEQ ID NO: 172 |
| | TGTTTAGCAAAACCTCATACAGAAAATTCATTTACTAACGTCTGGAAAGACGACAAAACTTTAGATCGTTACGCTA | CDR-3: SEQ ID NO: 173 |
| | ACTATGAGGGCTGTCTGTGAATGCTACAGGCGTTGTGATTTGTACTGTGACGAACTCAGTGGTACGGTACATGGC | |
| | TCTATTGGGCTGCTATCCCTGGAAATGAAGGTGGTGCTCTGAAGGGTGGGCGGATCTGGAGGGGTGGCGGACTT | |
| | GGAGGGGGTGGGGCGGA SEQ ID NO: 111 | |
| SIL6R- | AACCTATTGCCTACGGCGGCCGCTGGATTGTTATTACTCGCGGCCCAGCCGGCCATGGCCCAGGTGCAGCTGCAG | MAQVQLQESGGGSVQAGGSLRLSCAASGY |
| 1F2 | GAGTCTGGGGGAGGCTCGGTGCAGGCTGGAGGGTCTCTGAGACTCTCCTGTGCAGCCTCTGGATACACCTACAG | TYSSYCMGWFRQAPGKESEGVAAINSGGG |
| | TAGCTACTGCATGGGCTGGTTTCGCCAGGCTCCAGGGAAGGAGAGTGAGGGGGTCGCAGCTATTAATAGTGGT | STYYADSVKGRFTISQDNANTVYLQMNSLK |
| | GGTGGTAGCACATACTACGCCGACTCCGTGAAGGGCCGATTCACCATCTCCCAAGACAACGCCAACACGGTGTAT | PEDTAIYYCTAAPEDYCSGGLWSFGMDYW |
| | CTGCAAATGAACAGCCTGAAACCTGAGGACACGGCCATCTATTACTGTACGGCAGCTCCGGAAGATTATTGTAGT | GKGTQVTVSS GPGGQ SEQ ID NO: 131 |
| | GGTGGTCTCTGGTCTTTTGGCATGGACTACTGGGGCAAAGGGACCCAGGTCACCGTCTCCTCAGGCCCGGGAGG | CDR-1: SEQ ID NO: 174 |
| | CCAACACCATCACCACCATCATGGCGCATATCCGTATGATGTGCCGGACTATGCTTCTTAGGCCGAAACTGTTGAA | CDR-2: SEQ ID NO: 175 |
| | AGTTGTTTAGCAAAACCTCATACAGAAAATTCATTTACTAACGTCTGGAAAGACGACAAAACTTTAGATCGTTACG | CDR-3: SEQ ID NO: 176 |
| | CTAACTATGAGGGCTGTCTGTGGAATGCTACAGGCGTTGTGGTTTGTACTGGTGACGAACTCAGTGTTACGGTAC | |
| | ATGGTTCCTATTGGCTTGCTATCCCTGAAATTGAAGTGTTGCTCTGAGGTTGCGATCTGAGGGTGGCGGATTCTG | |
| | GAAGGGTGGGCCGGTATACCTAA SEQ ID NO: 112 | |
| SIL6R- | AACCCTAATTGCCTACGGCGGCCGCTGGATTGTTATTACTCGCGGCCCAGCCGGCCATGGCCCAGGTGCAGCTGC | MAQVQLQESGGGSVQAGGSLRLSCAAAG |
| 1F4 | AGGAGTCTGGGGGAGGCTCGGTGCAGGCTGGAGGGTCTCTGAGACTCTCCTGTGCAGCCGCTGGATACACCGA | YTDNRKCMGWFRQAPGKEREGVAVIFTS |
| | CAATAGGAAATGCATGGGCTGGTTCCGCCAGGCTCCAGGGAAGGAGCGCGAGGGGGTCGCGGTTATTTTTACG | DSSTAYTDSVKGRFTISQGVPKNTVYLQMN |
| | AGTGATAGTAGTACTGCCTATACCGACTCCGTGAAGGGCCGATTCACCATCTCCCAAGGGGTCCCCAAGAACACG | DLKPEDTAMYYCAAYVSPYVYGGCSLQDF |
| | GTGTATCTCCAAATGAACGACCTGAAACCTGAGGACACTGCCATGTACTACTGTGCAGCGTATGTGAGCCCGTAC | RYDYWGQGTQVTVSS GPGGQ SEQ ID |
| | GTGTACGGCGGTTGCTCCTTGCAGGATTTCAGGTATGACTACTGGGGCCAGGGGACCCAGGTCACCGTCTCCTCA | NO: 132 |
| | GGCCCGGGAGGCCAACACCATCACCACCATCATGGCGCATATCCGTATGATGTGCCGGACTATGCTTCTTAGGCC | CDR-1: SEQ ID NO: 177 |
| | GAAACTGTTGAAAGTTGTTTAGCAAAACCTCATACAGAAAATTCATTTACTAACGTCTGGAAAGACGACAAACTTT | CDR-2: SEQ ID NO: 178 |
| | AGATTTCGTTACGCCA SEQ ID NO: 113 | CDR-3: SEQ ID NO: 179 |
| SIL6R- | AACCTAATTGCCTACGGCGGCCGCTGGATTGTTATTACTCGCGGCCCAGCCGGCCATGGCCCAGGTGCAGCTGCA | MAQVQLQESGGGSVQAGGSLRLSCTASGF |
| 1F5 | GGAGTCTGGAGGAGGCTCGGTGCAGGCTGGAGGGTCTCTGAGACTCTCCTGTACAGCCTCTGGATTCACTTTTGC | TFAYSDMGWYHQAPGNECELVSTISSDGS |
| | GTATTCAGACATGGGCTGGTACCACCAGGCTCCAGGGAATGAGTGCGAGTTGGTCTCAACTATTAGTAGTGATG | TYYADSVKGRFTISRDNAKNTVYLQMNSLK |
| | GTAGCACATACTATGCAGACTCCGTGAAGGGCCGATTCACCATCTCCAGAGACAACGCCAAGAACACGGTGTATC | PEDTAMYYCAADHWLAERSDCLAPAVGY |
| | TGCAAATGAACAGCCTGAAACCTGAGGACACGGCCATGTATTACTGTGCGGCAGATCATTGGCTGGCCGAACGT | WGQGTQVTVSS GPGGQ SEQ ID NO: 133 |
| | AGTGATTGTTTAGCTCCTGCCGTTGGTTACTGGGGCCAGGGGACCCAGGTCACCGTCTCCTCAGGCCCGGGAGG | CDR-1: SEQ ID NO: 180 |
| | CCAACACCATCACCACCATCATGGCGCATATCCGTATGATGTGCCGGACTATGCTTCTTAGGCCGAAACTGTTGAA | CDR-2: SEQ ID NO: 181 |
| | AGTTGTTTAGCAAAACCTCATACAGAAAATTCATTTACTAACGTCTGGAAAGACGACAAAACTTTAGATCGTTACG | CDR-3: SEQ ID NO: 182 |
| | CTAACTATGAGGGCTGTCTGTGGAATGCTACAGGCGTTGTGGTTTGTACTGGTGACGAAACTCAGTGTACGGTAC | |
| | ATGGGTCCTATGGCTTGCTATCCCTGAATTGAAGTGTGCTCTGAGGTTGCCGATCTGAGGGTGGCGGGTTCTGGA | |
| | GGGGGGTGGCGGTACTCTAC SEQ ID NO: 114 | |
| SIL6R- | ATACCCTATTGCCTACGGCGGCCGCTGGATTGTTATTACTCGCGGCCCAGCCGGCCATGGCCCAGGTGCAGCTGC | MAQVQLQESGGGSVQAGGSLRLSCAASGY |
| 1F8 | AGGAGTCTGGAGGAGGCTCGGTGCAGGCTGGAGGGTCTCTGAGACTCTCCTGTGCAGCCTCTGGATACCGCTTC | RFSRYCMGWFRQAPGKEREAVAHIAGGG |
| | AGTCGTTATTGCATGGGCTGGTTCCGCCAGGCTCCAGGAAAGGAGCGTGAGGCGGTCGCACATATCGCTGGTGG | YGTYYLDSVKGRFTISQDSAKNTLYLQMNSL |
| | TGGATATGGAACATACTACCTCGACTCCGTGAAGGGCCGATTCACCATCTCCCAAGACAGCGCCAAGAACACGCT | KSEDTAIYYCAAGIIVSNRCESADFSYWGQ |
| | ATATCTACAAATGAACAGCCTGAAATCTGAGGACACGGCCATCTATTACTGTGCGGCTGGTATCATAGTGAGTAA | GTQVTVSS GPGGQ SEQ ID NO: 134 |
| | CCGGTGCGAGAGTGCCGACTTTAGTTACTGGGGCCAGGGGACCCAGGTCACCGTCTCCTCAGGCCCGGGAGGCC | CDR-1: SEQ ID NO: 183 |
| | AACACCATCACCACCATCATGGCGCATATCCGTATGATGTGCCGGACTATGCTTCTTAGGCCGAAACTGTTGAAA | CDR-2: SEQ ID NO: 184 |
| | GTTGTTTAGCAAAACCTCATACAGAAAATTCATTTACTAACGTCTGGAAAGACGACAAAACTTTAGATCGTTACGC | CDR-3: SEQ ID NO: 185 |
| | TAACTATGAGGGCTGTCTGTGGAATGCTACAGGCGTTGTGTTTGTACTGGTGACGAAACTCAGTGTACGTACATG | |
| | GGTTCTATTGGGCTGCTATCTGGAATTGAAGTTGGTTGCTCTGAGGGTGGCGGTTCTGGAGGGGTTGGGCGGTC | |
| | TGAGTTGC SEQ ID NO: 115 | |
| SIL6R- | AACCTATTGCCTACGGCGGCCGCTGGATTGTTATTACTCGCGGCCCAGCCGGCCATGGCCCAGGAGCAGCTGCA | MAQEQLQESGGGSVQAGGSLRLSCLASGY |
| 1G3 | GGAGTCTGGGGGAGGCTCGGTGCAGGCTGGAGGGTCTCTGAGACTCTCCTGTTTAGCCTCTGGATACACCTACA | TYTTYYMGWFRQAPGKGREGVASIHRGVE |
| | CTACCTACTACATGGGCTGGTTCCGCCAGGCTCCAGGGAAGGGGCGTGAGGGGGTCGCAAGTATTCATCGTGGT | TTYYADSVKGRFTIYRDTAKNTVYLQMNSL |
| | GTTGAAACCACATACTACGCCGACTCCGTGAAGGGCCGATTCACCATTTACCGAGACACCGCCAAAAACACGGTG | KPEDTAIYYCALELIGNLCYASDSHGQGTQV |
| | TATCTACAAATGAACAGCCTGAAACCTGAGGACACGGCCATCTATTACTGTGCGCTTGAACTGATCGGTAACTTGT | TVSS GPGGQ SEQ ID NO: 135 |
| | GCTATGCATCAGACTCTCACGGCCAGGGGACCCAGGTCACCGTCTCCTCAGGCCCGGGAGGCCAACACCATCACC | CDR-1: SEQ ID NO: 186 |
| | ACCATCATGGCGCATATCCGTATGATGTGCCGGACTATGCTTCTTAGGTGCGAAGACTGTTGAAAGTTGTTTAGA | CDR-2: SEQ ID NO: 187 |
| | AACTGCTCATACTTGTTAATTCATTTACTCACGTCTGAAAAGACTACAATAACTGTCTGTACGATACGACAAAAACT | CDR-3: SEQ ID NO: 188 |
| | TTAGATCGTTACGCA SEQ ID NO: 116 | |
| SIL6R- | AAACCTATTGCCTACGGCGGCCGCTGGATTGTTATTACTCGCGGCCCAGCCGGCCATGGCCCAGGTGCAGCTGCA | MAQVQLQESGGGLVQPGGSLRLSCAASGF |
| 1G10 | GGAGTCTGGGGGAGGCTTGGTGCAGCCTGGGGGGTCTCTGAGACTCTCCTGTGCAGCCTCCGGATTCACCTCTG | TSGFTFNDYDMSWVRQAPGKGLEWVSGI |
| | GATTCACATTCAATGACTACGACATGAGCTGGGTCCGCCAGGCTCCAGGGAAGGGGCTCGAGTGGGTCTCAGGT | NTADYTDYIDSVKGRFTISRDNAKNTLYLQ |
| | ATTAATACTGCTGATTACACAGACTATATAGACTCCGTGAAGGGCCGATTCACCATCTCCAGAGACAACGCCAAG | MNNLKPEDTAVYFCATDWNPLMPTGFLP |
| | AACACGCTGTATCTGCAAATGAACAATTTGAAACCTGAGGACACTGCCGTGTATTTCTGCGCCACAGATTGGAAT | RGQGTQVTVSS GPGGQ SEQ ID NO: 136 |
| | CCATTGATGCCTACGGGTTTTTTGCCCAGGGGCCAGGGGACCCAGGTCACCGTCTCCTCAGGCCCGGGAGGCCA | CDR-1: SEQ ID NO: 189 |
| | ACACCATCACCACCATCATGGCGCATATCCGTATGATGTGCCGGACTATGCTTCTTAGGCCGAAACTGTTGAAAGT | CDR-2: SEQ ID NO: 190 |
| | TGTTTAGCAAAACCTCATACAGAAAATTCATTTACTAACGTCTGGGAAAGACGACAAAACTTTAGATCGTTACGCT | CDR-3: SEQ ID NO: 191 |
| | AACTATGAGGGCTGTCTGTGAATGCTACAGCGTTGTGCTTGTACTGTGACGAACTCAGTGTACCGTACATGCTCT | |
| | ATGGTCTGCATCCTGAATGAGGTTGTGCTCTGAGCTGCCGTTCTGAAGGTGGCG SEQ ID NO: 117 | |
| SIL6R- | AAACCTATTGCCTACGGCGGCCGCTGGATTGTTATTACTCGCGGCCCAGCCGGCCATGGCCCAGGTGCAGCTGCA | MAQVQLQESGGGLVLPGGSLRLSCAASGF |
| 1G11 | GGAGTCTGGAGGAGGCTTGGTGCTGCCCGGGGGGTCTCTGAGACTCTCCTGTGCAGCCTCTGGATTCACATTCA | TFSNSVMSWVRQAPGKGIEWVSSISDGGD |
| | GTAACAGTGTCATGAGCTGGGTCCGCCAGGCTCCAGGGAAGGGCATCGAGTGGGTCTCATCTATTAGTGATGGA | YTDYADSVKGRFTISRDNAKNTLYLQLNSLK |
| | GGTGATTACACAGATTACGCGGACTCCGTGAAGGGCCGATTCACCATCTCCAGAGACAACGCCAAGAACACGCT | TVDTAMYYCATEWGWSAASSEYRGQGTQ |
| | GTATCTGCAATTGAACAGCCTGAAAACTGTCGACACGGCCATGTATTACTGTGCAACAGAGTGGGGTTGGTCCGC | VTVSS GPGGQ SEQ ID NO: 137 |
| | GGCCTCGTCTGAGTACAGGGGCCAGGGGACCCAGGTCACCGTCTCCTCAGGCCCGGGAGGCCAACACCATCACC | CDR-1: SEQ ID NO: 192 |
| | ACCATCATGGCGCATATCCGTATGATGTGCCGGACTATGCTTCTTAGGCCGAAACTGTTGAAAGTTGTTTAGCAA | CDR-2: SEQ ID NO: 193 |
| | AACCTCATACAGAAAATTCATTTACTAACGTCTGGAAAGACGACAAAACTTTAGATCGTTACGCTAACTATGAGG | CDR-3: SEQ ID NO: 194 |
| | GCTGTCTGTGGAATGCTACAGGCGTTGTGGTTTGTACTGGTGACGAAACTCAGTGTTACGGTACATGGGTTCCTA | |
| | TTGGCTGCTATCCCTGAAAATGAGGTGTGCTCTGAGGTTGCGTCTGAGTGCGCTTCTGAGGGTGGGCGGTACTAA | |
| | CCTTCCTGAG SEQ ID NO: 118 | |

TABLE 3
CTGF-specific VHH fragments
CDR regions are bold underlined.

| CTGF VHH clone | | |
|---|---|---|
| ID | Protein sequence of VHH fragment | SEQ ID NO |
| CTGF-A8 | MAEVQLVESG GGSVQAGGSL RLSCTASGST IDDADMGWYH | SEQ ID NO: 69 |
| | QAPGNECELV SSSTRDFSAY YSDSVKGRFT ISRDNAKNTI | |
| | FLQLNSLKPE DTGTYYCFLS KLEGDWYSSP ECRDTRSGSW | |
| | GQGTQVTVSS | |
| CTGF-A8-CDR1 | SGST IDDAD | SEQ ID NO: 70 |
| CTGF-A8-CDR2 | STRDESAY Y | SEQ ID NO: 71 |
| CTGF-A8-CDR3 | FLS KLEGDWYSSP ECRDTRSGSW | SEQ ID NO: 72 |
| CTGF-A9 | MAEVXLVESG GGSVQAGGSL RLSCTASGFT IDDADMGWYH | SEQ ID NO: 73 |
| | QAPGNECELV SSSTRDFSAY YSDSVKGRFT ISRDNAKNTI | |
| | FLQLNSLKPE DTGTYYCFLS KLEGDWYSSP ECRDTRFGSW | |
| | GQGTQSPS | |
| CTGF-A9-CDR1 | SGFT IDDAD | SEQ ID NO: 74 |
| CTGF-A9-CDR2 | STRDESAY Y | SEQ ID NO: 75 |
| CTGF-A9-CDR3 | FLS KLEGDWYSSP ECRDTRFGSW | SEQ ID NO: 76 |
| CTGF-C3 | MAEVQLVESG GGSVQAGGSL RLSCQASGYI FSSYTVGWER | SEQ ID NO: 77 |
| | QAPGKGCELV STLNSDGITD YTDSVKGRFT ISRDNTKNTV | |
| | YLQMNSLKPE DTAKYYCAAD PLNKIFLDGD CGEGNLEDWG | |
| | QGTQVTVSS | |
| CTGF-C3-CDR1 | SGYI FSSYT | SEQ ID NO: 78 |
| CTGF-C3-CDR2 | LNSDGITD Y | SEQ ID NO: 79 |
| CTGF-C3-CDR3 | AAD PLNKIFLDGD CGEGNLEDW | SEQ ID NO: 80 |
| CTGF-E5 | MAEVQLVESG GGSVQAGGSL RLSCASHEFT LTHYTMAWER | SEQ ID NO: 81 |
| | QIPGKRREGA AAITSGGETY YADSVKGRFT ISLDNNNRTV | |
| | DLQMNSLKLE DTAIYYCAAD VGQHIDASQA LSVGYSYWGQ | |
| | GTQVTVSS | |
| CTGF-E5-CDR1 | HEFT LTHYT | SEQ ID NO: 82 |
| CTGF-E5-CDR2 | ITSGGETY Y | SEQ ID NO: 83 |
| CTGF-E5-CDR3 | AAD VGQHIDASQA LSVGYSYW | SEQ ID NO: 84 |
| CTGF-E12 | MAEVQLVESG GGSVQAGGSL RLSCTASGFT IDDADMGWYH | SEQ ID NO: 85 |
| | QAPGNECELV SSSTRDFSAY YSDSVKGRFT ISRDNANNTM | |
| | YLQMNSLKPE DTAIYYCAAE RPAPCYSGSW SNAAGFKSWG | |
| | QGTQVTVSS | |
| CTGF-E12-CDR1 | SGFT IDDAD | SEQ ID NO: 86 |
| CTGF-E12-CDR2 | STRDESAY Y | SEQ ID NO: 87 |
| CTGF-E12-CDR3 | AAE RPAPCYSGSW SNAAGFKSWG | SEQ ID NO: 88 |
| CTGF-F1 | MAEVQLVESG GGSVQAGGSL RLTCDASGYT HSTNIMGWER | |
| | QRPGKEREGV AAIYTGDGST FYAYSVEGRV AISRDNAENV | |
| | VYLQMNSLKP EDTAMYYCAA GDGCSRSHAS WRTSDYAYWG | |
| | QGTQVTVSS | |
| CTGF-F1-CDR1 | SGYT HSTNI | SEQ ID NO: 90 |
| CTGF-F1-CDR2 | IYTGDGST F | SEQ ID NO: 91 |
| CTGF-F1-CDR3 | AA GDGCSRSHAS WRTSDYAYWG | SEQ ID NO: 92 |

Table 4 shows additional CTGF VHH sequences. The VHH constructs were generated using full length CTGF as the immunogen. The ATG start codons for the VHH construct are bolded in the DNA Seq column. The end (last codons) of the VHH is GTCTCCTCA in the DNA sequences. The TAG stop codon is bolded in the DNA Seq column. The GGCCCGGGAGGCCAA (SEQ ID NO: 288) in the DNA sequences is extra sequence generated when the VHH fragment is cloned into PADL22c vector using Bgl1 and Sfi1 restriction enzyme sites. The CACCATCACCACCATCAT (SEQ ID NO:289) in the DNA sequences is a HIS tag. The TATCCGTATGATGTGCCGGACTATGCT (SEQ ID NO:290) in the DNA sequence is a HA tag. In the protein sequences, the CDRs are bolded (CDR1, CDR2, and CDR3).

TABLE 4

| Clone ID | DNA Seq | Protein seq |
|---|---|---|
| FLCTGF- | GCCGCTGGATTGTTATTACTCGCGGCCCAGCCGGCCATGGCCCAGGTGCAGCTGCAGGAGTCTGGGGGAGGCT | MAQVQLQESGGGLVQPGGSLRLSCLASGFTF |
| 1A11 | TGGTGCAGCCCGGGGGGTCTCTGAGACTCTCCTGTTTGGCATCTGGATTCACCTTCAGTAGGTATGCCATGAACT | SRYAMNWVRQAPGKGLEWVSTIYSTGATYY |
| | GGGTCCGCCAGGCTCCAGGGAAGGGGCTCGAGTGGGTCTCAACAATTTATAGCACCGGTGCTACATACTATACG | TNSVEGRFTISRDNAKSTLYLQLNSLKTEDTGT |
| | AACTCTGTGGAGGGCCGATTCACCATCTCCAGAGACAACGCTAAGAGCACGCTGTATCTGCAATTGAACAGCCT | YYCTRADWKTGTRGQGTQVTVSS GPGGQ |
| | GAAAACTGAAGACACGGGCACGTATTACTGTACAAGGGCAGATTGGAAAACAGGCACTAGGGGCCAGGGGAC | SEQ ID NO: 214 |
| | CCAGGTCACCGTCTCCTCAGGCCCGGGAGGCCAACACCATCACCACCATCATGGCGCATATCCGTATGATGTGCC | CDR-1: SEQ ID NO: 233 |
| | GGACTATGCTTCTTAGGCCGAAACTGTTGAAAGTTGTTTAGCAAAACCTCATACAGAAAATTCATTTACTAACGT | CDR-2: SEQ ID NO: 234 |
| | CTGGAAAGACGACAAAACTTTAGATCGTTACGCTAACTATGAGGGCTGTCTGTGGAATGCTACAGGCGTTGTGT | CDR-3: SEQ ID NO: 235 |
| | TTGTACTGATGACGAAACTCAGTGTTACGGTACATGGTCTATGGGCTGCTATCCTGAAAATGAAGTTGTGCTCTG | |
| | AGGTGCGTCTGAGGTGCCGATCTGAGGTGCGTACTAGCTCTGAGTACCGTGATACATTATTCCGGCCTAA SEQ | |
| | ID NO: 195 | |
| FLCTGF- | ACCTATTGGCCTACGGCGGCCGCTGGATTGTTATTACTCGCGGCCCAGCCGGCCATGGCCCAGGTGCAGCTGCA | MAQVQLQESGGGSVQTGGSLRLSCAASGNT |
| 1B12 | GGAGTCTGGAGGAGGCTCGGTGCAGACTGGAGGGTCTCTGAGACTCTCCTGTGCAGCCTCCGGAAACACCTAT | YTDYIIGWLRQAPGKEREGVATIWSGSGITSY |
| | ACAGACTACATTATTGGCTGGCTCCGCCAGGCTCCAGGGAAGGAGCGTGAGGGGGTCGCAACTATTTGGAGTG | ADSVKGRFSISQDNAKNTVYLQMNSLKPEDT |
| | GAAGTGGTATTACATCGTACGCCGACTCCGTGAAGGGCCGATTCAGCATCTCCCAAGACAACGCCAAGAACACG | AIYFCAIRPRWYLEYQYWGQGTQVTVSS |
| | GTGTATCTTCAAATGAACAGCCTGAAACCTGAGGACACGGCCATCTATTTCTGTGCAATTCGGCCACGTTGGTAT | GPGGQ SEQ ID NO: 215 |
| | CTTGAATACCAGTATTGGGGCCAGGGAACCCAGGTCACCGTCTCCTCAGGCCCGGGAGGCCAACACCATCACC | CDR-1: SEQ ID NO: 236 |
| | ACCATCATGGCGCATATCCGTATGATGTGCCGGACTATGCTTCTTAGGCCGAAACTGTTGAAAGTTGTTTAGCAA | CDR-2: SEQ ID NO: 237 |
| | AACCTCATACAGAAAATTCATTTACTAACGTCTGGAAAGACGACAAAACTTTAGATCGTTACGCTAACTATGAGG | CDR-3: SEQ ID NO: 238 |
| | GCTGTCTGTGGAATGCTACAGGCGTTGTGGTTTGTACTGGTGACGAAACTCAGTGTTACGGTACATGGGTTCCC | |
| | TATGGCTTGCTATCCCTGAAAATGAGGGTGGTGCTCTGGAGCTGCGTCTGATTGCGTCTGAGGTGGCGGTACTA | |
| | AACCTCCTGGAGTACC SEQ ID NO: 196 | |
| FLCTGF- | ATACCTATTGCCTACGGCGGCCGCTGGATTGTTATTACTCGCGGCCCAGCCGGCCATGGCCCAGGTGCAGCTGC | MAQVQLQESGGGSVQPGGSLRLSCIVSGYTG |
| 1C2 | AGGAGTCTGGAGGAGGCTCGGTGCAGCCTGGAGGGTCTCTGAGACTCTCCTGTATAGTCTCTGGATACACTGG | GSDLMAWFRQAPGKEREGVATIYGVAGVTY |
| | TGGTAGCGACTTAATGGCCTGGTTCCGCCAGGCTCCAGGCAAGGAGCGCGAGGGGGTCGCAACTATTTATGGT | YADSVKGRFTITKDKNTVHLQMNSLKPEDTA |
| | GTCGCTGGTGTCACATATTATGCCGACTCCGTGAAGGGCCGATTCACCATCACGAAAGATAAGAACACGGTACAT | MYYCAVGRLGVVRSVLRSNGYSHWGQGTQ |
| | CTGCAAATGAACAGCCTGAAACCTGAGGACACTGCCATGTACTACTGTGCAGTAGGGGGGCTCGGTGTTGTACG | VTVSSGPGGQ SEQ ID NO: 216 |
| | TTCAGTCCTCCGCTCAAATGGGTATAGCCACTGGGGCCAGGGGACCCAGGTCACCGTCTCCTCAGGCCCGGGA | CDR-1: SEQ ID NO: 291 |
| | GGCCAACACCATCACCACCATCATGGCGCATATCCGTATGATGTGCCGGACTATGCTTCTTAGGCCGAAACTGTT | CDR-2: SEQ ID NO: 292 |
| | GAAAGTTGTTTAGCAAAACCTCATACAGAAAATTCATTTACTAACGTCTGGAAAGACGACAAAACTTTAGATCGT | CDR-3: SEQ ID NO: 293 |
| | TACGCTAACTATGAGGGCTGTCTGTGGAAATGCTACAGGCGTTGTGTTTGTACTGGTGACGAAACTCAGTGTAA | |
| | CGGTACATGGGTTCTATTGGGCTTGCTATCTTGAATTGAGGTTGGTGGCTCTGAGATGCGTTCTGAGGGTGGCG | |
| | GTTCTCTGAAG SEQ ID NO: 197 | |
| FLCTGF-1C9 | AACCTATTGCCTACGGCGGCCGCTGGATTGTTATTACTCGCGGCCCAGCCGGCCATGGCCCAGGTGCAGCTGCA | MAQVQLQESGGGLVQPGGSLRLSCAASGFTF |
| | GGAGTCTGGAGGAGGCTTGGTGCAGCCCGGGGGGTCTCTGAGACTCTCCTGTGCAGCCTCTGGATTCACCTTC | ANYAMNWVRQAPGKGLELVSTIYNSGTTYY |
| | GCTAATTATGCTATGAACTGGGTCCGCCAGGCTCCAGGAAAGGGGCTCGAGTTGGTCTCAACTATCTATAATAGT | PNSVKGRFTISRDNAKNTLYLQLNDLKTEDTA |
| | GGGACCACATACTATCCAAACTCCGTGAAGGGCCGATTCACCATCTCCAGAGACAACGCCAAGAACACACTATAT | MYYCVVPGLGNKRGQGTQVTVSS GPGGQ |
| | CTGCAATTGAACGACCTGAAAACTGAGGACACGGCCATGTATTACTGTGTTGTGCCTGGCCTCGGAAACAAGCG | SEQ ID NO: 217 |
| | GGGCCAGGGGACCCAGGTCACCGTCTCCTCAGGCCCGGGAGGCCAACACCATCACCACCATCATGGCGCATAT | |
| | CCGTATGATGTGCCGGACTATGCTTCTTAGGCCGAAACTGTTGAAAGTTGTTTAGCAAAACCTCATACAGAAAAT | CDR-1: SEQ ID NO: 239 |
| | TCATTTACTAACGTCTGGAAAGACGACAAAACTTTAGATCGTTACGCTAACTATGAGGGCTGTCTGTGGAATGCT | CDR-2: SEQ ID NO: 240 |
| | ACAGGCGTTGTGGTTTGTACTGGTGACGAAACTCAGTGTTACGGTACATGGGTTCCTATTGGCTTGCTATCCTGA | CDR-3: SEQ ID NO: 241 |
| | ATGAGGTGTGCTCTGAGTGCCGTCTGAGTGGCGTCTGAAGTGCCGTACTAACTCTGAGTACGGTGATACCACCT | |
| | A SEQ ID NO: 198 | |
| FLCTGF- | ATACCTATTGCCTACGGCGGCCGCTGGATTGTTATTACTCGCGGCCCAGCCGGCCATGGCCCAGGTGCAGCTGC | MAQVQLQESGGGSVQAGGSLKLSCSVSGYT |
| 1D1 | AGGAGTCTGGAGGAGGCTCGGTGCAGGCTGGAGGGTCTCTGAAACTCTCCTGTTCAGTCTCTGGATACACCTA | YSTKYMGWFRQAPGKEREGVAAIGTGGGRT |
| | CAGTACCAAGTACATGGGCTGGTTCCGCCAGGCTCCAGGGAAGGAGCGCGAGGGGGTCGCAGCTATTGGTAC | YYADSVKGRFTISLDNAKNTVYLQMNSLEPED |
| | TGGTGGTGGTAGGACATACTATGCCGACTCCGTGAAGGGCCGATTCACCATCTCCCTTGACAACGCCAAGAACA | TAMYYCAVDYTVWLLQPLDQSGYKYWGQG |
| | CGGTGTATCTGCAAATGAACAGCCTGGAGCCTGAGGATACTGCCATGTACTACTGTGCAGTAGATTACACTGTGT | TQVTVSS GPGGQ SEQ ID NO: 218 |
| | GGTTACTGCAACCCTTGGACCAATCTGGGTATAAGTACTGGGGCCAGGGGACCCAGGTCACCGTCTCCTCAGGC | CDR-1: SEQ ID NO: 242 |
| | CCGGGAGGCCAACACCATCACCACCATCATGGCGCATATCCGTATGATGTGCCGGACTATGCTTCTTAGGCCGAA | CDR-2: SEQ ID NO: 243 |
| | ACTGTTGAAAGTTGTTTAGCAAAACCTCATACAGAAAATTCATTTACTAACGTCTGGAAAGACGACAAAACTTTA | CDR-3: SEQ ID NO: 244 |
| | GATCGTTACGCTAACTATGAGGGCTGTCTGTGGAATGCTACAGGCGTTGTGGTTTTGTACTGGTGACGAAACTC | |
| | AGTGTTACGGTACATGGGTTCCTATTGGCTTGCTATCCCTGAAATGAAGGTGGTGCTCTGAGGTGGCGTTTCTGA | |
| | GGGGTGGGCGGCTTCTGGAGGGGTTGGGCGCGGT | |
| | SEQ ID NO: 199 | |
| FLCTGF-1E3 | ACCTTAATTGCCTACGGCGGCCGCTGGATTGTTATTACTCGCGGCCCAGCCGGCCATGGCCCAGGTGCAGCTGC | MAQVQLQESGGGSVQTGGSLRLSCAASGNT |
| | AGGAGTCTGGAGGAGGCTCGGTGCAGACTGGAGGGTCTCTGAGACTCTCCTGTGCAGCCTCCGGAAACACCT | YTDYIIGWLRQAPGKEREGVATIWSISGVTSY |
| | ATACAGACTACATTATTGGCTGGCTCCGCCAGGCTCCAGGGAAGGAGCGTGAGGGGGTCGCAACTATTTGGAG | ADSVKGRFSISQDNAKNTVYLQMNSLKPEDT |
| | TATAAGTGGTGTTACTTCGTACGCCGACTCCGTGAAGGGCCGATTCAGCATCTCCCAAGACAACGCCAAGAACA | AIYFCAIRPRWYVEYQYWGQGTQVTVSS |
| | CGGTGTATCTTCAAATGAACAGCCTGAAACCTGAGGACACGGCCATCTATTTCTGTGCAATTCGGCCACGTTGGT | GPGGQ SEQ ID NO: 219 |
| | ATGTTGAATACCAGTATTGGGGCCAGGGAACCCAGGTCACCGTCTCCTCAGGCCCGGGAGGCCAACACCATCA | CDR-1: SEQ ID NO: 245 |
| | CCACCATCATGGCGCATATCCGTATGATGTGCCGGACTATGCTTCTTAGGCCGAAACTGTTGAAAGTTGTTTAGCA | CDR-2: SEQ ID NO: 246 |
| | AAACCTCATACAGAAAATTCATTTACTAACGTCTGGAAAGACGACAAAACTTTAGATCGTTACGCTAACTATGAG | CDR-3: SEQ ID NO: 247 |
| | GGCTGTCTGTGGAATGCTACAGGCGTTGTGTTTTGTACTGTGACGAAACTCAGTGTTACGTACATGGGTTCCTAT | |
| | GGCTGCTATCCTGAAATGAGGTGGTGCTCTGAAGTGCCGTCTGAGGTTGGCGTTCTGAGGTGGCGTAACTTAAA | |
| | CCCTCCCTG SEQ ID NO: 200 | |
| FLCTGF-1E4 | CCCTATTTGCCTACGGCGGCCGCTGGATTGTTATTACTCGCGGCCCAGCCGGCCATGGCCCAGGTGCAGCTGCA | MAQVQLQESGGGSVQPGGSLRLSCTVSGYT |
| | GGAGTCTGGGGGAGGCTCGGTGCAGCCTGGAGGGTCTTTGAGACTCTCCTGTACAGTCTCTGGATACACTGGT | GGSDLMAWFRQAPGKEREGVAGIYGVAGV |
| | GGTAGCGACCTAATGGCCTGGTTCCGCCAGGCTCCAGGCAAGGAGCGCGAGGGGGTCGCAGGTATTTATGGT | TYYADSVKGRFTITRDKNTVHLQMNSLKPEDT |
| | GTCGCTGGTGTCACATATTATGCCGACTCCGTGAAGGGCCGATTCACCATCACCCGAGACAAGAACACGGTACA | AMYYCAVGRLGVVRSILRSNGV- |
| | TCTGCAAATGAACAGCCTGAAACCTGAGGACACTGCCATGTACTACTGTGCAGTAGGGCGGCTCGGTGTTGTAC | LLGPGDPGHRLLRPG SEQ ID NO: 220 |
| | GTTCGATCCTCCGCTCAAATGGGGTATAACTACTGGGGCCAGGGGACCCAGGTCACCGTCTCCTCAGGCCCGG | CDR-1: SEQ ID NO: 248 |
| | GAGGCCAACACCATCACCACCATCATGGCGCATATCCGTATGATGTGCCGGACTATGCTTCTTAGGCCGAAACTG | CDR-2: SEQ ID NO: 249 |
| | TTGAAAGTTGTTTAGCAAAACCTCATACAGAAAAATTCATTTACTACGTCTGGAAAGACGACAAACCTTTAGATC | CDR-3: SEQ ID NO: 250 |
| | GTTACGCTAACTATGAGGCCTGTCTGTGAAATGCTACAGGCGTGTGATGGTACTGTGACGACCTCAAGTACGGTA | |
| | CATTGTTCCTATGGCCTGCATTCTGATGAGGTGTGCTTGAGTGCCGTCCTAGGTGCGCA SEQ ID NO: 201 | |
| FLCTGF- | AAACCCTTATTGCCTACGGGCGGCCGCTGGATTGTTATTACTCGCGGCCCAGCCGGCCATGGCCCAGGTGCAGC | MAQVQLQESGGGSVQAGGSLRLSCTA |
| 1H9 | TGCAGGAGTCTGGGGGAGGCTCGGTACAGGCTGGAGGATCTCTGAGACTCTCCTGTACAGCCTCTGGATACAC | SGYTGDSDLMAWFRQAPGKEREGVA |
| | CGGTGATAGCGACTTAATGGCCTGGTTCCGCCAGGCTCCAGGCAAAGAGCGCGAGGGGGTCGCAGGTATTTAT | GIYGVAGVTYYADSVKGRFTITRDNTKN |
| | GGTGTCGCTGGTGTCACATATTATGCCGACTCCGTGAAGGGCCGATTCACCATCACCCGAGACAACACCAAGAA | TVHLQMNSLKPEDTAMYYCAVGRLGV |
| | CACGGTACATCTGCAAATGAACAGCCTGAAACCTGAGGACACTGCCATGTACTACTGTGCAGTAGGGCGGCTCG | VRSILRSNGYTYWGQGTQVTVSS |
| | GTGTTGTACGTTCGATCCTCCGCTCAAACGGGTATACCTACTGGGGCCAGGGGACCCAGGTCACCGTCTCCTCA | GPGGQ SEQ ID NO: 221 |
| | GGCCCGGGAGGCCAACACCATCACCACCATCATGGCGCATATCCGTATGATGTGCCGGACTATGCTTCTTAGGCC | CDR-1: SEQ ID NO: 251 |
| | GAAACTTGTGAAGTTGTTTAGCAAACCCTCAATACAGAAAAATTCATTTACAACCTCTCTGGGAAAGAACGGAC | CDR-2: SEQ ID NO: 252 |
| | AAACCTTTAAGATCGTACCGCTACATTAGAGGCTTTCTTGTGAAATGCCTACGGCGTTGTGTTGAACTGGTGACA | CDR-3: SEQ ID NO: 253 |
| | CCTAAGTTGACCGTATGGTCAATTGGCTGGCACATCTGAATAAGAGGGTTGGACTACTTGA SEQ ID NO: 202 | |
| FLCTGF- | CTAATTGCCTACGGGCGGCCGCTGGATTGTTATTACTCGCGGCCCAGCCGGCCATGGCCCAGGTGCAGCTGCAG | MAQVQLQESGGGSVQPGGSLRLSCTVSGYT |
| 2A11 | GAGTCTGGAGGAGGCTCGGTGCAGCCTGGAGGGTCTCTGAGACTCTCCTGTACAGTCTCTGGATACACTGGTG | GGNDLMAWFRQAPGKEREGVATVYGVAHV |
| | GTAACGACTTAATGGCCTGGTTCCGCCAGGCTCCAGGCAAGGAGCGCGAGGGGGTCGCAACTGTTTATGGTGT | TYYADSVKGRFTISQDKNTVHLQMNSLKPED |
| | CGCTCATGTCACATATTATGCCGACTCCGTGAAGGGCCGATTCACCATCTCCCAAGACAAGAACACGGTACATCT | TAMYYCAVGRLGLVRSVLRSNGYSYWGQGT |
| | CCAAATGAACAGCCTGAAACCTGAGGACACTGCCATGTACTACTGTGCAGTAGGGCGGCTCGGGCTTGTACGTT | QVTVSS GPGGQ SEQ ID NO: 222 |
| | CGGTCCTCCGCTCAAATGGGTATAGTTACTGGGGCCAGGGGACCCAGGTCACCGTCTCCTCAGGCCCGGGAGG | CDR-1: SEQ ID NO: 254 |
| | CCAACACCATCACCACCATCATGGCGCATATCCGTATGATGTGCCGGACTATGCTTCTTAGGCCGAAACTGTTGAA | CDR-2: SEQ ID NO: 255 |
| | AGTTGTTTAGCAAAACCTCATACAGAAAATTCATTTACTAACGTCTGGAAAGACGACAAAACTTTAGATCGTTAC | CDR-3: SEQ ID NO: 256 |
| | GCTAACTATGAGGGCTGTCTGTGGAATGCTACAGGCGTTGTGTTTGTACTGGTGACGAAACTCAGTGTTACGGT | |
| | ACATGGTCTATTGGCTGCTATCCCTGAAATGAGTGTGCTCTGAAGTGCGTCCTGAAGTTGCGTTCTGGGAGGTG | |
| | GGCGGGTATCTT SEQ ID NO: 203 | |
| FLCTGF-2F6 | TCCCTATTGCCTACGGCGGCCGCTGGATTGTTATTACTCGCGGCCCAGCCGGCCATGGCCCAGGTGCAGCTGCA | MAQVQLQESGGGSVQAGGSLRLSCAASGYT |
| | GGAGTCTGGGGGAGGCTCGGTGCAGGCTGGAGGATCTCTGAGACTCTCCTGTGCAGCCTCTGGATACACCGTC | VSSPYNYIGWFRQAPGKEREGVATINRDRLN |
| | AGTAGCCCCTACAACTACATCGGCTGGTTCCGCCAGGCTCCAGGGAAGGAGCGTGAGGGGGTCGCAACTATTA | TYYADSVKGRFTISQDTAKNTVYLQMNSLRPE |
| | ATCGTGATAGACTTAACACATACTACGCCGACTCCGTGAAGGGCCGATTCACCATCTCCCAAGACACCGCCAAGA | DTAIYYCAAAWTSWSTALTPAAYNYWGQGT |
| | ACACGGTGTATCTGCAAATGAACAGCCTGAGACCTGAGGACACGGCCATCTATTACTGTGCGGCAGCATGGACT | QVTVSS GPGGQ SEQ ID NO: 223 |
| | TCCTGGAGTACTGCCCTAACCCCGGCCGCGTATAACTACTGGGGCCAGGGGACCCAGGTCACCGTCTCCTCAGG | CDR-1: SEQ ID NO: 257 |
| | CCCGGGAGGCCAACACCATCACCACCATCATGGCGCATATCCGTATGATGTGCCGGACTATGCTTCTTAGGCCGA | CDR-2: SEQ ID NO: 258 |
| | AACTGTTGAAAGTTGTTTAGCAAAACCTCATACAGAAAATTCATTTACTAACGTCTGGAAAGACGACAAAACTTT | CDR-3: SEQ ID NO: 259 |
| | AGATCGTTACGCTAACTATGAGGGCTGTCTGTGGAATGCTACAGGCGTTGTGTTTGTACTGTGACGAAACTCAGT | |
| | GTACGTACATGGGTCTATGGCTGGCTATCTGAAATGAGGTGGTGCTCTGAAGGTGCCGTCTGAGGTGGCGGCTT | |
| | CTGAGTGC SEQ ID NO: 204 | |
| FLCTGF-3B2 | ACCTAATAGCCTACGGCGGCCGCTGGATTGTTATTACTCGCGGCCCAGCCGGCCATGGCCCAGGTGCAGCTG | MAQVQLQESGGGSVQPGGSLRLSCAASGDT |
| | CAGGAGTCTGGGGGAGGCTCGGTGCAGCCTGGAGGATCTCTAAGACTCTCCTGTGCAGCCTCTGGAGACACC | SIIEYMGWFHEAPGKERDGIATINVNDGNTY |
| | TCCATTATCGAATACATGGGCTGGTTCCACGAGGCTCCAGGAAAGGAGCGTGACGGTATCGCGACCATTAAT | YTDSVYGRFTISQDSAKKTIYLQMNSLKGDDT |
| | GTCAATGATGGTAACACATACTACACCGACTCCGTGTACGGCCGATTCACCATCTCCCAAGACAGCGCCAAGA | GIYTCAASPALDRGLSLDPSDYDYWGQGTQ |
| | AGACGATTTATCTGCAAATGAACAGCCTGAAAGGTGACGACACGGGCATCTATACCTGTGCAGCGTCTCCGG | VTVSS GPGGQ SEQ ID NO: 224 |
| | CGTTAGACCGCGGATTAAGTTTGGACCCAAGTGACTATGATTATTGGGGCCAGGGGACCCAGGTCACCGTCT | |
| | CCTCAGGCCCGGGAGGCCAACACCATCACCACCATCATGGCGCATATCCGTATGATGTGCCGGACTATGCTTC | CDR-1: SEQ ID NO: 260 |
| | TTAGGCCGAAACTGTTGAAAGTTGTTTAGCAAAACCTCATACAGAAAATTCATTTACTAACGTCTGGAAAGAC | CDR-2: SEQ ID NO: 261 |
| | GACAAAACTTTAGATCGTTACGCTAACTATGAGGGCTGTCTGTGGAATGCTACAGCGTTGTGTTTGTACTGCT | CDR-3: SEQ ID NO: 262 |
| | GACGAACTCAGTGTACGGTACATGGTTCTATTGGCTTGCTATCTGAATGAGGTGTGCTCTGAGATGCCGTCGT | |
| | GACGGATGGCGGCTC SEQ ID NO: 205 | |
| FLCTGF-3B5 | ATCCCTATTGCCTACGGCGGCCGCTGGATTGTTATTACTCGCGGCCCAGCCGGCCATGGCCCAGGTGCAGCTG | MAQVQLQESGGGSVQAGGSLRLSCAVSGYP |
| | CAGGAGTCTGGGGGAGGCTCGGTGCAGGCTGGAGGGTCTCTGAGACTCTCCTGTGCAGTCTCTGGATACCCC | RNRCITGWTRQAPGKGRELVSTITISGGRSYG |
| | AGGAACAGGTGCATCACGGGCTGGACCCGCCAGGCTCCAGGGAAGGGTCGCGAGTTGGTGTCGACTATTAC | NSVKGRFTISEDKDKNTVYLQMNSLKPEDTA |
| | AATTAGCGGTGGTCGCTCGTATGGAAACTCCGTGAAGGGCCGATTTACCATCTCCGAAGACAAGGACAAGAA | MYYCYIGLCGTSQQNYWGQGTQVTVSSG |
| | CACGGTGTATCTGCAAATGAATAGCCTGAAACCTGAGGACACGGCCATGTACTACTGTTACATAGGTCTATGT | SEQ ID NO: 225 |
| | GGTACGTCACAACAAAACTACTGGGGCCAGGGGACCCAGGTCACCGTCTCCTCAGGCCCGGGAGGCCAACA | CDR-1: SEQ ID NO: 263 |
| | CCATCACCACCATCATGGCGCATATCCGTATGATGTGCCGGACTATGCTTCTTAGGCCGAAACTGTGAAAGTG | CDR-2: SEQ ID NO: 264 |
| | TTTAGAAACCTCATCAGAAATTCATTTCTAACGCCTGGAAGACAACAACCTTGATCGTACGCTACATAAGGCG | CDR-3: SEQ ID NO: 265 |
| | TGTCTGTGATCTACAGGTTGTTGTACTGGTACAACTATGACGCATGGTCCATTGCTAAACTGAATAGGGGGCT | |
| | ATGTCTAAGGGCCTAGGGCTTTATCCTGTGGGGGAG SEQ ID NO: 206 | |
| FLCTGF- | AACCTATTGCCTACGGCGGCCGCTGGATTGTTATTACTCGCGGCCCAGCCGGCCATGGCCCAGGTGCAGCTGCA | MAQVQLQESGGGSVQAGGSLRLSCAASGNT |
| 3B11 | GGAGTCTGGGGGAGGCTCGGTGCAGGCTGGAGGGTCTCTGAGACTCTCCTGTGCAGCCTCCGGTAACACCTAT | YTDYVIGWLRQAPGKEREGVATIWSRSGTTS |
| | ACAGACTACGTTATTGGCTGGCTCCGCCAGGCTCCAGGGAAGGAGCGTGAGGGGGTCGCAACTATTTGGAGTA | YADSVKGRFSISQDNAKNTVYLQMNSLKPED |
| | GAAGTGGTACCACATCGTACGCCGACTCCGTAAAGGGCCGATTCAGCATCTCCCAAGACAACGCCAAGAACAC | TAIYFCAIRPRWHAEYKYWGQGTQVTVSS |
| | GGTGTATCTTCAAATGAACAGCCTGAAACCTGAGGACACGGCCATCTATTTCTGTGCGATTCGGCCACGTTGGC | GPGGQ SEQ ID NO: 226 |
| | ATGCTGAATATAAGTATTGGGGCCAGGGAACCCAGGTCACCGTCTCCTCAGGCCCGGGAGGCCAACACCATCAC | CDR-1: SEQ ID NO: 266 |
| | CACCATCATGGCGCATATCCGTATGATGTGCCGGACTATGCTTCTTAGGCCGAAACTGTTGAAAGTTGTTTAGCA | CDR-2: SEQ ID NO: 267 |
| | AAACCTCATACAGAAAATTCATTTACTAACGTCTGGAAAGACGACAAAACTTTAGATCGTTACGCTAACTATGAG | CDR-3: SEQ ID NO: 268 |
| | GGCTGTCTGTGGAATGCTACAGGCGTTGTGTTTGTACTGGTGACGAAACTCAGTGTTACGGTACATGGGTCTATG | |
| | GGCTGCTATCCTGAATGGAGGGGTGGTGGCTCTGAAGGTGGCGTCTGGAGGGTGGGCGGATCTGAGGGGGT | |
| | GGGCGGTACTTAAACATC SEQ ID NO: 207 | |
| FLCTGF- | TACCCCTATTGCCTACGGCGGCCGCTGGATTGTTATTACTCGCGGCCCAGCCGGCCATGGCCCAGGTGCAGCTG | MAQVQLQESGGGSVQAGGSLRLSCAASGYI |
| 3C12 | CAGGAGTCTGGGGGAGGCTCGGTGCAGGCTGGAGGGTCTCTGAGACTCTCCTGTGCAGCCTCTGGATACATCG | GSRYCMGWFRQAPGKDREGVAIINNFHLSA |
| | GCAGCCGCTACTGTATGGGCTGGTTCCGCCAGGCTCCAGGGAAGGACCGTGAGGGGGTCGCAATTATTAATAA | DRTTYADSVKGRFTISQDNAKNTVYLQMNSL |
| | TTTTCATTTGAGTGCTGATCGCACAACATACGCCGACTCCGTGAAGGGCCGATTCACCATCTCCCAAGACAACGC | KAEDTAIYYCAARGATYYDTCSVGSVGGYQY |
| | CAAGAATACGGTGTATCTACAAATGAACAGCCTGAAAGCTGAGGACACGGCCATCTATTACTGTGCGGCTCGGG | WGQGTQVTVSS GPGGQ SEQ ID NO: 227 |
| | GGGCGACTTACTATGACACGTGTTCCGTCGGTTCCGTCGGTGGGTATCAATACTGGGGCCAGGGGACCCAGGTC | CDR-1: SEQ ID NO: 269 |
| | ACCGTCTCCTCAGGCCCGGGAGGCCAACACCATCACCACCATCATGGCGCATATCCGTATGATGTGCCGGACTAT | CDR-2: SEQ ID NO: 270 |
| | GCTTCTTAGGCCGAAACTGTTGAAAGTTGTTTAGCAAAACCTCATACAGAAAATTCATTTACTAACGTCTGGAAA | CDR-3: SEQ ID NO: 271 |
| | GACGACAAAACTTTAGATCGTTACGCTAACTATGAGGGCTGTCTGTGAATGCTACAGGCGTTGTGTTTGTACTGA | |
| | TGACGAAACTCAGTGTACGGTACATGGCTCCTATGGCTGGCTATCCTGAAATGAAGTTGTGCTCTGAGGGTGGC | |
| | GTTCTGAGGTGGCC SEQ ID NO: 208 | |
| FLCTGF-3E1 | AACCCCCAATTTGGCCCTACGGCGGCCGCTGGATTGTTATTACTCGCGGCCCAGCCGGCCATGGCCCAGGTGCA | MAQVQLQESGGGSVQTGGSLRLSCAASGNT |
| | GCTGCAGGAGTCTGGGGGAGGCTCGGTGCAGACTGGAGGGTCTCTGAGACTCTCCTGTGCAGCCTCTGGAAA | YTDYIIGWLRQAPGKEREGVATIWSGSGITSY |
| | CACCTATACAGACTACATTATTGGCTGGCTCCGCCAGGCTCCAGGGAAGGAGCGTGAGGGGGTCGCAACTATTT | ADSVKGRFSISQDNVKNTVYLQMNSLKPEDT |
| | GGAGTGGAAGTGGTATTACATCGTACGCCGATTCCGTGAAGGGCCGATTCAGCATCTCCCAAGACAATGTCAAG | AIYFCALRPRWHIEYQYWGQGTQVTVSS |
| | AACACGGTGTATCTTCAAATGAACAGCCTGAAACCTGAGGACACGGCCATCTATTTCTGTGCGCTTCGGCCACG | GPGGQ SEQ ID NO: 228 |
| | TTGGCATATTGAATACCAATATTGGGGCCAGGGAACCCAGGTCACCGTCTCCTCAGGCCCGGGAGGCCAACACC | CDR-1: SEQ ID NO: 272 |
| | ATCACCACCATCATGGCGCATATCCGTATGATGTGCCGGACTATGCTTCTTAGGCCGAAACTGTTGAAAGTTGTTT | CDR-2: SEQ ID NO: 273 |
| | AGCAAAACCTCATACAGAAAATTCATTTACTAACGTCTGGAAAGACGACAAACTTTAGATCGTTACGCTAACTAT | CDR-3: SEQ ID NO: 274 |
| | GAGGCTGTCTGTGGAATGCTACAGGCGTTGTGTTTGTACTGTGACGAACTCATGTACGGTACATGGGTCTATGGC | |
| | TGCTATCCTGAAATGAGGTGTGCTCTGAAGTGGCGATCTGAGTGGCGTTCTGGAGGTTGGCGTACCTAC SEQ ID | |
| | NO: 209 | |
| FLCTGF- | CACCCTTATTGGGCCTTACGGCGGCCGCTGGATTGTTATTACTCGCGGCCCAGCCGGCCATGGCCCAGGTGCAG | MAQVQLQESGGGSVQAGGSLRLSCTASTDTF |
| 3E12 | CTGCAGGAGTCTGGGGGAGGCTCGGTGCAGGCTGGAGGGTCTCTGAGACTCTCCTGTACAGCCTCTACAGACA | SFSDYAVEWYRQAPGKTCEWVSSIFRTGNTD |
| | CCTTCAGTTTCAGTGACTATGCAGTGGAGTGGTACCGCCAGGCTCCAGGGAAGACGTGCGAATGGGTCTCAAG | YVDSVKGRFTISRDNAKATSYLQMNSLKPEDT |
| | TATTTTTCGTACTGGTAACACAGATTATGTCGACTCTGTGAAGGGCCGATTCACTATCTCCCGAGACAACGCCAA | AVYYCLIGSSGCQRTQGTQVTVSS GPGGQ |
| | GGCGACGAGCTATCTGCAAATGAACAGCCTGAAACCTGAGGACACGGCCGTGTATTATTGTCTGATAGGGTCGT | SEQ ID NO: 229 |
| | CCGGTTGTCAACGAACCCAGGGGACCCAGGTCACCGTCTCCTCAGGCCCGGGAGGCCAACACCATCACCACCA | CDR-1: SEQ ID NO: 275 |
| | TCATGGCGCATATCCGTATGATGTGCCGGACTATGCTTCTTAGGCCGAAACTGTTGAAAGTTGTTAGCAAAACCC | CDR-2: SEQ ID NO: 276 |
| | TCAATACAGAAAAATTCATTTACTAACGTTCTGGAAAGACGACAAACCTTTAGAATCGGTTACCGCTAACTATGA | CDR-3: SEQ ID NO: 277 |
| | GGGCTGTCTGTGGAATTGCCTACAGGCGTTGGTGATTGTACTGGTGACGACTCAGTGTAACGGTAATTGGTTCC | |
| | TATTGGCTGCTATCCTTGAAATGAGGGTGTGCCTAAGGTGGCGTCTGAGGTGGCGATTCCATAGTGGCCACCTC | |
| | SEQ ID NO: 210 | |
| FLCTGF- | AACCCTATTGCCTACGGCGGCCGCTGGATTGTTATTACTCGCGGCCCAGCCGGCCATGGCCCAGGTGCAGCTGC | MAQVQLQESGGGSVESGGSLRLSCAASGYT |
| 3G2 | AGGAGTCTGGGGGAGGCTCGGTGGAGTCTGGGGGGTCTCTGAGACTCTCCTGTGCAGCCTCTGGATACACCCA | HSSYCMGWYRQAPGKERELVSFIFSEGSTSYT |
| | CAGTAGCTACTGCATGGGCTGGTACCGCCAGGCTCCAGGGAAGGAGCGCGAGTTGGTCTCATTTATTTTTAGTG | DSVKGRFTISQDNAKNTVYLQMNSLKPEDTAI |
| | AGGGTAGCACATCCTATACAGACTCCGTGAAGGGCCGATTCACCATCTCCCAAGACAACGCCAAGAACACGGTG | YYCAADYGTPKYGDNWCDFNYWGQGTQVT |
| | TATCTGCAAATGAACAGCCTGAAACCCGAGGACACGGCCATCTATTACTGTGCGGCAGATTATGGAACCCCTAAA | VSS GPGGQ SEQ ID NO: 230 |
| | TACGGTGATAACTGGTGTGACTTTAATTACTGGGGCCAGGGGACCCAGGTCACCGTCTCCTCAGGCCCGGGAG | CDR-1: SEQ ID NO: 278 |
| | GCCAACACCATCACCACCATCATGGCGCATATCCGTATGATGTGCCGGACTATGCTTCTTAGGCCGAAACTGTTGA | CDR-2: SEQ ID NO: 279 |
| | AAGTTGTTTAGCAAAACCTCATACAGAAAATTCATTTACTAACGTCTGGAAAGACGACAAAACTTTAGATCGTTA | CDR-3: SEQ ID NO: 280 |
| | CGCTAACTATGAGGGCTGTCTGTGGAATGCTACAGGCGTTGTGTTTGTACTGTGACGAAACTCAGTGTTACGTAC | |
| | ATGGGTCTATGGGCTGCTATCCCTGAATGAGTGTGCTCTGAAGTGCGATTCTGAAGGTGGCGTCTGGAGGGGTG | |
| | GGGCGGTACACTA SEQ ID NO: 211 | |
| FLCTGF- | AACCTATTGCCTACGGCGGCCGCTGGATTGTTATTACTCGCGGCCCAGCCGGCCATGGCCCAGGTGCAGCTGCA | MAQVQLQESGGGSVQAGGSLRLSCTASGYT |
| 3G8 | GGAGTCTGGGGGAGGCTCGGTGCAGGCCGGAGGGTCTCTGAGACTCTCCTGTACAGCCTCTGGATACACCTGC | CSRYCMGWYRQALGKERELVSFIGSDGITHY |
| | AGTAGATACTGTATGGGCTGGTATCGCCAGGCTCTTGGGAAGGAGCGCGAATTGGTCTCATTTATTGGTAGTGAT | TDSVKGRFTVSLDNAKNTMYLQMNSLKPEDT |
| | GGTATCACACACTATACAGACTCCGTGAAGGGCCGATTCACCGTCTCCCTAGACAACGCCAAGAACACGATGTAT | AIYYCAGGYGSGGDCSFGSWGRGTQVTVSS |
| | CTGCAAATGAACAGCCTGAAACCTGAGGACACGGCCATCTATTACTGTGCGGGGGGTTACGGTAGTGGTGGTG | GPGGQ SEQ ID NO: 231 |
| | ACTGCAGTTTTGGTAGCTGGGGCCGGGGGACCCAGGTCACCGTCTCCTCAGGCCCGGGAGGCCAACACCATC | CDR-1: SEQ ID NO: 281 |
| | ACCACCATCATGGCGCATATCCGTATGATGTGCCGGACTATGCTTCTTAGGCCGAAACTGTTGAAAGTTGTTTAGC | |
| | AAAACCTCATACAGAAAATTCATTTACTAACGTCTGGAAAGACGACAAAACTTTAGATCGTTACGCTAACTATGA | CDR-2: SEQ ID NO: 282 |
| | GGGCTGTCTGTGGAATGCTACAGGCGTTGTGGTTTGTACTGGTGACGAAACTCAGTGTACGGTACATGGGTTCT | CDR-3: SEQ ID NO: 283 |
| | ATGGCTGCTATCCCTGAATGAGGGTGGGTGCTCTGAGTGCGTCTGGAGGGTGGGCGGTCTGGAGGGTGGGCG | |
| | GTACTAACCTCCTGGGAG SEQ ID NO: 212 | |
| FLCTGF- | AACCCTATTGCCTACGGCGGCCGCTGGATTGTTATTACTCGCGGCCCAGCCGGCCATGGCCCAGGTGCAGCTGC | MAQVQLQESGGGSVQAGGSLRLSCAASGYT |
| 3H2 | AGGAGTCTGGAGGAGGCTCGGTGCAGGCTGGAGGGTCTCTGAGACTCTCCTGTGCAGCCTCTGGATACACCTA | YSTYCMGWFRQAPGKEREGVACINSGDGSA |
| | CAGTACGTACTGCATGGGCTGGTTCCGCCAGGCTCCAGGGAAGGAACGTGAGGGGGTCGCATGTATTAATAGT | YYADSVKGRFTISQDNAKNTVYLQMNSLKPE |
| | GGTGACGGTAGCGCATACTACGCCGACTCCGTGAAGGGCCGATTCACCATCTCCCAAGACAACGCCAAGAACA | DTAMYYCAAWDGGYCHSAIWVPGYKYRGQ |
| | CGGTGTATCTGCAAATGAACAGCCTGAAACCTGAGGACACGGCCATGTATTACTGTGCGGCTTGGGATGGAGGT | GTQVTVSS GPGGQ SEQ ID NO: 232 |
| | TACTGTCACAGCGCGATTTGGGTCCCTGGGTATAAGTACCGGGGCCAGGGGACCCAGGTCACCGTCTCCTCAG | CDR-1: SEQ ID NO: 284 |
| | GCCCGGGAGGCCAACACCATCACCACCATCATGGCGCATATCCGTATGATGTGCCGGACTATGCTTCTTAGGCCG | CDR-2: SEQ ID NO: 285 |
| | AAACTGTTGAAAGTTGTTTAGCAAAACCTCATACAGAAAATTCATTTACTAACGTCTGGAAAGACGACAAAACTT | CDR-3: SEQ ID NO: 286 |
| | TAGATCGTTACGCTAACTATGAGGGCTGTCTGTGAATGCTACAGGCGTTGTGATTTGTACTGTGACGAAACTCAG | |
| | TGTACCGTACATGGCTCTATGGCTGCTATCTGAATGAGGTGTGCTCTGAAGGTGGCGTCCTGAAGTGGCGGATTC | |
| | TGAAGTGAGC SEQ ID NO: 213 | |

### Example 12: Anti-Inflammatory Effect

In ALS, astrocytes change their shape and molecular expression patterns and become reactive. Reactive astrocytes lose their beneficial functions and gain detrimental roles. The pathogenicity of these astroglial cells resides in their proinflammatory and profibrotic properties. Early in the disease changes in innate immunity leads to increased production and release of significant levels of cytokines. Recent evidence show that interleukin-6 (IL-6) and CTGF are elevated in ALS and mediate the activation of astrocytes and microglia, as well as increase production of serum amyloid and free radicals.

To test for the anti-inflammatory property of the VHH fragments, the expression of pSTAT3 in healthy human astroglial cells treated with IL-6 and IL-6R in the absence or presence of the VHH fragments were analyzed by western blotting. Tocilizumab that targets IL-6R was used as positive control.

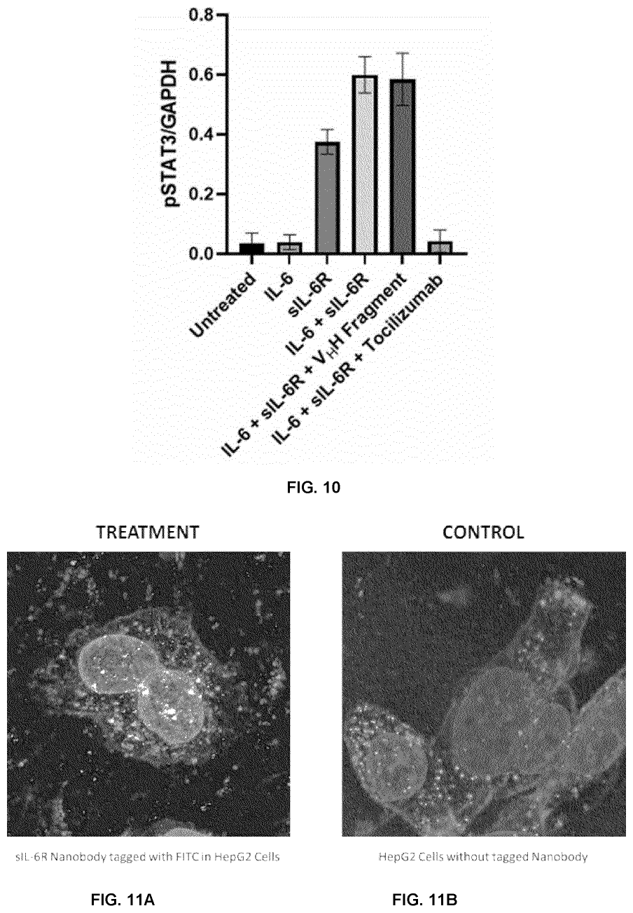

As shown in the FIG. 10, the anti-sIL-6R fragment reduced the IL-6 stimulated expression of pSTAT3, a cellular inflammatory marker, which indicates a potential to suppress inflammation.

### Example 13: Intracellular Delivery

The intracellular delivery of the VHH fragments was investigated. The fragments were labelled with a dye FITC that made it easy to track their movement. Astroglia cells exposed to the labelled fragments were visualized with confocal microscopes after 24h of exposure. The images in FIG. 11 show that the fragments entered the cells.

### Example 14: Delivery into the Brain

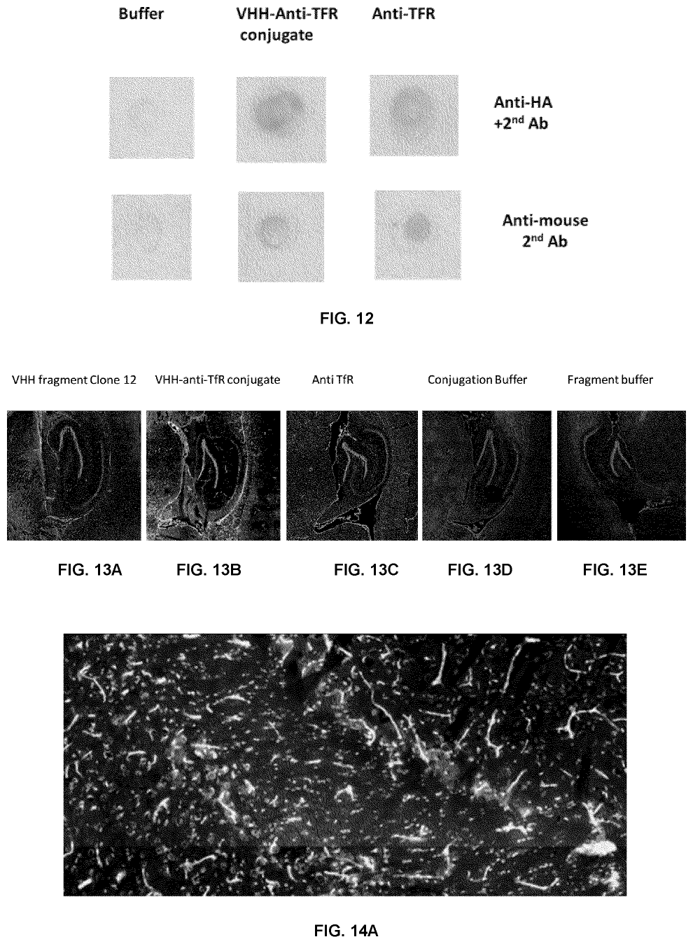

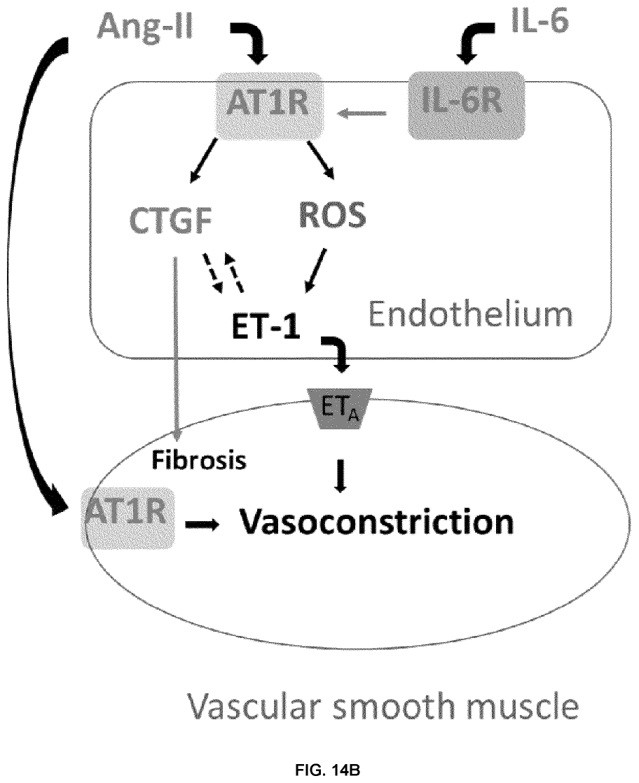

The ability of the VHH fragments to cross the blood-brain barrier and enter the brain tissue from circulation was tested by examining brain tissues 4 hours after the fragments or their conjugates with anti-transferrin receptors were injected into rats. Images were taken in the area around the hippocampus using the shape and size of the dentate gyrus as a reliable marker to allow samples to be directly compared. An anti-HA antibody was used to identify presence of VHH fragment in brain sections. An anti-mouse antibody was used to detect the mouse anti-transferrin receptor (anti-TfR) antibody. A dot blot analysis of the cerebrospinal fluid (CSF) confirmed the presence of the conjugate consistent with ability to cross the blood-brain barrier (FIG. 12). As shown in FIG. 13, the anti-IL-6R fragment and anti-TfR were detected in the hippocampal regions of rats injected with the anti sIL-6R, the conjugate and anti-TfR, indicating that they crossed the blood-brain barrier. No VHH fragment or anti-TfR antibody was detected in vehicles (conjugation and fragment preparation buffers). All sections showed clear nuclear staining.

### Example 15: Cerebrovascular Effect

Immunobiological analysis of the brain tissues following the injection of the conjugated fragments also showed the localization of the conjugate in cerebral micro vessels (FIG. 14A). This indicates the potential to alter cerebrovascular functions. Since a dysregulated IL-6-CTGF signaling would adversely affect cerebrovascular function as illustrated in FIG. 14B, the affinity of the VHH fragments for these vessels has the potential to counteract the adverse vascular effects of these cytokines. This can include reversing the vasoconstrictor effect of these cytokines, resulting in vasodilation with the potential to increase cerebral blood flow.

Although the invention has been described with reference to the above examples, it will be understood that modifications and variations are encompassed within the spirit and scope of the invention. Accordingly, the invention is limited only by the following claims.

What is claimed is:

1. A multispecific binding protein comprising:
a) a first binding protein that specifically binds to human soluble interleurkin-6 receptor (SIL-6R); and
b) a second binding protein that is operatively linked to the first binding protein and that specifically binds to human connective tissue growth factor (CTGF).

2. The multispecific binding protein of claim 1, wherein the first binding protein and the second binding protein comprise a VHH, humanized VHH, VH, single domain antibody, or variable new antigen receptor (VNAR).

3. The multispecific binding protein of claim 1, wherein the multispecific binding protein comprises an antibody or an antigen-binding fragment thereof, a chimeric antibody, a humanized antibody, or a camelid antibody.

4. The multispecific binding protein of claim 1, further comprising an anti-transferrin receptor binding protein.

5. The multispecific binding protein of claim 1, wherein the multispecific binding protein is mutated by polycationic resurfacing.

6. A binding protein that specifically binds to human soluble interleurkin-6 receptor (sIL-6R) comprising complementarity-determining regions (CDRs) as set forth in SEQ ID NOs: 2-4, SEQ ID NOs: 6-8, SEQ ID NOs: 10-12, SEQ ID NOs: 14-16, SEQ ID NOs: 18-20, SEQ ID NOs: 22-24, SEQ ID NOs: 26-28, SEQ ID NOs: 30-32, SEQ ID NOs: 34-36, SEQ ID NOs: 38-40, SEQ ID NOs: 42-44, SEQ ID NOs: 46-48, SEQ ID NOs: 50-52, SEQ ID NOs: 54-56, SEQ ID NOs: 58-60, SEQ ID NOs: 62-64, SEQ ID NOs: 66-68, or SEQ ID NO: 138-194, or a binding protein having CDRs with 80% or greater identity to the CDRs of the binding protein comprising SEQ ID NOs: 2-4, SEQ ID NOs: 6-8, SEQ ID NOs: 10-12, SEQ ID NOs: 14-16, SEQ ID NOs: 18-20, SEQ ID NOs: 22-24, SEQ ID NOs: 26-28, SEQ ID NOs: 30-32, SEQ ID NOs: 34-36, SEQ ID NOs: 38-40, SEQ ID NOs: 42-44, SEQ ID NOS: 46-48, SEQ ID NOs: 50-52, SEQ ID NOs: 54-56, SEQ ID NOs: 58-60, SEQ ID NOs: 62-64, SEQ ID NOs: 66-68, or SEQ ID NO: 138-194 and the antigen binding specificity thereof.

7. The binding protein of claim 6, wherein the binding protein has a better binding affinity to soluble IL-6R as compared to a binding affinity to membrane bound IL-6R.

8. A binding protein that specifically binds to human connective tissue growth factor (CTGF) comprising CDRs as set forth in SEQ ID NOs: 70-72, SEQ ID NOs: 74-76, SEQ ID NOs: 78-80, SEQ ID NOs: 82-84, SEQ ID NOs: 86-88, SEQ ID NOs: 90-92, SEQ ID NOS: 233-286, or SEQ ID NOs: 291-293 or a binding protein having CDRs with 80% or greater identity to the CDRs of the binding protein comprising SEQ ID NOs: 70-72, SEQ ID NOs: 74-76, SEQ ID NOs: 78-80, SEQ ID NOs: 82-84, SEQ ID NOs: 86-88, SEQ ID NOs: 90-92, SEQ ID NOs: 233-286, or SEQ ID NOs: 291-293 and the antigen binding specificity thereof.

9. The binding protein of claim 7 or 9, wherein the binding protein comprises a VHH, humanized VHH, VH, single domain antibody, or variable new antigen receptor (VNAR).

10. The binding protein of claim 9, wherein the binding protein comprises an antibody or an antigen-binding fragment thereof, a chimeric antibody, a humanized antibody, or a camelid antibody.

11. The binding protein of claim 4, wherein the VHH comprises an amino acid sequence of SEQ ID NO:1, SEQ ID NO:5, SEQ ID NO:9, SEQ ID NO: 13, SEQ ID NO: 17, SEQ ID NO:21, SEQ ID NO:25, SEQ ID NO:29, SEQ ID NO:33, SEQ ID NO:37, SEQ ID NO:41, SEQ ID NO:45, SEQ ID NO:49, SEQ ID NO:53, SEQ ID NO:57, SEQ ID NO:61, SEQ ID NO:65, SEQ ID NO:69, SEQ ID NO:73, SEQ ID NO:77, SEQ ID NO: 81, SEQ ID NO:85, SEQ ID NO:89, SEQ ID NO: 119-137, or SEQ ID NO:214-232.

12. The binding protein of claim 4, wherein the binding protein is conjugated to an anti-transferrin receptor antibody.

13. The binding protein of claim 4, wherein the antibody is mutated by polycationic resurfacing.

14. A multispecific binding protein that specifically binds to human sIL-6R and to human CTGF comprising a first set of three CDRs as set forth in:
SEQ ID NOs: 2-4, SEQ ID NOs: 6-8, SEQ ID NOs: 10-12, SEQ ID NOs: 14-16, SEQ ID NOs: 18-20, SEQ ID NOs: 22-24, SEQ ID NOs: 26-28, SEQ ID NOs: 30-32, SEQ ID NOs: 34-36, SEQ ID NOs: 38-40, SEQ ID NOs: 42-44, SEQ ID NOs: 46-48, SEQ ID NOs: 50-52, SEQ ID NOs: 54-56, SEQ ID NOs: 58-60, SEQ ID NOs: 62-64, SEQ ID NOs: 66-68, or SEQ ID NO: 138-194; and a second set of three CDRs as set forth in SEQ ID NOs: 70-72, SEQ ID NOs: 74-76, SEQ ID NOs: 78-80, SEQ ID NOs: 82-84, SEQ ID NOs: 86-88, SEQ ID NOs: 90-92, SEQ ID NOs: 233-286, or SEQ ID NOs: 291-293.

15. The multispecific binding protein of claim 14, wherein the multispecific binding protein comprises a first amino acid sequences selected from SEQ ID NO:1, SEQ ID NO:5, SEQ ID NO: 9, SEQ ID NO: 13, SEQ ID NO: 17, SEQ ID NO:21, SEQ ID NO:25, SEQ ID NO:29, SEQ ID NO: 33, SEQ ID NO:37, SEQ ID NO:41, SEQ ID NO:45, SEQ ID NO:49, SEQ ID NO:53, SEQ ID NO: 57, SEQ ID NO:61, SEQ ID NO:65, and SEQ ID NO: 119-137 and a second amino acid sequence selected from SEQ ID NO:69, SEQ ID NO:73, SEQ ID NO:77, SEQ ID NO:81, SEQ ID NO: 85, SEQ ID NO:89, and SEQ ID NO:214-232.

16. The multispecific binding protein of claim 14, wherein the multispecific binding protein has a better binding affinity to soluble IL-6R as compared to a binding affinity to membrane bound IL-6R.

17. The multispecific binding protein of claim 14, wherein the multispecific binding protein is conjugated to an anti-transferrin receptor antibody.

18. The multispecific binding protein of claim 14, wherein the multispecific binding protein is mutated by polycationic resurfacing.

19. The multispecific binding protein of claim 14, wherein the multispecific binding protein comprises a VHH, humanized VHH, VH, single domain antibody, or variable new antigen receptor (VNAR).

20. The multispecific binding protein of claim 14, wherein the multispecific binding protein comprises an antibody or an antigen-binding fragment thereof, a chimeric antibody, a humanized antibody, or a camelid antibody.

21. An isolated nucleic acid molecule encoding the binding protein of any one of claims 6-13 or the multispecific binding protein of any one of claims 1-5 and 14-20, or the fusion protein of any one of claim 16-20.

22. An expression vector comprising any one of the isolated nucleic acid molecules of claim 21.

23. An isolated host cell comprising any one of the expression vectors of claim 22.

24. An isolated amino acid sequence as set forth in SEQ ID NOs: 1-92 and SEQ ID NO: 100-286.

25. A pharmaceutical composition comprising one or more binding proteins of any one of claims 6-13 and/or one or more multispecific binding proteins of any one of claims 1-5 and 14-20, and a pharmaceutically acceptable carrier.

26. A method of treating a neurological disease or condition in a subject comprising administering to the subject a therapeutically effective amount of any one of the binding peptides of any one of claims 6-13, any one of the multispecific binding protein of any one of claims 1-5 and 14-20, or the pharmaceutical composition of claim 25,
thereby treating the neurological disease or condition in the subject.

27. The method of claim 26, wherein the neurological disease or condition is an inflammatory neurological disease or condition.

28. The method of claim 26, wherein the neurological disease or condition is amyotrophic lateral sclerosis (ALS), Parkinson disease, multiple sclerosis, Alzheimer's disease or Huntington's disease.

29. The method of claim 26, wherein administering comprises intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal, intrasternal, oral, sublingual buccal or nasal administrations, infusion, inhalation, or nebulization.

30. The method of claim 26, wherein administering comprises crossing blood brain barrier and delivering to intracellular space enter the brain hippocampal region.

31. The method of claim 26, wherein the treating the neurological disease comprises improving glial-neuronal communication and retarding neurodegenerative process.

32. The method of claim 26, wherein the treating the neurological disease comprises reducing cerebral inflammation, inhibiting cerebral fibrosis and/or improving cerebral vascular function.

33. The method of claim 32, wherein reducing inflammation comprises inhibiting reactive cells production of inflammatory cytokines and/or free radicals.

34. The method of claim 33, wherein inflammatory cytokines comprise IL-6 and monocyte chemoattractant protein-1 (MCP-1).

35. The method of claim 32, wherein reducing inflammation comprises reducing IL-6 levels.

36. The method of claim 35, wherein reducing IL-6 levels comprises suppressing pSTAT3 expression in healthy astroglial cells.

37. The method of claim 33, wherein reactive cells comprise non neuronal cells selected from the group consisting of astrocytes, microglia, macrophages and T lymphocytes.

38. The method of claim 32, wherein inhibiting cerebral fibrosis comprises inhibiting reactive cells production of serum amyloid.

39. The method of claim 38, wherein reactive cells comprise non neuronal cells selected from the group consisting of astrocytes, microglia, macrophages and T lymphocytes.

40. The method of claim 32, wherein improving cerebral vascular function comprises inhibiting cytokine-induced vasoconstriction, inducing vasodilation and/or increasing cerebral blood flow.

41. The method of claim 40, wherein inhibiting cytokine-induced vasoconstriction comprises inhibiting IL-6/AT1R mediated vasoconstriction of cerebral micro vessels.

42. A method of treating a neurological disease or condition in a subject comprising administering to the subject a therapeutically effective amount of a multispecific binding protein comprising a first binding protein that specifically binds to human sIL-6R operatively linked to a second binding protein that specifically binds to human CTGF, thereby treating the neurological disease.

43. The method of claim 42, wherein the first binding protein and the second binding protein are a VHH humanized VHH, VH, single domain antibody, or variable new antigen receptor (VNAR).

44. The method of claim 42, wherein the multispecific binding protein further comprises an anti-transferrin receptor binding protein.

45. An isolated variable domain of a single heavy chain antibody (VHH) that specifically binds to human sIL-6R,
wherein the VHH comprises CDRs as set forth in SEQ ID NOs: 2-4, SEQ ID NOS: 6-8, SEQ ID NOs: 10-12, SEQ ID NOs: 14-16, SEQ ID NOs: 18-20, SEQ ID NOs: 22-24, SEQ ID NOs: 26-28, SEQ ID NOs: 30-32, SEQ ID NOs: 34-36, SEQ ID NOs: 38-40, SEQ ID NOs: 42-44, SEQ ID NOs: 46-48, SEQ ID NOs: 50-52, SEQ ID NOs: 54-56, SEQ ID NOs: 58-60, SEQ ID NOs: 62-64, SEQ ID NOs: 66-68, or SEQ ID NO: 138-194, or a binding protein having CDRs with 80% or greater identity to the CDRs of the binding protein comprising SEQ ID NOs: 2-4, SEQ ID NOs: 6-8, SEQ ID NOs: 10-12, SEQ ID NOs: 14-16, SEQ ID NOs: 18-20, SEQ ID NOs: 22-24, SEQ ID NOs: 26-28, SEQ ID NOs: 30-32, SEQ ID NOs: 34-36, SEQ ID NOs: 38-40, SEQ ID NOs: 42-44, SEQ ID NOs: 46-48, SEQ ID NOs: 50-52, SEQ ID NOs: 54-56, SEQ ID NOs: 58-60, SEQ ID NOs: 62-64, SEQ ID NOs: 66-68, or SEQ ID NO: 138-194 and the antigen binding specificity thereof.

46. An isolated variable domain of a single heavy chain antibody (VHH) that specifically binds to human CTGF comprising CDRs as set forth in SEQ ID NOs: 70-72, SEQ ID NOs: 74-76, SEQ ID NOs: 78-80, SEQ ID NOs: 82-84, SEQ ID NOs: 86-88, SEQ ID NOs: 90-92, SEQ ID NOs: 233-286, or SEQ ID NOs: 291-293 or a binding protein having CDRs with 80% or greater identity to the CDRs of the binding protein comprising SEQ ID NOs: 70-72, SEQ ID NOs: 74-76, SEQ ID NOs: 78-80, SEQ ID NOs: 82-84, SEQ ID NOs: 86-88, SEQ ID NOs: 90-92, SEQ ID NOs: 233-286, or SEQ ID NOs: 291-293 and the antigen binding specificity thereof.


SEQUENCE LISTING (293 sequences)

SEQ ID NO: 1 - protein, 141 aa, synthetic construct
MAESGGGSVQAGGSLTLSCVGYGSKCMGWFRQAPGKEREGVAGITTDSGITYYADSVKGRFIISQDKVKNTVYLEMSSLKPEDTAMYYCAAHQNKLGSCGNWARYNEWGQGTQVTVSSGPGGQHHHHHHGAYPYDVPDYAS

SEQ ID NO: 2 - protein, 5 aa, synthetic construct
GWFRQ

SEQ ID NO: 3 - protein, 16 aa, synthetic construct
DSGITYYADSVKGRFI

SEQ ID NO: 4 - protein, 21 aa, synthetic construct
AHQNKLGSCGNWARYNEWGQG

SEQ ID NO: 5 - protein, 146 aa, synthetic construct
MAEVQLVESGGGSVQAGGSLTLSCVGYGSKCMGWFRQAPGKEREGVAGITTDSGITYYADSVKGRFIISQDKVKNTVYLEMSSLKPEDTAMYYCAAHQNKLGSCGNWARYNEWGQGTQVTVSSGPGGQHHHHHHGAYPYDVPDYAS

SEQ ID NO: 6 - protein, 5 aa, synthetic construct
GSKCM

SEQ ID NO: 7 - protein, 16 aa, synthetic construct
AGITTDSGITYYADSV

SEQ ID NO: 8 - protein, 19 aa, synthetic construct
YCAAHQNKLGSCGNWARYN

SEQ ID NO: 9 - protein, 146 aa, synthetic construct
MAESGGGSVLAGASLRLSCAASGFPSSGKCMGWFRQTPGKEREGVATINSRSGAIYYADSVKGRFTVSQDNASNTVYLQMNSLEPEDTAVYYCAARPTYVDYACSATPNYMDWGQGTQVTVSSGPGGQHHHHHHGAYPYDVPDYAS

SEQ ID NO: 10 - protein, 5 aa, synthetic construct
GKCMG

SEQ ID NO: 11 - protein, 16 aa, synthetic construct
TINSRSGAIYYADSVK

SEQ ID NO: 12 - protein, 19 aa, synthetic construct
CAARPTYVDYACSATPNYM

SEQ ID NO: 13 - protein, 145 aa, synthetic construct
MAEVQLVESGGGLVQPGGSLRLSCAATGFSFSSSYMNWVRQAPGKGLEWVSGINIEGSQTYYSDSAKGRFTISRDNAKSTVFLQMNSLKSEDTALYYCAPIWYCTNVVSCTYWGQGTQVTVCGPGGQHHHHHHGAYPYDVPDYAS

SEQ ID NO: 14 - protein, 5 aa, synthetic construct
GFSFS

SEQ ID NO: 15 - protein, 16 aa, synthetic construct
LEWVSGINIEGSQTYY

SEQ ID NO: 16 - protein, 19 aa, synthetic construct
TALYYCAPIWYCTNVVSCT

SEQ ID NO: 17 - protein, 143 aa, synthetic construct
  VARIANT 2: note = Any amino acid
MXEVQLVESGGGLVQPGGSLRLSCAASGFAFSSYYMSWVRQAPGKGLEWVSGIWHDGSNTYYAGSVKGRFTISGDNAKNTVYLQMNNLKPEDTAVYYCATAPWSSYGDNYWGQGTQVTVCGPGGQHHHHHHGAYPYDVPDYAS

SEQ ID NO: 18 - protein, 5 aa, synthetic construct
GFAFS

SEQ ID NO: 19 - protein, 16 aa, synthetic construct
LEWVSGIWHDGSNTYY

SEQ ID NO: 20 - protein, 20 aa, synthetic construct
TAVYYCATAPWSSYGDNYWG

SEQ ID NO: 21 - protein, 151 aa, synthetic construct
  VARIANT 1: note = Any amino acid
  VARIANT 2: note = Any amino acid
  VARIANT 3: note = Any amino acid
  VARIANT 4: note = Any amino acid
  VARIANT 5: note = Any amino acid
  VARIANT 6: note = Any amino acid
  VARIANT 7: note = Any amino acid
  VARIANT 8: note = Any amino acid
XXXXXXXXMAEVQPGGSLRLSCAASGFTFSDYDMSWVRQAPGKGLEWVSNVGSGGGSTYYTDSVAGRFAISRDDAENTLYLQMNNLKPEDTAVYYCATGRNPATPGWVPPALYEYNYWGQGTQVTVSSGPGGQHHHHHHGAYPYDVPDYAS

SEQ ID NO: 22 - protein, 5 aa, synthetic construct
GFTFS

SEQ ID NO: 23 - protein, 16 aa, synthetic construct
LEWVSNVGSGGGSTYY

SEQ ID NO: 24 - protein, 18 aa, synthetic construct
TAVYYCATGRNPATPGWV

SEQ ID NO: 25 - protein, 141 aa, synthetic construct
  VARIANT 2: note = Any amino acid
  VARIANT 3: note = Any amino acid
MXXVQLVESGGGLVQPGGSLRLSCAASGFTFSIDAMSWVRQAPGKGLEWVSAINAGGSTYYADSVKGRFTISRDNAKNTLYLQLNSLKTEDTAMYYCTNYNYGLNTLAGQGTQVTVSSGPGGQHHHHHHGAYPYDVPDYAS

SEQ ID NO: 26 - protein, 5 aa, synthetic construct
GFTFS

SEQ ID NO: 27 - protein, 15 aa, synthetic construct
LEWVSAINAGGSTYY

SEQ ID NO: 28 - protein, 19 aa, synthetic construct
TAMYYCTNYNYGLNTLAGQ

SEQ ID NO: 29 - protein, 147 aa, synthetic construct
MAEVQLVESGGGLVQPGGSLSVSCAASGFTFSAYYMSWVRQAPGKGLEWVSGINMDGSNTYYADSVKGRFTISRDNAKNTLYLQMNSLKSEDTARYYCAAPYGGNWYPTTSYNYWGQGTQVTVCGPGGQHHHHHHGAYPYDVPDYAS

SEQ ID NO: 30 - protein, 5 aa, synthetic construct
GFTFS

SEQ ID NO: 31 - protein, 16 aa, synthetic construct
LEWVSGINMDGSNTYY

SEQ ID NO: 32 - protein, 19 aa, synthetic construct
TARYYCAAPYGGNWYPTTS

SEQ ID NO: 33 - protein, 141 aa, synthetic construct
MAEVQLVESGGDLVQPEESLSVSCAAAGFTFSGYFMSWVRQAPGKGLQWVSGINTDGSKTYYTDSVKGRFTISRDNAKNTLYLQMNSLKSEDTALYYCVVDRYGGGYWGQGTQVTVSSGPGGQHHHHHHGAYPYDVPDYAS

SEQ ID NO: 34 - protein, 5 aa, synthetic construct
GFTFS

SEQ ID NO: 35 - protein, 16 aa, synthetic construct
LQWVSGINTDGSKTYY

SEQ ID NO: 36 - protein, 20 aa, synthetic construct
TALYYCVVDRYGGGYWGQGT

SEQ ID NO: 37 - protein, 151 aa, synthetic construct
  VARIANT 1: note = Any amino acid
XADVQLVESGGGLVQPGGSLTLSCAASGFTFSTYDMTWVRQAPGKGLEWVSRINSGGDTTYYADSVKGRFAVSRDIAKNTLYLQMNSLKPEDTAIYYCAASSVSGLLYRSLVSNDYNYWGQGTQVTVCGPGGQHHHHHHGAYPYDVPDYAS

SEQ ID NO: 38 - protein, 5 aa, synthetic construct
GFTFS

SEQ ID NO: 39 - protein, 16 aa, synthetic construct
LEWVSRINSGGDTTYY

SEQ ID NO: 40 - protein, 16 aa, synthetic construct
TAIYYCAASSVSGLLY

SEQ ID NO: 41 - protein, 140 aa, synthetic construct
  VARIANT 2: note = Any amino acid
  VARIANT 41: note = Any amino acid
  VARIANT 117: note = Any amino acid
  VARIANT 122: note = Any amino acid
  VARIANT 125: note = Any amino acid
  VARIANT 131: note = Any amino acid
  VARIANT 133: note = Any amino acid
  VARIANT 134: note = Any amino acid
  VARIANT 135: note = Any amino acid
  VARIANT 136: note = Any amino acid
  VARIANT 137: note = Any amino acid
  VARIANT 138: note = Any amino acid
  VARIANT 139: note = Any amino acid
  VARIANT 140: note = Any amino acid
PXADVQLVESGGGLVQPGGSLTLSCAASGFTFSTYDMTWVXQAPGKGLEWVSRINSGGDTTYYADSVKGQFTISRDNAKNMLYLQMNSLKPDDTAVYYCAPVWNWAFWGQGTQVTVXGPGGXHHXHHHGAXPXXXXXXXX

SEQ ID NO: 42 - protein, 5 aa, synthetic construct
GFTFS

SEQ ID NO: 43 - protein, 16 aa, synthetic construct
LEWVSRINSGGDTTYY

SEQ ID NO: 44 - protein, 21 aa, synthetic construct
TAVYYCAPVWNWAFWGQGTQV

SEQ ID NO: 45 - protein, 148 aa, synthetic construct
MAEVQLVESGGGLVQPGGSLRLSCAASGFTFSSYDMSWVRQAPGKGLEWVSAINSGGGSTYYADSVKGRFTISRDNAKNTLYLQMNSLKPEDTAVYYCATDDGGSWYPTSSFGYWGQGTQVTVSSGPGGQHHHHHHGAYPYDVPDYAS

SEQ ID NO: 46 - protein, 5 aa, synthetic construct
GFTFS

SEQ ID NO: 47 - protein, 16 aa, synthetic construct
LEWVSAINSGGGSTYY

SEQ ID NO: 48 - protein, 19 aa, synthetic construct
TAVYYCATDDGGSWYPTSS

SEQ ID NO: 49 - protein, 144 aa, synthetic construct
MAEVQLVESGGGLVQPGGSLRLSCAASGGFTFSNYWMHWVRQAPRRGLEWVCGINSDGSNTYYADSVKGRFTISRDSAKNTLYVQMNSLKSEDTALYYCARRPPYGYAYNYWGQGTQVTVCGPGGQHHHHHHGAYPYDVPDYAS

SEQ ID NO: 50 - protein, 5 aa, synthetic construct
GFTFS

SEQ ID NO: 51 - protein, 16 aa, synthetic construct
LEWVCGINSDGSNTYY

SEQ ID NO: 52 - protein, 22 aa, synthetic construct
TALYYCARRPPYGYAYNYWGQG

SEQ ID NO: 53 - protein, 140 aa, synthetic construct
LAEVQLVESGGGLVQPGGSLRLSCAASGFTFSNYYMTWVCQAPGKGLEWVSGINSDGTNTYYTDSVKGRFTISRDNARNTLYLQMNSLKSEDTALYYCATDPLFYAWGQGTQVTVSSGPGGQHHHHHHGAYPYDVPDYAS

SEQ ID NO: 54 - protein, 5 aa, synthetic construct
GFTFS

SEQ ID NO: 55 - protein, 16 aa, synthetic construct
LEWVSGINSDGTNTYY

SEQ ID NO: 56 - protein, 18 aa, synthetic construct
ALYYCATDPLFYAWGQGT

SEQ ID NO: 57 - protein, 144 aa, synthetic construct
  VARIANT 1: note = Any amino acid
XAEVQLVESGGGLVQPGGSLRLSCAASGFTFSTYYMSWVRQAPGKGLEWVSGIRSDGSTTWYGDSVKGRFTISRDNAENMLYLQMNSLKLEDTAVYYCAGDPIFTMSTMGYWGQGTQVTVCGPGGQHHHHHHGAYPYDVPDYAS

SEQ ID NO: 58 - protein, 5 aa, synthetic construct
GFTFS

SEQ ID NO: 59 - protein, 16 aa, synthetic construct
LEWVSGIRSDGSTTWY

SEQ ID NO: 60 - protein, 18 aa, synthetic construct
AVYYCAGDPIFTMSTMGY

SEQ ID NO: 61 - protein, 144 aa, synthetic construct; identical to SEQ ID NO: 57
  VARIANT 1: note = Any amino acid

SEQ ID NO: 62 - protein, 5 aa, synthetic construct
GFTFS

SEQ ID NO: 63 - protein, 16 aa, synthetic construct
LEWVSGIRSDGSTTWY

SEQ ID NO: 64 - protein, 18 aa, synthetic construct
AVYYCAGDPIFTMSTMGY

SEQ ID NO: 65 - protein, 139 aa, synthetic construct
MADVQLVESGGGLVQPGGSLTLSCAASGFTFSTYDMTWVRQAPGKGLEWVSRINSGGDTTYYADSVKGQFTISRDNAKNMLYLQMNSLKPDDTAVYYCAPVWNWAFWGQGTQVTVCGPGGQHHHHHHGAYPYDVPDYAS

SEQ ID NO: 66 - protein, 5 aa, synthetic construct
GFTFS

SEQ ID NO: 67 - protein, 16 aa, synthetic construct
LEWVSRINSGGDTTYY

SEQ ID NO: 68 - protein, 18 aa, synthetic construct
AVYYCAPVWNWAFWGQGT

SEQ ID NO: 69 - protein, 130 aa, synthetic construct
MAEVQLVESGGGSVQAGGSLRLSCTASGSTIDDADMGWYHQAPGNECELVSSSTRDFSAYYSDSVKGRFTISRDNAKNTIFLQLNSLKPEDTGTYYCFLSKLEGDWYSSPECRDTRSGSWGQGTQVTVSS

SEQ ID NO: 70 - protein, 9 aa, synthetic construct
SGSTIDDAD

SEQ ID NO: 71 - protein, 9 aa, synthetic construct
STRDFSAYY

SEQ ID NO: 72 - protein, 23 aa, synthetic construct
FLSKLEGDWYSSPECRDTRSGSW

SEQ ID NO: 73 - protein, 128 aa, synthetic construct
  VARIANT 5: note = Any amino acid
MAEVXLVESGGGSVQAGGSLRLSCTASGFTIDDADMGWYHQAPGNECELVSSSTRDFSAYYSDSVKGRFTISRDNAKNTIFLQLNSLKPEDTGTYYCFLSKLEGDWYSSPECRDTRFGSWGQGTQSPS

SEQ ID NO: 74 - protein, 9 aa, synthetic construct
SGFTIDDAD

SEQ ID NO: 75 - protein, 9 aa, synthetic construct
STRDFSAYY

SEQ ID NO: 76 - protein, 23 aa, synthetic construct
FLSKLEGDWYSSPECRDTRFGSW

SEQ ID NO: 77 - protein, 129 aa, synthetic construct
MAEVQLVESGGGSVQAGGSLRLSCQASGYIFSSYTVGWFRQAPGKGCELVSTLNSDGITDYTDSVKGRFTISRDNTKNTVYLQMNSLKPEDTAKYYCAADPLNKIFLDGDCGEGNLEDWGQGTQVTVSS

SEQ ID NO: 78 - protein, 9 aa, synthetic construct
SGYIFSSYT

SEQ ID NO: 79 - protein, 9 aa, synthetic construct
LNSDGITDY

SEQ ID NO: 80 - protein, 22 aa, synthetic construct
AADPLNKIFLDGDCGEGNLEDW

SEQ ID NO: 81 - protein, 128 aa, synthetic construct
MAEVQLVESGGGSVQAGGSLRLSCASHEFTLTHYTMAWFRQIPGKRREGAAAITSGGETYYADSVKGRFTISLDNNNRTVDLQMNSLKLEDTAIYYCAADVGQHIDASQALSVGYSYWGQGTQVTVSS

SEQ ID NO: 82 - protein, 9 aa, synthetic construct
HEFTLTHYT

SEQ ID NO: 83 - protein, 9 aa, synthetic construct
ITSGGETYY

SEQ ID NO: 84 - protein, 21 aa, synthetic construct
AADVGQHIDASQALSVGYSYW

SEQ ID NO: 85 - protein, 129 aa, synthetic construct
MAEVQLVESGGGSVQAGGSLRLSCTASGFTIDDADMGWYHQAPGNECELVSSSTRDFSAYYSDSVKGRFTISRDNANNTMYLQMNSLKPEDTAIYYCAAERPAPCYSGSWSNAAGFKSWGQGTQVTVSS

SEQ ID NO: 86 - protein, 9 aa, synthetic construct
SGFTIDDAD

SEQ ID NO: 87 - protein, 9 aa, synthetic construct
STRDFSAYY

SEQ ID NO: 88 - protein, 23 aa, synthetic construct
AAERPAPCYSGSWSNAAGFKSWG

SEQ ID NO: 89 - protein, 129 aa, synthetic construct
MAEVQLVESGGGSVQAGGSLRLTCDASGYTHSTNIMGWFRQRPGKEREGVAAIYTGDGSTFYAYSVEGRVAISRDNAENVVYLQMNSLKPEDTAMYYCAAGDGCSRSHASWRTSDYAYWGQGTQVTVSS

SEQ ID NO: 90 - protein, 9 aa, synthetic construct
SGYTHSTNI

SEQ ID NO: 91 - protein, 9 aa, synthetic construct
IYTGDGSTF

SEQ ID NO: 92 - protein, 22 aa, synthetic construct
AAGDGCSRSHASWRTSDYAYWG

SEQ ID NO: 93 - protein, 349 aa, Homo sapiens
MTAASMGPVRVAFVVLLALCSRPAVGQNCSGPCRCPDEPAPRCPAGVSLVLDGCGCCRVCAKQLGELCTERDPCDPHKGLFCDFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA

SEQ ID NO: 94 - protein, 97 aa, Homo sapiens
GKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA

SEQ ID NO: 95 - protein, 339 aa, Homo sapiens
LAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQD

SEQ ID NO: 96 - DNA, 23 bp, synthetic construct
  source 1..23: mol_type = other DNA
gtcctggctgctcttctacaagg

SEQ ID NO: 97 - DNA, 23 bp, synthetic construct
  source 1..23: mol_type = other DNA
ggtacgtgctgttgaactgttcc

SEQ ID NO: 98 - DNA, 42 bp, synthetic construct
  source 1..42: mol_type = other DNA
gtgcaggcccagccggccatggcccatggccgakgtscagct

SEQ ID NO: 99 - DNA, 40 bp, synthetic construct
  source 1..40: mol_type = other DNA
gtgattggcctcccgggcctgaggagacggtgacctgggt

SEQ ID NO: 100 - DNA, 725 bp, synthetic construct
  source 1..725: mol_type = other DNA
atacctattgcctacggcggccgctggattgttattactcgcggcccagccggccatggcccaggtgcagctgcaggagtctggaggaggctcggtgcaggctggagggtctctgagactctcctgtgcagcctctgaatataccgtgactagggcctggttccgccaggctccaggaaaggagcgtgagtgggtcgcaggcattaatagtggtggtggtagcacatactacaccgactccgtgaagggccgattcaccatctcccaagacaaggccaggaacacggtgtttctgcaaatgaacagcctgaaacctgaggacacggccatctacacctgtgcggcgccatcaagttccggcgactataagtactggggccaggggacccaggtcaccgtctcctcaggcccgggaggccaacaccatcaccaccatcatggcgcatatccgtatgatgtgccggactatgcttcttaggccgaaactgttgaaagttgtttagcaaaacctcatacagaaaattcatttactaacgtctggaaagacgacaaactttagatcgttacgctaactatgagggctgtctgtgaatgctacagcgttgtgtttgtactgatgacgaactcagtgtacgtacatggtctatgggctgctatcttgaaatgaggtgtgccttgaagtgcgtctgagtgccgtctgagttgccgttacataaccctcctga

SEQ ID NO: 101 - DNA, 767 bp, synthetic construct
  source 1..767: mol_type = other DNA
ataccctaattgcctacggcggccgctggattgttattactcgcggcccagccggccatggcccaggtgcagctgcaggagtctggaggaggcttggtgcagcctggggggtctctgagactctcctgtgcagcctctgtattcacattcagtggctaccacatgagctgggtccgccaggctccagggaaggggctcgagtgggtctcagctatagatagtggtggtggtagcacatactatgcagactccgtgaagggccgattcaccatctccagagacaacgccaagaactcgctgtatctgcaaatgaacagtttgaaacctgaggacactgccgtgtattactgcgccacagcgcccgacatctatgaggtggaaaccggtcttccccctagaagagactttggttactggggccaggggacccaggtcaccgtctcctcaggcccgggaggccaacaccatcaccaccatcatggcgcatatccgtatgatgtgccggactatgcttcttaggccgaaactgttgaaagttgtttagcaaaacctcatacagaaaattcatttactaacgtctggaaagacgacaaaactttagatcgttacgctaactatgagggctgtctgtggaatgctacaggcgttgtggtttgtactggtgacgaaactcagtgtacggtacatgggttcctattgggcctggctatcctgaattgaggttgtgctctgagggtggcgattctggaggatggccggctctctga

SEQ ID NO: 102 - DNA, 743 bp, synthetic construct
  source 1..743: mol_type = other DNA
atacctattgcctacggcggccgctggattgttattactcgcggcccagccggccatggcccaggtgcagctgcaggagtctgggggaggctcggtgcaggctggagggtctctgagactctcctgtttagtctctggatacacctacactaacgtctacatgggctacttccgccaggctccagggaaggggcgtgagggggtcgcaagtattcatcgtggtcttggaaccacagactacgccgactccgtgaagggccgattcgccatttaccaggacaccgccaaaaacacggtgtatctacaaatgaacagcctgaaacctgaggacacggacatctattactgtgcgcttgggccgatcgctaacttagcctatgcagctgactatgagggccaggggacccaggtcaccgtctcctcaggcccgggaggccaacaccatcaccaccatcatggcgcatatccgtatgatgtgccggactatgcttcttaggccgaaactgttgaaagttgtttagcaaaacctcatacagaaaattcatttactaacgtctggaaagacgacaaaactttagatcgttacgctaactatgagggctgtctgtggaatgctacaggcgttgtgtttgtactgatgacgaaactcagtgtacgtacatggtctatggctggctatcctgaaatgagtgtactctgaagtgcgtcctgaagttgccgtctgagggtggcggtaactaact

SEQ ID NO: 103 - DNA, 763 bp, synthetic construct
  source 1..763: mol_type = other DNA
tacctattgcctacggcggccgctggattgttattactcgcggcccagccggccatggcccaggtgcagctgcaggagtctggaggaggctcggtgcaggctggagggtctctgagactctcctgtgcagcctctggatacatcggcagtgcaggctgcatagcctggttccgccaggctccagggaaggagcgcgagggggtcgcagcagtcggcgctgcaggtagtagcagtagtagcacatactacgccgactccgtgaagggccgattcaccatctcccaagacagcgccaagaacacgatatatctacaaatgaacatcctgaaacctgaggacactgccatgtattattgtgcagcaggcccactgtggtcgaatggggggcgttgctcgaaccacattttcgaccgctcgtggtactactactggggccaggggacccaggtcaccgtctcctcaggcccgggaggccaacaccatcaccaccatcatggcgcatatccgtatgatgtgccggactatgcttcttaggccgaaactgttgaaagttgtttagcaaaacctcatacagaaaattcatttactaacgtctggaaagacgacaaaactttagatcgttacgctaactatgagggctgtctgtggaatgctacaggcgttgtgatttgtactgatgacgaaactcagtgtacggtacatggatctatgggctggcctatcccttgaaatgaagttgttgctcggagggtggcggcttctg

SEQ ID NO: 104 - DNA, 749 bp, synthetic construct
  source 1..749: mol_type = other DNA
atcctaattgcctacggcggccgctggattgttattactcgcggcccagccggccatggcccaggtgcagctgcaggagtctggaggaggctcggtgcagactggagggtctctgagactctcctgtgcagcctctggataccgcttcagtcgttattgcaagggctggttccgccaggctccaggaaaggagcgtgaggcggtcgcacatatctctggtggtggatatgaaacatactacctcgactccgtgaagggccgattcaccatctcccaggacagcgccgacaacacgctatatctacaaatgaacagcctgaaatctgaggacacggccatctattactgtgcggctggtatcgtagtgagtaaccggtgcgaaattaccgactttagttactggggccaggggacccaggtcaccgtctcctcaggcccgggaggccaacaccatcaccaccatcatggcgcatatccgtatgatgtgccggactatgcttcttaggccgaaactgttgaaagttgtttagcaaaacctcatacagaaaattcatttactaacgtctggaaagacgacaaaactttagatcgttacgctaactatgagggctgtctgtggaatgctacaggcgttgtgtttgtactgtgacgaaactcagtgtacgtacatggctctatgggctgctatctgaatgaggtggtgctctgaggttgcgtctgagggtggcggtttctggagggtgggccga

SEQ ID NO: 105 - DNA, 738 bp, synthetic construct
  source 1..738: mol_type = other DNA
aaaccctattgcctacggcggccgctggattgttattactcgcggcccagccggccatggcccaggtgcagctgcaggagtctgggggaggctcggtgcaggctggagggtctctgagactctcctgtgcagcctctgaatataccatcactagggcctggttccgccaggctccagggaaggcgcgtgagtgggtcgcaggtattaatagtggtggtggtagcacattctacgccgactccgtgaagggccgattcaccatctcccaagacaacgccaggaacacggtgtttctgcaaatgaccagcctgaaacctgaggacacggccatctatacctgtgcggcgccatcaagctccggcgactataagtactggggccttgggacccaggtcaccgtctcctcaggcccgggaggccaacaccatcaccaccatcatggcgcatatccgtatgatgtgccggactatgcttcttaggccgaaactgttgaaagttgtttagcaaaacctcatacagaaaattcatttactaacgtctggaaagacgacaaaactttagatcgttacgctaactatgagggctgtctgtgatgctacaggcgttgtgtttgtactggtgacgaactcagtgtacgtacatgggttctatgggctggctatccctgaaatgaagttgtgcctctgaagtgcgtctgagtggcgtctgaagtgcgtactaacctcctgagttacggtg

SEQ ID NO: 106 - DNA, 753 bp, synthetic construct
  source 1..753: mol_type = other DNA
ataacctaattgcctacggcggccgctggattgttattactcgcggcccagccggccatggcccaggtgcagctgcaggagtctgggggaggcttggtgcagcccggggggtctctgagactctcctgtgcagcctctggattcaccttcagtgactatgccatgagctgggtccgccaggctccagggaaggggctcgagtgggtctcatctattaatagtggtggtgatggcacagccgttgcagcgtccctagagggccgattcaccatctccagagacaacgccaagaacacgctatatctgcaattgaatagcctgaaaactgaagacacggccatgtattactgtgcaaaaacggggagggttcatatagcgctgttgcaagaggccgtccggggccaggggacccaggtcaccgtctcctcaggcccgggaggccaacaccatcaccaccatcatggcgcatatccgtatgatgtgccggactatgcttcttaggccgaaactgttgaaagttgtttagcaaaacctcatacagaaaattcatttactaacgtctggaaagacgacaaaactttagatcgttacgctaactatgagggctgtctgtggaatgctacaggcgttgtgtttgtactgtgacgaaactcagtgttacggtacatgggtctatggctgctatccctgaaaatgaagtggtgctctgagttgccgtctgaggtggcggttctgaggggtggcggtaactta

SEQ ID NO: 107 - DNA, 768 bp, synthetic construct
  source 1..768: mol_type = other DNA
taccctatggccttacggcggccgctggattgttattactcgcggcccagccggccatggcccaggtgcagctgcaggagtctgggggaggcgcggtgcaggctggagggtctctgagactctcctgtgcagcctctggatacacctacagtagctcctgcatgggctggttccgccaggctccagggaaggagcgcgaggcagtcgcagctatttatactgataatggtagcacatactatgccgactccgtgaagggccgattcaccatctcccaagacaacgccaagaatacggtgtatctgcaaatgaacagcctgaaacctgaggacactgccatgtactactgtgcagcagcggcggtaaccctccggatggggggggagtgcatagctgctattcaggctcttggtgttatgacgttcggagctgactttggttactggggccaggggacccaggtcaccgtctcctcaggcccgggaggccaacaccatcaccaccatcatggcgcatatccgtatgatgtgccggactatgcttcttaggccgaaactgttgaaagttgtttagcaaaacctcatacagaaaattcatttactaacgtctggaaagacgacaaaactttagatcgttacgctaactatgagggctgtctgtggaatgctacaggcgttgtggttgtactgggtgacgaaactcatgtaccgtacattggtctatgggcttgctatcctgaaatggagggtgggtggcctcttgaaggtg

SEQ ID NO: 108 - DNA, 635 bp, synthetic construct
  source 1..635: mol_type = other DNA
aaccctattgcctacggcggccgctggattgttattactcgcggcccagccggccatggcccaggagcagctgcaggagtctgggggaggctcggtgcaggctggagggtctctgagactctcctgtgcagcctctggatacatctacagtagcaactgcatggggtggttccgccaggctccagggaaggagcgcgagggggtcgcagctatttgtactggtggtggtagcacatactatgccgactccgtgaagggccgattcaccgtctcccaagacaacgccaagaacacggtgtatctgcaaatgagcagcctgaaacctgaggacactgccatgtactactgcgcaacagggggagcatatgcttggagatactatagcgactatgacccagcctcgtggtggggcagtttaaggtataacatggactactggggcaaagggacccgggtcaccgtctcctcaggcccgggaggccaacaccatcaccaccatcatggcgcatatccgtatgatgtgccggactatgcttcttaggccgaaactgctgaaagatgttcagctaaacctcatacgaaagatccatttactatcgtctggtaacgacagacaacttagattcgttaccgca

SEQ ID NO: 109 - DNA, 786 bp, synthetic construct
  source 1..786: mol_type = other DNA
aacctattgcctacggcggccgctggattgttattactcgcggcccagccggccatggcccaggtgcagctgcaggagtctgggggaggctcggtgcaggctggagggtctctgagactctcctgtacagcctctggattcacttttgatgattctgacatgggctggtaccaccaggctccagggaatgagtgcgagttggtctcaggtattattagtgatggtagcacatactatgcagactccgtgaagggccgattcaccatctccagagacaacgccaaaaacacggtgtatctgaaaatgaacagcctgaaacctgaggacacggccaactattattgtgcggcctacccccccgggtggcccggatgttctctccaaaaatttcattatgatcactggggccaggggacccaggtcaccgtctcctcaggcccgggaggccaacaccatcaccaccatcatggcgcatatccttatgatgtgccggactatgcttcttaggccgaagctgttgaaagttgtttagcaaaacctcatacagaatattcatttactaacgtctggaaagacgacaaaactttagatcgctacgctaactatgagggctgtctgtggaatgctacaggcgttggtgttttgtactgatgacaatactcagtgctgccactcgtgggcttctagtggtgctgctatccatgatatggaggtggtgctcatggagggtgggcgctttcaggagggggcggcgtctttctgggagggggtggggcggctactaca

SEQ ID NO: 110 - DNA, 757 bp, synthetic construct
  source 1..757: mol_type = other DNA
atacctaattgcctacggcggccgctggattgttattactcgcggcccagccggccatggcccaggtgcagctgcaggagtctgggggaggctcggtgcaggctggagggtctctgagactctcctgtttagtctctggatacacctacagtaacgtctacatgggctacttccgccaggctccagggaaggggcgtgaggcggtcgcaagtattcatcgtggtcttggaaccgcagactacgccgactccgtgaagggccgattcgccatttaccaggacaccgccaaaaacacggtgtatctacaaatgaacagcctaaaacctgaggacacggccatctattactgtgcgcttgggccgatcgctaacttggcctgggcatctgactatgagggccaggggacccaggtcaccgtctcctcaggcccgggaggccaacaccatcaccaccatcatggcgcatatccgtatgatgtgccggactatgcttcttaggccgaaactgttgaaagttgtttagcaaaacctcatacagaaaattcatttactaacgtctggaaagacgacaaaactttagatcgttacgctaactatgagggctgtctgtggaatgctacaggcgttgtggtttgtactggtgacgaaactcagtgttacggtacatgggtctattggctgctatcctgaaaatgaagtggtgctctgagttgccgttctgagggtgcgatctgagggttgggcgggtacttaagctc

SEQ ID NO: 111 - DNA, 766 bp, synthetic construct
  source 1..766: mol_type = other DNA
ataccctattgcctacggcggccgctggattgttattactcgcggcccagccggccatggcccaggagcagctgcaggagtctgggggaggcttggtgcagcccggggggtctctgagactctcctgtgcagcctccggattcacctctggattcacattcaatgactacgacatgagctgggtccgccaggctccagggaaggggctcgagtgggtctcaggtattaatactgctgattacacagactatatagactccgtgaagggccgattcaccatctccagagacaacgccaagaacacgctgtatctgcaaatgaacaatttgaaacctgaggacactgccgtgtatttctgcgccacagattggaatccattgatgcctacgggttttttgcccaggggccaggggacccaggtcaccgtctcctcaggcccgggaggccaacaccatcaccaccatcatggcgcatatccgtatgatgtgccggactatgcttcttaggccgaaactgttgaaagttgtttagcaaaacctcatacagaaaattcatttactaacgtctggaaagacgacaaaactttagatcgttacgctaactatgagggctgtctgtgaatgctacaggcgttgtgatttgtactgtgacgaactcagtggtacggtacatggctctattgggctgctatccctggaaatgaaggtggtgctctgaagggtgggcggatctggaggggtggcggacttggagggggtggggcgga

SEQ ID NO: 112 - DNA, 771 bp, synthetic construct
  source 1..771: mol_type = other DNA
aacctattgcctacggcggccgctggattgttattactcgcggcccagccggccatggcccaggtgcagctgcaggagtctgggggaggctcggtgcaggctggagggtctctgagactctcctgtgcagcctctggatacacctacagtagctactgcatgggctggtttcgccaggctccagggaaggagagtgagggggtcgcagctattaatagtggtggtggtagcacatactacgccgactccgtgaagggccgattcaccatctcccaagacaacgccaacacggtgtatctgcaaatgaacagcctgaaacctgaggacacggccatctattactgtacggcagctccggaagattattgtagtggtggtctctggtcttttggcatggactactggggcaaagggacccaggtcaccgtctcctcaggcccgggaggccaacaccatcaccaccatcatggcgcatatccgtatgatgtgccggactatgcttcttaggccgaaactgttgaaagttgtttagcaaaacctcatacagaaaattcatttactaacgtctggaaagacgacaaaactttagatcgttacgctaactatgagggctgtctgtggaatgctacaggcgttgtggtttgtactggtgacgaactcagtgttacggtacatggttcctattggcttgctatccctgaaattgaagtgttgctctgaggttgcgatctgagggtggcggattctggaagggtgggccggtatacctaa

SEQ ID NO: 113 - DNA, 613 bp, synthetic construct
  source 1..613: mol_type = other DNA
aaccctaattgcctacggcggccgctggattgttattactcgcggcccagccggccatggcccaggtgcagctgcaggagtctgggggaggctcggtgcaggctggagggtctctgagactctcctgtgcagccgctggatacaccgacaataggaaatgcatgggctggttccgccaggctccagggaaggagcgcgagggggtcgcggttatttttacgagtgatagtagtactgcctataccgactccgtgaagggccgattcaccatctcccaaggggtccccaagaacacggtgtatctccaaatgaacgacctgaaacctgaggacactgccatgtactactgtgcagcgtatgtgagcccgtacgtgtacggcggttgctccttgcaggatttcaggtatgactactggggccaggggacccaggtcaccgtctcctcaggcccgggaggccaacaccatcaccaccatcatggcgcatatccgtatgatgtgccggactatgcttcttaggccgaaactgttgaaagttgtttagcaaaacctcatacagaaaattcatttactaacgtctggaaagacgacaaactttagatttcgttacgcca

SEQ ID NO: 114 - DNA, 769 bp, synthetic construct
  source 1..769: mol_type = other DNA
aacctaattgcctacggcggccgctggattgttattactcgcggcccagccggccatggcccaggtgcagctgcaggagtctggaggaggctcggtgcaggctggagggtctctgagactctcctgtacagcctctggattcacttttgcgtattcagacatgggctggtaccaccaggctccagggaatgagtgcgagttggtctcaactattagtagtgatggtagcacatactatgcagactccgtgaagggccgattcaccatctccagagacaacgccaagaacacggtgtatctgcaaatgaacagcctgaaacctgaggacacggccatgtattactgtgcggcagatcattggctggccgaacgtagtgattgtttagctcctgccgttggttactggggccaggggacccaggtcaccgtctcctcaggcccgggaggccaacaccatcaccaccatcatggcgcatatccgtatgatgtgccggactatgcttcttaggccgaaactgttgaaagttgtttagcaaaacctcatacagaaaattcatttactaacgtctggaaagacgacaaaactttagatcgttacgctaactatgagggctgtctgtggaatgctacaggcgttgtggtttgtactggtgacgaaactcagtgtacggtacatgggtcctatggcttgctatccctgaattgaagtgtgctctgaggttgccgatctgagggtggcgggttctggaggggggtggcggtactctac

SEQ ID NO: 115 - DNA, 755 bp, synthetic construct
  source 1..755: mol_type = other DNA
ataccctattgcctacggcggccgctggattgttattactcgcggcccagccggccatggcccaggtgcagctgcaggagtctggaggaggctcggtgcaggctggagggtctctgagactctcctgtgcagcctctggataccgcttcagtcgttattgcatgggctggttccgccaggctccaggaaaggagcgtgaggcggtcgcacatatcgctggtggtggatatggaacatactacctcgactccgtgaagggccgattcaccatctcccaagacagcgccaagaacacgctatatctacaaatgaacagcctgaaatctgaggacacggccatctattactgtgcggctggtatcatagtgagtaaccggtgcgagagtgccgactttagttactggggccaggggacccaggtcaccgtctcctcaggcccgggaggccaacaccatcaccaccatcatggcgcatatccgtatgatgtgccggactatgcttcttaggccgaaactgttgaaagttgtttagcaaaacctcatacagaaaattcatttactaacgtctggaaagacgacaaaactttagatcgttacgctaactatgagggctgtctgtggaatgctacaggcgttgtgtttgtactggtgacgaaactcagtgtacgtacatgggttctattgggctgctatctggaattgaagttggttgctctgagggtggcggttctggaggggttgggcggtctgagttgc

SEQ ID NO: 116 - DNA, 615 bp, synthetic construct
  source 1..615: mol_type = other DNA
aacctattgcctacggcggccgctggattgttattactcgcggcccagccggccatggcccaggagcagctgcaggagtctgggggaggctcggtgcaggctggagggtctctgagactctcctgtttagcctctggatacacctacactacctactacatgggctggttccgccaggctccagggaaggggcgtgagggggtcgcaagtattcatcgtggtgttgaaaccacatactacgccgactccgtgaagggccgattcaccatttaccgagacaccgccaaaaacacggtgtatctacaaatgaacagcctgaaacctgaggacacggccatctattactgtgcgcttgaactgatcggtaacttgtgctatgcatcagactctcacggccaggggacccaggtcaccgtctcctcaggcccgggaggccaacaccatcaccaccatcatggcgcatatccgtatgatgtgccggactatgcttcttaggtgcgaagactgttgaaagttgtttagaaactgctcatacttgttaattcatttactcacgtctgaaaagactacaataactgtctgtacgatacgacaaaaactttagatcgttacgca

SEQ ID NO: 117 - DNA, 728 bp, synthetic construct
  source 1..728: mol_type = other DNA
aaacctattgcctacggcggccgctggattgttattactcgcggcccagccggccatggcccaggtgcagctgcaggagtctgggggaggcttggtgcagcctggggggtctctgagactctcctgtgcagcctccggattcacctctggattcacattcaatgactacgacatgagctgggtccgccaggctccagggaaggggctcgagtgggtctcaggtattaatactgctgattacacagactatatagactccgtgaagggccgattcaccatctccagagacaacgccaagaacacgctgtatctgcaaatgaacaatttgaaacctgaggacactgccgtgtatttctgcgccacagattggaatccattgatgcctacgggttttttgcccaggggccaggggacccaggtcaccgtctcctcaggcccgggaggccaacaccatcaccaccatcatggcgcatatccgtatgatgtgccggactatgcttcttaggccgaaactgttgaaagttgtttagcaaaacctcatacagaaaattcatttactaacgtctgggaaagacgacaaaactttagatcgttacgctaactatgagggctgtctgtgaatgctacagcgttgtgcttgtactgtgacgaactcagtgtaccgtacatgctctatggtctgcatcctgaatgaggttgtgctctgagctgccgttctgaaggtggcg

SEQ ID NO: 118 - DNA, 756 bp, synthetic construct
  source 1..756: mol_type = other DNA
aaacctattgcctacggcggccgctggattgttattactcgcggcccagccggccatggcccaggtgcagctgcaggagtctggaggaggcttggtgctgcccggggggtctctgagactctcctgtgcagcctctggattcacattcagtaacagtgtcatgagctgggtccgccaggctccagggaagggcatcgagtgggtctcatctattagtgatggaggtgattacacagattacgcggactccgtgaagggccgattcaccatctccagagacaacgccaagaacacgctgtatctgcaattgaacagcctgaaaactgtcgacacggccatgtattactgtgcaacagagtggggttggtccgcggcctcgtctgagtacaggggccaggggacccaggtcaccgtctcctcaggcccgggaggccaacaccatcaccaccatcatggcgcatatccgtatgatgtgccggactatgcttcttaggccgaaactgttgaaagttgtttagcaaaacctcatacagaaaattcatttactaacgtctggaaagacgacaaaactttagatcgttacgctaactatgagggctgtctgtggaatgctacaggcgttgtggtttgtactggtgacgaaactcagtgttacggtacatgggttcctattggctgctatccctgaaaatgaggtgtgctctgaggttgcgtctgagtgcgcttctgagggtgggcggtactaaccttcctgag

SEQ ID NO: 119 - protein, 122 aa, synthetic construct
MAQVQLQESGGGSVQAGGSLRLSCAASEYTVTRAWFRQAPGKEREWVAGINSGGGSTYYTDSVKGRFTISQDKARNTVFLQMNSLKPEDTAIYTCAAPSSSGDYKYWGQGTQVTVSSGPGGQ

SEQ ID NO: 120 - protein, 135 aa, synthetic construct
MAQVQLQESGGGLVQPGGSLRLSCAASVFTFSGYHMSWVRQAPGKGLEWVSAIDSGGGSTYYADSVKGRFTISRDNAKNSLYLQMNSLKPEDTAVYYCATAPDIYEVETGLPPRRDFGYWGQGTQVTVSSGPGGQ

SEQ ID NO: 121 - protein, 128 aa, synthetic construct
MAQVQLQESGGGSVQAGGSLRLSCLVSGYTYTNVYMGYFRQAPGKGREGVASIHRGLGTTDYADSVKGRFAIYQDTAKNTVYLQMNSLKPEDTDIYYCALGPIANLAYAADYEGQGTQVTVSSGPGGQ

SEQ ID NO: 122 - protein, 141 aa, synthetic construct
MAQVQLQESGGGSVQAGGSLRLSCAASGYIGSAGCIAWFRQAPGKEREGVAAVGAAGSSSSSTYYADSVKGRFTISQDSAKNTIYLQMNILKPEDTAMYYCAAGPLWSNGGRCSNHIFDRSWYYYWGQGTQVTVSSGPGGQ

SEQ ID NO: 123 - protein, 131 aa, synthetic construct
MAQVQLQESGGGSVQTGGSLRLSCAASGYRFSRYCKGWFRQAPGKEREAVAHISGGGYETYYLDSVKGRFTISQDSADNTLYLQMNSLKSEDTAIYYCAAGIVVSNRCEITDFSYWGQGTQVTVSSGPGGQ

SEQ ID NO: 124 - protein, 122 aa, synthetic construct
MAQVQLQESGGGSVQAGGSLRLSCAASEYTITRAWFRQAPGKAREWVAGINSGGGSTFYADSVKGRFTISQDNARNTVFLQMTSLKPEDTAIYTCAAPSSSGDYKYWGLGTQVTVSSGPGGQ

SEQ ID NO: 125 - protein, 129 aa, synthetic construct
MAQVQLQESGGGLVQPGGSLRLSCAASGFTFSDYAMSWVRQAPGKGLEWVSSINSGGDGTAVAASLEGRFTISRDNAKNTLYLQLNSLKTEDTAMYYCAKTGRVHIALLQEAVRGQGTQVTVSSGPGGQ

SEQ ID NO: 126 - protein, 145 aa, synthetic construct
MAQVQLQESGGGAVQAGGSLRLSCAASGYTYSSSCMGWFRQAPGKEREAVAAIYTDNGSTYYADSVKGRFTISQDNAKNTVYLQMNSLKPEDTAMYYCAAAAVTLRMGGECIAAIQALGVMTFGADFGYWGQGTQVTVSSGPGGQ

SEQ ID NO: 127 - protein, 143 aa, synthetic construct
MAQEQLQESGGGSVQAGGSLRLSCAASGYIYSSNCMGWFRQAPGKEREGVAAICTGGGSTYYADSVKGRFTVSQDNAKNTVYLQMSSLKPEDTAMYYCATGGAYAWRYYSDYDPASWWGSLRYNMDYWGKGTRVTVSSGPGGQ

SEQ ID NO: 128 - protein, 132 aa, synthetic construct
MAQVQLQESGGGSVQAGGSLRLSCTASGFTFDDSDMGWYHQAPGNECELVSGIISDGSTYYADSVKGRFTISRDNAKNTVYLKMNSLKPEDTANYYCAAYPPGWPGCSLQKFHYDHWGQGTQVTVSSGPGGQ

SEQ ID NO: 129 - protein, 128 aa, synthetic construct
MAQVQLQESGGGSVQAGGSLRLSCLVSGYTYSNVYMGYFRQAPGKGREAVASIHRGLGTADYADSVKGRFAIYQDTAKNTVYLQMNSLKPEDTAIYYCALGPIANLAWASDYEGQGTQVTVSSGPGGQ

SEQ ID NO: 130 - protein, 131 aa, synthetic construct
MAQEQLQESGGGLVQPGGSLRLSCAASGFTSGFTFNDYDMSWVRQAPGKGLEWVSGINTADYTDYIDSVKGRFTISRDNAKNTLYLQMNNLKPEDTAVYFCATDWNPLMPTGFLPRGQGTQVTVSSGPGGQ

SEQ ID NO: 131 - protein, 132 aa, synthetic construct
MAQVQLQESGGGSVQAGGSLRLSCAASGYTYSSYCMGWFRQAPGKESEGVAAINSGGGSTYYADSVKGRFTISQDNANTVYLQMNSLKPEDTAIYYCTAAPEDYCSGGLWSFGMDYWGKGTQVTVSSGPGGQ

SEQ ID NO: 132 - protein, 135 aa, synthetic construct
MAQVQLQESGGGSVQAGGSLRLSCAAAGYTDNRKCMGWFRQAPGKEREGVAVIFTSDSSTAYTDSVKGRFTISQGVPKNTVYLQMNDLKPEDTAMYYCAAYVSPYVYGGCSLQDFRYDYWGQGTQVTVSSGPGGQ

SEQ ID NO: 133 - protein, 132 aa, synthetic construct
MAQVQLQESGGGSVQAGGSLRLSCTASGFTFAYSDMGWYHQAPGNECELVSTISSDGSTYYADSVKGRFTISRDNAKNTVYLQMNSLKPEDTAMYYCAADHWLAERSDCLAPAVGYWGQGTQVTVSSGPGGQ

SEQ ID NO: 134 - protein, 131 aa, synthetic construct
MAQVQLQESGGGSVQAGGSLRLSCAASGYRFSRYCMGWFRQAPGKEREAVAHIAGGGYGTYYLDSVKGRFTISQDSAKNTLYLQMNSLKSEDTAIYYCAAGIIVSNRCESADFSYWGQGTQVTVSSGPGGQ

SEQ ID NO: 135 - protein, 128 aa, synthetic construct
MAQEQLQESGGGSVQAGGSLRLSCLASGYTYTTYYMGWFRQAPGKGREGVASIHRGVETTYYADSVKGRFTIYRDTAKNTVYLQMNSLKPEDTAIYYCALELIGNLCYASDSHGQGTQVTVSSGPGGQ

SEQ ID NO: 136 - protein, 131 aa, synthetic construct
MAQVQLQESGGGLVQPGGSLRLSCAASGFTSGFTFNDYDMSWVRQAPGKGLEWVSGINTADYTDYIDSVKGRFTISRDNAKNTLYLQMNNLKPEDTAVYFCATDWNPLMPTGFLPRGQGTQVTVSSGPGGQ

SEQ ID NO: 137 - protein, 127 aa, synthetic construct
MAQVQLQESGGGLVLPGGSLRLSCAASGFTFSNSVMSWVRQAPGKGIEWVSSISDGGDYTDYADSVKGRFTISRDNAKNTLYLQLNSLKTVDTAMYYCATEWGWSAASSEYRGQGTQVTVSSGPGGQ

SEQ ID NO: 138 - protein, 7 aa, synthetic construct
EYTVTRA

SEQ ID NO: 139 - protein, 9 aa, synthetic construct
INSGGGSTY

SEQ ID NO: 140 - protein, 11 aa, synthetic construct
AAPSSSGDYKY

SEQ ID NO: 141 - protein, 10 aa, synthetic construct
VFTFSGYHMS

SEQ ID NO: 142 - protein, 9 aa, synthetic construct
IDSGGGSTY

SEQ ID NO: 143 - protein, 21 aa, synthetic construct
ATAPDIYEVETGLPPRRDFGY

SEQ ID NO: 144 - protein, 10 aa, synthetic construct
GYTYTNVYMG

SEQ ID NO: 145 - protein, 9 aa, synthetic construct
IHRGLGTTD

SEQ ID NO: 146 - protein, 14 aa, synthetic construct
ALGPIANLAYAADY

SEQ ID NO: 147 - protein, 10 aa, synthetic construct
GYIGSAGCIA

SEQ ID NO: 148 - protein, 12 aa, synthetic construct
VGAAGSSSSSTY

SEQ ID NO: 149 - protein, 24 aa, synthetic construct
AAGPLWSNGGRCSNHIFDRSWYYY

SEQ ID NO: 150 - protein, 10 aa, synthetic construct
GYRFSRYCKG

SEQ ID NO: 151 - protein, 9 aa, synthetic construct
ISGGGYETY

SEQ ID NO: 152 - protein, 17 aa, synthetic construct
AAGIVVSNRCEITDFSY

SEQ ID NO: 153 - protein, 7 aa, synthetic construct
EYTITRA

SEQ ID NO: 154 - protein, 9 aa, synthetic construct
INSGGGSTF

SEQ ID NO: 155 - protein, 11 aa, synthetic construct
AAPSSSGDYKY

SEQ ID NO: 156 - protein, 10 aa, synthetic construct
GFTFSDYAMS

SEQ ID NO: 157 - protein, 10 aa, synthetic construct
SINSGGDGTA

SEQ ID NO: 158 - protein, 15 aa, synthetic construct
AKTGRVHIALLQEAV

SEQ ID NO: 159 - protein, 10 aa, synthetic construct
GYTYSSSCMG

SEQ ID NO: 160 - protein, 10 aa, synthetic construct
AIYTDNGSTY

SEQ ID NO: 161 - protein, 31 aa, synthetic construct
AAAAVTLRMGGECIAAIQALGVMTFGADFGY

SEQ ID NO: 162 - protein, 10 aa, synthetic construct
GYIYSSNCMG

SEQ ID NO: 163 - protein, 9 aa, synthetic construct
ICTGGGSTY

SEQ ID NO: 164 - protein, 19 aa, synthetic construct
ATGGAYAWRYYSDYDPASW

SEQ ID NO: 165 - protein, 10 aa, synthetic construct
GFTFDDSDMG

SEQ ID NO: 166 - protein, 9 aa, synthetic construct
GIISDGSTY

SEQ ID NO: 167 - protein, 19 aa, synthetic construct
AAYPPGWPGCSLQKFHYDH

SEQ ID NO: 168 - protein, 9 aa, synthetic construct
GYTYSNVYM

SEQ ID NO: 169 - protein, 9 aa, synthetic construct
IHRGLGTAD

SEQ ID NO: 170 - protein, 14 aa, synthetic construct
ALGPIANLAWASDY

SEQ ID NO: 171 - protein, 14 aa, synthetic construct
GFTSGFTFNDYDMS

SEQ ID NO: 172 - protein, 7 aa, synthetic construct
NTADYTD

SEQ ID NO: 173 - protein, 15 aa, synthetic construct
ATDWNPLMPTGFLPR

SEQ ID NO: 174 - protein, 10 aa, synthetic construct
GYTYSSYCMG

SEQ ID NO: 175 - protein, 9 aa, synthetic construct
INSGGGSTY

SEQ ID NO: 176 - protein, 19 aa, synthetic construct
TAAPEDYCSGGLWSFGMDY

SEQ ID NO: 177 - protein, 10 aa, synthetic construct
GYTDNRKCMG

SEQ ID NO: 178 - protein, 11 aa, synthetic construct
VIFTSDSSTAY

SEQ ID NO: 179 - protein, 21 aa, synthetic construct
AAYVSPYVYGGCSLQDFRYDY

SEQ ID NO: 180 - protein, 10 aa, synthetic construct
GFTFAYSDMG

SEQ ID NO: 181 - protein, 9 aa, synthetic construct
TISSDGSTY

SEQ ID NO: 182 - protein, 19 aa, synthetic construct
AADHWLAERSDCLAPAVGY

SEQ ID NO: 183 - protein, 10 aa, synthetic construct
GYRFSRYCMG

SEQ ID NO: 184 - protein, 10 aa, synthetic construct
HIAGGGYGTY

SEQ ID NO: 185 - protein, 17 aa, synthetic construct
AAGIIVSNRCESADFSY

SEQ ID NO: 186 - protein, 10 aa, synthetic construct
GYTYTTYYMG

SEQ ID NO: 187 - protein, 9 aa, synthetic construct
IHRGVETTY

SEQ ID NO: 188 - protein, 14 aa, synthetic construct
ALELIGNLCYASDS

SEQ ID NO: 189 - protein, 14 aa, synthetic construct
GFTSGFTFNDYDMS

SEQ ID NO: 190 - protein, 9 aa, synthetic construct
GINTADYTD

SEQ ID NO: 191 - protein, 14 aa, synthetic construct
ATDWNPLMPTGFLP

SEQ ID NO: 192 - protein, 10 aa, synthetic construct
GFTFSNSVMS

SEQ ID NO: 193 - protein, 10 aa, synthetic construct
SISDGGDYTD

SEQ ID NO: 194 - protein, 13 aa, synthetic construct
ATEWGWSAASSEY

SEQ ID NO: 195 - DNA, 737 bp, synthetic construct
  source 1..737: mol_type = other DNA
gccgctggattgttattactcgcggcccagccggccatggcccaggtgcagctgcaggagtctgggggaggcttggtgcagcccggggggtctctgagactctcctgtttggcatctggattcaccttcagtaggtatgccatgaactgggtccgccaggctccagggaaggggctcgagtgggtctcaacaatttatagcaccggtgctacatactatacgaactctgtggagggccgattcaccatctccagagacaacgctaagagcacgctgtatctgcaattgaacagcctgaaaactgaagacacgggcacgtattactgtacaagggcagattggaaaacaggcactaggggccaggggacccaggtcaccgtctcctcaggcccgggaggccaacaccatcaccaccatcatggcgcatatccgtatgatgtgccggactatgcttcttaggccgaaactgttgaaagttgtttagcaaaacctcatacagaaaattcatttactaacgtctggaaagacgacaaaactttagatcgttacgctaactatgagggctgtctgtggaatgctacaggcgttgtgtttgtactgatgacgaaactcagtgttacggtacatggtctatgggctgctatcctgaaaatgaagttgtgctctgaggtgcgtctgaggtgccgatctgaggtgcgtactagctctgagtaccgtgatacattattccggcctaa

SEQ ID NO: 196 - DNA, 756 bp, synthetic construct
  source 1..756: mol_type = other DNA
acctattggcctacggcggccgctggattgttattactcgcggcccagccggccatggcccaggtgcagctgcaggagtctggaggaggctcggtgcagactggagggtctctgagactctcctgtgcagcctccggaaacacctatacagactacattattggctggctccgccaggctccagggaaggagcgtgagggggtcgcaactatttggagtggaagtggtattacatcgtacgccgactccgtgaagggccgattcagcatctcccaagacaacgccaagaacacggtgtatcttcaaatgaacagcctgaaacctgaggacacggccatctatttctgtgcaattcggccacgttggtatcttgaataccagtattggggccagggaacccaggtcaccgtctcctcaggcccgggaggccaacaccatcaccaccatcatggcgcatatccgtatgatgtgccggactatgcttcttaggccgaaactgttgaaagttgtttagcaaaacctcatacagaaaattcatttactaacgtctggaaagacgacaaaactttagatcgttacgctaactatgagggctgtctgtggaatgctacaggcgttgtggtttgtactggtgacgaaactcagtgttacggtacatgggttccctatggcttgctatccctgaaaatgagggtggtgctctggagctgcgtctgattgcgtctgaggtggcggtactaaacctcctggagtacc

SEQ ID NO: 197 - DNA, 751 bp, synthetic construct
  source 1..751: mol_type = other DNA
atacctattgcctacggcggccgctggattgttattactcgcggcccagccggccatggcccaggtgcagctgcaggagtctggaggaggctcggtgcagcctggagggtctctgagactctcctgtatagtctctggatacactggtggtagcgacttaatggcctggttccgccaggctccaggcaaggagcgcgagggggtcgcaactatttatggtgtcgctggtgtcacatattatgccgactccgtgaagggccgattcaccatcacgaaagataagaacacggtacatctgcaaatgaacagcctgaaacctgaggacactgccatgtactactgtgcagtagggcggctcggtgttgtacgttcagtcctccgctcaaatgggtatagccactggggccaggggacccaggtcaccgtctcctcaggcccgggaggccaacaccatcaccaccatcatggcgcatatccgtatgatgtgccggactatgcttcttaggccgaaactgttgaaagttgtttagcaaaacctcatacagaaaattcatttactaacgtctggaaagacgacaaaactttagatcgttacgctaactatgagggctgtctgtggaaatgctacaggcgttgtgtttgtactggtgacgaaactcagtgtaacggtacatgggttctattgggcttgctatcttgaattgaggttggtggctctgagatgcgttctgagggtggcggttctctgaag

SEQ ID NO: 198 - DNA, 744 bp, synthetic construct
  source 1..744: mol_type = other DNA
aacctattgcctacggcggccgctggattgttattactcgcggcccagccggccatggcccaggtgcagctgcaggagtctggaggaggcttggtgcagcccggggggtctctgagactctcctgtgcagcctctggattcaccttcgctaattatgctatgaactgggtccgccaggctccaggaaaggggctcgagttggtctcaactatctataatagtgggaccacatactatccaaactccgtgaagggccgattcaccatctccagagacaacgccaagaacacactatatctgcaattgaacgacctgaaaactgaggacacggccatgtattactgtgttgtgcctggcctcggaaacaagcggggccaggggacccaggtcaccgtctcctcaggcccgggaggccaacaccatcaccaccatcatggcgcatatccgtatgatgtgccggactatgcttcttaggccgaaactgttgaaagttgtttagcaaaacctcatacagaaaattcatttactaacgtctggaaagacgacaaaactttagatcgttacgctaactatgagggctgtctgtggaatgctacaggcgttgtggtttgtactggtgacgaaactcagtgttacggtacatgggttcctattggcttgctatcctgaatgaggtgtgctctgagtgccgtctgagtggcgtctgaagtgccgtactaactctgagtacggtgataccaccta

SEQ ID NO: 199 - DNA, 775 bp, synthetic construct
  source 1..775: mol_type = other DNA
atacctattgcctacggcggccgctggattgttattactcgcggcccagccggccatggcccaggtgcagctgcaggagtctggaggaggctcggtgcaggctggagggtctctgaaactctcctgttcagtctctggatacacctacagtaccaagtacatgggctggttccgccaggctccagggaaggagcgcgagggggtcgcagctattggtactggtggtggtaggacatactatgccgactccgtgaagggccgattcaccatctcccttgacaacgccaagaacacggtgtatctgcaaatgaacagcctggagcctgaggatactgccatgtactactgtgcagtagattacactgtgtggttactgcaacccttggaccaatctgggtataagtactggggccaggggacccaggtcaccgtctcctcaggcccgggaggccaacaccatcaccaccatcatggcgcatatccgtatgatgtgccggactatgcttcttaggccgaaactgttgaaagttgtttagcaaaacctcatacagaaaattcatttactaacgtctggaaagacgacaaaactttagatcgttacgctaactatgagggctgtctgtggaatgctacaggcgttgtggttttgtactggtgacgaaactcagtgttacggtacatgggttcctattggcttgctatccctgaaatgaaggtggtgctctgaggtggcgtttctgaggggtgggcggcttctggaggggttgggcgcggt

SEQ ID NO: 200 - DNA, 750 bp, synthetic construct
  source 1..750: mol_type = other DNA
accttaattgcctacggcggccgctggattgttattactcgcggcccagccggccatggcccaggtgcagctgcaggagtctggaggaggctcggtgcagactggagggtctctgagactctcctgtgcagcctccggaaacacctatacagactacattattggctggctccgccaggctccagggaaggagcgtgagggggtcgcaactatttggagtataagtggtgttacttcgtacgccgactccgtgaagggccgattcagcatctcccaagacaacgccaagaacacggtgtatcttcaaatgaacagcctgaaacctgaggacacggccatctatttctgtgcaattcggccacgttggtatgttgaataccagtattggggccagggaacccaggtcaccgtctcctcaggcccgggaggccaacaccatcaccaccatcatggcgcatatccgtatgatgtgccggactatgcttcttaggccgaaactgttgaaagttgtttagcaaaacctcatacagaaaattcatttactaacgtctggaaagacgacaaaactttagatcgttacgctaactatgagggctgtctgtggaatgctacaggcgttgtgttttgtactgtgacgaaactcagtgttacgtacatgggttcctatggctgctatcctgaaatgaggtggtgctctgaagtgccgtctgaggttggcgttctgaggtggcgtaacttaaaccctccctg

SEQ ID NO: 201 - DNA, 724 bp, synthetic construct
  source 1..724: mol_type = other DNA
ccctatttgcctacggcggccgctggattgttattactcgcggcccagccggccatggcccaggtgcagctgcaggagtctgggggaggctcggtgcagcctggagggtctttgagactctcctgtacagtctctggatacactggtggtagcgacctaatggcctggttccgccaggctccaggcaaggagcgcgagggggtcgcaggtatttatggtgtcgctggtgtcacatattatgccgactccgtgaagggccgattcaccatcacccgagacaagaacacggtacatctgcaaatgaacagcctgaaacctgaggacactgccatgtactactgtgcagtagggcggctcggtgttgtacgttcgatcctccgctcaaatggggtataactactggggccaggggacccaggtcaccgtctcctcaggcccgggaggccaacaccatcaccaccatcatggcgcatatccgtatgatgtgccggactatgcttcttaggccgaaactgttgaaagttgtttagcaaaacctcatacagaaaaattcatttactacgtctggaaagacgacaaacctttagatcgttacgctaactatgaggcctgtctgtgaaatgctacaggcgtgtgatggtactgtgacgacctcaagtacggtacattgttcctatggcctgcattctgatgaggtgtgcttgagtgccgtcctaggtgcgca

SEQ ID NO: 202 - DNA, 727 bp, synthetic construct
  source 1..727: mol_type = other DNA
aaacccttattgcctacgggcggccgctggattgttattactcgcggcccagccggccatggcccaggtgcagctgcaggagtctgggggaggctcggtacaggctggaggatctctgagactctcctgtacagcctctggatacaccggtgatagcgacttaatggcctggttccgccaggctccaggcaaagagcgcgagggggtcgcaggtatttatggtgtcgctggtgtcacatattatgccgactccgtgaagggccgattcaccatcacccgagacaacaccaagaacacggtacatctgcaaatgaacagcctgaaacctgaggacactgccatgtactactgtgcagtagggcggctcggtgttgtacgttcgatcctccgctcaaacgggtatacctactggggccaggggacccaggtcaccgtctcctcaggcccgggaggccaacaccatcaccaccatcatggcgcatatccgtatgatgtgccggactatgcttcttaggccgaaacttgtgaagttgtttagcaaaccctcaatacagaaaaattcatttacaacctctctgggaaagaacggacaaacctttaagatcgtaccgctacattagaggctttcttgtgaaatgcctacggcgttgtgttgaactggtgacacctaagttgaccgtatggtcaattggctggcacatctgaataagagggttggactacttga

SEQ ID NO: 203 - DNA, 753 bp, synthetic construct
  source 1..753: mol_type = other DNA
ctaattgcctacgggcggccgctggattgttattactcgcggcccagccggccatggcccaggtgcagctgcaggagtctggaggaggctcggtgcagcctggagggtctctgagactctcctgtacagtctctggatacactggtggtaacgacttaatggcctggttccgccaggctccaggcaaggagcgcgagggggtcgcaactgtttatggtgtcgctcatgtcacatattatgccgactccgtgaagggccgattcaccatctcccaagacaagaacacggtacatctccaaatgaacagcctgaaacctgaggacactgccatgtactactgtgcagtagggcggctcgggcttgtacgttcggtcctccgctcaaatgggtatagttactggggccaggggacccaggtcaccgtctcctcaggcccgggaggccaacaccatcaccaccatcatggcgcatatccgtatgatgtgccggactatgcttcttaggccgaaactgttgaaagttgtttagcaaaacctcatacagaaaattcatttactaacgtctggaaagacgacaaaactttagatcgttacgctaactatgagggctgtctgtggaatgctacaggcgttgtgtttgtactggtgacgaaactcagtgttacggtacatggtctattggctgctatccctgaaatgagtgtgctctgaagtgcgtcctgaagttgcgttctgggaggtgggcgggtatctt

SEQ ID NO: 204 - DNA, 750 bp, synthetic construct
  source 1..750: mol_type = other DNA
tccctattgcctacggcggccgctggattgttattactcgcggcccagccggccatggcccaggtgcagctgcaggagtctgggggaggctcggtgcaggctggaggatctctgagactctcctgtgcagcctctggatacaccgtcagtagcccctacaactacatcggctggttccgccaggctccagggaaggagcgtgagggggtcgcaactattaatcgtgatagacttaacacatactacgccgactccgtgaagggccgattcaccatctcccaagacaccgccaagaacacggtgtatctgcaaatgaacagcctgagacctgaggacacggccatctattactgtgcggcagcatggacttcctggagtactgccctaaccccggccgcgtataactactggggccaggggacccaggtcaccgtctcctcaggcccgggaggccaacaccatcaccaccatcatggcgcatatccgtatgatgtgccggactatgcttcttaggccgaaactgttgaaagttgtttagcaaaacctcatacagaaaattcatttactaacgtctggaaagacgacaaaactttagatcgttacgctaactatgagggctgtctgtggaatgctacaggcgttgtgtttgtactgtgacgaaactcagtgtacgtacatgggtctatggctggctatctgaaatgaggtggtgctctgaaggtgccgtctgaggtggcggcttctgagtgc

SEQ ID NO: 205 - DNA, 740 bp, synthetic construct
  source 1..740: mol_type = other DNA
acctaatagcctacggcggccgctggattgttattactcgcggcccagccggccatggcccaggtgcagctgcaggagtctgggggaggctcggtgcagcctggaggatctctaagactctcctgtgcagcctctggagacacctccattatcgaatacatgggctggttccacgaggctccaggaaaggagcgtgacggtatcgcgaccattaatgtcaatgatggtaacacatactacaccgactccgtgtacggccgattcaccatctcccaagacagcgccaagaagacgatttatctgcaaatgaacagcctgaaaggtgacgacacgggcatctatacctgtgcagcgtctccggcgttagaccgcggattaagtttggacccaagtgactatgattattggggccaggggacccaggtcaccgtctcctcaggcccgggaggccaacaccatcaccaccatcatggcgcatatccgtatgatgtgccggactatgcttcttaggccgaaactgttgaaagttgtttagcaaaacctcatacagaaaattcatttactaacgtctggaaagacgacaaaactttagatcgttacgctaactatgagggctgtctgtggaatgctacagcgttgtgtttgtactgctgacgaactcagtgtacggtacatggttctattggcttgctatctgaatgaggtgtgctctgagatgccgtcgtgacggatggcggctc

SEQ ID NO: 206 - DNA, 687 bp, synthetic construct
  source 1..687: mol_type = other DNA
atccctattgcctacggcggccgctggattgttattactcgcggcccagccggccatggcccaggtgcagctgcaggagtctgggggaggctcggtgcaggctggagggtctctgagactctcctgtgcagtctctggataccccaggaacaggtgcatcacgggctggacccgccaggctccagggaagggtcgcgagttggtgtcgactattacaattagcggtggtcgctcgtatggaaactccgtgaagggccgatttaccatctccgaagacaaggacaagaacacggtgtatctgcaaatgaatagcctgaaacctgaggacacggccatgtactactgttacataggtctatgtggtacgtcacaacaaaactactggggccaggggacccaggtcaccgtctcctcaggcccgggaggccaacaccatcaccaccatcatggcgcatatccgtatgatgtgccggactatgcttcttaggccgaaactgtgaaagtgtttagaaacctcatcagaaattcatttctaacgcctggaagacaacaaccttgatcgtacgctacataaggcgtgtctgtgatctacaggttgttgtactggtacaactatgacgcatggtccattgctaaactgaatagggggctatgtctaagggcctagggctttatcctgtgggggag

SEQ ID NO: 207 - DNA, 755 bp, synthetic construct
  source 1..755: mol_type = other DNA
aacctattgcctacggcggccgctggattgttattactcgcggcccagccggccatggcccaggtgcagctgcaggagtctgggggaggctcggtgcaggctggagggtctctgagactctcctgtgcagcctccggtaacacctatacagactacgttattggctggctccgccaggctccagggaaggagcgtgagggggtcgcaactatttggagtagaagtggtaccacatcgtacgccgactccgtaaagggccgattcagcatctcccaagacaacgccaagaacacggtgtatcttcaaatgaacagcctgaaacctgaggacacggccatctatttctgtgcgattcggccacgttggcatgctgaatataagtattggggccagggaacccaggtcaccgtctcctcaggcccgggaggccaacaccatcaccaccatcatggcgcatatccgtatgatgtgccggactatgcttcttaggccgaaactgttgaaagttgtttagcaaaacctcatacagaaaattcatttactaacgtctggaaagacgacaaaactttagatcgttacgctaactatgagggctgtctgtggaatgctacaggcgttgtgtttgtactggtgacgaaactcagtgttacggtacatgggtctatgggctgctatcctgaatggaggggtggtggctctgaaggtggcgtctggagggtgggcggatctgagggggtgggcggtacttaaacatc

SEQ ID NO: 208 - DNA, 756 bp, synthetic construct
  source 1..756: mol_type = other DNA
tacccctattgcctacggcggccgctggattgttattactcgcggcccagccggccatggcccaggtgcagctgcaggagtctgggggaggctcggtgcaggctggagggtctctgagactctcctgtgcagcctctggatacatcggcagccgctactgtatgggctggttccgccaggctccagggaaggaccgtgagggggtcgcaattattaataattttcatttgagtgctgatcgcacaacatacgccgactccgtgaagggccgattcaccatctcccaagacaacgccaagaatacggtgtatctacaaatgaacagcctgaaagctgaggacacggccatctattactgtgcggctcggggggcgacttactatgacacgtgttccgtcggttccgtcggtgggtatcaatactggggccaggggacccaggtcaccgtctcctcaggcccgggaggccaacaccatcaccaccatcatggcgcatatccgtatgatgtgccggactatgcttcttaggccgaaactgttgaaagttgtttagcaaaacctcatacagaaaattcatttactaacgtctggaaagacgacaaaactttagatcgttacgctaactatgagggctgtctgtgaatgctacaggcgttgtgtttgtactgatgacgaaactcagtgtacggtacatggctcctatggctggctatcctgaaatgaagttgtgctctgagggtggcgttctgaggtggcc

SEQ ID NO: 209 - DNA, 737 bp, synthetic construct
  source 1..737: mol_type = other DNA
aacccccaatttggccctacggcggccgctggattgttattactcgcggcccagccggccatggcccaggtgcagctgcaggagtctgggggaggctcggtgcagactggagggtctctgagactctcctgtgcagcctctggaaacacctatacagactacattattggctggctccgccaggctccagggaaggagcgtgagggggtcgcaactatttggagtggaagtggtattacatcgtacgccgattccgtgaagggccgattcagcatctcccaagacaatgtcaagaacacggtgtatcttcaaatgaacagcctgaaacctgaggacacggccatctatttctgtgcgcttcggccacgttggcatattgaataccaatattggggccagggaacccaggtcaccgtctcctcaggcccgggaggccaacaccatcaccaccatcatggcgcatatccgtatgatgtgccggactatgcttcttaggccgaaactgttgaaagttgtttagcaaaacctcatacagaaaattcatttactaacgtctggaaagacgacaaactttagatcgttacgctaactatgaggctgtctgtggaatgctacaggcgttgtgtttgtactgtgacgaactcatgtacggtacatgggtctatggctgctatcctgaaatgaggtgtgctctgaagtggcgatctgagtggcgttctggaggttggcgtacctac

SEQ ID NO: 210 - DNA, 740 bp, synthetic construct
  source 1..740: mol_type = other DNA
cacccttattgggccttacggcggccgctggattgttattactcgcggcccagccggccatggcccaggtgcagctgcaggagtctgggggaggctcggtgcaggctggagggtctctgagactctcctgtacagcctctacagacaccttcagtttcagtgactatgcagtggagtggtaccgccaggctccagggaagacgtgcgaatgggtctcaagtatttttcgtactggtaacacagattatgtcgactctgtgaagggccgattcactatctcccgagacaacgccaaggcgacgagctatctgcaaatgaacagcctgaaacctgaggacacggccgtgtattattgtctgatagggtcgtccggttgtcaacgaacccaggggacccaggtcaccgtctcctcaggcccgggaggccaacaccatcaccaccatcatggcgcatatccgtatgatgtgccggactatgcttcttaggccgaaactgttgaaagttgttagcaaaaccctcaatacagaaaaattcatttactaacgttctggaaagacgacaaacctttagaatcggttaccgctaactatgagggctgtctgtggaattgcctacaggcgttggtgattgtactggtgacgactcagtgtaacggtaattggttcctattggctgctatccttgaaatgagggtgtgcctaaggtggcgtctgaggtggcgattccatagtggccacctc

SEQ ID NO: 211 - DNA, 756 bp, synthetic construct
  source 1..756: mol_type = other DNA
aaccctattgcctacggcggccgctggattgttattactcgcggcccagccggccatggcccaggtgcagctgcaggagtctgggggaggctcggtggagtctggggggtctctgagactctcctgtgcagcctctggatacacccacagtagctactgcatgggctggtaccgccaggctccagggaaggagcgcgagttggtctcatttatttttagtgagggtagcacatcctatacagactccgtgaagggccgattcaccatctcccaagacaacgccaagaacacggtgtatctgcaaatgaacagcctgaaacccgaggacacggccatctattactgtgcggcagattatggaacccctaaatacggtgataactggtgtgactttaattactggggccaggggacccaggtcaccgtctcctcaggcccgggaggccaacaccatcaccaccatcatggcgcatatccgtatgatgtgccggactatgcttcttaggccgaaactgttgaaagttgtttagcaaaacctcatacagaaaattcatttactaacgtctggaaagacgacaaaactttagatcgttacgctaactatgagggctgtctgtggaatgctacaggcgttgtgtttgtactgtgacgaaactcagtgttacgtacatgggtctatgggctgctatccctgaatgagtgtgctctgaagtgcgattctgaaggtggcgtctggaggggtggggcggtacacta

SEQ ID NO: 212 - DNA, 757 bp, synthetic construct
  source 1..757: mol_type = other DNA
aacctattgcctacggcggccgctggattgttattactcgcggcccagccggccatggcccaggtgcagctgcaggagtctgggggaggctcggtgcaggccggagggtctctgagactctcctgtacagcctctggatacacctgcagtagatactgtatgggctggtatcgccaggctcttgggaaggagcgcgaattggtctcatttattggtagtgatggtatcacacactatacagactccgtgaagggccgattcaccgtctccctagacaacgccaagaacacgatgtatctgcaaatgaacagcctgaaacctgaggacacggccatctattactgtgcggggggttacggtagtggtggtgactgcagttttggtagctggggccgggggacccaggtcaccgtctcctcaggcccgggaggccaacaccatcaccaccatcatggcgcatatccgtatgatgtgccggactatgcttcttaggccgaaactgttgaaagttgtttagcaaaacctcatacagaaaattcatttactaacgtctggaaagacgacaaaactttagatcgttacgctaactatgagggctgtctgtggaatgctacaggcgttgtggtttgtactggtgacgaaactcagtgtacggtacatgggttctatggctgctatccctgaatgagggtgggtgctctgagtgcgtctggagggtgggcggtctggagggtgggcggtactaacctcctgggag

SEQ ID NO: 213 - DNA, 750 bp, synthetic construct
  source 1..750: mol_type = other DNA
aaccctattgcctacggcggccgctggattgttattactcgcggcccagccggccatggcccaggtgcagctgcaggagtctggaggaggctcggtgcaggctggagggtctctgagactctcctgtgcagcctctggatacacctacagtacgtactgcatgggctggttccgccaggctccagggaaggaacgtgagggggtcgcatgtattaatagtggtgacggtagcgcatactacgccgactccgtgaagggccgattcaccatctcccaagacaacgccaagaacacggtgtatctgcaaatgaacagcctgaaacctgaggacacggccatgtattactgtgcggcttgggatggaggttactgtcacagcgcgatttgggtccctgggtataagtaccggggccaggggacccaggtcaccgtctcctcaggcccgggaggccaacaccatcaccaccatcatggcgcatatccgtatgatgtgccggactatgcttcttaggccgaaactgttgaaagttgtttagcaaaacctcatacagaaaattcatttactaacgtctggaaagacgacaaaactttagatcgttacgctaactatgagggctgtctgtgaatgctacaggcgttgtgatttgtactgtgacgaaactcagtgtaccgtacatggctctatggctgctatctgaatgaggtgtgctctgaaggtggcgtcctgaagtggcggattctgaagtgagc

SEQ ID NO: 214 - protein, 122 aa, synthetic construct
MAQVQLQESGGGLVQPGGSLRLSCLASGFTFSRYAMNWVRQAPGKGLEWVSTIYSTGATYYTNSVEGRFTISRDNAKSTLYLQLNSLKTEDTGTYYCTRADWKTGTRGQGTQVTVSSGPGGQ

SEQ ID NO: 215 - protein, 126 aa, synthetic construct
MAQVQLQESGGGSVQTGGSLRLSCAASGNTYTDYIIGWLRQAPGKEREGVATIWSGSGITSYADSVKGRFSISQDNAKNTVYLQMNSLKPEDTAIYFCAIRPRWYLEYQYWGQGTQVTVSSGPGGQ

SEQ ID NO: 216 - protein, 131 aa, synthetic construct
MAQVQLQESGGGSVQPGGSLRLSCIVSGYTGGSDLMAWFRQAPGKEREGVATIYGVAGVTYYADSVKGRFTITKDKNTVHLQMNSLKPEDTAMYYCAVGRLGVVRSVLRSNGYSHWGQGTQVTVSSGPGGQ

SEQ ID NO: 217 - protein, 121 aa, synthetic construct
MAQVQLQESGGGLVQPGGSLRLSCAASGFTFANYAMNWVRQAPGKGLELVSTIYNSGTTYYPNSVKGRFTISRDNAKNTLYLQLNDLKTEDTAMYYCVVPGLGNKRGQGTQVTVSSGPGGQ

SEQ ID NO: 218 - protein, 133 aa, synthetic construct
MAQVQLQESGGGSVQAGGSLKLSCSVSGYTYSTKYMGWFRQAPGKEREGVAAIGTGGGRTYYADSVKGRFTISLDNAKNTVYLQMNSLEPEDTAMYYCAVDYTVWLLQPLDQSGYKYWGQGTQVTVSSGPGGQ

SEQ ID NO: 219 - protein, 126 aa, synthetic construct
MAQVQLQESGGGSVQTGGSLRLSCAASGNTYTDYIIGWLRQAPGKEREGVATIWSISGVTSYADSVKGRFSISQDNAKNTVYLQMNSLKPEDTAIYFCAIRPRWYVEYQYWGQGTQVTVSSGPGGQ

SEQ ID NO: 220 - protein, 128 aa, synthetic construct
MAQVQLQESGGGSVQPGGSLRLSCTVSGYTGGSDLMAWFRQAPGKEREGVAGIYGVAGVTYYADSVKGRFTITRDKNTVHLQMNSLKPEDTAMYYCAVGRLGVVRSILRSNGVLLGPGDPGHRLLRPG

SEQ ID NO: 221 - protein, 133 aa, synthetic construct
MAQVQLQESGGGSVQAGGSLRLSCTASGYTGDSDLMAWFRQAPGKEREGVAGIYGVAGVTYYADSVKGRFTITRDNTKNTVHLQMNSLKPEDTAMYYCAVGRLGVVRSILRSNGYTYWGQGTQVTVSSGPGGQ

SEQ ID NO: 222 - protein, 131 aa, synthetic construct
MAQVQLQESGGGSVQPGGSLRLSCTVSGYTGGNDLMAWFRQAPGKEREGVATVYGVAHVTYYADSVKGRFTISQDKNTVHLQMNSLKPEDTAMYYCAVGRLGLVRSVLRSNGYSYWGQGTQVTVSSGPGGQ

SEQ ID NO: 223 - protein, 134 aa, synthetic construct
MAQVQLQESGGGSVQAGGSLRLSCAASGYTVSSPYNYIGWFRQAPGKEREGVATINRDRLNTYYADSVKGRFTISQDTAKNTVYLQMNSLRPEDTAIYYCAAAWTSWSTALTPAAYNYWGQGTQVTVSSGPGGQ

SEQ ID NO: 224 - protein, 133 aa, synthetic construct
MAQVQLQESGGGSVQPGGSLRLSCAASGDTSIIEYMGWFHEAPGKERDGIATINVNDGNTYYTDSVYGRFTISQDSAKKTIYLQMNSLKGDDTGIYTCAASPALDRGLSLDPSDYDYWGQGTQVTVSSGPGGQ

SEQ ID NO: 225 - protein, 121 aa, synthetic construct
MAQVQLQESGGGSVQAGGSLRLSCAVSGYPRNRCITGWTRQAPGKGRELVSTITISGGRSYGNSVKGRFTISEDKDKNTVYLQMNSLKPEDTAMYYCYIGLCGTSQQNYWGQGTQVTVSSG

SEQ ID NO: 226 - protein, 126 aa, synthetic construct
MAQVQLQESGGGSVQAGGSLRLSCAASGNTYTDYVIGWLRQAPGKEREGVATIWSRSGTTSYADSVKGRFSISQDNAKNTVYLQMNSLKPEDTAIYFCAIRPRWHAEYKYWGQGTQVTVSSGPGGQ

SEQ ID NO: 227 - protein, 138 aa, synthetic construct
MAQVQLQESGGGSVQAGGSLRLSCAASGYIGSRYCMGWFRQAPGKDREGVAIINNFHLSADRTTYADSVKGRFTISQDNAKNTVYLQMNSLKAEDTAIYYCAARGATYYDTCSVGSVGGYQYWGQGTQVTVSSGPGGQ

SEQ ID NO: 228 - protein, 126 aa, synthetic construct
MAQVQLQESGGGSVQTGGSLRLSCAASGNTYTDYIIGWLRQAPGKEREGVATIWSGSGITSYADSVKGRFSISQDNVKNTVYLQMNSLKPEDTAIYFCALRPRWHIEYQYWGQGTQVTVSSGPGGQ

SEQ ID NO: 229 - protein, 123 aa, synthetic construct
MAQVQLQESGGGSVQAGGSLRLSCTASTDTFSFSDYAVEWYRQAPGKTCEWVSSIFRTGNTDYVDSVKGRFTISRDNAKATSYLQMNSLKPEDTAVYYCLIGSSGCQRTQGTQVTVSSGPGGQ

SEQ ID NO: 230 - protein, 131 aa, synthetic construct
MAQVQLQESGGGSVESGGSLRLSCAASGYTHSSYCMGWYRQAPGKERELVSFIFSEGSTSYTDSVKGRFTISQDNAKNTVYLQMNSLKPEDTAIYYCAADYGTPKYGDNWCDFNYWGQGTQVTVSSGPGGQ

SEQ ID NO: 231 - protein, 127 aa, synthetic construct
MAQVQLQESGGGSVQAGGSLRLSCTASGYTCSRYCMGWYRQALGKERELVSFIGSDGITHYTDSVKGRFTVSLDNAKNTMYLQMNSLKPEDTAIYYCAGGYGSGGDCSFGSWGRGTQVTVSSGPGGQ

SEQ ID NO: 232 - protein, 133 aa, synthetic construct
MAQVQLQESGGGSVQAGGSLRLSCAASGYTYSTYCMGWFRQAPGKEREGVACINSGDGSAYYADSVKGRFTISQDNAKNTVYLQMNSLKPEDTAMYYCAAWDGGYCHSAIWVPGYKYRGQGTQVTVSSGPGGQ

SEQ ID NO: 233 - protein, 10 aa, synthetic construct
GFTFSRYAMN

SEQ ID NO: 234 - protein, 8 aa, synthetic construct
IYSTGATY

SEQ ID NO: 235 - protein, 10 aa, synthetic construct
TRADWKTGTR

SEQ ID NO: 236 - protein, 10 aa, synthetic construct
GNTYTDYIIG

SEQ ID NO: 237 - protein, 9 aa, synthetic construct
IWSGSGITS

SEQ ID NO: 238 - protein, 12 aa, synthetic construct
AIRPRWYLEYQY

SEQ ID NO: 239 - protein, 10 aa, synthetic construct
GFTFANYAMN

SEQ ID NO: 240 - protein, 8 aa, synthetic construct
IYNSGTTY

SEQ ID NO: 241 - protein, 10 aa, synthetic construct
VVPGLGNKRG

SEQ ID NO: 242 - protein, 10 aa, synthetic construct
GYTYSTKYMG

SEQ ID NO: 243 - protein, 9 aa, synthetic construct
IGTGGGRTY

SEQ ID NO: 244 - protein, 13 aa, synthetic construct
AVDYTVWLLQPLD

SEQ ID NO: 245 - protein, 10 aa, synthetic construct
GNTYTDYIIG

SEQ ID NO: 246 - protein, 9 aa, synthetic construct
IWSISGVTS

SEQ ID NO: 247 - protein, 12 aa, synthetic construct
AIRPRWYVEYQY

SEQ ID NO: 248 - protein, 10 aa, synthetic construct
GYTGGSDLMA

SEQ ID NO: 249 - protein, 9 aa, synthetic construct
IYGVAGVTY

SEQ ID NO: 250 - protein, 15 aa, synthetic construct
AVGRLGVVRSILRSN

SEQ ID NO: 251 - protein, 10 aa, synthetic construct
GYTGDSDLMA

SEQ ID NO: 252 - protein, 10 aa, synthetic construct
IYGVAGVTYY

SEQ ID NO: 253 - protein, 19 aa, synthetic construct
AVGRLGVVRSILRSNGYTY

SEQ ID NO: 254 - protein, 10 aa, synthetic construct
GYTGGNDLMA

SEQ ID NO: 255 - protein, 10 aa, synthetic construct
VYGVAHVTYY

SEQ ID NO: 256 - protein, 19 aa, synthetic construct
AVGRLGLVRSVLRSNGYSY

SEQ ID NO: 257 - protein, 12 aa, synthetic construct
GYTVSSPYNYIG

SEQ ID NO: 258 - protein, 9 aa, synthetic construct
INRDRLNTY

SEQ ID NO: 259 - protein, 18 aa, synthetic construct
AAAWTSWSTALTPAAYNY

SEQ ID NO: 260 - protein, 10 aa, synthetic construct
GDTSIIEYMG

SEQ ID NO: 261 - protein, 9 aa, synthetic construct
INVNDGNTY

SEQ ID NO: 262 - protein, 19 aa, synthetic construct
AASPALDRGLSLDPSDYDY

SEQ ID NO: 263 - protein, 10 aa, synthetic construct
GYPRNRCITG

SEQ ID NO: 264 - protein, 8 aa, synthetic construct
ITISGGRS

SEQ ID NO: 265 - protein, 12 aa, synthetic construct
YIGLCGTSQQNY

SEQ ID NO: 266 - protein, 10 aa, synthetic construct
GNTYTDYVIG

SEQ ID NO: 267 - protein, 9 aa, synthetic construct
IWSRSGTTS

SEQ ID NO: 268 - protein, 12 aa, synthetic construct
AIRPRWHAEYKY

SEQ ID NO: 269 - protein, 10 aa, synthetic construct
GYIGSRYCMG

SEQ ID NO: 270 - protein, 13 aa, synthetic construct
INNFHLSADRTTY

SEQ ID NO: 271 - protein, 22 aa, synthetic construct
AARGATYYDTCSVGSVGGYQYW

SEQ ID NO: 272 - protein, 10 aa, synthetic construct
GNTYTDYIIG

SEQ ID NO: 273 - protein, 9 aa, synthetic construct
IWSGSGITS

SEQ ID NO: 274 - protein, 12 aa, synthetic construct
ALRPRWHIEYQY

SEQ ID NO: 275 - protein, 12 aa, synthetic construct
TDTFSFSDYAVE

SEQ ID NO: 276 - protein, 9 aa, synthetic construct
IFRTGNTDY

SEQ ID NO: 277 - protein, 10 aa, synthetic construct
LIGSSGCQRT

SEQ ID NO: 278 - protein, 10 aa, synthetic construct
GYTHSSYCMG

SEQ ID NO: 279 - protein, 9 aa, synthetic construct
IFSEGSTSY

SEQ ID NO: 280 - protein, 20 aa, synthetic construct
AADYGTPKYGDNWCDFNYWG

SEQ ID NO: 281 - protein, 10 aa, synthetic construct
GYTCSRYCMG

SEQ ID NO: 282 - protein, 9 aa, synthetic construct
IGSDGITHY

SEQ ID NO: 283 - protein, 14 aa, synthetic construct
AGGYGSGGDCSFGS

SEQ ID NO: 284 - protein, 10 aa, synthetic construct
GYTYSTYCMG

SEQ ID NO: 285 - protein, 9 aa, synthetic construct
INSGDGSAY

SEQ ID NO: 286 - protein, 19 aa, synthetic construct
AAWDGGYCHSAIWVPGYKY

SEQ ID NO: 287 - skipped

SEQ ID NO: 288 - DNA, 15 bp, synthetic construct
  source 1..15: mol_type = other DNA
ggcccgggaggccaa

SEQ ID NO: 289 - DNA, 18 bp, synthetic construct
  source 1..18: mol_type = other DNA
caccatcaccaccatcat

SEQ ID NO: 290 - DNA, 27 bp, synthetic construct
  source 1..27: mol_type = other DNA
tatccgtatgatgtgccggactatgct

SEQ ID NO: 291 - protein, 10 aa, synthetic construct
GYTGGSDLMA

SEQ ID NO: 292 - protein, 9 aa, synthetic construct
IYGVAGVTY

SEQ ID NO: 293 - protein, 21 aa, synthetic construct
AVGRLGVVRSVLRSNGYSHWG